(19) United States
(12) Patent Application Publication
(10) Pub. No.: US 2025/0009889 A1
(43) Pub. Date: Jan. 9, 2025

(54) CYCLIC PEROXIDES AS PRODRUGS FOR SELECTIVE DELIVERY OF AGENTS

(71) Applicant: The Regents of the University of California, Oakland, CA (US)

(72) Inventors: Adam R. Renslo, Oakland, CA (US); Jun Chen, San Francisco, CA (US); Ryan L. Gonciarz, San Francisco, CA (US)

(21) Appl. No.: 18/693,913

(22) PCT Filed: Sep. 23, 2022
(86) PCT No.: PCT/US2022/076913

§ 371 Date: Mar. 20, 2024

Related U.S. Application Data
(60) Provisional application No. 63/248,279, filed on Sep. 24, 2021.

(51) Int. Cl. A61K 47/54; C07D 323/02; C07D 405/12; C07D 405/14; C07D 491/22

(52) U.S. Cl. CPC A61K 47/545; C07D 323/02; C07D 405/12; C07D 405/14; C07D 491/22

(57) ABSTRACT

Described herein, inter alia, are prodrug compositions and methods of using the same for treatment and detection of disease.

### CROSS-REFERENCES TO RELATED APPLICATIONS

This application claims the benefit of U.S. Provisional Application No. 63/248,279, filed Sep. 24, 2021, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. R01 AI105106 awarded by The National Institutes of Health. The government has certain rights in the invention.

### BACKGROUND

Many chemotherapeutic agents used to treat cancer exhibit serious toxicity, resulting in undesired side effects for patients and reducing efficacy by limiting the doses that can be safely administered. Similarly, many of the therapeutics used to treat infectious diseases, including bacterial infections, confer undesirable side effects. It would be preferable if such agents could be administered in a prodrug form that masked the inherent toxicity of the agent from irrelevant, non-diseased tissues, and yet released the fully active drug species at the desired site of action. Such a technology would have the potential to increase the therapeutic window of a variety of drugs, possibly allowing them to be used safely at a more efficacious dose, and with reduced incidence of undesired side effects for the patient.

In normal cells and tissues, iron remains mostly sequestered in forms that are non-toxic to the cell, bound to the iron storage and transport proteins ferritin and transferrin, respectively or bound as heme within hemoglobin and other iron-dependent enzymes. Diseased tissues and cells, on the other hand, can contain higher than normal concentrations of loosely bound Fe^{II} (labile) iron. Many neoplastic cells for example over-express the transferrin receptor to increase their uptake of iron and similarly over-express ferrireductases that elevate Fe^{II} specifically. Increased iron uptake has been proposed to explain the increased toxicity that endoperoxides like artemisinin exhibit towards cancer cell lines as compared to normal cells (Efferth, T. Drug Resistance Updates, 2005, 8:85-97). Artemisinin and its derivatives are believed to exert their cytotoxic effect via reaction with Fe^{II} and the resulting generation of reactive oxygen and carbon centered radical species. The cytotoxicity of artemisinin derivatives towards leukemia, astrocytoma, and breast cancer cell lines can be potentiated by the addition of exogenous Fe^{II} salts or transferrin (Efferth, T. et al. Free Radical Biology & Medicine, 2004, 37, 998-1009; Singh, N. P. et al. Life Sciences, 2001, 70, 49-56). U.S. Pat. No. 5,578,637 describes the use of an endoperoxide moiety (i.e., an artemisinin) to kill cancer cells under conditions that enhance intracellular iron concentrations. None of these prior works teach or suggest how higher than normal concentrations of iron in such cells could be exploited for selective delivery of a drug species via an iron-sensitive prodrug moiety. Disclosed herein, inter alia, are solutions to these and other problems in the art.

### BRIEF SUMMARY

In an aspect is provided a compound, or a pharmaceutically acceptable salt thereof, having the formula:

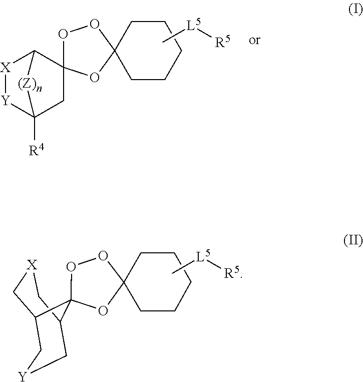

X is NR^{1.1} or C(R^{1.1}R^{1.2}). Y is NR^{2.1} or C(R^{2.1}R^{2.2}). Z is C(R^{3.1}R^{3.2}.

The symbol n is 1 or 2.

L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker.

L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R^{1.1} and R^{1.2} are independently hydrogen, oxo, halogen, —CX¹³, —CHX¹², —CH₂X¹, —OCX¹³, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)RiC, —C(O)OR^{1C}, —C(O)NR^{1A}R^{1B}, —OR^{1D}, —SR^{1D}, —NR^{1A}SO₂R^{1D}, —NR^{1A}C(O)R^{1C}—NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is hydrogen, oxo, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{1A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a protein moiety, a detectable moiety, a siderophore moiety, or a drug moiety.

Each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl.

X¹, X², X³, X⁴, and X⁵ are independently —F, —Cl, —Br, or —I.

The symbols n1, n2, n3, n4, and n5 are independently an integer from 0 to 4. The symbols m1, m2, m3, m4, m5, v1, v2, v3, v4, and v5 are independently 1 or 2.

In an aspect is provided a pharmaceutical composition including a compound described herein, or a pharmaceutically acceptable salt thereof, and a pharmaceutically acceptable excipient.

In an aspect is provided a method of treating a disease in a subject in need thereof, the method including administering to the subject in need thereof a therapeutically effective amount of a compound described herein, or a pharmaceutically acceptable salt thereof.

In an aspect is provided a method of identifying a subject having a disease associated with a cell or organism having an increased level of a reductant (e.g., biological reductant, Fe^{II}) compared to a standard control (e.g., subject without the disease or sample from a subject without the disease), the method including administering to the subject an effective amount of a compound described herein, or a pharmaceutically acceptable salt thereof.

In an aspect is provided a method of identifying a subject having a disease associated with an increased level of a reductant (e.g., biological reductant, Fe^{II}) compared to a standard control (e.g., subject without the disease or sample from a subject without the disease), the method including: (i) obtaining a biological sample from the subject; (ii) contacting the biological sample with an effective amount of a compound described herein, or a pharmaceutically acceptable salt thereof, wherein the compound includes a detectable moiety; and (iii) detecting an increased level of the detectable moiety or a detectable agent resulting from cleavage of the detectable moiety relative to the level of the detectable moiety or detectable agent in the standard control.

### BRIEF DESCRIPTION OF THE DRAWINGS

FIG. 1. Structures of recently described probes of ferrous iron employing a 1,2,4-trioxolane-based sensor of ferrous iron, inspired by the synthetic antimalarial arterolane.

FIG. 2. Representative low-energy conformations of putative bridged bicyclic trioxolane adducts, modelled as the N,N-dimethyl carbamates, using MarvinSketch (v19.10).

FIGS. 3A-3C. FIG. 3A: Synthetic scheme to form conjugates. FIG. 3B: Structures of conjugates 6f-6h, 7f-7h, and 8r-8s. In vitro antiplasmodial activity of 6f-6h, 7f-7h and known comparator 6a against W2 P. falciparum parasites (IC₅₀±SEM) are also shown. Reported IC₅₀ values are the means of at least three determinations. IC₅₀ values for artefenomel and chloroquine controls are indicated at bottom left. The superior potency of conjugates 6 vs. 7 imply their activity is derived from mefloquine (MFQ) release. FIG. 3C: Structures of conjugates 9r-9s, 10r-10s, 11r-11s, 12r, 13s, and 14r. Drugs are abbreviated as follows: MFQ is mefloquine, XTC is exatecan, ASN is ASN007, CIP is ciprofloxacin, COBI is cobimetinib. Site of chemical conjugation is at a secondary amine function (MFQ, CIP, COBI) or a primary amine function (ASN007, exatecan).

FIG. 4. In vitro iron fragmentation studies of the previously described, in vivo efficacious mefloquine conjugate 6a and the new congener 6f bearing a bicyclo[2.1.1]heptane ring in place of the adamantane. Fragmentation of the trioxolane with FAS is rapid for both conjugates, with β-elimination from common intermediate A being the rate limiting step in mefloquine (MFQ) release.

FIGS. 5A-5B. FIG. 5A: Synthetic schemes of intermediates. FIG. 5B: Synthetic scheme to form cyclic peroxide compounds.

FIGS. 6A-6D. Examples of cyclic peroxide compounds. FIG. 6A: Examples wherein R⁵ is a monovalent form of exatecan. FIG. 6B: Examples wherein R⁵ is a monovalent form of ASN007. FIG. 6C: Examples wherein R⁵ is a monovalent form of cobimetinib. FIG. 6D: Examples wherein R⁵ is a monovalent form of ciprofloxacin.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

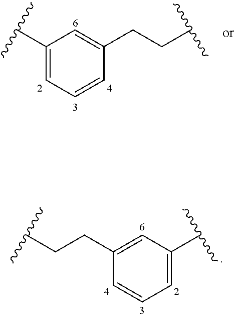 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, =NR′, =N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)=NR″″, —NRC(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆—C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{1.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A-1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

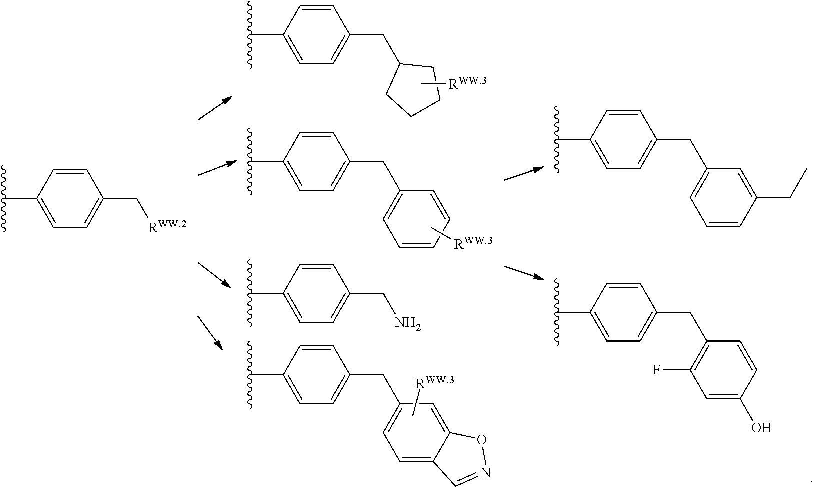

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D} etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent. Prodrugs described herein include compounds that readily undergo chemical changes under select physiological conditions (e.g. increased Fe^{II} concentration relative to normal physiological levels, increased reductant levels relative to normal physiological levels) to provide agents (e.g., compounds, proteins, drugs, detectable agents, therapeutic agents) to a biological system (e.g., in a subject, in an infected cell, in a cancer cell, in the extracellular space near an infected cell, in the extracellular space near a cancer cell from the moieties (e.g., moiety of a protein, drug, detectable agent) attached to the prodrug moiety and included in the prodrug (e.g., compound of formula I or II, including embodiments, compound described herein, examples)).

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In some embodiments, the disease is a disease having the symptom of an increased amount of Fe^{II} relative to normal Fe^{II} amounts in a subject (e.g., human). In some embodiments, the disease is a disease having the symptom of an increased amount of a reductant (e.g., biological reductant, Fe^{II}) relative to normal reductant (e.g., biological reductant, Fe^{II}) amounts in a subject (e.g., human). In embodiments, the disease is an infectious disease. In embodiments, the disease is a bacterial disease. In embodiments, the disease is a parasitic disease. In embodiments, the disease is a viral disease. In embodiments, the disease is malaria. In embodiments, the disease is drug-resistant malaria. In some embodiments, the disease is a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma. In some embodiments, the disease is a disease related to (e.g., caused by) an infectious agent (e.g., bacteria) Examples of diseases, disorders, or conditions include, but are not limited to, infectious diseases, bacterial infectious diseases, nosocomial infections, nosocomial bacterial infections, ventilator associated pneumonias, bacterial blood stream infections, Cutaneous anthrax, Pulmonary anthrax, Gastrointestinal anthrax, Whooping cough, bacterial pneumonia, Lyme disease, Brucellosis, Acute enteritis, Community-acquired respiratory infection, Nongonococcal urethritis (NGU), Lymphogranuloma venereum (LGV), Trachoma, Inclusion conjunctivitis of the newborn (ICN), Psittacosis, Botulism, Pseudomembranous colitis, Gas gangrene, Acute food poisoning, Anaerobic cellulitis, Tetanus, Diphtheria, Nosocomial infections, Urinary tract infections (UTI), Diarrhea, Meningitis in infants, Traveller's diarrhea, Diarrhea in infants, Hemorrhagic colitis, Hemolytic-uremic syndrome, Tularemia, Bacterial meningitis, Upper respiratory tract infections, Pneumonia, bronchitis, Peptic ulcer, gastric carcinoma, gastric B-cell lymphoma, Legionnaire's Disease, Pontiac fever, Leptospirosis, Listeriosis, Leprosy (Hansen's disease), Tuberculosis, Mycoplasma pneumonia, Gonorrhea, Ophthalmia neonatorum, Septic arthritis, Meningococcal disease, Waterhouse-Friderichsen syndrome, Pseudomonas infection, Bacteremia, endocarditis, Rocky mountain spotted fever, Typhoid fever type salmonellosis (dysentery, colitis), Salmonellosis, gastroenteritis, enterocolitis, Bacillary dysentery/Shigellosis, Coagulase-positive staphylococcal infections, Impetigo, Acute infective endocarditis, Septicemia, Necrotizing pneumonia, Toxinoses, Toxic shock syndrome, Staphylococcal food poisoning, Cystitis, Meningitis, septicemia, Endometritis, Opportunistic infections, Acute bacterial pneumonia, Otitis media, sinusitis, Streptococcal pharyngitis, Scarlet fever, Rheumatic fever, erysipelas, Puerperal fever, Necrotizing fasciitis, Syphilis, Congenital syphilis, Cholera, Plague, Bubonic plague, Pneumonic plague, sepsis, Iraq war infection caused by Acinetobacter baumannii (i.e. Iraq war-related Acinetobacter baumannii infection), skin diseases or conditions, acne, acne vulgaris, keratosis pilaris, acne rosacea, harlequin ichthyosis, xeroderma pigmentosum, keratoses, eczema, rosacea, necrotizing fasciitis, tuberculosis, hospital-acquired pneumonia, gastroenteritis, or bacteremia.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “infectious disease” refers to a disease or condition related to the presence of an organism (the agent or infectious agent) within or contacting the subject or patient. Examples include a bacterium, fungus, virus, or other microorganism. A “bacterial infectious disease” or “bacterial disease” is an infectious disease wherein the organism is a bacterium. A “viral infectious disease” or “viral disease” is an infectious disease wherein the organism is a virus. An “antibiotic resistant bacterial infectious disease” or “antibiotic resistant bacterial disease” is an infectious disease wherein the organism is a bacterium resistant to one or more antibiotics effective in treating a disease caused by the non-antibiotic resistant strains of the bacterium. A “penicillin resistant bacterial infectious disease” or “penicillin resistant bacterial disease” is an antibiotic resistant bacterial infectious disease wherein the disease is not treated as effectively by a penicillin or penicillin-related compounds as a similar disease caused by a bacterial strain that is not penicillin resistant. A “cephalosporin resistant bacterial infectious disease” or “cephalosporin resistant bacterial disease” is an antibiotic resistant bacterial infectious disease wherein the disease is not treated as effectively by a cephalosporin or cephalosporin-related compounds as a similar disease caused by a bacterial strain that is not cephalosporin resistant. A “beta-lactam antibiotic resistant bacterial infectious disease” or “beta-lactam antibiotic resistant bacterial disease” is a an antibiotic resistant bacterial infectious disease wherein the disease is not treated as effectively by beta-lactam containing antibiotics as a similar disease caused by a bacterial strain that is not beta-lactam antibiotic resistant. Examples of infectious diseases that may be treated with a compound or method described herein include nosocomial infections, bacteremia, Cutaneous anthrax, Pulmonary anthrax, Gastrointestinal anthrax, Whooping cough, bacterial pneumonia, bacteremia, Lyme disease, Brucellosis, Acute enteritis, Community-acquired respiratory infection, Nongonococcal urethritis (NGU), Lymphogranuloma venereum (LGV), Trachoma, Inclusion conjunctivitis of the newborn (ICN), Psittacosis, Botulism, Pseudomembranous colitis, Gas gangrene, Acute food poisoning, Anaerobic cellulitis, Tetanus, Diphtheria, Nosocomial infections, Urinary tract infections (UTI), Diarrhea, Meningitis in infants, Traveller's diarrhea, Diarrhea in infants, Hemorrhagic colitis, Hemolytic-uremic syndrome, Tularemia, Bacterial meningitis, Upper respiratory tract infections, Pneumonia, bronchitis, Peptic ulcer, gastric carcinoma, gastric B-cell lymphoma, Legionnaire's Disease, Pontiac fever, Leptospirosis, Listeriosis, Leprosy (Hansen's disease), Tuberculosis, Mycoplasma pneumonia, Gonorrhea, Ophthalmia neonatorum, Septic arthritis, Meningococcal disease, Waterhouse-Friderichsen syndrome, Pseudomonas infection, Bacteremia, endocarditis, Rocky mountain spotted fever, Typhoid fever type salmonellosis (dysentery, colitis), Salmonellosis, gastroenteritis, enterocolitis, Bacillary dysentery/Shigellosis, Coagulase-positive staphylococcal infections, Impetigo, Acute infective endocarditis, Septicemia, Necrotizing pneumonia, Toxinoses, Toxic shock syndrome, Staphylococcal food poisoning, Cystitis, Meningitis, septicemia, Endometritis, Opportunistic infections, Acute bacterial pneumonia, Otitis media, sinusitis, Streptococcal pharyngitis, Scarlet fever, Rheumatic fever, erysipelas, Puerperal fever, Necrotizing fasciitis, Syphilis, Congenital syphilis, Cholera, Plague, Bubonic plague, Pneumonic plague, Iraq war infection caused by Acinetobacter baumannii (i.e., Iraq war-related Acinetobacter baumannii infection), necrotizing fasciitis, tuberculosis, hospital-acquired pneumonia, gastroenteritis, or sepsis.

“Infectious agent” refers to an organism that is associated with (in or contacting) patients with an infectious disease but not in patients without the infectious disease and wherein contacting a patient without the infectious disease with the organism results in the patient having the infectious disease. In some embodiments, the infectious agent associated with a disease that may be treated by the compounds and/or methods described herein is a bacterium. In some embodiments, the bacteria is of a genera selected from Stenotrophomonas, Clostridium, Acinetobacter, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, Klebsiella, Enterobacter, Citrobacter, or Yersinia. In some embodiments, the bacteria is selected from Stenotrophomonas maltophilia, Clostridium difficile, Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli (ETEC), Enteropathogenic E. coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Klebsiella pneumoniae, Enterobacter cloacae, Citrobacter freundii, Acinetobacter baumannii, or Yersinia pestis. In some embodiments, the bacteria is gram negative. In some embodiments, the bacteria is gram positive.

The term “drug” is used in accordance with its common meaning and refers to a substance which has a physiological effect (e.g. beneficial effect, is useful for treating a subject) when introduced into or to a subject (e.g. in or on the body of a subject or patient). A drug moiety is a radical of a drug.

“Anti-cancer agent” or “anti-cancer drug” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gudrin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g., tomaymycin), carboplatin, CC-1065 and CC-1065 analogs including amino-CBIs, nitrogen mustards (such as chlorambucil and melphalan), dolastatin and dolastatin analogs (including auristatins: e.g., monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g., mertansine), methotrexate, mitomycin C, taxoids, vinca alkaloids (such as vinblastine and vincristine), epothilones (e.g., epothilone B), camptothecin and its clinical analogs topotecan and irinotecan, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

The terms “anti-infective agent” or “anti-infectious agent” or “anti-infective drug” or “anti-infective” are interchangeable and are used in accordance with their plain ordinary meaning and refer to a composition (e.g., compound, drug, antagonist, inhibitor, modulator) having anti-infectious agent properties or the ability to inhibit the growth or proliferation of an infectious agent (e.g., parasite (e.g., protozoa), bacterium, virus, fungus, or microorganism) or treat a symptom of a disease caused by an infectious agent. In some embodiments, an anti-infective agent is an agent approved by the FDA or similar regulatory agency of a country other than the USA, for treating infection by an infectious agent or a disease associated with an infectious agent. In embodiments, an anti-infective agent is an agent with anti-infectious agent properties that has not (e.g., yet) been approved by the FDA or similar regulatory agency of a country other than the USA, for treating infection by an infectious agent or a disease associated with an infectious agent. Examples of anti-infective agents include, but are not limited to, anti-viral agents, anti-bacterial agents, antibiotics, anti-parasitic (e.g., anti-protozoan) agents, anti-malarial agents, and anti-fungal agents.

The terms “anti-bacterial agent” or “anti-bacterial drug” or “anti-bacterial” or “antibiotic” are interchangeable and are used in accordance with their plain ordinary meaning and refer to a composition (e.g., compound, drug, antagonist, inhibitor, modulator) having anti-bacterial properties or the ability to inhibit the growth or proliferation of bacteria (e.g., bacteria that infect humans). In some embodiments, an anti-bacterial agent is an agent approved by the FDA or similar regulatory agency of a country other than the USA, for treating a bacterial infection. In embodiments, an anti-bacterial agent is an agent with the ability to inhibit the growth or proliferation of bacteria that has not (e.g., yet) been approved by the FDA or similar regulatory agency of a country other than the USA, for treating a bacterial infection. Examples of anti-bacterial agents include, but are not limited to, Penicillins (e.g., penicillins, antistaphylococcal penicillins, aminopenicillins, antipseudomonal penicillins), cephalosporins, polymyxins, rifamycins, lipiarmycins, quinolones, sulfonamides. macrolides, lincosamides, tetracyclines, aminoglycosides, cyclic lipopeptides (e.g., daptomycin, sufactin, echinocandins, caspofungin), glycylcyclines (e.g., tigecycline), oxazolidinones (e.g., linezolid, posizolid, tedizolid, radezolid, cycloserine), lipiarmycins (e.g., fidaxomicin), mecillinams, and carbapenems. An anti-bacterial moiety is a radical of an anti-bacterial. Non-limiting examples of an anti-bacterial agent include Amikacin, Gentamicin, Kanamycin, Neomycin, Netilmicin, Tobramycin, Paromomycin, Streptomycin, Spectinomycin, Geldanamycin, Herbimycin, Rifaximin, Loracarbef, Ertapenem, Doripenem, Imipenem/Cilastatin, Meropenem, Cefadroxil, Cefazolin, Cefalotin or Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), Sulfonamidochrysoidine, Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin (Rifampin), Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, and Trimethoprim.

The terms “anti-malarial agent” or “anti-malarial drug” or “anti-malarial” are interchangeable and are used in accordance with their plain ordinary meaning and refer to a composition (e.g., compound, drug, antagonist, inhibitor, modulator) having anti-malarial properties or the ability to inhibit the growth or proliferation of Plasmodium that infect humans (e.g., P. vivax, P. ovale, P. malariae P. falciparum, P. knowlesi, P. brasilianum, P. cynomolgi, P. cynomolgi bastianellii, P. inui, P. rhodiani, P. schweitzi, P. semiovale, or P. simium). In embodiments, an anti-malarial agent treats infection with P. vivax, P. ovale, P. malariae, and/or P. falciparum. In embodiments, an anti-malarial agent treats infection with P. vivax. In embodiments, an anti-malarial agent treats infection with P. ovale. In embodiments, an anti-malarial agent treats infection with P. malariae. In embodiments, an anti-malarial agent treats infection with P. falciparum. In some embodiments, an anti-malarial agent is an agent approved by the FDA or similar regulatory agency of a country other than the USA, for treating malaria. In embodiments, an anti-malarial agent is an agent with the ability to inhibit the growth or proliferation of Plasmodium that infect humans that has not (e.g., yet) been approved by the FDA or similar regulatory agency of a country other than the USA, for treating malaria. Examples of anti-malarial agents include, but are not limited to, amodiaquine, atovaquone, chloroquine, clardribine, clindamycin, cytarabine, daunorubicin, docetaxel, doxorubicin, doxycycline, etoposide, fansidar, fludarabine, halofantrine, idarubicin, imiquimod, irinotecan, mefloquine, methotrexate, mitomycin, oxamniquine, paclitaxel, plicamycin, primaquine, proquanil, pyrimethamine, quinidine, quinine, topotecan, vinblastine, vincristine, KA609, KAF156, tafenoquine, and pyronaridine. An anti-malarial moiety is a radical of an anti-malarial.

The term “siderophore” is used in accordance with its common meaning and refers to a high-affinity iron chelating compound that may be secreted by a microorganism (e.g., bacteria, fungi, grasses). Non-limiting examples of siderophores include catecholates (e.g., phenolates), hydroxamates, carboxylates (e.g., derivatives of citric acid), ferrichrome, desferrioxamine B (deferoxamine), desferrioxamine E, fusarinine C, ornibactin, rhodotorulic acid, enterobactin, bacillibactin, vibriobactin. azotobactin, pyoverdine, yersiniabactin, aerobactin, sinochelin, alcaligin, mycobactin, staphyloferrin A, and petrobactin. In embodiments, a siderophore may chelate a non-iron metal (e.g., aluminum, gallium, chromium, copper, zinc, lead, manganese, cadmium vanadium indium, plutonium, or uranium). A sideropohore moiety is a radical of a siderophore. Additional non-limiting examples of a siderophore include Achromobactin, Acinetobactin, Acinetoferrin, Aerobactin, Aeruginic, Agrobactin, Agrobactin A, Albomycin 271, Alcaligin 230, Alterobactin A, Alterobactin B, Aminochelin 262, Amonabactin P693, Amonabactin P750, Amonabactin T732, Amonabactin T789, Amphibactin B, Amphibactin C, Amphibactin D, Amphibactin E, Amphibactin F, Amphibactin G, Amphibactin H, Amphibactin I, Amycolachrome 235, Anachelin 1, Anachelin 2, Anguibactin 247, Aquachelin A, Aquachelin B, Aquachelin C, 2, Aquachelin D, Arthrobactin 199, Asperchrome A, Asperchrome B1, Asperchrome B2, Asperchrome B3, Asperchrome C, Asperchrome D1, Asperchrome D2, Asperchrome D3, Asperchrome E, Asperchrome F1, Asperchrome F2, Asperchrome F3, Aspergillic acid, Avenic acid, Azotobactin 236, Azotobactin D, Azotobactin 87, Azotochelin 236, Azoverdin 174, Bacillibactin 85, Basidiochrome 46, Bisucaberin 232, Carboxymycobactin 107, Carboxymycobactin 1, Carboxymycobactin 2, Carboxymycobactin 3, Carboxymycobactin 4, Cepabactin 266, Chrysobactin 261, Citrate 260, Coelichelin 72, 3, Coprogen 51, Coprogen B, Corynebactin 84, Deoxydistichonic acid, 2′-Deoxymugineic acid, Deoxyschizokinen 251, Des(diserylglycyl)-ferrirhodin 45, Desacetylcoprogen 52, Desferrioxamine A1, Desferrioxamine A2, Desferrioxamine B, Desferrioxamine D1, Desferrioxamine D2, Desferrioxamine E, Desferrioxamine Et1 21A, Desferrioxamine Et2 21B, Desferrioxamine Et3 21C, Desferrioxamine G1, Desferrioxamine G2A, Desferrioxamine G2B, Desferrioxamine G2C, Desferrioxamine H, Desferrioxamine P1, Desferrioxamine T1, Desferrioxamine T2, Desferrioxamine T3, Desferrioxamine T7, Desferrioxamine T8, Desferrioxamine Te1 21D, Desferrioxamine Te2 21E Desferrioxamine Te3 21F, Desferrioxamine X1, Desferrioxamine X2, 4, Desferrioxamine X3, Desferrioxamine X4, Desferrithiocin, 2,3-Dihydroxybenzoylserine, Dimerum acid, Dimethylcoprogen, Dimethylneocoprogen I, Dimethyltriornicin, Distichonic acid, Enantio Rhizoferrin, Enantio-Pyochelin, Enterobactin, Enterochelin, Exochelin MN, Exochelin MS, Ferrichrome, Ferrichrome A, Ferrichrome C, Ferrichrysin, Ferricrocin, Ferrimycin A, Ferrirhodin, Ferrirubin, Ferrocin A, Fluvibactin, Formobactin, Fusarinine A, Fusarinine B, Fusarinine C, Heterobactin A, Heterobactin B, Hydroxycopropen, Hydroxyisoneocoprogen I, 3-Hydroxymugineic acid, 5, Hydroxy-neocoprogen I, Isoneocoprogen I, Isopyoverdin BTP1, Isopyoverdin 6.7, Isopyoverdin 7.13, Isopyoverdin 90-33, Isopyoverdin 90-44, Isopyoverdin 10.7, Isotriornicin, Itoic acid, Loihichelin A, Loihichelin B, Loihichelin C, Loihichelin D, Loihichelin E, Loihichelin F, Maduraferrin, Malonichrome, Marinobactin A, Marinobactin B, Marinobactin C, Marinobactin D1, Marinobactin D2, Marinobactin E, Micacocidin, Mugineic acid, Mycobactin A, Mycobactin Av, Mycobactin F, Mycobactin H, Mycobactin J, Mycobactin M, Mycobactin N, 6, Mycobactin NA, Mycobactin P, Mycobactin R, Mycobactin S, Mycobactin T, Myxochelin, Nannochelin A, Nannochelin B, Nannochelin C, Neocoprogen I, Neocoprogen II, Neurosporin, Nocobactin, Nocobactin NA, Ochrobactin A, Ochrobactin B, Ochrobactin C, Ornibactin—C4, Ornibactin—C6, Ornibactin—C8, Ornicorrugatin, palmitoylcoprogen, Parabactin, Parabactin A, Petrobactin, Petrobactin disulphonate, Petrobactin sulphonate, Pistillarin, Protochelin, Pseudoalterobactin A, Pseudoalterobactin B, Pseudobactin 112, Pseudobactin 589A, 7, Putrebactin, Pyochelin, Pyoverdin A214, Pyoverdin BTP2, Pyoverdin C, Pyoverdin CHAO, Pyoverdin D-TR133, Pyoverdin E, Pyoverdin G R Pyoverdin GM, Pyoverdin I-III, Pyoverdin P19, Pyoverdin Pau, Pyoverdin PL8, Pyoverdin PVD, Pyoverdin R′, Pyoverdin Thai, Pyoverdin TII, Pyoverdin 1, Pyoverdin 11370, Pyoverdin 13525, Pyoverdin 1547, Pyoverdin 17400, Pyoverdin 18-1, Pyoverdin 19310, Pyoverdin 2192, Pyoverdin 2392, Pyoverdin 2461, Pyoverdin 2798, Pyoverdin 51W, Pyoverdin 9AW, Pyoverdin 90-51, Pyoverdin 95-275, Pyoverdin 96-312, Pyoverdin 96-318, Pyoverdin, Pyoverdin 6.1, Pyoverdin 6.2, Pyoverdin 6.3, Pyoverdin 6.4, Pyoverdin 6.5, Pyoverdin 6.6, Pyoverdin 6.8, Pyoverdin 7.1, Pyoverdin 7.2, Pyoverdin 7.3, Pyoverdin 7.4, Pyoverdin 7.5, Pyoverdin 7.6, Pyoverdin 7.7, Pyoverdin 7.8, Pyoverdin 7.9, Pyoverdin 7.10, Pyoverdin 7.11, Pyoverdin 7.12, Pyoverdin 7.14, Pyoverdin 7.15, Pyoverdin 7.16, Pyoverdin 7.17, Pyoverdin 7.18, Pyoverdin 7.19, Pyoverdin 8.1, Pyoverdin 8.2, Pyoverdin 8.3, Pyoverdin 8.4, Pyoverdin 8.5, Pyoverdin 8.6, Pyoverdin 8.7, Pyoverdin 8.8, Pyoverdin 8.9, Pyoverdin 9.1, Pyoverdin 9.2, Pyoverdin 9.3, Pyoverdin 9.4, Pyoverdin 9.5, Pyoverdin 9.6, Pyoverdin 9.7, Pyoverdin 9.8, Pyoverdin 9.9, Pyoverdin 9.10, Pyoverdin 9.11, Pyoverdin 9.12, Pyoverdin 10.1, Pyoverdin 10.2, Pyoverdin 10.3, Pyoverdin 10.4, Pyoverdin 10.5, Pyoverdin 10.6, Pyoverdin 10.8, Pyoverdin 10.9, Pyoverdin 10.10, Pyoverdin 11.1, Pyoverdin 11.2, Pyoverdin 12, Pyoverdin 12.1, Pyoverdin 12.2, Pyridoxatin, Quinolobactin, Rhizobactin, 10, Rhizobactin, Rhizoferrin, Rhizoferrin analogues 88A-88E, Rhodotrulic acid, Salmochelin Si, Salmochelin S2, Salmochelin S4, Salmochelin SX, Salmycin A, Schizokinen, Serratiochelin, Siderochelin A, Snychobactin A, Snychobactin B, Snychobactin C, Staphyloferrin A, Staphyloferrin B, Tetraglycine ferrichrome, Thiazostatin, Triacetylfusarinine, Triornicin, Vibriobactin, Vibrioferrin, Vicibactin, Vulnibactin, and Yersiniabactin.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y⁸⁹Sr, ⁸⁹Z, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴-¹⁵⁸¹Gd, ¹⁶¹Tb, ¹⁶⁶Dy, ¹⁶⁶Ho, ¹⁶⁹Er, ¹⁷⁵Lu, ¹⁷⁷Lu ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴-¹⁵⁸¹Gd ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ^{194, 198}Au, ¹⁹⁹Au, ²¹¹At, ²¹¹Pb ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

The terms “fluorophore” or “fluorescent agent” are used interchangeably and refer to a composition (e.g., compound) that can absorb light at one or more wavelenghs and re-emit light at one or more longer wavelengths, relative to the one or more wavelengths of absorbed light. Examples of fluorophores that may be included in the compositions described herein include fluorescent proteins, xanthene derivatives (e.g., fluorescein, rhodamine, Oregon green, eosin, or Texas red), cyanine and derivatives (e.g., cyanine, indocarbocyanine, oxacarbocyanine, thiacarbocyanine or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g. anthraquinones, DRAQ5, DRAQ7, or CyTRAK Orange), pyrene derivatives (e.g., cascade blue and derivatives), oxazine derivatives (e.g., Nile red, Nile blue, cresyl violet, oxazine 170), acridine derivatives (e.g., proflavin, acridine orange, acridine yellow), arylmethine derivatives (e.g., auramine, crystal violet, malachite green), tetrapyrrole derivatives (e.g., porphin, phthalocyanine, bilirubin), CF dye™, DRAQ™, CyTRAK™, BODIPY™, Alexa Fluor™, DyLight Fluor™, Atto™, Tracy™, FluoProbes™, Abberior Dyes™, DY™ dyes, MegaStokes Dyes™, SUlfo Cy™, Seta™ dyes, SeTau™ dyes, Square Dyes™, Quasar™ dyes, Cal Fluor™ dyes, SureLight Dyes™, PerCP™, Phycobilisomes™, APC™, APCXL™, RPE™, and/or BPE™. A fluorescent moiety is a radical of a fluorescent agent.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Pharmaceutical compositions provided by the present invention include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms (e.g., symptoms of cancer, an infectious disease, or malaria). Determination of a therapeutically effective amount of a compound of the invention is well within the capabilities of those skilled in the art, especially in light of the detailed disclosure herein.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer, infectious disease, bacterial disease, parasitic disease, viral disease, malaria, drug-resistant malaria) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer, infectious disease, bacterial disease, parasitic disease, viral disease, malaria, drug-resistant malaria) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds. A protein moiety is a radical of a protein.

The term “peptidyl” and “peptidyl moiety” means a monovalent peptide.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An antibody moiety is a radical of an antibody. Non-limiting examples of antibodies (or functional fragments thereof or antigen-binding fragments thereof, derived from such antibodies) that may be included in the compounds described herein include 3F8, 8H9, Abagovomab, Abciximab, Abrilumab, Actoxumab, Adalimumab, Adecatumumab, Aducanumab, Afelimomab, Afutuzumab, Alacizumab pegol, ALD518, Alemtuzumab, Alirocumab, Altumomab pentetate, Amatuximab, Anatumomab mafenatox, Anifrolumab, Anrukinzumab (IMA-638), Apolizumab, Arcitumomab, Aselizumab, Atinumab, Atlizumab (tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, cBR96-doxorubicin immunoconjugate, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dinutuximab, Diridavumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elotuzumab, Elsilimomab, Emibetuzumab, Enavatuzumab, Enfortumab vedotin, Enlimomab pegol, Enokizumab, Enoticumab, Ensituximab, Epitumomab cituxetan, Epratuzumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, IMAB362, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Lifastuzumab vedotin, Ligelizumab, Lintuzumab, Lirilumab, Lodelcizumab, Lorvotuzumab mertansine, Lucatumumab, Lulizumab pegol, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Obiltoxaximab, Ocaratuzumab, Ocrelizumab, Odulimomab, Ofatumumab, Olaratumab, Olokizumab, Omalizumab, Onartuzumab, Ontuxizumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teplizumab, Teprotumumab, TGN1412, Ticilimumab (tremelimumab), Tildrakizumab, Tigatuzumab, TNX-650, Tocilizumab (atlizumab), Toralizumab, Tositumomab, Tovetumab, Tralokinumab, Trastuzumab, TRBS07, Tregalizumab, Tremelimumab, Tucotuzumab celmoleukin, Tuvirumab, Ublituximab, Urelumab, Urtoxazumab, Ustekinumab, Vantictumab, Vapaliximab, Varlilumab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, and Zolimomab aritox.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments (e.g., antigen-binding fragments) include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

### II. Compounds

In an aspect is provided a compound, or a pharmaceutically acceptable salt thereof, having the formula:

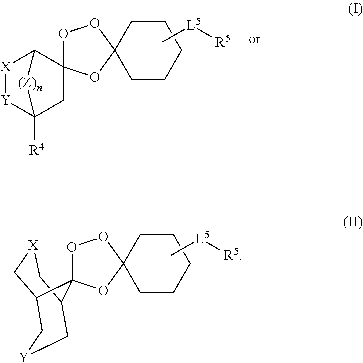

X is NR^{1.1} or C(R^{1.1}R^{1.2}). In embodiments, C(R^{1.1}R^{1.2}) is equivalent to or alternatively referred to herein as C(R^{1.1})(R^{1.2}).

Y is NR^{2.1} or C(R^{2.1}R^{2.2}). In embodiments, C(R^{2.1}R^{2.2}) is equivalent to or alternatively referred to herein as C(R^{2.1})(R^{2.2}).

Z is C(R^{3.1}R^{3.2}). In embodiments, C(R′R^{3.2}) is equivalent to or alternatively referred to herein as C(R^{3.1})(R^{3.2}).

The symbol n is 1 or 2.

L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R^{1.1} and R^{1.2} are independently hydrogen, oxo, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is hydrogen, oxo, halogen, —CX⁵₃, —CHX⁵₂, —CH₂X⁵, —OCX⁵₃, —OCH₂X⁵, —OCHX⁵², —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), a protein moiety, a detectable moiety, a siderophore moiety, or a drug moiety.

Each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹, X², X³, X⁴, and X⁵ are independently —F, —Cl, —Br, or —I.

The symbols n1, n2, n3, n4, and n5 are independently an integer from 0 to 4.

The symbols m1, m2, m3, m4, m5, v1, v2, v3, v4, and v5 are independently 1 or 2.

The compounds described herein (e.g., formulae I, II, and embodiments thereof) are prodrugs. The term “compound” when referring to a compound of the invention and the term “prodrug” when referring to a prodrug of the invention (e.g., compound including a drug moiety or those compounds including a detectable moiety, protein moiety, or other moiety in place of a drug moiety or in addition to a drug moiety) are interchangeable. In embodiments, the compounds described herein (e.g., formula I, II, and/or embodiments thereof) are prodrugs, wherein the prodrug moiety is the component of the compound that is not a drug moiety/detectable moiety/protein moiety and is released from the drug moiety/detectable moiety/protein moiety upon degradation of the prodrug in the presence of a high level of reductant (e.g., biological reductant, Fe^{II}). In embodiments, degradation of the prodrug in the presence of a high level of reductant (e.g., biological reductant, Fe^{II}) includes opening of the peroxide containing ring (e.g., trioxolane) in the prodrug moiety and release of an active drug/detectable agent/protein (e.g., where the monovalent moiety is cleaved to form a compound with full valency). A person having ordinary skill in the art would understand that the drug/detectable agent/protein and drug moiety/detectable moiety/protein moiety include only those compounds compatible with the chemistry provided herein for connecting the drug moiety/detectable moiety/protein moiety to the prodrug moiety and for release of the drug/detectable agent/protein from the compound (prodrug) by the presence of a high level of reductant (e.g., biological reductant, Fe^{II}). In embodiments, degradation of the prodrug to release an active agent (e.g., drug, protein, detectable agent, active compound) may result in an active agent including a linker or portion of the peroxide containing ring in the active agent. In such compounds, the resulting active agent includes a higher level of activity compared to the level of activity of the intact prodrug.

In embodiments, a drug moiety is (i) a radical composition that upon release (cleavage of the bond connecting the drug moiety to the prodrug moiety) from a compound (i.e., prodrugs) described herein, forms a drug (e.g., therapeutic agent); and (ii) is connected to the prodrug moiety by a bond to an N atom of the drug moiety. In embodiments, a drug moiety is (i) a radical composition that upon release (cleavage of the bond connecting the drug moiety to the prodrug moiety) from a compound (i.e., prodrugs) described herein, forms a drug (e.g., therapeutic agent); and (ii) is connected to the prodrug moiety by a bond to an O atom of the drug moiety. In embodiments, a drug moiety is (i) a radical composition that upon release (cleavage of the bond connecting the drug moiety to the prodrug moiety) from a compound (i.e., prodrugs) described herein, forms a drug (e.g., therapeutic agent); and (ii) is connected to the prodrug moiety by a bond to an S atom of the drug moiety. In embodiments, a drug moiety is (i) a radical composition that upon release (cleavage of the bond connecting the drug moiety to the prodrug moiety) from a compound (i.e., prodrugs) described herein, forms a drug (e.g., therapeutic agent); and (ii) is connected to the prodrug moiety by a bond to a —OC(O)-(remainder of drug moiety) of the drug moiety. In embodiments, the drug moiety is an anti-cancer agent moiety (e.g., described herein). In embodiments, the drug moiety is an anti-infective agent moiety (e.g., described herein). In embodiments, the drug moiety is an anti-malaria agent moiety (e.g., described herein). In embodiments, the drug moiety is an anti-bacterial agent moiety (e.g., described herein). In embodiments, the drug moiety is an antibiotic moiety (e.g., described herein). In embodiments, the drug moiety is an anti-parasitic agent moiety (e.g., described herein).

In embodiments, a detectable moiety is (i) a radical composition that upon release (cleavage of the bond connecting the detectable moiety to the prodrug moiety) from a compound (i.e., prodrugs) described herein, forms a detectable agent (e.g., fluorescent agent); and (ii) is connected to the prodrug moiety by a bond to an N atom of the detectable moiety. In embodiments, a detectable moiety is (i) a radical composition that upon release (cleavage of the bond connecting the detectable moiety to the prodrug moiety) from a compound (i.e., prodrugs) described herein, forms a detectable agent (e.g., fluorescent agent); and (ii) is connected to the prodrug moiety by a bond to an O atom of the detectable moiety. In embodiments, a detectable moiety is (i) a radical composition that upon release (cleavage of the bond connecting the detectable moiety to the prodrug moiety) from a compound (i.e., prodrugs) described herein, forms a detectable agent (e.g., fluorescent agent); and (ii) is connected to the prodrug moiety by a bond to an S atom of the detectable moiety. In embodiments, a drug moiety is (i) a radical composition that upon release (cleavage of the bond connecting the drug moiety to the prodrug moiety) from a compound (i.e., prodrugs) described herein, forms a drug (e.g., therapeutic agent); and (ii) is connected to the prodrug moiety by a bond to a —OC(O)-(remainder of detectable moiety) of the detectable moiety.

In embodiments, a protein moiety is (i) a radical composition that upon release (cleavage of the bond connecting the protein moiety to the prodrug moiety) from a compound (i.e., prodrugs) described herein, forms a protein (e.g., antibody); and (ii) is connected to the prodrug moiety by a bond to an N atom of the protein moiety. In embodiments, a protein moiety is (i) a radical composition that upon release (cleavage of the bond connecting the protein moiety to the prodrug moiety) from a compound (i.e., prodrugs) described herein, forms a protein (e.g., antibody); and (ii) is connected to the prodrug moiety by a bond to an O atom of the protein moiety. In embodiments, a protein moiety is (i) a radical composition that upon release (cleavage of the bond connecting the protein moiety to the prodrug moiety) from a compound (i.e., prodrugs) described herein, forms a protein (e.g., antibody); and (ii) is connected to the prodrug moiety by a bond to an S atom of the protein moiety. In embodiments, a drug moiety is (i) a radical composition that upon release (cleavage of the bond connecting the drug moiety to the prodrug moiety) from a compound (i.e., prodrugs) described herein, forms a drug (e.g., therapeutic agent); and (ii) is connected to the prodrug moiety by a bond to a —OC(O)-(remainder of protein moiety) of the protein moiety.

In embodiments, a compound described herein (prodrug described herein) including a drug moiety is less active than the corresponding free drug. In embodiments, a compound described herein does not have the activity of the free drug. In embodiments, a compound described herein has less than 0.9 times the activity of the free drug (e.g., less than 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 times the activity of the free drug). Drug moieties that form part of the prodrugs described herein may obtain functionality due to chemical changes in the prodrugs that occur under physiological conditions.

In embodiments, a compound described herein (prodrug described herein) including a detectable moiety is less detectable than the corresponding free detectable agent. In embodiments, a prodrug compound including a detectable moiety described herein cannot be detected using an identical method capable of detecting the free detectable agent. In embodiments, a prodrug compound including a detectable moiety described herein is less than 0.9 times as detectable as the free detectable moiety (e.g., less than 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 times as detectable as the free detectable moiety using the same method (e.g., assay)). In embodiments, a prodrug compound including a detectable moiety described herein is at least 0.9 times as detectable as the free detectable moiety (e.g., at least 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 times as detectable as the free detectable moiety using the same method (e.g., assay)). In embodiments, a compound described herein can be detected with the same sensitivity as the free detectable agent using an identical method of detection.

In embodiments, the compound has the formula:

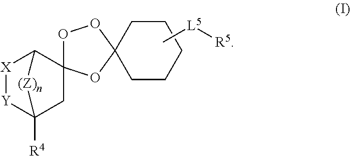

X, Y, Z, n, R⁴, R⁵, and L⁵ are as described herein, including in embodiments.

In embodiments, the compound has the formula:

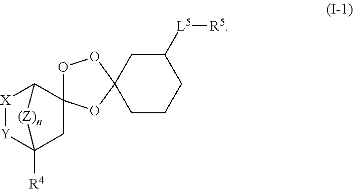

X, Y, Z, n, R⁴, R⁵, and L⁵ are as described herein, including in embodiments.

In embodiments, the compound has the formula:

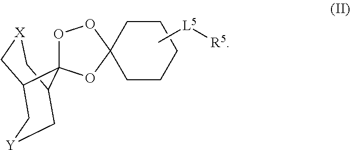

X, Y, R⁵, and L⁵ are as described herein, including in embodiments.

In embodiments, the compound has the formula:

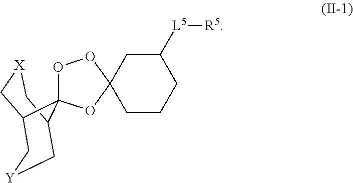

X, Y, R⁵, and L⁵ are as described herein, including in embodiments.

In embodiments, when X is NR^{1.1}, then Y is C(R^{2.1}R^{2.2}). In embodiments, C(R^{2.1}R^{2.2}) is equivalent to or alternatively referred to herein as C(R^{2.1})(R^{2.2}). In embodiments, when Y is NR^{2.1}, then X is C(R^{1.1}R^{1.2}). In embodiments, C(R^{1.1}R^{1.2}) is equivalent to or alternatively referred to herein as C(R^{1.1})(R^{1.2}).

In embodiments, X is NR^{1.1}. In embodiments, X is NH. In embodiments, X is C(R^{1.1}R^{1.2}). In embodiments, C(R^{1.1}R^{1.2}) is equivalent to or alternatively referred to herein as C(R^{1.1})(R^{1.2}). In embodiments, X is CHR^{1.2}. In embodiments, X is CH₂.

In embodiments, Y is NR²¹. In embodiments, Y is NH. In embodiments, Y is C(R^{2.1}R^{2.2}). In embodiments, C(R^{2.1}R^{2.2}) is equivalent to or alternatively referred to herein as C(R^{2.1})(R^{2.2}). In embodiments, Y is CHR^{2.2}. In embodiments, Y is CH₂.

In embodiments, Z is CHR^{3.2}. In embodiments, Z is CH₂.

In embodiments, a substituted R^{1.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1.1} is substituted with a plurality of groups selected from substituent groups, size-limited substituent groups, and lower substituent groups; each substituent group, size-limited substituent group, and/or lower substituent group may optionally be different. In embodiments, when R^{1.1} is substituted, it is substituted with at least one substituent group. In embodiments, when R^{1.1} is substituted, it is substituted with at least one size-limited substituent group. In embodiments, when R^{1.1} is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R^{1.1} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1.1} is hydrogen. In embodiments, R^{1.1} is —C(O)OR^{1C}, wherein R^{1C} is as described herein, including in embodiments. In embodiments, R^{1.1} is —C(O)OR^{1C}, wherein R^{1C} is hydrogen or unsubstituted C₁-C₄ alkyl. In embodiments, R^{1.1} is —C(O)OH. In embodiments, R^{1.1} is —C(O)OCH₃. In embodiments, R^{1.1} is —C(O)NR^{1A}R^{1B}, wherein R^{1A} and R^{1B} are as described herein, including in embodiments. In embodiments, R^{1.1} is —C(O)NH(OH). In embodiments, R^{1.1} is substituted or unsubstituted C₁-C₄ alkyl. In embodiments, R^{1.1} is substituted or unsubstituted methyl. In embodiments, R^{1.1} is substituted or unsubstituted ethyl. In embodiments, R^{1.1} is substituted or unsubstituted propyl. In embodiments, R^{1.1} is substituted or unsubstituted n-propyl. In embodiments, R^{1.1} is substituted or unsubstituted isopropyl. In embodiments, R^{1.1} is substituted or unsubstituted butyl. In embodiments, R^{1.1} is substituted or unsubstituted n-butyl. In embodiments, R^{1.1} is substituted or unsubstituted isobutyl. In embodiments, R^{1.1} is substituted or unsubstituted tert-butyl. In embodiments, R^{1.1} is substituted or unsubstituted 2 to 6 membered heteroalkyl. In embodiments, R^{1.1} is substituted 2 to 6 membered heteroalkyl. In embodiments, R^{1.1} is oxo-substituted 2 to 6 membered heteroalkyl. In embodiments, R^{1.1} is

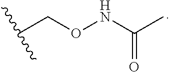 [SMILES: *CONC(C)=O]

In embodiments, a substituted R^{1.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1.2} is substituted with a plurality of groups selected from substituent groups, size-limited substituent groups, and lower substituent groups; each substituent group, size-limited substituent group, and/or lower substituent group may optionally be different. In embodiments, when R^{1.2} is substituted, it is substituted with at least one substituent group. In embodiments, when R^{1.2} is substituted, it is substituted with at least one size-limited substituent group. In embodiments, when R^{1.2} is substituted, it is substituted with at least one lower substituent group.

In embodiments, R^{1.2} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1.2} is hydrogen. In embodiments, R^{1.2} is —C(O)OR^{1C}, wherein R^{1C} is as described herein, including in embodiments. In embodiments, R^{1.2} is —C(O)OR^{1C}, wherein R^{1C} is hydrogen or unsubstituted C₁-C₄ alkyl. In embodiments, R^{1.2} is —C(O)OH. In embodiments, R^{1.2} is —C(O)OCH₃. In embodiments, R^{1.2} is —C(O)NR^{1A}R^{1B}, wherein R^{1A} and R^{1B} are as described herein, including in embodiments. In embodiments, R^{1.2} is —C(O)NH(OH). In embodiments, R^{1.2} is substituted or unsubstituted C₁-C₄ alkyl. In embodiments, R^{1.2} is substituted or unsubstituted methyl. In embodiments, R^{1.2} is substituted or unsubstituted ethyl. In embodiments, R^{1.2} is substituted or unsubstituted propyl. In embodiments, R^{1.2} is substituted or unsubstituted n-propyl. In embodiments, R^{1.2} is substituted or unsubstituted isopropyl. In embodiments, R^{1.2} is substituted or unsubstituted butyl. In embodiments, R^{1.2} is substituted or unsubstituted n-butyl. In embodiments, R^{1.2} is substituted or unsubstituted isobutyl. In embodiments, R^{1.2} is substituted or unsubstituted tert-butyl. In embodiments, R^{1.2} is substituted or unsubstituted 2 to 6 membered heteroalkyl. In embodiments, R^{1.2} is substituted 2 to 6 membered heteroalkyl. In embodiments, R^{1.2} is oxo-substituted 2 to 6 membered heteroalkyl. In embodiments, R^{1.2} is

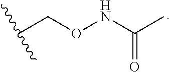 [SMILES: *CONC(C)=O]

In embodiments, R^{1A} is hydrogen or unsubstituted C₁-C₄ alkyl. In embodiments, R^{1A} is hydrogen. In embodiments, R^{1A} is unsubstituted C₁-C₄ alkyl. In embodiments, R^{1A} is unsubstituted methyl. In embodiments, R^{1A} is unsubstituted ethyl. In embodiments, R^{1A} is unsubstituted propyl. In embodiments, R^{1A} is unsubstituted n-propyl. In embodiments, R^{1A} is unsubstituted isopropyl. In embodiments, R^{1A} is unsubstituted butyl. In embodiments, R^{1A} is unsubstituted n-butyl. In embodiments, R^{1A} is unsubstituted isobutyl. In embodiments, R^{1A} is unsubstituted tert-butyl. In embodiments, R^{1A} is —OH.

In embodiments, R^{1B} is hydrogen or unsubstituted C₁-C₄ alkyl. In embodiments, R^{1B} is hydrogen. In embodiments, R^{1B} is unsubstituted C₁-C₄ alkyl. In embodiments, R^{1B} is unsubstituted methyl. In embodiments, R^{1B} is unsubstituted ethyl. In embodiments, R^{1B} is unsubstituted propyl. In embodiments, R^{1B} is unsubstituted n-propyl. In embodiments, R^{1B} is unsubstituted isopropyl. In embodiments, R^{1B} is unsubstituted butyl. In embodiments, R^{1B} is unsubstituted n-butyl. In embodiments, R^{1B} is unsubstituted isobutyl. In embodiments, R^{1B} is unsubstituted tert-butyl. In embodiments, R^{1B} is —OH.

In embodiments, R^{1C} is hydrogen or unsubstituted C₁-C₄ alkyl. In embodiments, R^{1C} is hydrogen. In embodiments, R^{1C} is unsubstituted C₁-C₄ alkyl. In embodiments, R^{1C} is unsubstituted methyl. In embodiments, R^{1C} is unsubstituted ethyl. In embodiments, R^{1C} is unsubstituted propyl. In embodiments, R^{1C} is unsubstituted n-propyl. In embodiments, R^{1C} is unsubstituted isopropyl. In embodiments, R^{1C} is unsubstituted butyl. In embodiments, R^{1C} is unsubstituted n-butyl. In embodiments, R^{1C} is unsubstituted isobutyl. In embodiments, R^{1C} is unsubstituted tert-butyl.

In embodiments, R^{1D} is hydrogen or unsubstituted C₁-C₄ alkyl. In embodiments, R^{1D} is hydrogen. In embodiments, R^{1D} is unsubstituted C₁-C₄ alkyl. In embodiments, R^{1D} is unsubstituted methyl. In embodiments, R^{1D} is unsubstituted ethyl. In embodiments, R^{1D} is unsubstituted propyl. In embodiments, R^{1D} is unsubstituted n-propyl. In embodiments, R^{1D} is unsubstituted isopropyl. In embodiments, R^{1D} is unsubstituted butyl. In embodiments, R^{1D} is unsubstituted n-butyl. In embodiments, R^{1D} is unsubstituted isobutyl. In embodiments, R^{1D} is unsubstituted tert-butyl.

In embodiments, a substituted R^{2.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.1} is substituted with a plurality of groups selected from substituent groups, size-limited substituent groups, and lower substituent groups; each substituent group, size-limited substituent group, and/or lower substituent group may optionally be different. In embodiments, when R^{2.1} is substituted, it is substituted with at least one substituent group. In embodiments, when R^{2.1} is substituted, it is substituted with at least one size-limited substituent group. In embodiments, when R^{2.1} is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2.1} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2.1} is hydrogen. In embodiments, R^{2.1} is substituted or unsubstituted C₁-C₄ alkyl. In embodiments, R^{2.1} is substituted or unsubstituted methyl. In embodiments, R^{2.1} is substituted or unsubstituted ethyl. In embodiments, R^{2.1} is substituted or unsubstituted propyl. In embodiments, R^{2.1} is substituted or unsubstituted n-propyl. In embodiments, R^{2.1} is substituted or unsubstituted isopropyl. In embodiments, R^{2.1} is substituted or unsubstituted butyl. In embodiments, R^{2.1} is substituted or unsubstituted n-butyl. In embodiments, R^{2.1} is substituted or unsubstituted isobutyl. In embodiments, R^{2.1} is substituted or unsubstituted tert-butyl. In embodiments, R^{2.1} is substituted or unsubstituted 2 to 6 membered heteroalkyl. In embodiments, R^{2.1} is oxo-substituted 2 to 6 membered heteroalkyl.

In embodiments, a substituted R^{2.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.2} is substituted with a plurality of groups selected from substituent groups, size-limited substituent groups, and lower substituent groups; each substituent group, size-limited substituent group, and/or lower substituent group may optionally be different. In embodiments, when R^{2.2} is substituted, it is substituted with at least one substituent group. In embodiments, when R^{2.2} is substituted, it is substituted with at least one size-limited substituent group. In embodiments, when R^{2.2} is substituted, it is substituted with at least one lower substituent group.

In embodiments, R^{2.2} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2.2} is hydrogen. In embodiments, R^{2.2} is substituted or unsubstituted C₁-C₄ alkyl. In embodiments, R^{2.2} is substituted or unsubstituted methyl. In embodiments, R^{2.2} is substituted or unsubstituted ethyl. In embodiments, R^{2.2} is substituted or unsubstituted propyl. In embodiments, R^{2.2} is substituted or unsubstituted n-propyl. In embodiments, R^{2.2} is substituted or unsubstituted isopropyl. In embodiments, R^{2.2} is substituted or unsubstituted butyl. In embodiments, R^{2.2} is substituted or unsubstituted n-butyl. In embodiments, R^{2.2} is substituted or unsubstituted isobutyl. In embodiments, R^{2.2} is substituted or unsubstituted tert-butyl. In embodiments, R^{2.2} is substituted or unsubstituted 2 to 6 membered heteroalkyl. In embodiments, R^{2.2} is oxo-substituted 2 to 6 membered heteroalkyl.

In embodiments, R^{2A} is hydrogen or unsubstituted C₁-C₄ alkyl. In embodiments, R^{2A} is hydrogen. In embodiments, R^{2A} is unsubstituted C₁-C₄ alkyl. In embodiments, R^{2A} is unsubstituted methyl. In embodiments, R^{2A} is unsubstituted ethyl. In embodiments, R^{2A} is unsubstituted propyl. In embodiments, R^{2A} is unsubstituted n-propyl. In embodiments, R^{2A} is unsubstituted isopropyl. In embodiments, R^{2A} is unsubstituted butyl. In embodiments, R^{2A} is unsubstituted n-butyl. In embodiments, R^{2A} is unsubstituted isobutyl. In embodiments, R^{2A} is unsubstituted tert-butyl.

In embodiments, R^{2B} is hydrogen or unsubstituted C₁-C₄ alkyl. In embodiments, R^{2B} is hydrogen. In embodiments, R^{2B} is unsubstituted C₁-C₄ alkyl. In embodiments, R^{2B} is unsubstituted methyl. In embodiments, R^{2B} is unsubstituted ethyl. In embodiments, R^{2B} is unsubstituted propyl. In embodiments, R^{2B} is unsubstituted n-propyl. In embodiments, R^{2B} is unsubstituted isopropyl. In embodiments, R^{2B} is unsubstituted butyl. In embodiments, R^{2B} is unsubstituted n-butyl. In embodiments, R^{2B} is unsubstituted isobutyl. In embodiments, R^{2B} is unsubstituted tert-butyl.

In embodiments, R^{2C} is hydrogen or unsubstituted C₁-C₄ alkyl. In embodiments, R^{2C} is hydrogen. In embodiments, R^{2C} is unsubstituted C₁-C₄ alkyl. In embodiments, R^{2C} is unsubstituted methyl. In embodiments, R^{2C} is unsubstituted ethyl. In embodiments, R^{2C} is unsubstituted propyl. In embodiments, R^{2C} is unsubstituted n-propyl. In embodiments, R^{2C} is unsubstituted isopropyl. In embodiments, R^{2C} is unsubstituted butyl. In embodiments, R^{2C} is unsubstituted n-butyl. In embodiments, R^{2C} is unsubstituted isobutyl. In embodiments, R^{2C} is unsubstituted tert-butyl.

In embodiments, R^{2D} is hydrogen or unsubstituted C₁-C₄ alkyl. In embodiments, R^{2D} is hydrogen. In embodiments, R^{2D} is unsubstituted C₁-C₄ alkyl. In embodiments, R^{2D} is unsubstituted methyl. In embodiments, R^{2D} is unsubstituted ethyl. In embodiments, R^{2D} is unsubstituted propyl. In embodiments, R^{2D} is unsubstituted n-propyl. In embodiments, R^{2D} is unsubstituted isopropyl. In embodiments, R^{2D} is unsubstituted butyl. In embodiments, R^{2D} is unsubstituted n-butyl. In embodiments, R^{2D} is unsubstituted isobutyl. In embodiments, R^{2D} is unsubstituted tert-butyl.

In embodiments, a substituted R^{3.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.1} is substituted with a plurality of groups selected from substituent groups, size-limited substituent groups, and lower substituent groups; each substituent group, size-limited substituent group, and/or lower substituent group may optionally be different. In embodiments, when R^{3.1} is substituted, it is substituted with at least one substituent group. In embodiments, when R^{3.1} is substituted, it is substituted with at least one size-limited substituent group. In embodiments, when R^{3.1} is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, each R^{3.1} is the same. In embodiments, each R^{3.1} is different.

In embodiments, R^{3.1} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{3.1} is hydrogen. In embodiments, R^{3.1} is substituted or unsubstituted C₁-C₄ alkyl. In embodiments, R^{3.1} is substituted or unsubstituted methyl. In embodiments, R^{3.1} is substituted or unsubstituted ethyl. In embodiments, R^{3.1} is substituted or unsubstituted propyl. In embodiments, R^{3.1} is substituted or unsubstituted n-propyl. In embodiments, R^{3.1} is substituted or unsubstituted isopropyl. In embodiments, R^{3.1} is substituted or unsubstituted butyl. In embodiments, R^{3.1} is substituted or unsubstituted n-butyl. In embodiments, R^{3.1} is substituted or unsubstituted isobutyl. In embodiments, R^{3.1} is substituted or unsubstituted tert-butyl. In embodiments, R^{3.1} is substituted or unsubstituted 2 to 6 membered heteroalkyl. In embodiments, R^{3.1} is oxo-substituted 2 to 6 membered heteroalkyl.

In embodiments, a substituted R^{3.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.2} is substituted with a plurality of groups selected from substituent groups, size-limited substituent groups, and lower substituent groups; each substituent group, size-limited substituent group, and/or lower substituent group may optionally be different. In embodiments, when R^{3.2} is substituted, it is substituted with at least one substituent group. In embodiments, when R^{3.2} is substituted, it is substituted with at least one size-limited substituent group. In embodiments, when R^{3.2} is substituted, it is substituted with at least one lower substituent group.

In embodiments, each R^{3.2} is the same. In embodiments, each R^{3.2} is different.

In embodiments, R^{3.2} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{3.2} is hydrogen. In embodiments, R^{3.2} is substituted or unsubstituted C₁-C₄ alkyl. In embodiments, R^{3.2} is substituted or unsubstituted methyl. In embodiments, R^{3.2} is substituted or unsubstituted ethyl. In embodiments, R^{3.2} is substituted or unsubstituted propyl. In embodiments, R^{3.2} is substituted or unsubstituted n-propyl. In embodiments, R^{3.2} is substituted or unsubstituted isopropyl. In embodiments, R^{3.2} is substituted or unsubstituted butyl. In embodiments, R^{3.2} is substituted or unsubstituted n-butyl. In embodiments, R^{3.2} is substituted or unsubstituted isobutyl. In embodiments, R^{3.2} is substituted or unsubstituted tert-butyl. In embodiments, R^{3.2} is substituted or unsubstituted 2 to 6 membered heteroalkyl. In embodiments, R^{3.2} is oxo-substituted 2 to 6 membered heteroalkyl.

In embodiments, R^{3A} is hydrogen or unsubstituted C₁-C₄ alkyl. In embodiments, R^{3A} is hydrogen. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl.

In embodiments, R^{3B} is hydrogen or unsubstituted C₁-C₄ alkyl. In embodiments, R^{3B} is hydrogen. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl.

In embodiments, R^{3C} is hydrogen or unsubstituted C₁-C₄ alkyl. In embodiments, R^{3C} is hydrogen. In embodiments, R^{3C} is unsubstituted C₁-C₄ alkyl. In embodiments, R^{3C} is unsubstituted methyl. In embodiments, R^{3C} is unsubstituted ethyl. In embodiments, R^{3C} is unsubstituted propyl. In embodiments, R^{3C} is unsubstituted n-propyl. In embodiments, R^{3C} is unsubstituted isopropyl. In embodiments, R^{3C} is unsubstituted butyl. In embodiments, R^{3C} is unsubstituted n-butyl. In embodiments, R^{3C} is unsubstituted isobutyl. In embodiments, R^{3C} is unsubstituted tert-butyl.

In embodiments, R^{3D} is hydrogen or unsubstituted C₁-C₄ alkyl. In embodiments, R^{3D} is hydrogen. In embodiments, R^{3D} is unsubstituted C₁-C₄ alkyl. In embodiments, R^{3D} is unsubstituted methyl. In embodiments, R^{3D} is unsubstituted ethyl. In embodiments, R^{3D} is unsubstituted propyl. In embodiments, R^{3D} is unsubstituted n-propyl. In embodiments, R^{3D} is unsubstituted isopropyl. In embodiments, R^{3D} is unsubstituted butyl. In embodiments, R^{3D} is unsubstituted n-butyl. In embodiments, R^{3D} is unsubstituted isobutyl. In embodiments, R^{3D} is unsubstituted tert-butyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted 2 to 6 membered heteroalkyl. In embodiments, R⁴ is unsubstituted methoxy. In embodiments, R⁴ is unsubstituted ethoxy. In embodiments, R⁴ is unsubstituted propoxy. In embodiments, R⁴ is unsubstituted n-propoxy. In embodiments, R⁴ is unsubstituted isopropoxy. In embodiments, R⁴ is unsubstituted butoxy.

In embodiments, R^{4A} is hydrogen or unsubstituted C₁-C₄ alkyl. In embodiments, R^{4A} is hydrogen. In embodiments, R^{4A} is unsubstituted C₁-C₄ alkyl. In embodiments, R^{4A} is unsubstituted methyl. In embodiments, R^{4A} is unsubstituted ethyl. In embodiments, R^{4A} is unsubstituted propyl. In embodiments, R^{4A} is unsubstituted n-propyl. In embodiments, R^{4A} is unsubstituted isopropyl. In embodiments, R^{4A} is unsubstituted butyl. In embodiments, R^{4A} is unsubstituted n-butyl. In embodiments, R^{4A} is unsubstituted isobutyl. In embodiments, R^{4A} is unsubstituted tert-butyl.

In embodiments, R^{4B} is hydrogen or unsubstituted C₁-C₄ alkyl. In embodiments, R^{4B} is hydrogen. In embodiments, R^{4B} is unsubstituted C₁-C₄ alkyl. In embodiments, R^{4B} is unsubstituted methyl. In embodiments, R^{4B} is unsubstituted ethyl. In embodiments, R^{4B} is unsubstituted propyl. In embodiments, R^{4B} is unsubstituted n-propyl. In embodiments, R^{4B} is unsubstituted isopropyl. In embodiments, R^{4B} is unsubstituted butyl. In embodiments, R^{4B} is unsubstituted n-butyl. In embodiments, R^{4B} is unsubstituted isobutyl. In embodiments, R^{4B} is unsubstituted tert-butyl.

In embodiments, R^{4C} is hydrogen or unsubstituted C₁-C₄ alkyl. In embodiments, R^{4C} is hydrogen. In embodiments, R^{4C} is unsubstituted C₁-C₄ alkyl. In embodiments, R^{4C} is unsubstituted methyl. In embodiments, R^{4C} is unsubstituted ethyl. In embodiments, R^{4C} is unsubstituted propyl. In embodiments, R^{4C} is unsubstituted n-propyl. In embodiments, R^{4C} is unsubstituted isopropyl. In embodiments, R^{4C} is unsubstituted butyl. In embodiments, R^{4C} is unsubstituted n-butyl. In embodiments, R^{4C} is unsubstituted isobutyl. In embodiments, R^{4C} is unsubstituted tert-butyl.

In embodiments, R^{4D} is hydrogen or unsubstituted C₁-C₄ alkyl. In embodiments, R^{4D} is hydrogen. In embodiments, R^{4D} is unsubstituted C₁-C₄ alkyl. In embodiments, R^{4D} is unsubstituted methyl. In embodiments, R^{4D} is unsubstituted ethyl. In embodiments, R^{4D} is unsubstituted propyl. In embodiments, R^{4D} is unsubstituted n-propyl. In embodiments, R^{4D} is unsubstituted isopropyl. In embodiments, R^{4D} is unsubstituted butyl. In embodiments, R^{4D} is unsubstituted n-butyl. In embodiments, R^{4D} is unsubstituted isobutyl. In embodiments, R^{4D} is unsubstituted tert-butyl.

In embodiments, the compound has the formula:

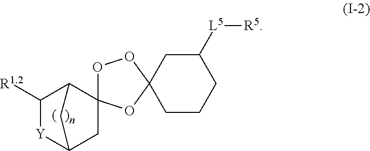

R^{1.2}, R⁵, L⁵, Y, and n are as described herein, including in embodiments.

In embodiments, the compound has the formula:

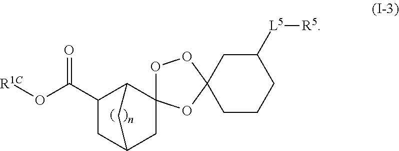

R^{1C}, R⁵, L⁵, and n are as described herein, including in embodiments.

In embodiments, the compound has the formula:

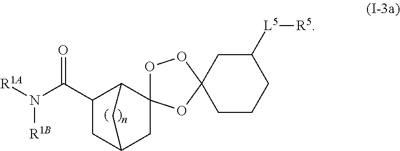

R^{1A}, R^{1B}, R⁵, L⁵, and n are as described herein, including in embodiments.

In embodiments, the compound has the formula:

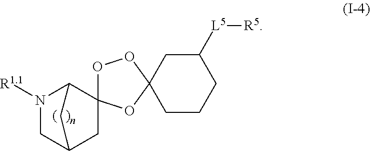

R^{1.1}, R⁵, L⁵, and n are as described herein, including in embodiments.

In embodiments, the compound has the formula:

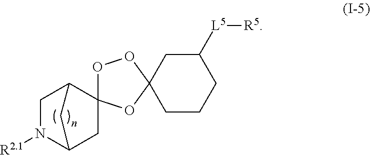

R^{2.1}, R⁵, L⁵, and n are as described herein, including in embodiments.

In embodiments, the compound has the formula:

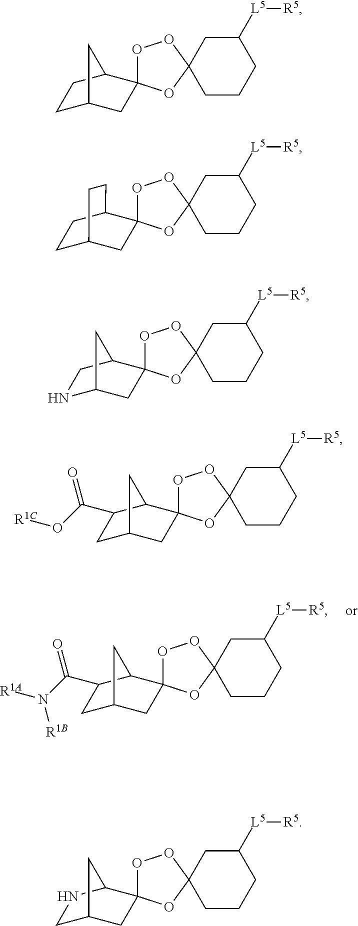

R^{1A}, R^{1B}, R^{1C}, R⁵, and L⁵ are as described herein, including in embodiments.

In embodiments, the compound has the formula:

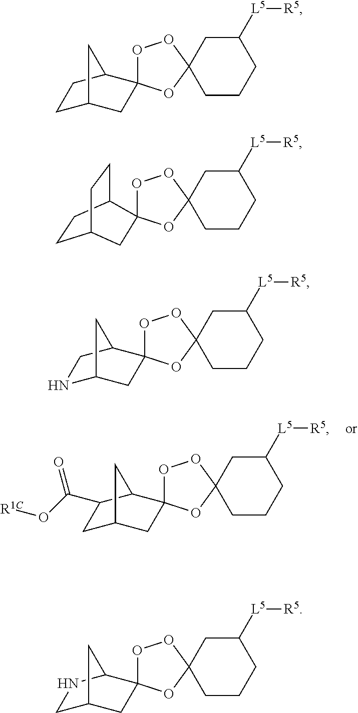

R^{1C}, R⁵, and L⁵ are as described herein, including in embodiments. In embodiments, the compound has the formula:

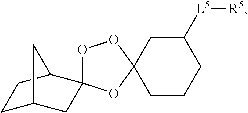

wherein R⁵ and L⁵ are as described herein, including in embodiments. In embodiments, the compound has the formula:

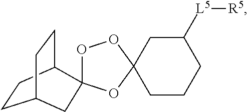

wherein R⁵ and L⁵ are as described herein, including in embodiments. In embodiments, the compound has the formula:

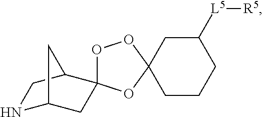

wherein R⁵ and L⁵ are as described herein, including in embodiments. In embodiments, the compound has the formula:

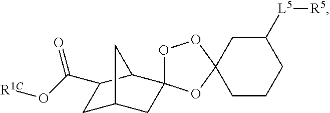

wherein R^{1C}, R⁵, and L⁵ are as described herein, including in embodiments. In embodiments, the compound has the formula:

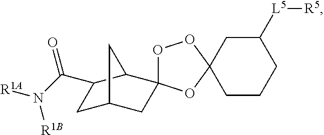

wherein R^{1A}, R^{1B}, R⁵, and L⁵ are as described herein, including in embodiments. In embodiments, the compound has the formula:

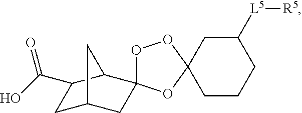

wherein R⁵ and L⁵ are as described herein, including in embodiments. In embodiments, the compound has the formula:

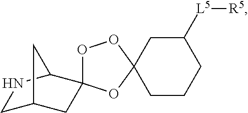

wherein R⁵ and L⁵ are as described herein, including in embodiments.

In embodiments, the compound has the formula:

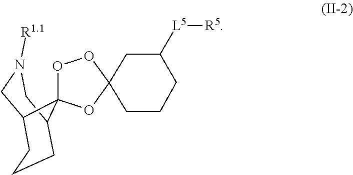

R^{1.1}, R⁵, and L⁵ are as described herein, including in embodiments.

In embodiments, the compound has the formula:

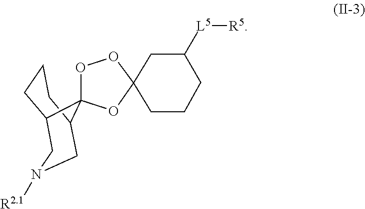

R^{2.1}, R⁵, and L⁵ are as described herein, including in embodiments.

In embodiments, the compound has the formula:

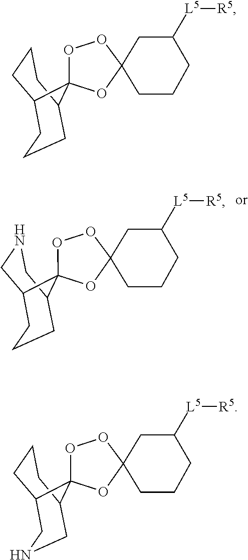

R⁵ and ^{L5} are as described herein, including in embodiments. In embodiments, the compound has the formula:

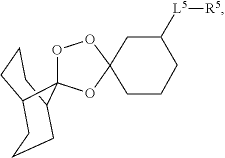

wherein R⁵ and L⁵ are as described herein, including in embodiments. In embodiments, the compound has the formula:

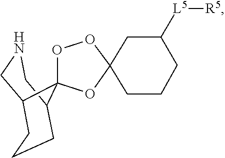

wherein R⁵ and L⁵ are as described herein, including in embodiments. In embodiments, the compound has the formula:

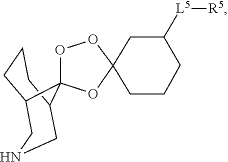

wherein R⁵ and L⁵ are as described herein, including in embodiments.

In embodiments, a substituted L⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁵ is substituted with a plurality of groups selected from substituent groups, size-limited substituent groups, and lower substituent groups; each substituent group, size-limited substituent group, and/or lower substituent group may optionally be different. In embodiments, when L⁵ is substituted, it is substituted with at least one substituent group. In embodiments, when L⁵ is substituted, it is substituted with at least one size-limited substituent group. In embodiments, when L⁵ is substituted, it is substituted with at least one lower substituent group.

In embodiments, L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L⁵ is a bond, —N(R¹⁷)—, —N(R¹⁷)C(O)O—, —O—, —S—, —OC(O)—, —OC(O)N(R¹⁷)—, —OC(O)O—, —SO₂—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L⁵ is a bond, —N(R¹⁷)-L¹³-L¹⁴-, —O-L¹³-L¹⁴-, —OC(O)-L¹³-L¹⁴-, or —OC(O)N(R¹⁷)-L¹³-L¹⁴-. In embodiments, L⁵ is a bond, —N(R¹⁷)—, —O—, —OC(O)—, or —OC(O)N(R¹⁷)—. In embodiments, L⁵ is a bond. In embodiments, L⁵ is —N(R¹⁷)-L¹³-L¹⁴-. In embodiments L⁵ is —NH-Ph-CH₂—. In embodiments L⁵ is —NH-Ph-CH₂—OC(O)—. In embodiments, L⁵ is —N(R¹⁷)—. In embodiments, L⁵ is —NH—. In embodiments, L⁵ is —N(R¹⁷)C(O)O-L¹³-L¹⁴-. In embodiments, L⁵ is —N(R¹⁷)C(O)O—. In embodiments, L⁵ is —NHC(O)O—. In embodiments, L⁵ is —O-L¹³-L¹⁴-. In embodiments L⁵ is —O-Ph-CH₂—. In embodiments L⁵ is —O-Ph-CH₂—OC(O)—. In embodiments, L⁵ is —O—. In embodiments, L⁵ is —S-L¹³L¹⁴-. In embodiments L⁵ is —S-Ph-CH₂—. In embodiments L⁵ is —S-Ph-CH₂—OC(O)—. In embodiments, L⁵ is —S—. In embodiments, L⁵ is —OC(O)-L¹³L¹⁴-. In embodiments, L⁵ is —OC(O)—. In embodiments, L⁵ is —OC(O)N(R¹⁷)-L¹³-L¹⁴-. In embodiments L⁵ is —OC(O)NH-Ph-CH₂—. In embodiments L⁵ is —OC(O)NH-Ph-CH₂—OC(O)—. In embodiments, L⁵ is —OC(O)N(R¹⁷)—. In embodiments, L⁵ is —OC(O)NH—. In embodiments, L⁵ is —OC(O)O-L¹³L¹⁴-. In embodiments, L⁵ is —OC(O)O—. In embodiments, L⁵ is —SO₂-L¹³-L¹⁴-. In embodiments, L⁵ is —SO₂—. In embodiments, L⁵ is —OSO₂-L¹³-L¹⁴-. In embodiments, L⁵ is —OSO₂—. In embodiments, L⁵ is —C(O)N(R¹⁷)-L¹³-L¹⁴-. In embodiments, L⁵ is —C(O)N(R¹⁷)—. In embodiments, L⁵ is —C(O)NH—. In embodiments, L⁵ is —N(R¹⁷)C(O)-L¹³-L¹⁴-. In embodiments, L⁵ is —N(R¹⁷)C(O)—. In embodiments, L⁵ is —NHC(O)—. In embodiments, L⁵ is —S(O)₂N(R¹⁷)-L¹³-L¹⁴-. In embodiments, L⁵ is —S(O)₂N(R¹⁷)—. In embodiments, L⁵ is —S(O)₂NH—. In embodiments, L⁵ is —N(R¹⁷)S(O)₂-L¹³-L¹⁴-. In embodiments, L⁵ is —N(R¹⁷)S(O)₂—. In embodiments, L⁵ is —NHS(O)₂—. In embodiments, L⁵ is a substituted or unsubstituted C₁-C₂₀ alkylene. In embodiments, L⁵ is a substituted or unsubstituted 2 to 20 membered heteroalkylene. In embodiments, L⁵ is a substituted or unsubstituted C₃-C₂₀ cycloalkylene. In embodiments, L⁵ is a substituted or unsubstituted 3 to 20 membered heterocycloalkylene. In embodiments, L⁵ is a substituted or unsubstituted C₆-C₂₀ arylene. In embodiments, L⁵ is a substituted or unsubstituted 5 to 20 membered heteroarylene. In embodiments, L⁵ is a substituted C₁-C₂₀ alkylene. In embodiments, L⁵ is a substituted 2 to 20 membered heteroalkylene. In embodiments, L⁵ is a substituted C₃-C₂₀ cycloalkylene. In embodiments, L⁵ is a substituted 3 to 20 membered heterocycloalkylene. In embodiments, L⁵ is a substituted C₆-C₂₀ arylene. In embodiments, L⁵ is a substituted 5 to 20 membered heteroarylene. In embodiments, L⁵ is an unsubstituted C₁-C₂₀ alkylene. In embodiments, L⁵ is an unsubstituted 2 to 20 membered heteroalkylene. In embodiments, L⁵ is an unsubstituted C₃-C₂₀ cycloalkylene. In embodiments, L⁵ is an unsubstituted 3 to 20 membered heterocycloalkylene. In embodiments, L⁵ is an unsubstituted C₆-C₂₀ arylene. In embodiments, L⁵ is an unsubstituted 5 to 20 membered heteroarylene. In embodiments, L⁵ is a substituted or unsubstituted C₁-C₁₄ alkylene. In embodiments, L⁵ is a substituted or unsubstituted 2 to 14 membered heteroalkylene. In embodiments, L⁵ is a substituted or unsubstituted C₃-C₁₄ cycloalkylene. In embodiments, L⁵ is a substituted or unsubstituted 3 to 14 membered heterocycloalkylene. In embodiments, L⁵ is a substituted or unsubstituted C₆-C₁₄ arylene. In embodiments, L⁵ is a substituted or unsubstituted 5 to 14 membered heteroarylene. In embodiments, L⁵ is a substituted C₁-C₁₄ alkylene. In embodiments, L⁵ is a substituted 2 to 14 membered heteroalkylene. In embodiments, L⁵ is a substituted C₃-C₁₄ cycloalkylene. In embodiments, L⁵ is a substituted 3 to 14 membered heterocycloalkylene. In embodiments, L⁵ is a substituted C₆-C₁₄ arylene. In embodiments, L⁵ is a substituted 5 to 14 membered heteroarylene. In embodiments, L⁵ is an unsubstituted C₁-C₁₄ alkylene. In embodiments, L⁵ is an unsubstituted 2 to 14 membered heteroalkylene. In embodiments, L⁵ is an unsubstituted C₃-C₁₄ cycloalkylene. In embodiments, L⁵ is an unsubstituted 3 to 14 membered heterocycloalkylene. In embodiments, L⁵ is an unsubstituted C₆-C₁₄ arylene. In embodiments, L⁵ is an unsubstituted 5 to 14 membered heteroarylene. In embodiments, L⁵ is a substituted or unsubstituted C₁-C₈ alkylene. In embodiments, L⁵ is a substituted or unsubstituted 2 to 8 membered heteroalkylene. In embodiments, L⁵ is a substituted or unsubstituted C₃-C₈ cycloalkylene. In embodiments, L⁵ is a substituted or unsubstituted 3 to 8 membered heterocycloalkylene. In embodiments, L⁵ is a substituted or unsubstituted C₆-C₁₀ arylene. In embodiments, L⁵ is a substituted or unsubstituted 5 to 10 membered heteroarylene. In embodiments, L⁵ is a substituted C₁-C₈ alkylene. In embodiments, L⁵ is a substituted 2 to 8 membered heteroalkylene. In embodiments, L⁵ is a substituted C₃-C₈ cycloalkylene. In embodiments, L⁵ is a substituted 3 to 8 membered heterocycloalkylene. In embodiments, L⁵ is a substituted C₆-C₁₀ arylene. In embodiments, L⁵ is a substituted 5 to 10 membered heteroarylene. In embodiments, L⁵ is an unsubstituted C₁-C₈ alkylene. In embodiments, L⁵ is an unsubstituted 2 to 8 membered heteroalkylene. In embodiments, L⁵ is an unsubstituted C₃-C₈ cycloalkylene. In embodiments, L⁵ is an unsubstituted 3 to 8 membered heterocycloalkylene. In embodiments, L⁵ is an unsubstituted C₆-C₁₀ arylene. In embodiments, L⁵ is an unsubstituted 5 to 10 membered heteroarylene.

In embodiments, L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, or —N(R¹⁷)S(O)₂-L¹³-L¹⁴-; and R⁵ is a protein moiety, drug moiety, or a detectable moiety. In embodiments, L⁵ is a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, or —N(R¹⁷)S(O)₂—; and R⁵ is a protein moiety, drug moiety, or a detectable moiety.

In embodiments, a substituted L¹³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹³ is substituted with a plurality of groups selected from substituent groups, size-limited substituent groups, and lower substituent groups; each substituent group, size-limited substituent group, and/or lower substituent group may optionally be different. In embodiments, when L¹³ is substituted, it is substituted with at least one substituent group. In embodiments, when L¹³ is substituted, it is substituted with at least one size-limited substituent group. In embodiments, when L¹³ is substituted, it is substituted with at least one lower substituent group.

In embodiments, L¹³ is a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹³ is a bond, —NH—, —NHC(O)O—, —O—, —S—, —OC(O)—, —OC(O)NH—, —OC(O)O—, —OSO₂—, —C(O)NH—, —NHC(O)—, —S(O)₂NH—, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹³ is a bond or substituted or unsubstituted arylene. In embodiments, L¹³ is a bond or substituted or unsubstituted phenylene. In embodiments, L¹³ is a bond. In embodiments, L¹³ is a substituted or unsubstituted arylene. In embodiments, L¹³ is a substituted arylene. In embodiments, L¹³ is an unsubstituted arylene. In embodiments, L¹³ is a substituted or unsubstituted phenylene. In embodiments, L¹³ is an unsubstituted phenylene.

In embodiments, L¹³ is —NHC(O)—(CH₂)_{w1}—NHC(O)O—(CH₂)_{y1}—, wherein w1 and y1 are as described herein, including in embodiments. In embodiments, L¹³ is —NHC(O)—(CH₂)_{w1}—C(O)NH—(CH₂)_{y1}—, wherein w1 and y1 are as described herein, including in embodiments. In embodiments, L¹³ is —NHC(O)—(CH₂)_{w1}—C(O)—, wherein w1 is as described herein, including in embodiments. In embodiments, L¹³ is —NHC(O)—(CH₂)_{w1}—NH—, wherein w1 is as described herein, including in embodiments. In embodiments, L¹³ is —NHC(O)—(CH₂)_{w1}—NHC(O)—, wherein w1 is as described herein, including in embodiments. In embodiments, L¹³ is —NHC(O)—(CH₂)_{w1}—C(O)NH—, wherein w1 is as described herein, including in embodiments. In embodiments, L¹³ is —NHC(O)—(CH₂)_{w1}—NHC(O)O—, wherein w1 is as described herein, including in embodiments. In embodiments, L¹³ is —NHC(O)—(CH₂)_{w1}—(OCH₂CH₂)ₜ₁—C(O)NH—(CH₂)_{y1}—, wherein w1, t1, and y1 are as described herein, including in embodiments. In embodiments, L¹³ is —NHC(O)—(CH₂)_{w1}—(OCH₂CH₂)ₜ₁—C(O)NH—(CH₂)_{y1}—C(O)—, wherein w1, t1, and y1 are as described herein, including in embodiments. In embodiments, L¹³ is a substituted or unsubstituted C₁-C₂₀ alkylene. In embodiments, L¹³ is a substituted or unsubstituted 2 to 20 membered heteroalkylene. In embodiments, L¹³ is a substituted or unsubstituted C₃-C₂₀ cycloalkylene. In embodiments, L¹³ is a substituted or unsubstituted 3 to 20 membered heterocycloalkylene. In embodiments, L¹³ is a substituted or unsubstituted C₆-C₂₀ arylene. In embodiments, L¹³ is a substituted or unsubstituted 5 to 20 membered heteroarylene. In embodiments, L¹³ is a substituted C₁-C₂₀ alkylene. In embodiments, L¹³ is a substituted 2 to 20 membered heteroalkylene. In embodiments, L¹³ is a substituted C₃-C₂₀ cycloalkylene. In embodiments, L¹³ is a substituted 3 to 20 membered heterocycloalkylene. In embodiments, L¹³ is a substituted C₆-C₂₀ arylene. In embodiments, L¹³ is a substituted 5 to 20 membered heteroarylene. In embodiments, L¹³ is an unsubstituted C₁-C₂₀ alkylene. In embodiments, L¹³ is an unsubstituted 2 to 20 membered heteroalkylene. In embodiments, L¹³ is an unsubstituted C₃-C₂₀ cycloalkylene. In embodiments, L¹³ is an unsubstituted 3 to 20 membered heterocycloalkylene. In embodiments, L¹³ is an unsubstituted C₆-C₂₀ arylene. In embodiments, L¹³ is an unsubstituted 5 to 20 membered heteroarylene. In embodiments, L¹³ is a substituted or unsubstituted C₁-C₁₄ alkylene. In embodiments, L¹³ is a substituted or unsubstituted 2 to 14 membered heteroalkylene. In embodiments, L¹³ is a substituted or unsubstituted C₃-C₁₄ cycloalkylene. In embodiments, L¹³ is a substituted or unsubstituted 3 to 14 membered heterocycloalkylene. In embodiments, L¹³ is a substituted or unsubstituted C₆-C₁₄ arylene. In embodiments, L¹³ is a substituted or unsubstituted 5 to 14 membered heteroarylene. In embodiments, L¹³ is a substituted C₁-C₁₄ alkylene. In embodiments, L¹³ is a substituted 2 to 14 membered heteroalkylene. In embodiments, L¹³ is a substituted C₃-C₁₄ cycloalkylene. In embodiments, L¹³ is a substituted 3 to 14 membered heterocycloalkylene. In embodiments, L¹³ is a substituted C₆-C₁₄ arylene. In embodiments, L¹³ is a substituted 5 to 14 membered heteroarylene. In embodiments, L¹³ is an unsubstituted C₁-C₁₄ alkylene. In embodiments, L¹³ is an unsubstituted 2 to 14 membered heteroalkylene. In embodiments, L¹³ is an unsubstituted C₃-C₁₄ cycloalkylene. In embodiments, L¹³ is an unsubstituted 3 to 14 membered heterocycloalkylene. In embodiments, L¹³ is an unsubstituted C₆-C₁₄ arylene. In embodiments, L¹³ is an unsubstituted 5 to 14 membered heteroarylene. In embodiments, L¹³ is a substituted or unsubstituted C₁-C₈ alkylene. In embodiments, L¹³ is a substituted or unsubstituted 2 to 8 membered heteroalkylene. In embodiments, L¹³ is a substituted or unsubstituted C₃-C₈ cycloalkylene. In embodiments, L¹³ is a substituted or unsubstituted 3 to 8 membered heterocycloalkylene. In embodiments, L¹³ is a substituted or unsubstituted C₆-C₁₀ arylene. In embodiments, L¹³ is a substituted or unsubstituted 5 to 10 membered heteroarylene. In embodiments, L¹³ is a substituted C₁-C₈ alkylene. In embodiments, L¹³ is a substituted 2 to 8 membered heteroalkylene. In embodiments, L¹³ is a substituted C₃-C₈ cycloalkylene. In embodiments, L¹³ is a substituted 3 to 8 membered heterocycloalkylene. In embodiments, L¹³ is a substituted C₆-C₁₀ arylene. In embodiments, L¹³ is a substituted 5 to 10 membered heteroarylene. In embodiments, L¹³ is an unsubstituted C₁-C₈ alkylene. In embodiments, L¹³ is an unsubstituted 2 to 8 membered heteroalkylene. In embodiments, L¹³ is an unsubstituted C₃-C₈ cycloalkylene. In embodiments, L¹³ is an unsubstituted 3 to 8 membered heterocycloalkylene. In embodiments, L¹³ is an unsubstituted C₆-C₁₀ arylene. In embodiments, L¹³ is an unsubstituted 5 to 10 membered heteroarylene. In embodiments, L¹³ includes a substituted or unsubstituted cyclooctynyl. In embodiments, L¹³ includes a substituted cyclooctenyl. In embodiments, L¹³ includes a product of a click chemistry reaction. In embodiments, L¹³ includes a product of a click chemistry reaction including the product of the reaction of a cyclooctyne and an azide.

The symbol w1 is an integer from 0 to 10. In embodiments, w1 is an integer from 1 to 10. In embodiments, w1 is 0. In embodiments, w1 is 1. In embodiments, w1 is 2. In embodiments, w1 is 3. In embodiments, w1 is 4. In embodiments, w1 is 5. In embodiments, w1 is 6. In embodiments, w1 is 7. In embodiments, w1 is 8. In embodiments, w1 is 9. In embodiments, w1 is 10.

The symbol y1 is an integer from 0 to 10. In embodiments, y1 is an integer from 1 to 10. In embodiments, y1 is 0. In embodiments, y1 is 1. In embodiments, y1 is 2. In embodiments, y1 is 3. In embodiments, y1 is 4. In embodiments, y1 is 5. In embodiments, y1 is 6. In embodiments, y1 is 7. In embodiments, y1 is 8. In embodiments, y1 is 9. In embodiments, y1 is 10.

The symbol t1 is an integer from 0 to 10. In embodiments, t1 is integer from 1 to 10. In embodiments, t1 is 0. In embodiments, t1 is 1. In embodiments, t1 is 2. In embodiments, t1 is 3. In embodiments, t1 is 4. In embodiments, t1 is 5. In embodiments, t1 is 6. In embodiments, t1 is 7. In embodiments, t1 is 8. In embodiments, t1 is 9. In embodiments, t1 is 10.

In embodiments, a substituted L¹⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁴ is substituted with a plurality of groups selected from substituent groups, size-limited substituent groups, and lower substituent groups; each substituent group, size-limited substituent group, and/or lower substituent group may optionally be different. In embodiments, when L¹⁴ is substituted, it is substituted with at least one substituent group. In embodiments, when L¹⁴ is substituted, it is substituted with at least one size-limited substituent group. In embodiments, when L¹⁴ is substituted, it is substituted with at least one lower substituent group.

In embodiments, L¹⁴ is a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹⁴ is a bond, —NH—, —NHC(O)O—, —O—, —S—, —OC(O)—, —OC(O)NH—, —OC(O)O—, —OSO₂—, —C(O)NH—, —NHC(O)—, —S(O)₂NH—, —NHS(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁴ is a bond, substituted or unsubstituted alkylene, or substituted or unsubstituted heteroalkylene. In embodiments, L¹⁴ is a bond, —(CH₂)_{w}—, or —(CH₂)_{w}—OC(O)—; and w is an integer from 1 to 4. In embodiments, L¹⁴ is a bond. In embodiments, L¹⁴ is substituted or unsubstituted alkylene. In embodiments, L¹⁴ is unsubstituted C₁-C₄ alkylene. In embodiments, L¹⁴ is unsubstituted methylene. In embodiments, L¹⁴ is unsubstituted ethylene. In embodiments, L¹⁴ is unsubstituted propylene. In embodiments, L¹⁴ is unsubstituted n-propylene. In embodiments, L¹⁴ is unsubstituted butylene. In embodiments, L¹⁴ is unsubstituted n-butylene. In embodiments, L¹⁴ is substituted or unsubstituted heteroalkylene. In embodiments, L¹⁴ is —(CH₂)_{w}—, and w is an integer from 1 to 4. In embodiments, L¹⁴ is —(CH₂)_{w}—OC(O)—, and w is an integer from 1 to 4. In embodiments, L¹⁴ is —(CH₂)—OC(O)—. In embodiments, L¹⁴ is —(CH₂)₂—OC(O)—. In embodiments, L¹⁴ is —(CH₂)₃—OC(O)—. In embodiments, L¹⁴ is —(CH₂)₄—OC(O)—.

In embodiments, L¹⁴ is

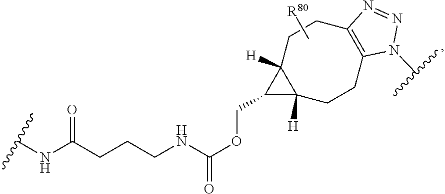

wherein R⁸⁰ is as described herein, including in embodiments. In embodiments, L¹⁴ is

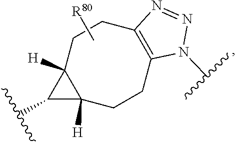

wherein R⁸⁰ is as described herein, including in embodiments. In embodiments, L¹⁴ is

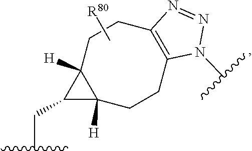

wherein R⁸⁰ is as described herein, including in embodiments. In embodiments, L¹⁴ is

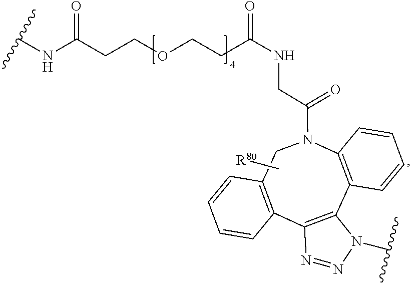

wherein R⁸⁰ is as described herein, including in embodiments. In embodiments, L¹⁴ is

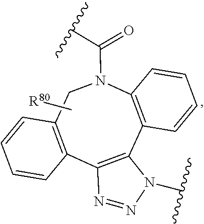

wherein R⁸⁰ is as described herein, including in embodiments. In embodiments, L¹⁴ is

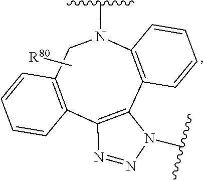

wherein R⁸⁰ is as described herein, including in embodiments. In embodiments, L¹⁴ is

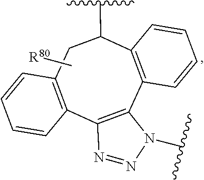

wherein R⁸⁰ is as described herein, including in embodiments. In embodiments, L¹⁴ is

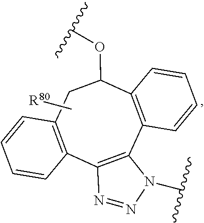

wherein R⁸⁰ is as described herein, including in embodiments. In embodiments, L¹⁴ is —NHC(O)—(CH₂)_{w2}—NHC(O)O—(CH₂)_{y2}—, wherein w2 and y2 are as described herein, including in embodiments. In embodiments, L¹⁴ is —NHC(O)—(CH₂)_{w2}—C(O)NH—(CH₂)_{y2}—, wherein w2 and y2 are as described herein, including in embodiments. In embodiments, L¹⁴ is —NHC(O)—(CH₂)_{w2}—C(O)—, wherein w2 is as described herein, including in embodiments. In embodiments, L¹⁴ is —NHC(O)—(CH₂)_{w2}—NH—, wherein w2 is as described herein, including in embodiments. In embodiments, L¹⁴ is —NHC(O)—(CH₂)_{w2}—NHC(O)—, wherein w2 is as described herein, including in embodiments. In embodiments, L¹⁴ is —NHC(O)—(CH₂)_{w2}—C(O)NH—, wherein w2 is as described herein, including in embodiments. In embodiments, L¹⁴ is —NHC(O)—(CH₂)_{w2}—NHC(O)O—, wherein w2 is as described herein, including in embodiments. In embodiments, L¹⁴ is —NHC(O)—(CH₂)_{w2}—(OCH₂CH₂)ₜ₂—C(O)NH—(CH₂)_{y2}—, wherein w2, t2, and y2 are as described herein, including in embodiments. In embodiments, L¹⁴ is —NHC(O)—(CH₂)_{w2}—(OCH₂CH₂)ₜ₂—C(O)NH—(CH₂)_{y2}—C(O)—, wherein w2, t2, and y2 are as described herein, including in embodiments. In embodiments, L¹⁴ is a substituted or unsubstituted C₁-C₂₀ alkylene. In embodiments, L¹⁴ is a substituted or unsubstituted 2 to 20 membered heteroalkylene. In embodiments, L¹⁴ is a substituted or unsubstituted C₃-C₂₀ cycloalkylene. In embodiments, L¹⁴ is a substituted or unsubstituted 3 to 20 membered heterocycloalkylene. In embodiments, L¹⁴ is a substituted or unsubstituted C₆-C₂₀ arylene. In embodiments, L¹⁴ is a substituted or unsubstituted 5 to 20 membered heteroarylene. In embodiments, L¹⁴ is a substituted C₁-C₂₀ alkylene. In embodiments, L¹⁴ is a substituted 2 to 20 membered heteroalkylene. In embodiments, L¹⁴ is a substituted C₃-C₂₀ cycloalkylene. In embodiments, L¹⁴ is a substituted 3 to 20 membered heterocycloalkylene. In embodiments, L¹⁴ is a substituted C₆-C₂₀ arylene. In embodiments, L¹⁴ is a substituted 5 to 20 membered heteroarylene. In embodiments, L¹⁴ is an unsubstituted C₁-C₂₀ alkylene. In embodiments, L¹⁴ is an unsubstituted 2 to 20 membered heteroalkylene. In embodiments, L¹⁴ is an unsubstituted C₃-C₂₀ cycloalkylene. In embodiments, L¹⁴ is an unsubstituted 3 to 20 membered heterocycloalkylene. In embodiments, L¹⁴ is an unsubstituted C₆-C₂₀ arylene. In embodiments, L¹⁴ is an unsubstituted 5 to 20 membered heteroarylene. In embodiments, L¹⁴ is a substituted or unsubstituted C₁-C₁₄ alkylene. In embodiments, L¹⁴ is a substituted or unsubstituted 2 to 14 membered heteroalkylene. In embodiments, L¹⁴ is a substituted or unsubstituted C₃-C₁₄ cycloalkylene. In embodiments, L¹⁴ is a substituted or unsubstituted 3 to 14 membered heterocycloalkylene. In embodiments, L¹⁴ is a substituted or unsubstituted C₆-C₁₄ arylene. In embodiments, L¹⁴ is a substituted or unsubstituted 5 to 14 membered heteroarylene. In embodiments, L¹⁴ is a substituted C₁-C₁₄ alkylene. In embodiments, L¹⁴ is a substituted 2 to 14 membered heteroalkylene. In embodiments, L¹⁴ is a substituted C₃-C₁₄ cycloalkylene. In embodiments, L¹⁴ is a substituted 3 to 14 membered heterocycloalkylene. In embodiments, L¹⁴ is a substituted C₆-C₁₄ arylene. In embodiments, L¹⁴ is a substituted 5 to 14 membered heteroarylene. In embodiments, L¹⁴ is an unsubstituted C₁-C₁₄ alkylene. In embodiments, L¹⁴ is an unsubstituted 2 to 14 membered heteroalkylene. In embodiments, L¹⁴ is an unsubstituted C₃-C₁₄ cycloalkylene. In embodiments, L¹⁴ is an unsubstituted 3 to 14 membered heterocycloalkylene. In embodiments, L¹⁴ is an unsubstituted C₆-C₁₄ arylene. In embodiments, L¹⁴ is an unsubstituted 5 to 14 membered heteroarylene. In embodiments, L¹⁴ is a substituted or unsubstituted C₁-C₈ alkylene. In embodiments, L¹⁴ is a substituted or unsubstituted 2 to 8 membered heteroalkylene. In embodiments, L¹⁴ is a substituted or unsubstituted C₃-C₈ cycloalkylene. In embodiments, L¹⁴ is a substituted or unsubstituted 3 to 8 membered heterocycloalkylene. In embodiments, L¹⁴ is a substituted or unsubstituted C₆-C₁₀ arylene. In embodiments, L¹⁴ is a substituted or unsubstituted 5 to 10 membered heteroarylene. In embodiments, L¹⁴ is a substituted C₁-C₈ alkylene. In embodiments, L¹⁴ is a substituted 2 to 8 membered heteroalkylene. In embodiments, L¹⁴ is a substituted C₃-C₈ cycloalkylene. In embodiments, L¹⁴ is a substituted 3 to 8 membered heterocycloalkylene. In embodiments, L¹⁴ is a substituted C₆-C₁₀ arylene. In embodiments, L¹⁴ is a substituted 5 to 10 membered heteroarylene. In embodiments, L¹⁴ is an unsubstituted C₁-C₈ alkylene. In embodiments, L¹⁴ is an unsubstituted 2 to 8 membered heteroalkylene. In embodiments, L¹⁴ is an unsubstituted C₃-C₈ cycloalkylene. In embodiments, L¹⁴ is an unsubstituted 3 to 8 membered heterocycloalkylene. In embodiments, L¹⁴ is an unsubstituted C₆-C₁₀ arylene. In embodiments, L¹⁴ is an unsubstituted 5 to 10 membered heteroarylene. In embodiments, L¹⁴ includes a substituted or unsubstituted cyclooctynyl. In embodiments, L¹⁴ includes a substituted cyclooctenyl. In embodiments, L¹⁴ includes a product of a click chemistry reaction. In embodiments, L¹⁴ includes a product of a click chemistry reaction including the product of the reaction of a cyclooctyne and an azide.

R⁸⁰ is hydrogen, oxo, halogen, —CF₃, —CN, —OH, —NH₂, —COOH, —CONH₂, —NO₂, —SH, —SO₂Cl, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R⁸⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸⁰ is substituted with a plurality of groups selected from substituent groups, size-limited substituent groups, and lower substituent groups; each substituent group, size-limited substituent group, and/or lower substituent group may optionally be different. In embodiments, when R⁸⁰ is substituted, it is substituted with at least one substituent group. In embodiments, when R⁸⁰ is substituted, it is substituted with at least one size-limited substituent group. In embodiments, when R⁸⁰ is substituted, it is substituted with at least one lower substituent group.

In embodiments, R⁸⁰ is hydrogen. In embodiments, R⁸⁰ is oxo. In embodiments, R⁸⁰ is halogen. In embodiments, R⁸⁰ is —F. In embodiments, R⁸⁰ is —Cl. In embodiments, R⁸⁰ is —Br. In embodiments, R⁸⁰ is —I. In embodiments, R⁸⁰ is —CF₃. In embodiments, R⁸⁰ is —CN. In embodiments, R⁸⁰ is —OH. In embodiments, R⁸⁰ is —NH₂. In embodiments, R⁸⁰ is —COOH. In embodiments, R⁸⁰ is —CONH₂. In embodiments, R⁸⁰ is —NO₂. In embodiments, R⁸⁰ is —SH. In embodiments, R⁸⁰ is —SO₂Cl. In embodiments, R⁸⁰ is —SO₃H. In embodiments, R⁸⁰ is —OSO₃H. In embodiments, R⁸⁰ is —SO₂NH₂. In embodiments, R⁸⁰ is —NHNH₂. In embodiments, R⁸⁰ is —ONH₂. In embodiments, R⁸⁰ is —NHC(O)NHNH₂. In embodiments, R⁸⁰ is —NHC(O)NH₂. In embodiments, R⁸⁰ is —NHSO₂H. In embodiments, R⁸⁰ is —NHC(O)H. In embodiments, R⁸⁰ is —NHC(O)OH. In embodiments, R⁸⁰ is —NHOH. In embodiments, R⁸⁰ is —OCF₃. In embodiments, R⁸⁰ is —OCHF₂. In embodiments, R⁸⁰ is substituted or unsubstituted C₁-C₄ alkyl. In embodiments, R⁸⁰ is substituted or unsubstituted methyl. In embodiments, R⁸⁰ is substituted or unsubstituted ethyl. In embodiments, R⁸⁰ is substituted or unsubstituted propyl. In embodiments, R⁸⁰ is substituted or unsubstituted n-propyl. In embodiments, R⁸⁰ is substituted or unsubstituted isopropyl. In embodiments, R⁸⁰ is substituted or unsubstituted butyl. In embodiments, R⁸⁰ is substituted or unsubstituted n-butyl. In embodiments, R⁸⁰ is substituted or unsubstituted isobutyl. In embodiments, R⁸⁰ is substituted or unsubstituted tert-butyl. In embodiments, R⁸⁰ is substituted or unsubstituted 2 to 6 membered heteroalkyl. In embodiments, R⁸⁰ is substituted or unsubstituted C₃-C₈ cycloalkyl. In embodiments, R⁸⁰ is substituted or unsubstituted 3 to 8 membered heterocycloalkyl. In embodiments, R⁸⁰ is substituted or unsubstituted phenyl. In embodiments, R⁸⁰ is substituted or unsubstituted 5 to 6 membered heteroaryl.

The symbol w2 is an integer from 0 to 10. In embodiments, w2 is an integer from 1 to 10. In embodiments, w2 is 0. In embodiments, w2 is 1. In embodiments, w2 is 2. In embodiments, w2 is 3. In embodiments, w2 is 4. In embodiments, w2 is 5. In embodiments, w2 is 6. In embodiments, w2 is 7. In embodiments, w2 is 8. In embodiments, w2 is 9. In embodiments, w2 is 10.

The symbol y2 is an integer from 0 to 10. In embodiments, y2 is an integer from 1 to 10. In embodiments, y2 is 0. In embodiments, y2 is 1. In embodiments, y2 is 2. In embodiments, y2 is 3. In embodiments, y2 is 4. In embodiments, y2 is 5. In embodiments, y2 is 6. In embodiments, y2 is 7. In embodiments, y2 is 8. In embodiments, y2 is 9. In embodiments, y2 is 10.

The symbol t2 is an integer from 0 to 10. In embodiments, t2 is integer from 1 to 10. In embodiments, t2 is 0. In embodiments, t2 is 1. In embodiments, t2 is 2. In embodiments, t2 is 3. In embodiments, t2 is 4. In embodiments, t2 is 5. In embodiments, t2 is 6. In embodiments, t2 is 7. In embodiments, t2 is 8. In embodiments, t2 is 9. In embodiments, t2 is 10.

In embodiments, -L¹³-L¹⁴- is a bond, -Ph-(CH₂)_{w}—, or -Ph-(CH₂)_{w}—OC(O)—; and w is an integer from 1 to 4. In embodiments, -L¹³-L¹⁴- is a bond. In embodiments, -L¹³-L¹⁴- is -Ph-(CH₂)_{w}—; and w is an integer from 1 to 4. In embodiments, -L¹³-L¹⁴- is -Ph-(CH₂)_{w}—OC(O)—; and w is an integer from 1 to 4. In embodiments, -L¹³-L¹⁴- is -Ph-CH₂—. In embodiments, -L¹³-L¹⁴- is -Ph-CH₂—OC(O)—.

In embodiments, w is 1. In embodiments, w is 2. In embodiments, w is 3. In embodiments, w is 4.

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁷ is hydrogen. In embodiments, R¹⁷ is unsubstituted C₁-C₄ alkyl. In embodiments, R¹⁷ is unsubstituted methyl. In embodiments, R¹⁷ is unsubstituted ethyl. In embodiments, R¹⁷ is unsubstituted propyl. In embodiments, R¹⁷ is unsubstituted n-propyl. In embodiments, R¹⁷ is unsubstituted isopropyl. In embodiments, R¹⁷ is unsubstituted butyl. In embodiments, R¹⁷ is unsubstituted n-butyl. In embodiments, R¹⁷ is unsubstituted isobutyl. In embodiments, R¹⁷ is unsubstituted tert-butyl.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when R^{5A} and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R^{5A} is hydrogen or unsubstituted C₁-C₄ alkyl. In embodiments, R^{5A} is hydrogen. In embodiments, R^{5A} is unsubstituted C₁-C₄ alkyl. In embodiments, R^{5A} is unsubstituted methyl. In embodiments, R^{5A} is unsubstituted ethyl. In embodiments, R^{5A} is unsubstituted propyl. In embodiments, R^{5A} is unsubstituted n-propyl. In embodiments, R^{5A} is unsubstituted isopropyl. In embodiments, R^{5A} is unsubstituted butyl. In embodiments, R^{5A} is unsubstituted n-butyl. In embodiments, R^{5A} is unsubstituted isobutyl. In embodiments, R^{5A} is unsubstituted tert-butyl.

In embodiments, R^{5B} is hydrogen or unsubstituted C₁-C₄ alkyl. In embodiments, R^{5B} is hydrogen. In embodiments, R^{5B} is unsubstituted C₁-C₄ alkyl. In embodiments, R^{5B} is unsubstituted methyl. In embodiments, R^{5B} is unsubstituted ethyl. In embodiments, R^{5B} is unsubstituted propyl. In embodiments, R^{5B} is unsubstituted n-propyl. In embodiments, R^{5B} is unsubstituted isopropyl. In embodiments, R^{5B} is unsubstituted butyl. In embodiments, R^{5B} is unsubstituted n-butyl. In embodiments, R^{5B} is unsubstituted isobutyl. In embodiments, R^{5B} is unsubstituted tert-butyl.

In embodiments, R^{5C} is hydrogen or unsubstituted C₁-C₄ alkyl. In embodiments, R^{5C} is hydrogen. In embodiments, R^{5C} is unsubstituted C₁-C₄ alkyl. In embodiments, R^{5C} is unsubstituted methyl. In embodiments, R^{5C} is unsubstituted ethyl. In embodiments, R^{5C} is unsubstituted propyl. In embodiments, R^{5C} is unsubstituted n-propyl. In embodiments, R^{5C} is unsubstituted isopropyl. In embodiments, R^{5C} is unsubstituted butyl. In embodiments, R^{5C} is unsubstituted n-butyl. In embodiments, R^{5C} is unsubstituted isobutyl. In embodiments, R^{5C} is unsubstituted tert-butyl.

In embodiments, R^{5D} is hydrogen or unsubstituted C₁-C₄ alkyl. In embodiments, R^{5D} is hydrogen. In embodiments, R^{5D} is unsubstituted C₁-C₄ alkyl. In embodiments, R^{5D} is unsubstituted methyl. In embodiments, R^{5D} is unsubstituted ethyl. In embodiments, R^{5D} is unsubstituted propyl. In embodiments, R^{5D} is unsubstituted n-propyl. In embodiments, R^{5D} is unsubstituted isopropyl. In embodiments, R^{5D} is unsubstituted butyl. In embodiments, R^{5D} is unsubstituted n-butyl. In embodiments, R^{5D} is unsubstituted isobutyl. In embodiments, R^{5D} is unsubstituted tert-butyl.

In embodiments, R⁵ is a drug moiety. In embodiments, R⁵ is a drug moiety bonded to L⁵ through an N of the drug moiety. In embodiments, R⁵ is a drug moiety bonded to L⁵ through an O of the drug moiety. In embodiments, R⁵ is a drug moiety bonded to L⁵ through an S of the drug moiety. In embodiments, R⁵ is a drug moiety bonded to L⁵ through an O of an —OC(O)— of the drug moiety. In embodiments, the drug moiety is a monovalent form of an anti-cancer agent. In embodiments, the drug moiety is a monovalent form of an anti-cancer agent described herein having an N, O, S, or OC(O) group capable of binding the prodrug moiety (e.g., component of the compounds described herein not including a drug moiety, detectable moiety, or protein moiety). In embodiments, the drug moiety is a monovalent form of a topoisomerase inhibitor. In embodiments, the drug moiety is a monovalent form of a topoisomerase I inhibitor. In embodiments, the drug moiety is a monovalent form of a topoisomerase II inhibitor. In embodiments, the drug moiety is a monovalent form of exatecan. In embodiments, the drug moiety is

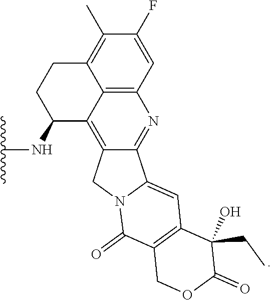 [SMILES: *N[C@H]1CCc2c(C)c(F)cc3nc4c(c1c23)Cn1c-4cc2c(c1=O)COC(=O)[C@]2(O)CC]

In embodiments, the drug moiety is a monovalent form of an ERK inhibitor. In embodiments, the drug moiety is a monovalent form of ASN007. In embodiments, the drug moiety is

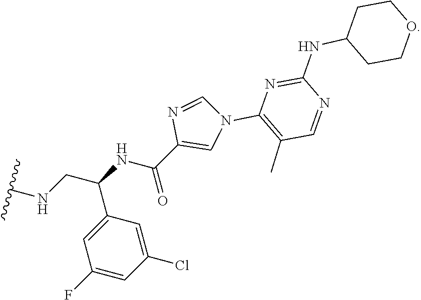 [SMILES: *NC[C@@H](NC(=O)c1cn(-c2nc(NC3CCOCC3)ncc2C)cn1)c1cc(F)cc(Cl)c1]

In embodiments, the drug moiety is a monovalent form of a MEK inhibitor. In embodiments, the drug moiety is a monovalent form of cobimetinib. In embodiments, the drug moiety is

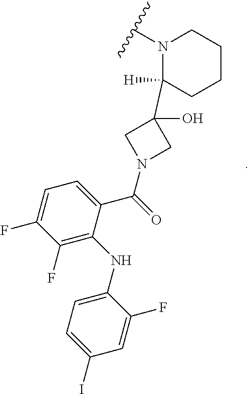 [SMILES: *N1CCCC[C@H]1C1(O)CN(C(=O)c2ccc(F)c(F)c2Nc2ccc(I)cc2F)C1]

In embodiments, the drug moiety is a monovalent form of a PARP inhibitor. In embodiments, the drug moiety is a monovalent form of rucaparib. In embodiments, the drug moiety is

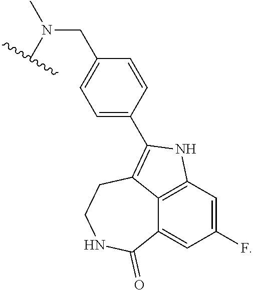 [SMILES: *N(C)Cc1ccc(-c2[nH]c3cc(F)cc4c3c2CCNC4=O)cc1]

In embodiments, the drug moiety is a monovalent form of an anti-infective agent. In embodiments, the drug moiety is a monovalent form of an anti-infective agent described herein having an N, O, S, or OC(O) group capable of binding the prodrug moiety (e.g., component of the compounds described herein not including a drug moiety, detectable moiety, or protein moiety). In embodiments, the anti-infective agent is an anti-parasitic agent. In embodiments, the anti-infective agent is an anti-malarial drug. In embodiments, the drug moiety is a monovalent form of mefloquine. In embodiments, the drug moiety is

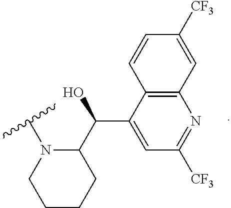 [SMILES: *N1CCCCC1[C@@H](O)c1cc(C(F)(F)F)nc2cc(C(F)(F)F)ccc12]

In embodiments, the anti-infective agent is an anti-bacterial drug. In embodiments, the drug moiety is a monovalent form of ciprofloxacin. In embodiments, the drug moiety is

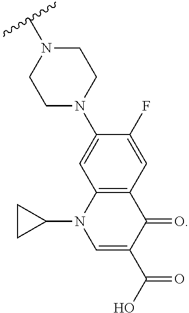 [SMILES: *N1CCN(c2cc3c(cc2F)c(=O)c(C(=O)O)cn3C2CC2)CC1]

In embodiments, R⁵ is

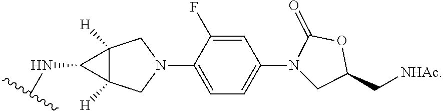 [SMILES: *N[C@H]1[C@@H]2CN(c3ccc(N4C[C@H](CNC(C)=O)OC4=O)cc3F)C[C@@H]21]

In embodiments, R⁵ is

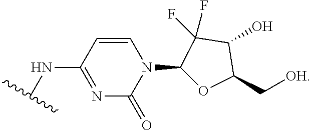 [SMILES: *Nc1ccn([C@@H]2O[C@H](CO)[C@@H](O)C2(F)F)c(=O)n1]

In embodiments, R⁵ is

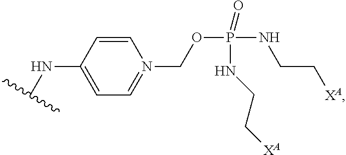

wherein X^{A} is halogen (e.g., Cl or Br). In embodiments, R⁵ is

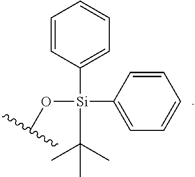 [SMILES: *O[Si](c1ccccc1)(c1ccccc1)C(C)(C)C]

In embodiments, the drug moiety is a monovalent form of a pyrrolo benzodiazepine (e.g., tomaymycin), carboplatin, CC-1065, CC-1065 analog (e.g., amino-CBIs), nitrogen mustard (such as chlorambucil or melphalan), phosphoroamidate mustard, combretastatin, combretastatin analog, puromycin, centanamycin, gemcitabine, dolastatin, dolastatin analog (including auristatin (e.g., monomethyl auristatin E), anthracycline antibiotic (e.g., doxorubicin or daunorubicin), a duocarmycin, duocarmycin analog, enediynes (e.g., neocarzinostatin or calicheamicins), leptomycin derivaties, maytansinoid, maytansinoid analog (e.g., mertansine), methotrexate, mitomycin C, a taxoid, a vinca alkaloid (e.g., vinblastine or vincristine), epothilones, camptothecin, camptothecin analog, topotecan, or irinotecan.

In embodiments, the drug moiety is a monovalent form of amodiaquine, atovaquone, chloroquine, clardribine, clindamycin, cytarabine, daunorubicin, docetaxel, doxorubicin, doxycycline, etoposide, fansidar, fludarabine, halofantrine, idarubicin, imiquimod, irinotecan, mefloquine, methotrexate, mitomycin, oxamniquine, paclitaxel, plicamycin, primaquine, proquanil, pyrimethamine, quinidine, quinine, topotecan, vinblastine, vincristine, KA609, KAF156, tafenoquine, or pyronaridine. In embodiments, the drug moiety is a monovalent form of an anti-bacterial agent described herein. In embodiments, the drug moiety is a monovalent form of an anti-cancer agent described herein. In embodiments, the drug moiety is a monovalent form of an antibody or antigen-binding fragment thereof described herein. In embodiments, the drug moiety is a monovalent form of an anti-malarial agent described herein.

In some embodiments, the agent moiety (e.g., drug moiety, detectable moiety, protein moiety) that forms part of the prodrug is chemically changed under physiological conditions to form an agent (e.g., drug, detectable agent, protein) selected from an anti-cancer agent or anti-infective agent (e.g., antibiotic, anti-parasitic agent, anti-viral agent), detectable agent (e.g., fluorescent agent), or protein (e.g., antibody). Examples of agents include amodiaquine, mefloquine, chloroquine, primaquine, imiquimod, oxamniquine, doxycycline, clindamycin, quinine, quinidine, halofantrine, artesunate, fansidar, atovaquone, pyrimethamine, proguanil, vinblastine, vincristine, daunorubicin, docetaxel, paclitaxel, irinotecan, etoposide, doxorubicin, idarubicin, mitomycin, plicamycin, topotecan, clardribine, cytarabine, fludarabine, and methotrexate. In embodiments, the agent (e.g., drug, detectable agent, protein) moiety that forms part of the prodrug is a moiety as described herein.

In embodiments, R⁵ is a detectable moiety. In embodiments, the detectable moiety is a monovalent form of a fluorophore. In embodiment, R⁵ is a detectable moiety bonded to L⁵ through an N of the detectable moiety. In embodiment, R⁵ is a detectable moiety bonded to L⁵ through an O of the detectable moiety. In embodiment, R⁵ is a detectable moiety bonded to L⁵ through an S of the detectable moiety. In embodiment, R⁵ is a detectable moiety bonded to L⁵ through an O of a —OC(O)— of the detectable moiety.

In embodiments, the detectable moiety is a monovalent form of a fluorescent protein, a xanthene derivative (e.g., fluorescein, rhodamine, Oregon green, eosin, or Texas red), cyanine, a cyanine derivative (e.g., cyanine, indocarbocyanine, oxacarbocyanine, thiacarbocyanine or merocyanine), a naphthalene derivative (e.g., dansyl or prodan or derivatives), coumarin, a coumarin derivative, an oxadiazole derivative (e.g., pyridyloxazole, nitrobenzoxadiazole or benzoxadiazole), an anthracene derivative (e.g., anthraquinones, DRAQ5, DRAQ7, or CyTRAK Orange), a pyrene derivative (e.g., cascade blue and derivatives), an oxazine derivative (e.g., Nile red, Nile blue, cresyl violet, oxazine 170), an acridine derivative (e.g., proflavin, acridine orange, acridine yellow), am arylmethine derivative (e.g., auramine, crystal violet, malachite green), tetrapyrrole derivative (e.g., porphin, phthalocyanine, bilirubin), CF dye™, DRAQ™, CyTRAK™, BODIPY™, an Alexa Fluor™, DyLight Fluor™, Atto™, Tracy™, FluoProbes™, Abberior Dyes™, DY™ dyes, MegaStokes Dyes™, SUlfo Cy™, Seta™ dyes, SeTau™ dyes, Square Dyes™, QuaSar™ dyes, Cal Fluor™ dyes, SureLight Dyes™, PerCP™, Phycobilisomes™, APC™, APCXL™, RPE™, or BPE™. In embodiments, the detectable moiety is a monovalent form of a detectable agent described herein having an N, O, S, or OC(O) group capable of binding the prodrug moiety (e.g., component of the compounds described herein not including a drug moiety, detectable moiety, or protein moiety). In embodiments, the detectable moiety is a moiety described herein.

In embodiments, R⁵ is a protein moiety. In embodiments, the protein moiety is a monovalent form of an antibody. In embodiments, the protein moiety is a peptide moiety. In embodiments, the protein moiety is a modified peptide moiety such as a peptide moiety including folate. In embodiment, R⁵ is a protein moiety bonded to L⁵ through an N of the protein moiety. In embodiment, R⁵ is a protein moiety bonded to L⁵ through an O of the protein moiety. In embodiment, R⁵ is a protein moiety bonded to L⁵ through an S of the protein moiety. In embodiment, R⁵ is a protein moiety bonded to L⁵ through an O of an —OC(O)— of the protein moiety.

In embodiments, the protein moiety is an antibody moiety. In embodiments, the antibody moiety is a monovalent form of bevacizumab, cetuximab, denosumab, ipilimumab, panitumumab, trastuzumab, or catumaxomab. In embodiments, the protein moiety is a monovalent form of a protein described herein having an N, O, S, or OC(O) group capable of binding the prodrug moiety (e.g., component of the compounds described herein not including a drug moiety, detectable moiety, or protein moiety). In embodiments, the protein moiety is a monovalent form of an antibody, or an antigen-binding fragment thereof, described herein.

In embodiments, R⁵ is a siderophore moiety. In embodiments, R⁵ is folate. In embodiments, R⁵ is a folate moiety. In embodiments, R⁵ is a folate derivative. In embodiments, R⁵ is a folate derivative moiety.

In embodiments, when R^{1.1} is substituted, R^{1.1} is substituted with one or more first substituent groups denoted by R^{1.11} as explained in the definitions section above in the description of “first substituent group(s)”. In embodiments, when an R^{1.1.1} substituent group is substituted, the R^{1.11} substituent group is substituted with one or more second substituent groups denoted by R^{1.1.2} as explained in the definitions section above in the description of “first substituent group(s)”. In embodiments, when an R^{1.1.2} substituent group is substituted, the R^{1.1.2} substituent group is substituted with one or more third substituent groups denoted by R^{1.1}. as explained in the definitions section above in the description of “first substituent group(s)”. In the above embodiments, R^{1.1}, R^{1.1.1}, R^{1.1.2}, and R^{1.13} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1.1}, R^{1.1.1}, R^{1.1.2}, and R^{1.13}, respectively.

In embodiments, when R^{1.2} is substituted, R^{1.2} is substituted with one or more first substituent groups denoted by R^{1.2.1} as explained in the definitions section above in the description of “first substituent group(s)”. In embodiments, when an R^{1.2.1} substituent group is substituted, the R^{1.2.1} substituent group is substituted with one or more second substituent groups denoted by R^{1.2.2} as explained in the definitions section above in the description of “first substituent group(s)”. In embodiments, when an R^{1.2.2} substituent group is substituted, the R^{1.2.2} substituent group is substituted with one or more third substituent groups denoted by R^{1.2.3} as explained in the definitions section above in the description of “first substituent group(s)”. In the above embodiments, R^{1.2}, R^{1.2.1}, R^{1.2.2}, and R^{1.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1.2}, R^{1.2.1}, R^{1.2.2}, and R^{1.2.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R^{2.1} is substituted, R^{2.1} is substituted with one or more first substituent groups denoted by R^{2.1.1} as explained in the definitions section above in the description of “first substituent group(s)”. In embodiments, when an R^{2.1.1} substituent group is substituted, the R^{2.1.1} substituent group is substituted with one or more second substituent groups denoted by R^{2.1.2} as explained in the definitions section above in the description of “first substituent group(s)”. In embodiments, when an R^{2.1.2} substituent group is substituted, the R^{2.1.2} substituent group is substituted with one or more third substituent groups denoted by R^{2.1.3} as explained in the definitions section above in the description of “first substituent group(s)”. In the above embodiments, R^{2.1}, R^{2.1.1}, R^{2.1.2}, and R^{2.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.1}, R^{2.1.1}, R^{2.1.2}, and R^{2.1.3}, respectively.

In embodiments, when R^{2.2} is substituted, R^{2.2} is substituted with one or more first substituent groups denoted by R^{2.2.1} as explained in the definitions section above in the description of “first substituent group(s)”. In embodiments, when an R^{2.2.1} substituent group is substituted, the R^{2.2.1} substituent group is substituted with one or more second substituent groups denoted by R^{2.2.2} as explained in the definitions section above in the description of “first substituent group(s)”. In embodiments, when an R^{2.2.2} substituent group is substituted, the R^{2.2.2} substituent group is substituted with one or more third substituent groups denoted by R^{2.2.3} as explained in the definitions section above in the description of “first substituent group(s)”. In the above embodiments, R^{2.2}, R^{2.2.1}, R^{2.2.2}, and R^{2.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.2}, R^{2.2.1}, R^{2.2.2}, and R^{2.2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R^{3.1} is substituted, R^{3.1} is substituted with one or more first substituent groups denoted by R^{1.11} as explained in the definitions section above in the description of “first substituent group(s)”. In embodiments, when an R^{3.1.1} substituent group is substituted, the R^{3.1.1} substituent group is substituted with one or more second substituent groups denoted by R^{3.1.2} as explained in the definitions section above in the description of “first substituent group(s)”. In embodiments, when an R^{3.1.2} substituent group is substituted, the R^{3.1.2} substituent group is substituted with one or more third substituent groups denoted by R^{3.1.3} as explained in the definitions section above in the description of “first substituent group(s)”. In the above embodiments, R^{3.1}, R^{3.1.1}, R^{3.1.2}, and R^{3.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.1}, R^{3.1.1}, R^{3.1.2}, and R^{3.1.3}, respectively.

In embodiments, when R^{3.2} is substituted, R^{3.2} is substituted with one or more first substituent groups denoted by R^{3.2.1} as explained in the definitions section above in the description of “first substituent group(s)”. In embodiments, when an R^{3.2.1} substituent group is substituted, the R^{3.2.1} substituent group is substituted with one or more second substituent groups denoted by R^{3.2.2} as explained in the definitions section above in the description of “first substituent group(s)”. In embodiments, when an R^{3.2.2} substituent group is substituted, the R^{3.2.2} substituent group is substituted with one or more third substituent groups denoted by R^{3.2.3} as explained in the definitions section above in the description of “first substituent group(s)”. In the above embodiments, R^{3.2}, R^{3.2.1}, R^{3.2.2}, and R^{3.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2}, R^{3.2.1}, R^{3.2.2}, and R^{3.2.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R⁵ as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, when R¹⁷ is substituted, R¹⁷ is substituted with one or more first substituent groups denoted by R^{17.1} as explained in the definitions section above in the description of “first substituent group(s)”. In embodiments, when an R^{17.1} substituent group is substituted, the R^{17.1} substituent group is substituted with one or more second substituent groups denoted by R^{17.2} as explained in the definitions section above in the description of “first substituent group(s)”. In embodiments, when an R^{17.2} substituent group is substituted, the R^{17.2} substituent group is substituted with one or more third substituent groups denoted by R^{17.3} as explained in the definitions section above in the description of “first substituent group(s)”. In the above embodiments, R¹⁷, R^{17.1}, R^{17.2}, and R^{17.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁷, R^{17.1}, R^{17.2}, and R^{17.3}, respectively.

In embodiments, when R⁸⁰ is substituted, R⁸⁰ is substituted with one or more first substituent groups denoted by R^{80.1} as explained in the definitions section above in the description of “first substituent group(s)”. In embodiments, when an R^{80.1} substituent group is substituted, the R^{80.1} substituent group is substituted with one or more second substituent groups denoted by R^{80.2} as explained in the definitions section above in the description of “first substituent group(s)”. In embodiments, when an R^{80.2} substituent group is substituted, the R^{80.2} substituent group is substituted with one or more third substituent groups denoted by R^{80.3} as explained in the definitions section above in the description of “first substituent group(s)”. In the above embodiments, R⁸⁰, R^{80.1}, R^{80.2}, and R^{80.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁸⁰, R^{80.1}, R^{80.2}, and R^{80.3}, respectively.

In embodiments, when L⁵ is substituted, L⁵ is substituted with one or more first substituent groups denoted by R^{L5.1} as explained in the definitions section above in the description of “first substituent group(s)”. In embodiments, when an R^{L5.1} substituent group is substituted, the R^{L5.1} substituent group is substituted with one or more second substituent groups denoted by R^{L5.2} as explained in the definitions section above in the description of “first substituent group(s)”. In embodiments, when an R^{L5.2} substituent group is substituted, the R^{L5.2} substituent group is substituted with one or more third substituent groups denoted by R^{L5.3} as explained in the definitions section above in the description of “first substituent group(s)”. In the above embodiments, L⁵, R^{L5.1}, R^{L5.2}, and R^{L5.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L⁵, R^{L5.1}, R^{L5.2}, and R^{L5.3}, respectively.

In embodiments, when L¹³ is substituted, L¹³ is substituted with one or more first substituent groups denoted by R^{L13.1} as explained in the definitions section above in the description of “first substituent group(s)”. In embodiments, when an R^{L13.1} substituent group is substituted, the R^{L13.1} substituent group is substituted with one or more second substituent groups denoted by R^{L13.2} as explained in the definitions section above in the description of “first substituent group(s)”. In embodiments, when an R^{L13.2} substituent group is substituted, the R^{L13.2} substituent group is substituted with one or more third substituent groups denoted by R^{L13.3} as explained in the definitions section above in the description of “first substituent group(s)”. In the above embodiments, L¹³, R^{L13.1}, R^{L13.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹³, R^{L13.1}, R^{L13.2}, and R^{L13.1}, respectively.

In embodiments, when L¹⁴ is substituted, L¹⁴ is substituted with one or more first substituent groups denoted by R^{L14.1} as explained in the definitions section above in the description of “first substituent group(s)”. In embodiments, when an R^{L14.1} substituent group is substituted, the R^{L14.1} substituent group is substituted with one or more second substituent groups denoted by R^{L14.2} as explained in the definitions section above in the description of “first substituent group(s)”. In embodiments, when an R^{L14.2} substituent group is substituted, the R^{L14.2} substituent group is substituted with one or more third substituent groups denoted by R^{L14.3} as explained in the definitions section above in the description of “first substituent group(s)”. In the above embodiments, L¹⁴, R^{L14.1}, R^{L14.2}, and R^{L14.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2} and R^{LWW.3} are L¹⁴, R^{L14.1}, R^{L14.2} and R^{L14.3}, respectively.

In embodiments, the compound is

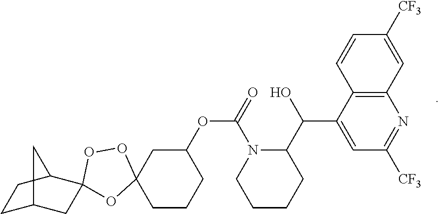 [SMILES: O=C(OC1CCCC2(C1)OOC1(CC3CCC1C3)O2)N1CCCCC1C(O)c1cc(C(F)(F)F)nc2cc(C(F)(F)F)ccc12]

In embodiments, the compound is

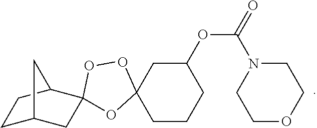 [SMILES: O=C(OC1CCCC2(C1)OOC1(CC3CCC1C3)O2)N1CCOCC1]

In embodiments, the compound is

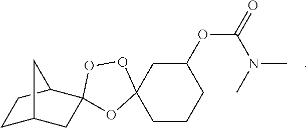 [SMILES: CN(C)C(=O)OC1CCCC2(C1)OOC1(CC3CCC1C3)O2]

In embodiments, the compound is

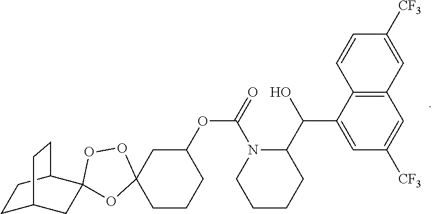 [SMILES: O=C(OC1CCCC2(C1)OOC1(CC3CCC1CC3)O2)N1CCCCC1C(O)c1cc(C(F)(F)F)cc2cc(C(F)(F)F)ccc12]

In embodiments, the compound is

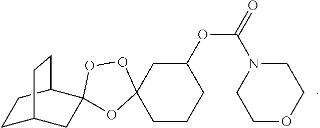 [SMILES: O=C(OC1CCCC2(C1)OOC1(CC3CCC1CC3)O2)N1CCOCC1]

In embodiments, the compound is

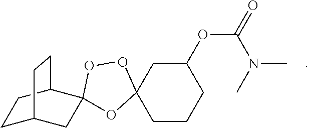 [SMILES: CN(C)C(=O)OC1CCCC2(C1)OOC1(CC3CCC1CC3)O2]

In embodiments, the compound is

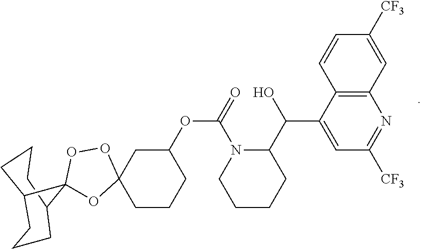 [SMILES: O=C(OC1CCCC2(C1)OOC1(O2)C2CCCC1CCC2)N1CCCCC1C(O)c1cc(C(F)(F)F)nc2cc(C(F)(F)F)ccc12]

In embodiments, the compound is

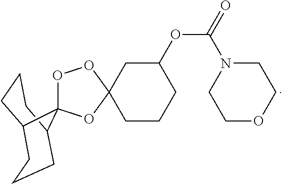 [SMILES: O=C(OC1CCCC2(C1)OOC1(O2)C2CCCC1CCC2)N1CCOCC1]

In embodiments, the compound is

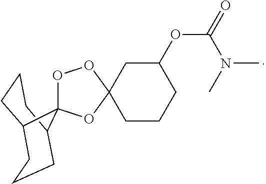 [SMILES: CN(C)C(=O)OC1CCCC2(C1)OOC1(O2)C2CCCC1CCC2]

In embodiments, the compound is

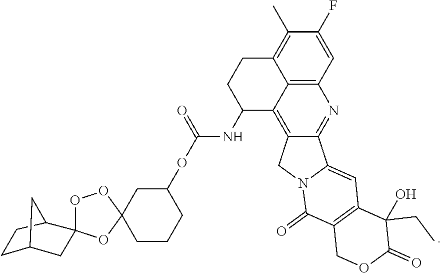 [SMILES: CCC1(O)C(=O)OCc2c1cc1n(c2=O)Cc2c-1nc1cc(F)c(C)c3c1c2C(NC(=O)OC1CCCC2(C1)OOC1(CC4CCC1C4)O2)CC3]

In embodiments, the compound is

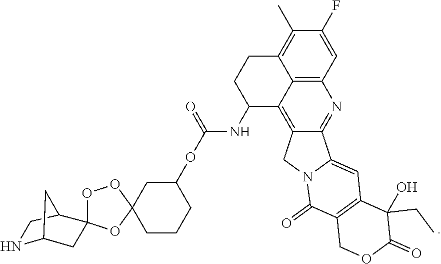 [SMILES: CCC1(O)C(=O)OCc2c1cc1n(c2=O)Cc2c-1nc1cc(F)c(C)c3c1c2C(NC(=O)OC1CCCC2(C1)OOC1(CC4CC1CN4)O2)CC3]

In embodiments, the compound is

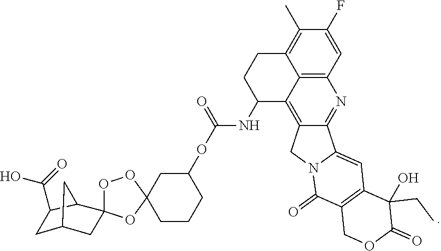 [SMILES: CCC1(O)C(=O)OCc2c1cc1n(c2=O)Cc2c-1nc1cc(F)c(C)c3c1c2C(NC(=O)OC1CCCC2(C1)OOC1(CC4CC(C(=O)O)C1C4)O2)CC3]

In embodiments, the compound is

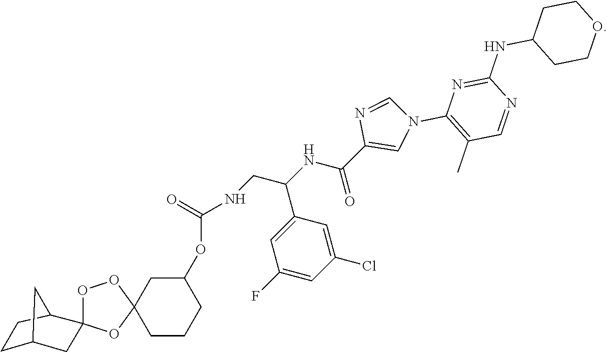 [SMILES: Cc1cnc(NC2CCOCC2)nc1-n1cnc(C(=O)NC(CNC(=O)OC2CCCC3(C2)OOC2(CC4CCC2C4)O3)c2cc(F)cc(Cl)c2)c1]

In embodiments, the compound is

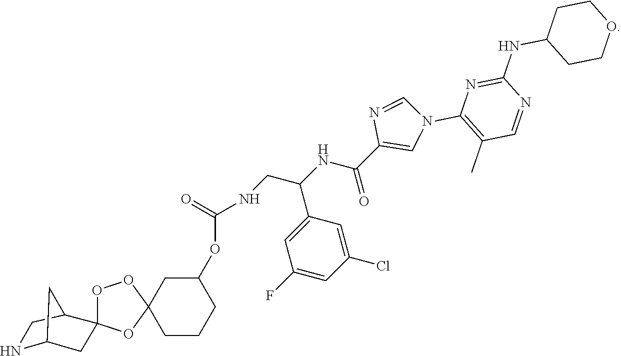 [SMILES: Cc1cnc(NC2CCOCC2)nc1-n1cnc(C(=O)NC(CNC(=O)OC2CCCC3(C2)OOC2(CC4CC2CN4)O3)c2cc(F)cc(Cl)c2)c1]

In embodiments, the compound is

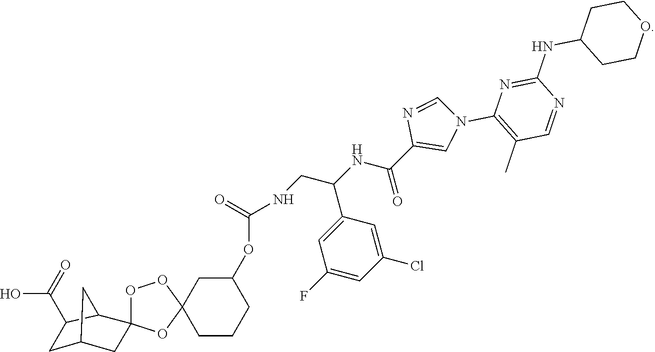 [SMILES: Cc1cnc(NC2CCOCC2)nc1-n1cnc(C(=O)NC(CNC(=O)OC2CCCC3(C2)OOC2(CC4CC(C(=O)O)C2C4)O3)c2cc(F)cc(Cl)c2)c1]

In embodiments, the compound is

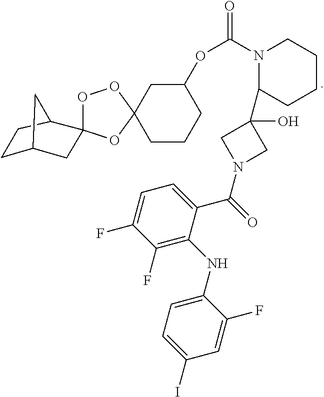 [SMILES: O=C(c1ccc(F)c(F)c1Nc1ccc(I)cc1F)N1CC(O)(C2CCCCN2C(=O)OC2CCCC3(C2)OOC2(CC4CCC2C4)O3)C1]

In embodiments, the compound is

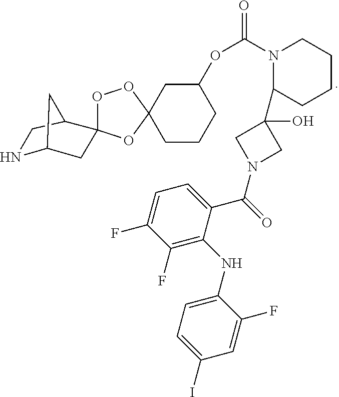 [SMILES: O=C(c1ccc(F)c(F)c1Nc1ccc(I)cc1F)N1CC(O)(C2CCCCN2C(=O)OC2CCCC3(C2)OOC2(CC4CC2CN4)O3)C1]

In embodiments, the compound is

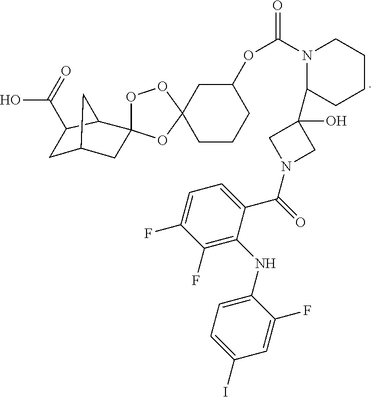 [SMILES: O=C(O)C1CC2CC1C1(C2)OOC2(CCCC(OC(=O)N3CCCCC3C3(O)CN(C(=O)c4ccc(F)c(F)c4Nc4ccc(I)cc4F)C3)C2)O1]

In embodiments, the compound is

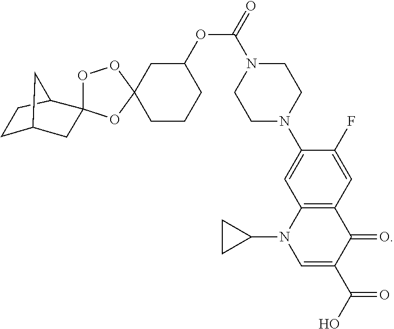 [SMILES: O=C(O)c1cn(C2CC2)c2cc(N3CCN(C(=O)OC4CCCC5(C4)OOC4(CC6CCC4C6)O5)CC3)c(F)cc2c1=O]

In embodiments, the compound is

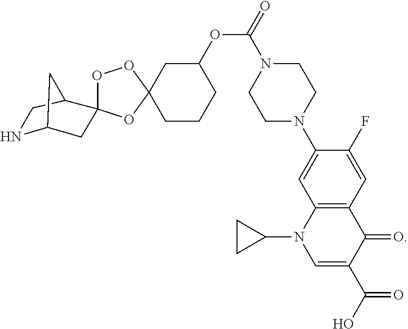 [SMILES: O=C(O)c1cn(C2CC2)c2cc(N3CCN(C(=O)OC4CCCC5(C4)OOC4(CC6CC4CN6)O5)CC3)c(F)cc2c1=O]

In embodiments, the compound is

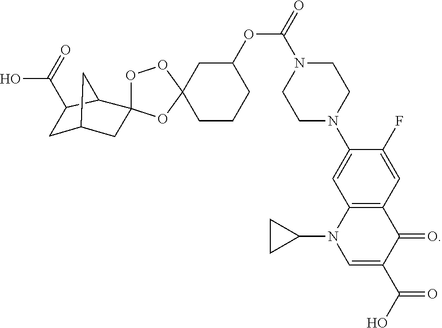 [SMILES: O=C(O)c1cn(C2CC2)c2cc(N3CCN(C(=O)OC4CCCC5(C4)OOC4(CC6CC(C(=O)O)C4C6)O5)CC3)c(F)cc2c1=O]

In embodiments, the compound is

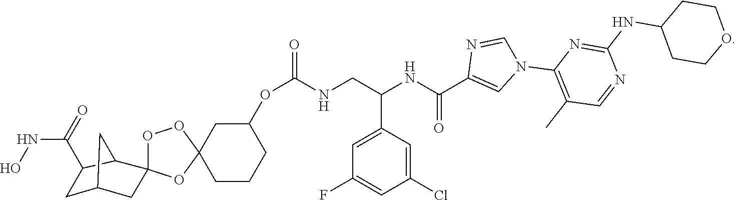 [SMILES: Cc1cnc(NC2CCOCC2)nc1-n1cnc(C(=O)NC(CNC(=O)OC2CCCC3(C2)OOC2(CC4CC(C(=O)NO)C2C4)O3)c2cc(F)cc(Cl)c2)c1]

In embodiments, the compound is

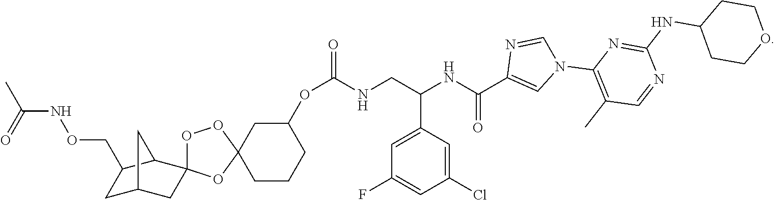 [SMILES: CC(=O)NOCC1CC2CC1C1(C2)OOC2(CCCC(OC(=O)NCC(NC(=O)c3cn(-c4nc(NC5CCOCC5)ncc4C)cn3)c3cc(F)cc(Cl)c3)C2)O1]

In embodiments, the compound is

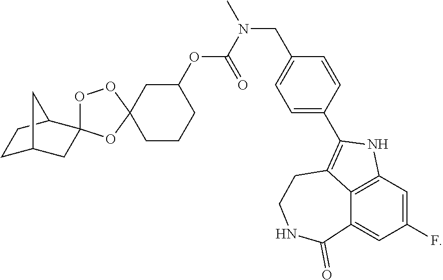 [SMILES: CN(Cc1ccc(-c2[nH]c3cc(F)cc4c3c2CCNC4=O)cc1)C(=O)OC1CCCC2(C1)OOC1(CC3CCC1C3)O2]

In embodiments, the compound is

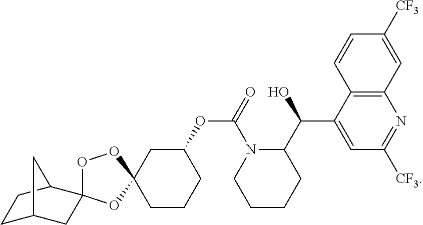

In embodiments, the compound is

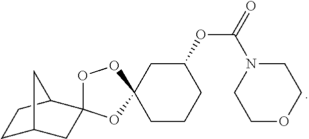

In embodiments, the compound is

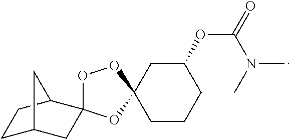

In embodiments, the compound is

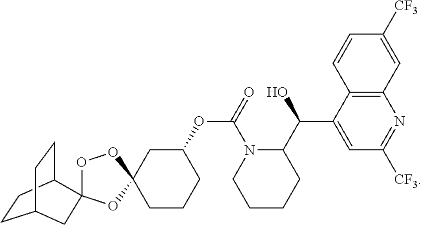

In embodiments, the compound is

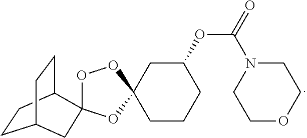

In embodiments, the compound is

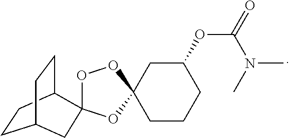

In embodiments, the compound is

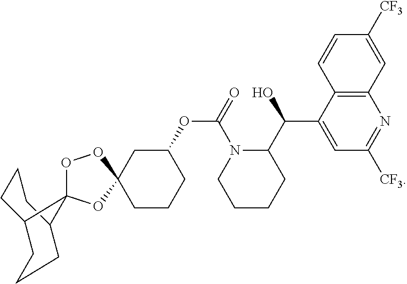

In embodiments, the compound is

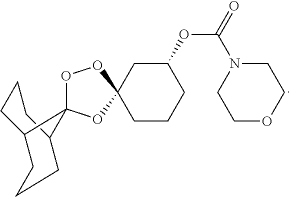

In embodiments, the compound is

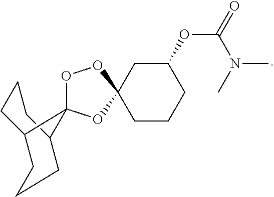

In embodiments, the compound is

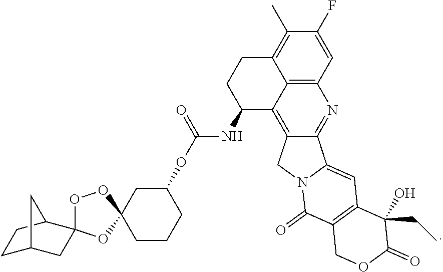

In embodiments, the compound is

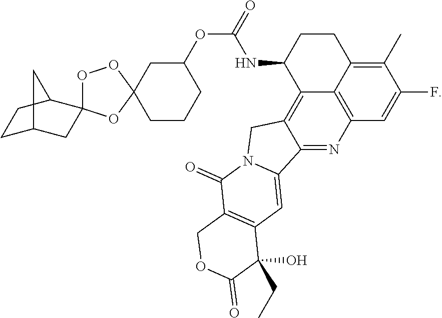 [SMILES: CC[C@@]1(O)C(=O)OCc2c1cc1n(c2=O)Cc2c-1nc1cc(F)c(C)c3c1c2[C@@H](NC(=O)OC1CCCC2(C1)OOC1(CC4CCC1C4)O2)CC3]

In embodiments, the compound is

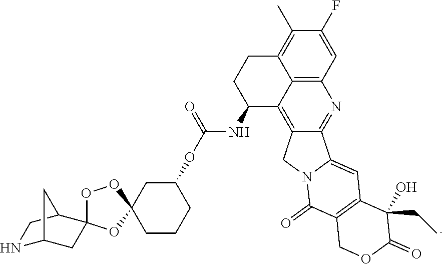

In embodiments, the compound is

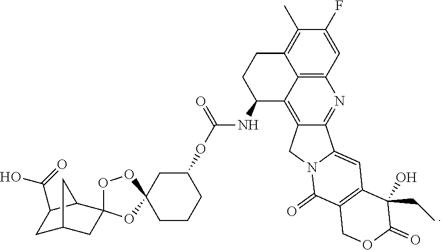

In embodiments, the compound is

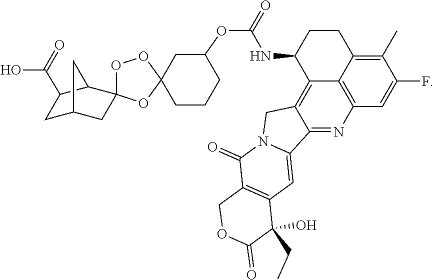 [SMILES: CC[C@@]1(O)C(=O)OCc2c1cc1n(c2=O)Cc2c-1nc1cc(F)c(C)c3c1c2[C@@H](NC(=O)OC1CCCC2(C1)OOC1(CC4CC(C(=O)O)C1C4)O2)CC3]

In embodiments, the compound is

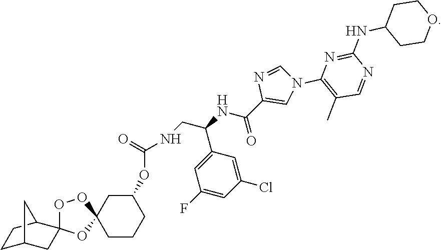

In embodiments, the compound is

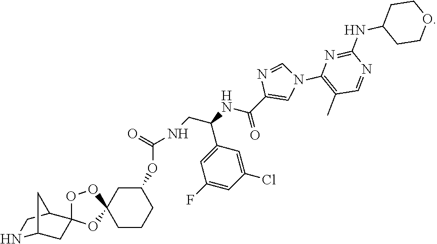

In embodiments, the compound is

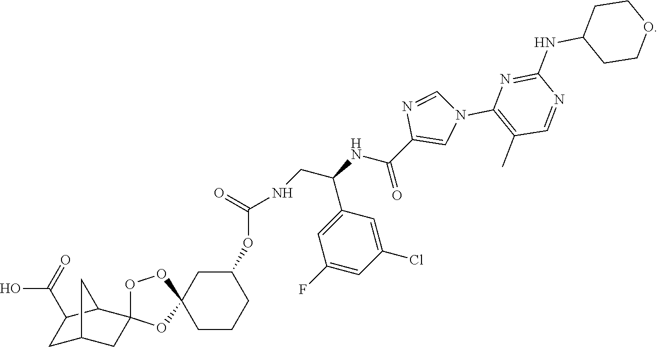

In embodiments, the compound is

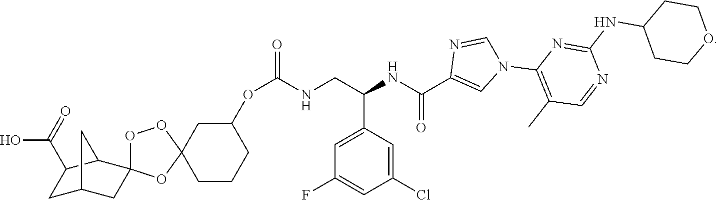 [SMILES: Cc1cnc(NC2CCOCC2)nc1-n1cnc(C(=O)N[C@H](CNC(=O)OC2CCCC3(C2)OOC2(CC4CC(C(=O)O)C2C4)O3)c2cc(F)cc(Cl)c2)c1]

In embodiments, the compound is

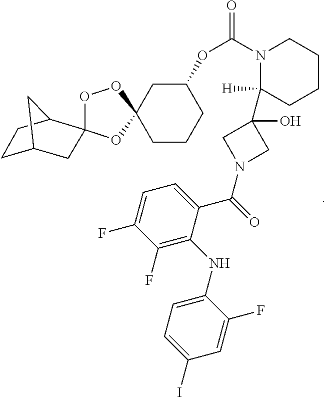

In embodiments, the compound is

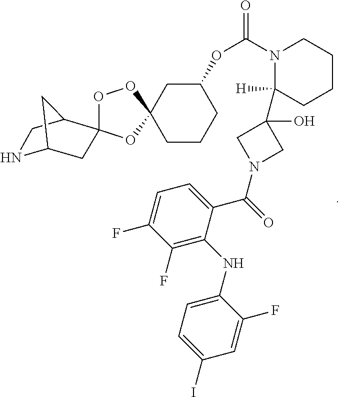

In embodiments, the compound is

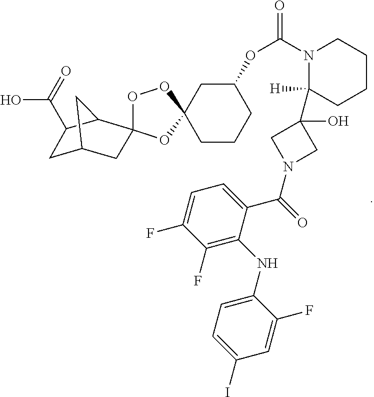

In embodiments, the compound is

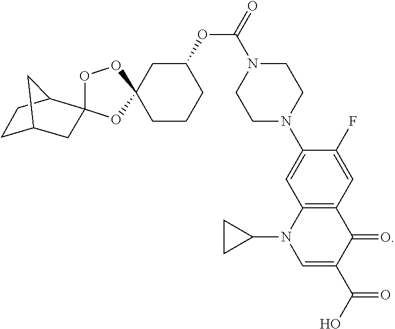

In embodiments, the compound is

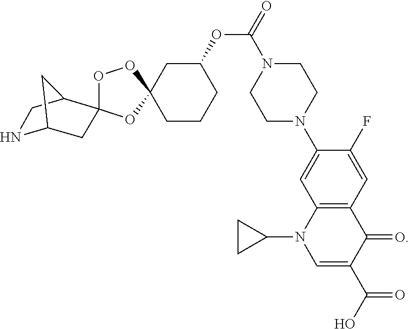

In embodiments, the compound is

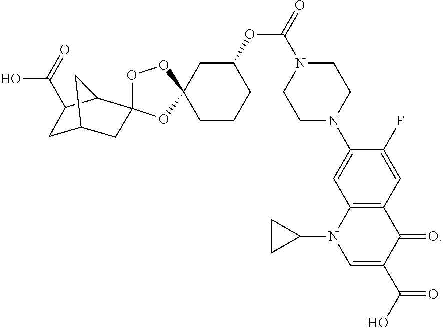

In embodiments, the compound is

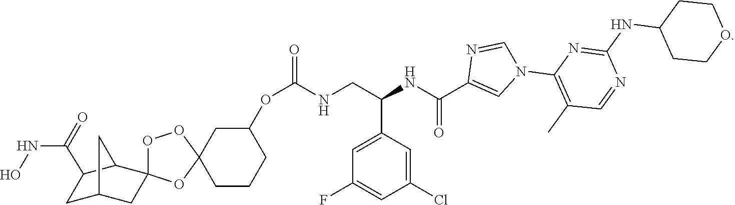 [SMILES: Cc1cnc(NC2CCOCC2)nc1-n1cnc(C(=O)N[C@H](CNC(=O)OC2CCCC3(C2)OOC2(CC4CC(C(=O)NO)C2C4)O3)c2cc(F)cc(Cl)c2)c1]

In embodiments, the compound is

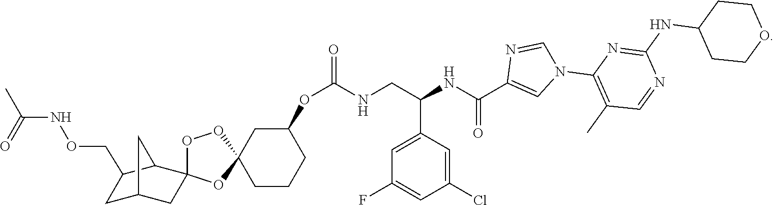

In embodiments, the compound is

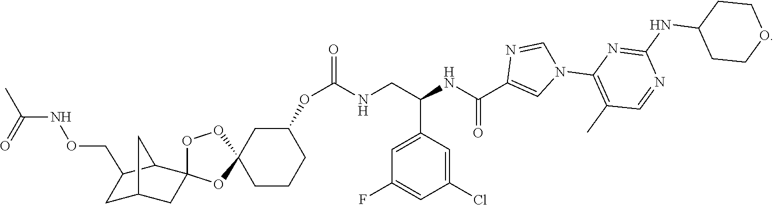

In embodiments, at least one of R^{1.1}, R^{1.2}, R^{2.1}, R^{2.2}, R^{3.1}, R^{3.2}, or R⁴ is not hydrogen.

In embodiments, X is NR^{1.1} and/or Y is NR²¹. In embodiments, X is not CH₂ and Y is not CH₂.

In embodiments, L⁵ is not —OC(O)—.

In embodiments, R⁵ is not

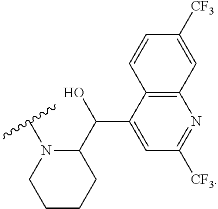 [SMILES: *N1CCCCC1C(O)c1cc(C(F)(F)F)nc2cc(C(F)(F)F)ccc12]

In embodiments, R⁵ is not morpholinyl. In embodiments, R⁵ is not

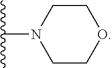 [SMILES: *N1CCOCC1]

In embodiments, the compound is not

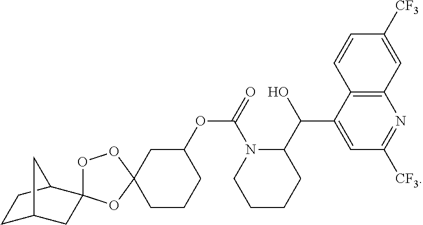 [SMILES: O=C(OC1CCCC2(C1)OOC1(CC3CCC1C3)O2)N1CCCCC1C(O)c1cc(C(F)(F)F)nc2cc(C(F)(F)F)ccc12]

In embodiments, the compound is not

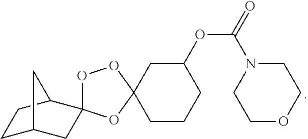 [SMILES: O=C(OC1CCCC2(C1)OOC1(CC3CCC1C3)O2)N1CCOCC1]

In embodiments, the compound is not

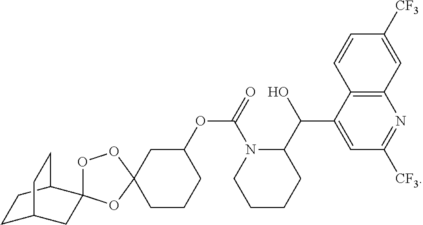 [SMILES: O=C(OC1CCCC2(C1)OOC1(CC3CCC1CC3)O2)N1CCCCC1C(O)c1cc(C(F)(F)F)nc2cc(C(F)(F)F)ccc12]

In embodiments, the compound is not

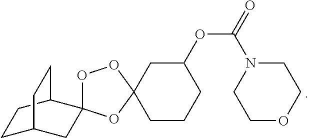 [SMILES: O=C(OC1CCCC2(C1)OOC1(CC3CCC1CC3)O2)N1CCOCC1]

In embodiments, the compound is not

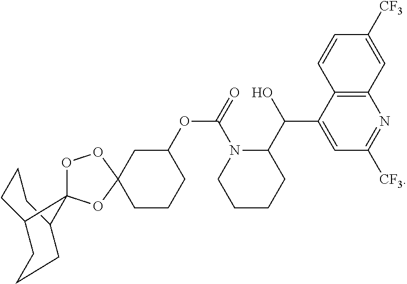 [SMILES: O=C(OC1CCCC2(C1)OOC1(O2)C2CCCC1CCC2)N1CCCCC1C(O)c1cc(C(F)(F)F)nc2cc(C(F)(F)F)ccc12]

In embodiments, the compound is not

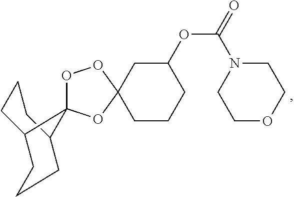 [SMILES: O=C(OC1CCCC2(C1)OOC1(O2)C2CCCC1CCC2)N1CCOCC1]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the compound is a compound of formula (I), (I-1), (1-2), (1-3), (I-4), (I-5), (II), (II-1), (II-2), or (II-3).

### IV. Methods of Use

In an aspect is provided a method of treating a disease in a subject in need thereof, the method including administering to the subject in need thereof a therapeutically effective amount of a compound described herein, or a pharmaceutically acceptable salt thereof.

In embodiments, the compound is a compound of formula (I), (I-1), (1-2), (1-3), (I-4), (I-5), (II), (II-1), (II-2), or (II-3).

In embodiments, the disease is associated with a cell or organism having an increased level of a reductant (e.g., biological reductant, Fe^{II}) compared to a standard control (e.g., subject without the disease or sample from a subject without the disease). In embodiments, the disease is associated with a cell or organism having an increased Fe^{II} level compared to a standard control (e.g., subject without the disease or sample from a subject without the disease). In some embodiments, the method of treating is a method of preventing.

In embodiments, the disease is cancer. In embodiments, the cancer is a hematological cancer. In embodiments, the cancer is a non-hematological cancer. In embodiments, the cancer is pancreatic cancer. In embodiments, the cancer is colon cancer. In embodiments, the cancer is gastrointestinal cancer. In embodiments, the cancer is lung cancer. In embodiments, the cancer is brain cancer. In embodiments, the cancer is leukemia. In embodiments, the cancer is cervical cancer. In embodiments, the cancer is breast cancer. In embodiments, the cancer is ovarian cancer. In embodiments, the cancer is prostate cancer. In embodiments, the cancer is thyroid cancer. In embodiments, the cancer is glioblastoma. In embodiments, the cancer is melanoma.

In embodiments, the disease is a parasitic disease. In embodiments, the parasitic disease is malaria. In embodiments, the parasitic disease is schistosomiasis. In embodiments, the parasitic disease is trypanosomiasis. In embodiments, the parasitic disease is caused by blood-feasting parasites.

In embodiments, the disease is a bacterial disease. In embodiments, the bacterial disease is an Enterococcus spp. bacterial disease, a Staphylococcus spp. bacterial disease, a Klebsiella spp. bacterial disease, an Acinetobacter spp. bacterial disease, a Pseudomonas spp. bacterial disease, or an Enterobacter spp. bacterial disease. In embodiments, the bacterial disease is an Enterococcus faecium bacterial disease. In embodiments, the bacterial disease is a Staphylococcus aureus bacterial disease. In embodiments, the bacterial disease is a Klebsiella pneumoniae bacterial disease. In embodiments, the bacterial disease is an Acinetobacter baumannii bacterial disease. In embodiments, the bacterial disease is a Pseudomonas aeruginosa bacterial disease.

Drug moieties that form part of the prodrugs described herein obtain functionality due to chemical changes in the prodrugs that occur under physiological conditions. For example, the trioxolane ring moiety of prodrugs described herein (i.e., compounds described herein) may react with Fe^{II}, leading to the formation of a cyclohexanone species. The cyclohexanone then undergoes a beta-elimination reaction to release the agent (e.g., drug, detectable agent, protein, sideropohore, or antibody) and a cyclohexenone compound (e.g., side product). The agent (e.g., drug, detectable agent, protein, sideropohore, or antibody) obtained from the prodrug due to chemical changes under physiological conditions may be capable of use in treating or detecting mammalian disease caused by a cell or organism having increased reductant (e.g., biological reductant, Fe^{II}) levels compared to reductant (e.g., biological reductant, Fe^{II}) levels in mammalian plasma. In embodiments, the agent (e.g., drug, detectable agent, protein, sideropohore, or antibody) obtained from the prodrug due to chemical changes under physiological conditions is capable of use in treating or detecting mammalian disease caused by a cell or organism having increased Fe^{II} levels compared to Fe^{II} levels in normal mammalian cells or plasma. The mammalian disease may be a human disease. In some embodiments, the human disease may be a parasitic disease or a cancer. In embodiments, the disease may be malaria, schistosomiasis, trypanosomiasis, leukemia, cervical cancer, breast cancer, colon cancer, ovarian cancer, prostate cancer, thyroid cancer, lung cancer, glioblastoma, or melanoma. In embodiments, the disease may be a cancer where transferrin receptor (CD71) or ferrireductase (STEAP3) are over-expressed as compared to normal cells. In embodiments, the disease may be a bacterial disease. In embodiments, the disease may be an infectious disease.

In another aspect, the prodrug compounds (compounds described herein, including formula I, II, and embodiments) can be employed in methods to treat a disease that is associated with a cell or organism that has increased reductant (e.g., biological reductant, Fe^{II}) levels compared to reductant (e.g., biological reductant, Fe^{II}) levels in the same location in a mammal without the disease (e.g., in mammalian plasma).

In another aspect, the prodrug compounds (compounds described herein, including formula I and embodiments) can be employed in methods to treat a disease that is associated with a cell or organism that has increased Fe^{II} levels compared to Fe^{II} levels in normal mammalian cells or plasma.

In an aspect is provided a method of identifying a subject having a disease associated with a cell or organism having an increased level of a reductant (e.g., biological reductant, Fe^{II}) compared to a standard control (e.g., subject without the disease or sample from a subject without the disease), the method including administering to the subject an effective amount of a compound described herein, or a pharmaceutically acceptable salt thereof.

In an aspect is provided a method of identifying a subject having a disease associated with an increased level of a reductant (e.g., biological reductant, Fe^{II}) compared to a standard control (e.g., subject without the disease or sample from a subject without the disease), the method including: (i) obtaining a biological sample from the subject; (ii) contacting the biological sample with an effective amount of a compound described herein, or a pharmaceutically acceptable salt thereof, wherein the compound includes a detectable moiety; and (iii) detecting an increased level of the detectable moiety or a detectable agent resulting from cleavage of the detectable moiety relative to the level of the detectable moiety or detectable agent in the standard control.

In an aspect is provided a method of detecting a detectable agent (e.g., fluorescent agent) in an organism, by administering a compound described herein to an organism, allowing the organism to metabolize the compound thereby producing a detectable agent (e.g., fluorescent agent), and detecting the detectable agent (e.g., fluorescent agent) in a sample from the organism.

In an aspect is provided a method of detecting a detectable agent (e.g., fluorescent agent) in a sample from an organism, by administering a compound described herein to the sample from an organism, allowing the sample to metabolize the compound thereby producing a detectable agent (e.g., fluorescent agent or reporter), and detecting the detectable agent (e.g., fluorescent agent or reporter) in the sample.

### V. Embodiments

Embodiment P1. A compound, or a pharmaceutically acceptable salt thereof, having the formula:

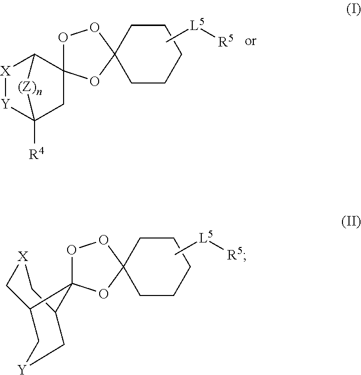

wherein
X is NR^{1.1} or C(R^{1.1}R^{1.2});
Y is NR^{2.1} or C(R^{2.1}R^{2.2});
Z is C(R^{3.1}R^{3.2});
n is 1 or 2;
L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker;
L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1.1} and R^{1.2} are independently hydrogen, oxo, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, oxo, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a protein moiety, a detectable moiety, a siderophore moiety, or a drug moiety;
each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, X⁴, and X⁵ are independently —F, —Cl, —Br, or —I;
n1, n2, n3, n4, and n5 are independently an integer from 0 to 4; and
m1, m2, m3, m4, m5, v1, v2, v3, v4, and v5 are independently 1 or 2.

Embodiment P2. A compound, or a pharmaceutically acceptable salt thereof, having the formula:

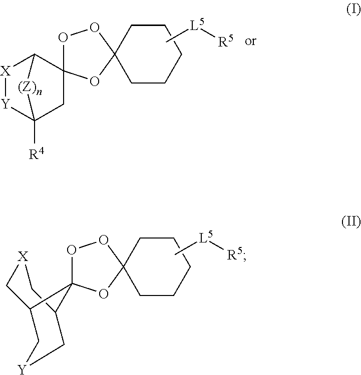

wherein
X is NR^{1.1} or C(R^{1.1}R^{1.2});
Y is NR^{2.1} or C(R^{2.1}R^{2.2});
Z is C(R^{3.1}R^{3.2});
n is 1 or 2;
L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker;
L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1.1} and R^{1.2} are independently hydrogen, oxo, halogen, —CX¹³, —CHX¹², —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)RiC, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, oxo, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a protein moiety, a detectable moiety, or a drug moiety;
each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, X⁴, and X⁵ are independently —F, —Cl, —Br, or —I;
n1, n2, n3, n4, and n5 are independently an integer from 0 to 4; and
m1, m2, m3, m4, m5, v1, v2, v3, v4, and v5 are independently 1 or 2;
wherein the compound is not

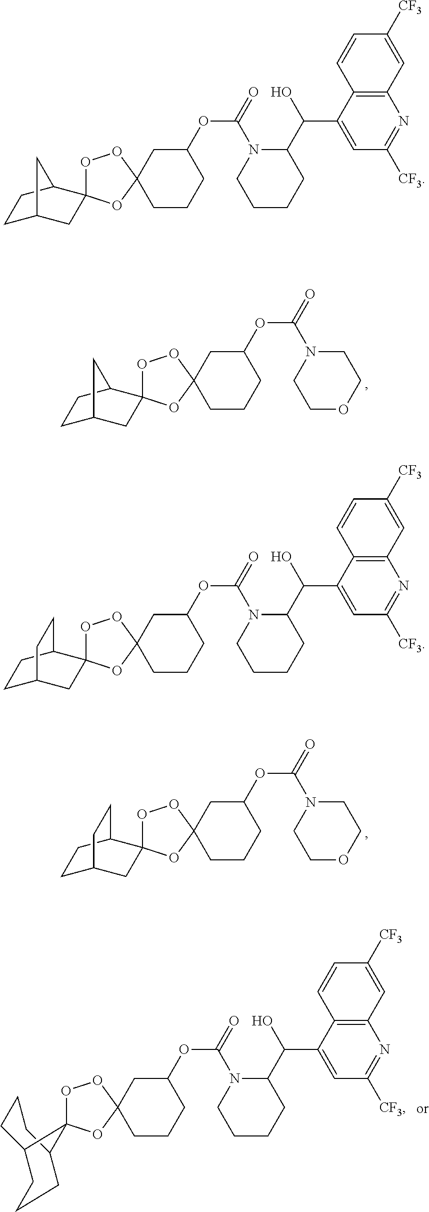 [SMILES: O=C(OC1CCCC2(C1)OOC1(CC3CCC1C3)O2)N1CCCCC1C(O)c1cc(C(F)(F)F)nc2cc(C(F)(F)F)ccc12 ; O=C(OC1CCCC2(C1)OOC1(CC3CCC1C3)O2)N1CCOCC1 ; O=C(OC1CCCC2(C1)OOC1(CC3CCC1CC3)O2)N1CCCCC1C(O)c1cc(C(F)(F)F)nc2cc(C(F)(F)F)ccc12 ; O=C(OC1CCCC2(C1)OOC1(CC3CCC1CC3)O2)N1CCOCC1 ; O=C(OC1CCCC2(C1)OOC1(O2)C2CCCC1CCC2)N1CCCCC1C(O)c1cc(C(F)(F)F)nc2cc(C(F)(F)F)ccc12]

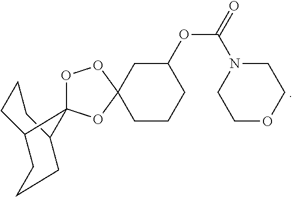 [SMILES: O=C(OC1CCCC2(C1)OOC1(O2)C2CCCC1CCC2)N1CCOCC1]

Embodiment P3. The compound of embodiment P2, wherein at least one of R^{1.1}, R^{1.2}, R^{2.1}, R^{2.2}, R^{3.1}, R^{3.2}, or R⁴ is not hydrogen.

Embodiment P4. The compound of embodiment P2, wherein X is NR^{1.1} and/or Y is NR^{2.1}.

Embodiment P5. The compound of embodiment P2, wherein X is not CH₂ and Y is not CH₂.

Embodiment P6. The compound of one of embodiments P2 to P5, wherein L⁵ is not —OC(O)—.

Embodiment P7. The compound of one of embodiments P2 to P6, wherein R⁵ is not

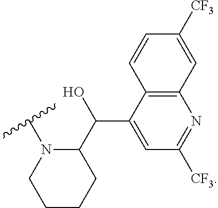 [SMILES: *N1CCCCC1C(O)c1cc(C(F)(F)F)nc2cc(C(F)(F)F)ccc12]

Embodiment P8. The compound of one of embodiments P2 to P6, wherein R⁵ is not morpholinyl.

Embodiment P9. The compound of one of embodiments P2 to P6, wherein R⁵ is not

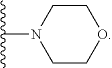 [SMILES: *N1CCOCC1]

Embodiment P10. The compound of one of embodiments P1 to P2, having the formula:

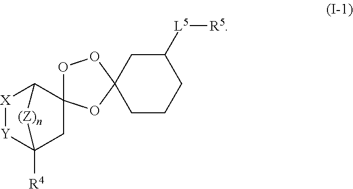

Embodiment P11. The compound of one of embodiments P1 to P2, having the formula:

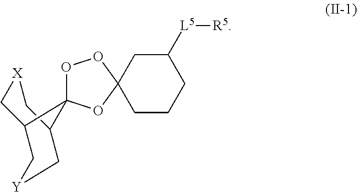

Embodiment P12. The compound of one of embodiments P1 to P2, wherein when X is NR^{1.1}, then Y is C(R^{2.1}R^{2.2}); and when Y is NR^{2.1}, then X is C(R^{1.1}R^{1.2}).

Embodiment P13. The compound of one of embodiments P1 to P12, wherein X is NH.

Embodiment P14. The compound of one of embodiments P1 to P12, wherein X is CHR^{1.2.}

Embodiment P15. The compound of one of embodiments P1 to P14, wherein Y is NH.

Embodiment P16. The compound of one of embodiments P1 to P14, wherein Y is CH₂.

Embodiment P17. The compound of one of embodiments P1 to P16, wherein Z is CH₂.

Embodiment P18. The compound of one of embodiments P1 to P17, wherein R^{1.1} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P19. The compound of one of embodiments P1 to P17, wherein R^{1.1} is hydrogen.

Embodiment P20. The compound of one of embodiments P1 to P19, wherein R^{1.2} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P21. The compound of one of embodiments P1 to P19, wherein R^{1.2} is —C(O)OR^{1C}.

Embodiment P22. The compound of embodiment P21, wherein R^{1C} is hydrogen or unsubstituted C₁-C₄ alkyl.

Embodiment P23. The compound of one of embodiments P1 to P19, wherein R^{1.2} is —C(O)OH.

Embodiment P24. The compound of one of embodiments P1 to P2₃, wherein R^{2.1} and R^{2.2} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P25. The compound of one of embodiments P1 to P23, wherein R^{2.1} and R^{2.2} are hydrogen.

Embodiment P26. The compound of one of embodiments P1 to P25, wherein R^{3.1} and R^{3.2} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P27. The compound of one of embodiments P1 to P25, wherein R^{3.1} and R^{3.2} are hydrogen.

Embodiment P28. The compound of one of embodiments P1 to P27, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P29. The compound of one of embodiments P1 to P27, wherein R⁴ is hydrogen.

Embodiment P30. The compound of one of embodiments P1 to P2, having the formula:

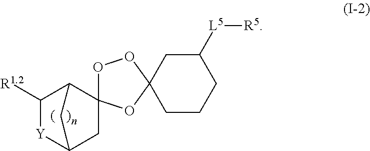

Embodiment P31. The compound of one of embodiments P1 to P2, having the formula:

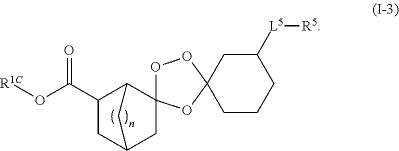

Embodiment P3₂. The compound of one of embodiments P1 to P2, having the formula:

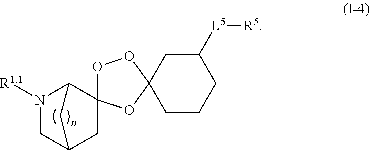

Embodiment P3₃. The compound of one of embodiments P1 to P2, having the formula:

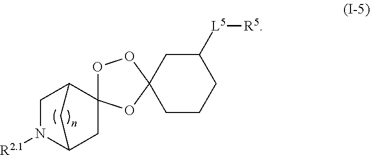

Embodiment P34. The compound of one of embodiments P1 to P2, having the formula:

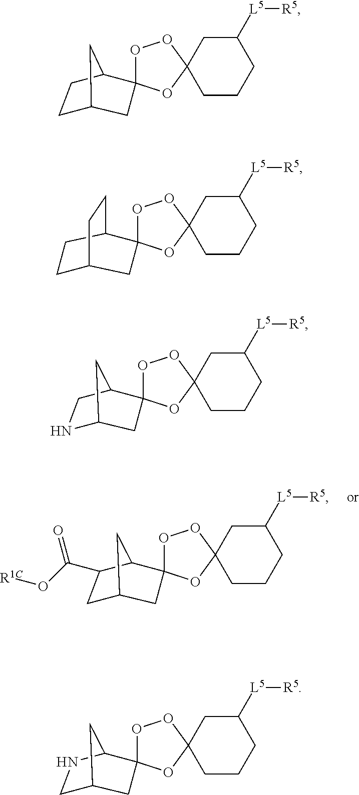

Embodiment P35. The compound of one of embodiments P1 to P2, having the formula:

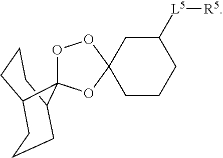

Embodiment P36. The compound of one of embodiments P1 to P2, having the formula:

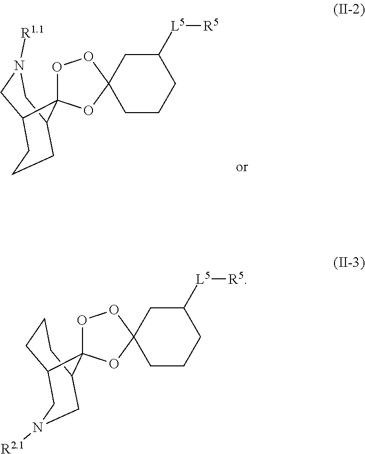

Embodiment P37. The compound of one of embodiments P1 to P2, having the formula:

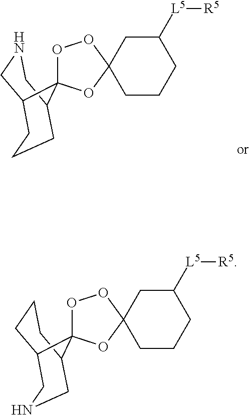

Embodiment P38. The compound of one of embodiments P1 to P35, wherein L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, or —N(R¹⁷)S(O)₂-L¹³-L¹⁴-; and R⁵ is a protein moiety, drug moiety, or a detectable moiety.

Embodiment P39. The compound of one of embodiments P1 to P38, wherein L⁵ is a bond, —N(R¹⁷)-L¹³-L¹⁴-, —O-L¹³-L¹⁴-, —OC(O)-L¹³-L¹⁴-, or —OC(O)N(R¹⁷)-L¹³-L¹⁴-.

Embodiment P40. The compound of one of embodiments P1 to P39, wherein L¹³ is a bond or substituted or unsubstituted arylene.

Embodiment P41. The compound of one of embodiments P1 to P39, wherein L¹³ is a bond or substituted or unsubstituted phenylene.

Embodiment P42. The compound of one of embodiments P1 to P41, wherein L¹⁴ is a bond, substituted or unsubstituted alkylene, or substituted or unsubstituted heteroalkylene.

Embodiment P43. The compound of one of embodiments P1 to P41, wherein L¹⁴ is a bond, —(CH₂)_{w}—, or —(CH₂)_{w}—OC(O)—; and w is an integer from 1 to 4.

Embodiment P44. The compound of embodiment P43, wherein w is 1.

Embodiment P45. The compound of one of embodiments P1 to P39, wherein -L¹³-L¹⁴- is a bond, -Ph-(CH₂)_{w}—, or -Ph-(CH₂)_{w}—OC(O)—; and w is an integer from 1 to 4.

Embodiment P46. The compound of one of embodiments P1 to P39, wherein -L¹³-L¹⁴- is a bond.

Embodiment P47. The compound of one of embodiments P1 to P39, wherein -L¹³-L¹⁴- is -Ph-(CH₂)_{w}—; and w is an integer from 1 to 4.

Embodiment P48. The compound of one of embodiments P1 to P39, wherein -L¹³-L¹⁴- is -Ph-(CH₂)_{w}—OC(O)—; and w is an integer from 1 to 4.

Embodiment P49. The compound of one of embodiments P1 to P38, wherein L⁵ is a bond, —N(R¹⁷)—, —O—, —OC(O)—, or —OC(O)N(R¹⁷)—.

Embodiment P50. The compound of one of embodiments P1 to P49, wherein R⁵ is a drug moiety.

Embodiment P51. The compound of embodiment P50, wherein the drug moiety is a monovalent form of an anti-cancer agent.

Embodiment P52. The compound of embodiment P50, wherein the drug moiety is a monovalent form of an anti-infective agent.

Embodiment P53. The compound of embodiment P52, wherein the anti-infective agent is an anti-parasitic agent.

Embodiment P54. The compound of embodiment P52, wherein the anti-infective agent is an anti-malarial drug.

Embodiment P55. The compound of embodiment P52, wherein the anti-infective agent is an anti-bacterial drug.

Embodiment P56. The compound of one of embodiments P1 to P49, wherein R⁵ is a detectable moiety.

Embodiment P57. The compound of embodiment P56, wherein the detectable moiety is a monovalent form of a fluorophore.

Embodiment P58. The compound of one of embodiments P1 to P49, wherein R⁵ is a protein moiety.

Embodiment P59. The compound of embodiment P58, wherein the protein moiety is a monovalent form of an antibody.

Embodiment P60. A pharmaceutical composition comprising a compound of one of embodiments P1 to P59, or a pharmaceutically acceptable salt thereof, and a pharmaceutically acceptable excipient.

Embodiment P61. A method of treating a disease in a subject in need thereof, said method comprising administering to the subject in need thereof a therapeutically effective amount of a compound of one of embodiments P1 to P59, or a pharmaceutically acceptable salt thereof.

Embodiment P62. The method of embodiment P61, wherein the disease is associated with a cell or organism having an increased Fe^{II} level compared to a standard control.

Embodiment P63. The method of embodiment P61, wherein the disease is cancer.

Embodiment P64. The method of embodiment P63, wherein the cancer is a hematological cancer.

Embodiment P65. The method of embodiment P63, wherein the cancer is a non-hematological cancer.

Embodiment P66. The method of embodiment P63, wherein the cancer is a pancreatic cancer, colon cancer, gastrointestinal cancer, lung cancer, or brain cancer.

Embodiment P67. The method of embodiment P61, wherein the disease is a parasitic disease.

Embodiment P68. The method of embodiment P67, wherein the parasitic disease is malaria.

Embodiment P69. The method of embodiment P67, wherein the parasitic disease is schistosomiasis.

Embodiment P70. The method of embodiment P67, wherein the parasitic disease is caused by blood-feasting parasites.

Embodiment P71. The method of embodiment P61, wherein the disease is a bacterial disease.

Embodiment P72. The method of embodiment P71, wherein the bacterial disease is an Enterococcus spp. bacterial disease, a Staphylococcus spp. bacterial disease, a Klebsiella spp. bacterial disease, an Acinetobacter spp. bacterial disease, a Pseudomonas spp. bacterial disease, or an Enterobacter spp. bacterial disease.

Embodiment P73. The method of embodiment P72, wherein the bacterial disease is an Enterococcus faecium bacterial disease.

Embodiment P74. The method of embodiment P72, wherein the bacterial disease is a Staphylococcus aureus bacterial disease.

Embodiment P75. The method of embodiment P72, wherein the bacterial disease is a Klebsiella pneumoniae bacterial disease.

Embodiment P76. The method of embodiment P72, wherein the bacterial disease is an Acinetobacter baumannii bacterial disease.

Embodiment P77. The method of embodiment P72, wherein the bacterial disease is a Pseudomonas aeruginosa bacterial disease.

Embodiment P78. A method of identifying a subject having a disease associated with a cell or organism having an increased Fe^{II} level compared to a standard control, said method comprising administering to the subject an effective amount of a compound of one of embodiments P1 to P59, or a pharmaceutically acceptable salt thereof.

Embodiment P79. A method of identifying a subject having a disease associated with an increased reductant level compared to a standard control, said method comprising:
(i) obtaining a biological sample from said subject;
(ii) contacting said biological sample with an effective amount of a compound of one of embodiments P1 to P59, or a pharmaceutically acceptable salt thereof, wherein said compound comprises a detectable moiety; and
(iii) detecting an increased level of said detectable moiety or a detectable agent resulting from cleavage of said detectable moiety relative to the level of said detectable moiety or detectable agent in the standard control.

### VI. Additional Embodiments

Embodiment 1. A compound, or a pharmaceutically acceptable salt thereof, having the formula:

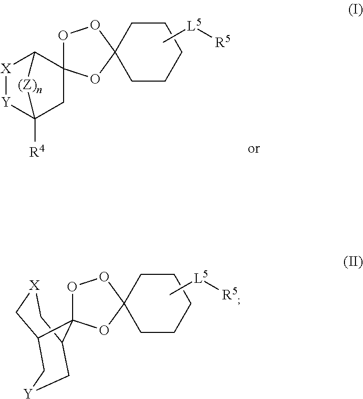

wherein
X is NR^{1.1} or C(R^{1.1}R^{1.2});
Y is NR^{2.1} or C(R^{2.1}R^{2.2});
Z is C(R^{3.1}R^{3.2});
n is 1 or 2;
L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker;
L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1.1} and R^{1.2} are independently hydrogen, oxo, halogen, —CX¹³, —CHX¹², —CH₂X¹, —OCX¹³, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)RiC, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2c}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, oxo, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a protein moiety, a detectable moiety, a siderophore moiety, or a drug moiety;
each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, X⁴, and X⁵ are independently —F, —Cl, —Br, or —I;
n1, n2, n3, n4, and n5 are independently an integer from 0 to 4; and
m1, m2, m3, m4, m5, v1, v2, v3, v4, and v5 are independently 1 or 2.

Embodiment 2. A compound, or a pharmaceutically acceptable salt thereof, having the formula:

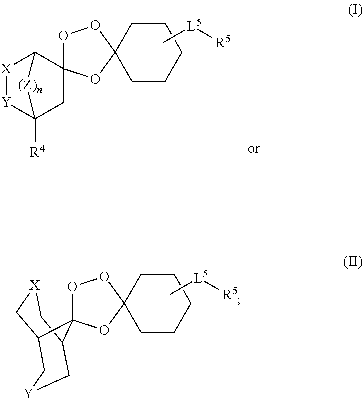

wherein
X is NR^{1.1} or C(R^{1.1}R^{1.2});
Y is NR^{2.1} or C(R^{2.1}R^{2.2});
Z is C(R^{3.1}R^{3.2});
n is 1 or 2;
L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker;
L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1.1} and R^{1.2} are independently hydrogen, oxo, halogen, —CX¹³, —CHX¹², —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, oxo, halogen, —CX⁵₃, —CHX⁵₂, —CH₂X⁵, —OCX⁵₃, —OCH₂X⁵, —OCHX⁵², —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a protein moiety, a detectable moiety, or a drug moiety;
each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, X⁴, and X⁵ are independently —F, —Cl, —Br, or —I;
n1, n2, n3, n4, and n5 are independently an integer from 0 to 4; and
m1, m2, m3, m4, m5, v1, v2, v3, v4, and v5 are independently 1 or 2;
wherein the compound is not

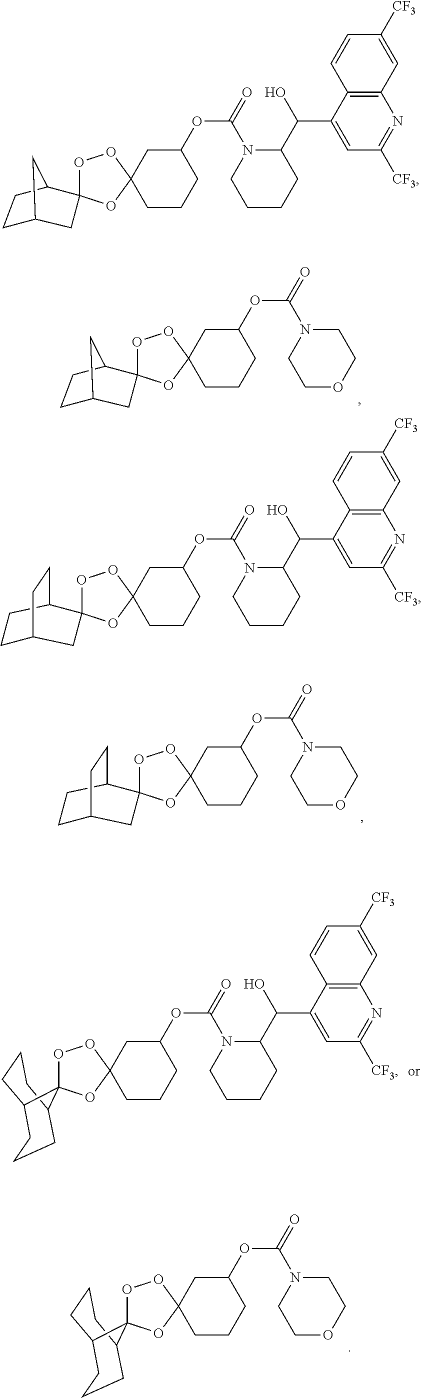 [SMILES: O=C(OC1CCCC2(C1)OOC1(CC3CCC1C3)O2)N1CCCCC1C(O)c1cc(C(F)(F)F)nc2cc(C(F)(F)F)ccc12 ; O=C(OC1CCCC2(C1)OOC1(CC3CCC1C3)O2)N1CCOCC1 ; O=C(OC1CCCC2(C1)OOC1(CC3CCC1CC3)O2)N1CCCCC1C(O)c1cc(C(F)(F)F)nc2cc(C(F)(F)F)ccc12 ; O=C(OC1CCCC2(C1)OOC1(CC3CCC1CC3)O2)N1CCOCC1 ; O=C(OC1CCCC2(C1)OOC1(O2)C2CCCC1CCC2)N1CCCCC1C(O)c1cc(C(F)(F)F)nc2cc(C(F)(F)F)ccc12 ; O=C(OC1CCCC2(C1)OOC1(O2)C2CCCC1CCC2)N1CCOCC1]

Embodiment 3. The compound of embodiment 2, wherein at least one of R¹, R^{1.2}, R^{2.1}, R^{2.2}, R^{3.1}, R^{3.2}, or R⁴ is not hydrogen.

Embodiment 4. The compound of embodiment 2, wherein X is NR^{1.1} and/or Y is NR²¹.

Embodiment 5. The compound of embodiment 2, wherein X is not CH₂ and Y is not CH₂.

Embodiment 6. The compound of one of embodiments 2 to 5, wherein L⁵ is not —OC(O)—.

Embodiment 7. The compound of one of embodiments 2 to 6, wherein R⁵ is not

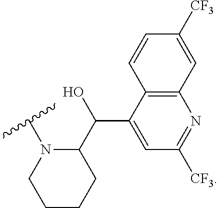 [SMILES: *N1CCCCC1C(O)c1cc(C(F)(F)F)nc2cc(C(F)(F)F)ccc12]

Embodiment 8. The compound of one of embodiments 2 to 6, wherein R⁵ is not morpholinyl.

Embodiment 9. The compound of one of embodiments 2 to 6, wherein R⁵ is not

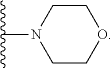 [SMILES: *N1CCOCC1]

Embodiment 10. The compound of one of embodiments 1 to 2, having the formula:

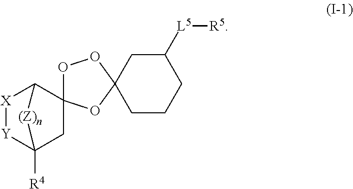

Embodiment 11. The compound of one of embodiments 1 to 2, having the formula:

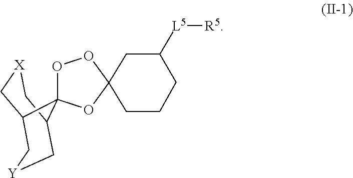

Embodiment 12. The compound of one of embodiments 1 to 2, wherein when X is NR^{1.1}, then Y is C(R^{2.1}R^{2.2}); and when Y is NR^{2.1}, then X is C(R^{1.1}R^{1.2}).

Embodiment 13. The compound of one of embodiments 1 to 12, wherein X is NH.

Embodiment 14. The compound of one of embodiments 1 to 12, wherein X is CHR¹².

Embodiment 15. The compound of one of embodiments 1 to 14, wherein Y is NH.

Embodiment 16. The compound of one of embodiments 1 to 14, wherein Y is CH₂.

Embodiment 17. The compound of one of embodiments 1 to 16, wherein Z is CH₂.

Embodiment 18. The compound of one of embodiments 1 to 17, wherein R^{1.1} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 19. The compound of one of embodiments 1 to 17, wherein R^{1.1} is hydrogen.

Embodiment 20. The compound of one of embodiments 1 to 19, wherein R^{1.2} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 21. The compound of one of embodiments 1 to 19, wherein R^{1.2} is —C(O)OR^{1C}, —C(O)NR^{1A}R^{1B}, or substituted 2 to 6 membered heteroalkyl.

Embodiment 22. The compound of embodiment 21, wherein R^{1C} is hydrogen or unsubstituted C₁-C₄ alkyl.

Embodiment 23. The compound of one of embodiments 1 to 19, wherein R^{1.2} is —C(O)OH, —C(O)NH(OH), or

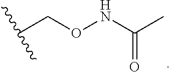 [SMILES: *CONC(C)=O]

Embodiment 24. The compound of one of embodiments 1 to 23, wherein R^{2.1} and R^{2.2} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 25. The compound of one of embodiments 1 to 23, wherein R^{2.1} and R^{2.2} are hydrogen.

Embodiment 26. The compound of one of embodiments 1 to 25, wherein R^{3.1} and R^{3.2} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 27. The compound of one of embodiments 1 to 25, wherein R^{3.1} and R^{3.2} are hydrogen.

Embodiment 28. The compound of one of embodiments 1 to 27, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 29. The compound of one of embodiments 1 to 27, wherein R⁴ is hydrogen.

Embodiment 30. The compound of one of embodiments 1 to 2, having the formula:

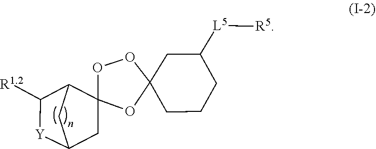

Embodiment 31. The compound of one of embodiments 1 to 2, having the formula:

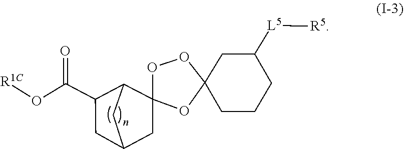

Embodiment 32. The compound of one of embodiments 1 to 2, having the formula:

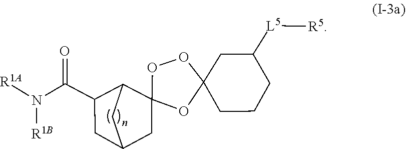

Embodiment 33. The compound of one of embodiments 1 to 2, having the formula:

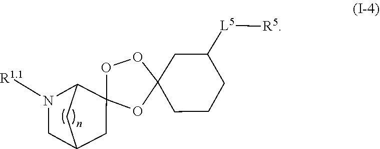

Embodiment 34. The compound of one of embodiments 1 to 2, having the formula:

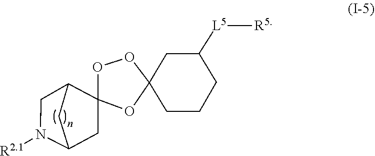

Embodiment 35. The compound of one of embodiments 1 to 2, having the formula:

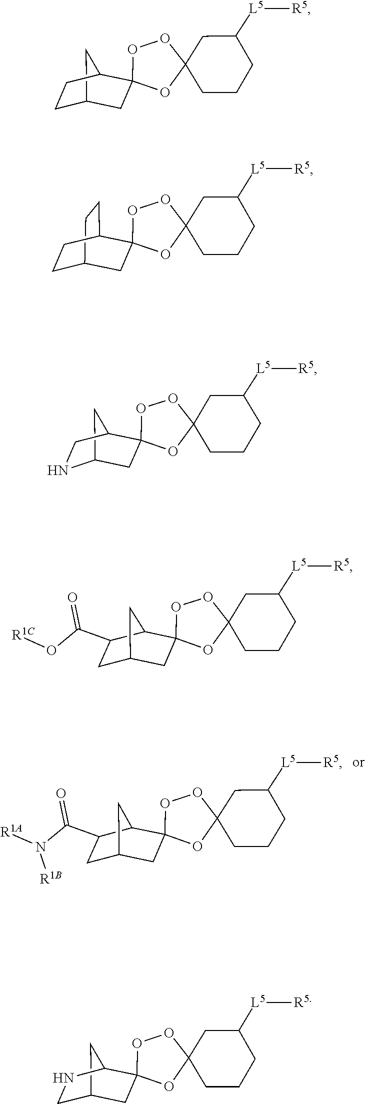

Embodiment 36. The compound of one of embodiments 1 to 2, having the formula:

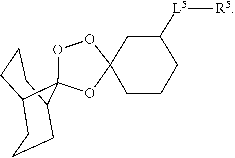

Embodiment 37. The compound of one of embodiments 1 to 2, having the formula:

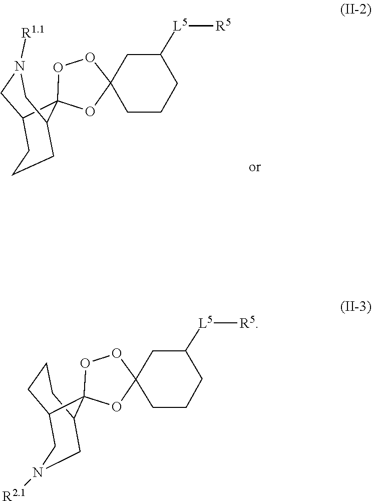

Embodiment 38. The compound of one of embodiments 1 to 2, having the formula:

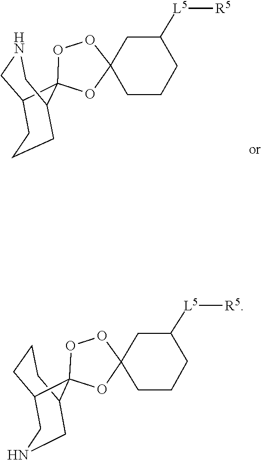

Embodiment 39. The compound of one of embodiments 1 to 36, wherein L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, or —N(R¹⁷)S(O)₂-L¹³-L¹⁴-; and R⁵ is a protein moiety, drug moiety, or a detectable moiety.

Embodiment 40. The compound of one of embodiments 1 to 39, wherein L⁵ is a bond, —N(R¹⁷)-L¹³-L¹⁴-, —O-L¹³-L¹⁴-, —OC(O)-L¹³-L¹⁴-, or —OC(O)N(R¹⁷)-L¹³-L¹⁴-.

Embodiment 41. The compound of one of embodiments 1 to 40, wherein L¹³ is a bond or substituted or unsubstituted arylene.

Embodiment 42. The compound of one of embodiments 1 to 40, wherein L¹³ is a bond or substituted or unsubstituted phenylene.

Embodiment 43. The compound of one of embodiments 1 to 42, wherein L¹⁴ is a bond, substituted or unsubstituted alkylene, or substituted or unsubstituted heteroalkylene.

Embodiment 44. The compound of one of embodiments 1 to 42, wherein L¹⁴ is a bond, —(CH₂)_{w}—, or —(CH₂)_{w}—OC(O)—; and w is an integer from 1 to 4.

Embodiment 45. The compound of embodiment 44, wherein w is 1.

Embodiment 46. The compound of one of embodiments 1 to 40, wherein -L¹³-L¹⁴- is a bond, -Ph-(CH₂)_{w}—, or -Ph-(CH₂)_{w}—OC(O)—; and w is an integer from 1 to 4.

Embodiment 47. The compound of one of embodiments 1 to 40, wherein -L¹³-L¹⁴- is a bond.

Embodiment 48. The compound of one of embodiments 1 to 40, wherein -L¹³-L¹⁴- is -Ph-(CH₂)_{w}—; and w is an integer from 1 to 4.

Embodiment 49. The compound of one of embodiments 1 to 40, wherein -L¹³-L¹⁴- is -Ph-(CH₂)_{w}—OC(O)—; and w is an integer from 1 to 4.

Embodiment 50. The compound of one of embodiments 1 to 39, wherein L⁵ is a bond, —N(R¹⁷)—, —O—, —OC(O)—, or —OC(O)N(R¹⁷)—.

Embodiment 51. The compound of one of embodiments 1 to 50, wherein R⁵ is a drug moiety.

Embodiment 52. The compound of embodiment 51, wherein the drug moiety is a monovalent form of an anti-cancer agent.

Embodiment 53. The compound of embodiment 51, wherein the drug moiety is a monovalent form of an anti-infective agent.

Embodiment 54. The compound of embodiment 53, wherein the anti-infective agent is an anti-parasitic agent.

Embodiment 55. The compound of embodiment 53, wherein the anti-infective agent is an anti-malarial drug.

Embodiment 56. The compound of embodiment 53, wherein the anti-infective agent is an anti-bacterial drug.

Embodiment 57. The compound of one of embodiments 1 to 50, wherein R⁵ is a detectable moiety.

Embodiment 58. The compound of embodiment 57, wherein the detectable moiety is a monovalent form of a fluorophore.

Embodiment 59. The compound of one of embodiments 1 to 50, wherein R⁵ is a protein moiety.

Embodiment 60. The compound of embodiment 59, wherein the protein moiety is a monovalent form of an antibody.

Embodiment 61. A pharmaceutical composition comprising a compound of one of embodiments 1 to 60, or a pharmaceutically acceptable salt thereof, and a pharmaceutically acceptable excipient.

Embodiment 62. A method of treating a disease in a subject in need thereof, said method comprising administering to the subject in need thereof a therapeutically effective amount of a compound of one of embodiments 1 to 60, or a pharmaceutically acceptable salt thereof.

Embodiment 63. The method of embodiment 62, wherein the disease is associated with a cell or organism having an increased Fe^{II} level compared to a standard control.

Embodiment 64. The method of embodiment 62, wherein the disease is cancer.

Embodiment 65. The method of embodiment 64, wherein the cancer is a hematological cancer.

Embodiment 66. The method of embodiment 64, wherein the cancer is a non-hematological cancer.

Embodiment 67. The method of embodiment 64, wherein the cancer is a pancreatic cancer, colon cancer, gastrointestinal cancer, lung cancer, or brain cancer.

Embodiment 68. The method of embodiment 62, wherein the disease is a parasitic disease.

Embodiment 69. The method of embodiment 68, wherein the parasitic disease is malaria.

Embodiment 70. The method of embodiment 68, wherein the parasitic disease is schistosomiasis.

Embodiment 71. The method of embodiment 68, wherein the parasitic disease is caused by blood-feasting parasites.

Embodiment 72. The method of embodiment 62, wherein the disease is a bacterial disease.

Embodiment 73. The method of embodiment 72, wherein the bacterial disease is an Enterococcus spp. bacterial disease, a Staphylococcus spp. bacterial disease, a Klebsiella spp. bacterial disease, an Acinetobacter spp. bacterial disease, a Pseudomonas spp. bacterial disease, or an Enterobacter spp. bacterial disease.

Embodiment 74. The method of embodiment 73, wherein the bacterial disease is an Enterococcus faecium bacterial disease.

Embodiment 75. The method of embodiment 73, wherein the bacterial disease is a Staphylococcus aureus bacterial disease.

Embodiment 76. The method of embodiment 73, wherein the bacterial disease is a Klebsiella pneumoniae bacterial disease.

Embodiment 77. The method of embodiment 73, wherein the bacterial disease is an Acinetobacter baumannii bacterial disease.

Embodiment 78. The method of embodiment 73, wherein the bacterial disease is a Pseudomonas aeruginosa bacterial disease.

Embodiment 79. A method of identifying a subject having a disease associated with a cell or organism having an increased Fe^{II} level compared to a standard control, said method comprising administering to the subject an effective amount of a compound of one of embodiments 1 to 60, or a pharmaceutically acceptable salt thereof.

Embodiment 80. A method of identifying a subject having a disease associated with an increased reductant level compared to a standard control, said method comprising:
(i) obtaining a biological sample from said subject;
(ii) contacting said biological sample with an effective amount of a compound of one of embodiments 1 to 60, or a pharmaceutically acceptable salt thereof, wherein said compound comprises a detectable moiety; and
(iii) detecting an increased level of said detectable moiety or a detectable agent resulting from cleavage of said detectable moiety relative to the level of said detectable moiety or detectable agent in the standard control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Griesbaum Co-Ozonolysis for the Preparation of Structurally Diverse Sensors of Ferrous Iron

Sterically shielded 1,2,4-trioxolanes prepared by Griesbaum co-ozonolysis have been utilized as chemical sensors of ferrous iron in several recently described chemical probes of labile iron. Here we disclose, inter alia, optimized conditions for co-ozonolysis that proceed efficiently and with high diastereoselectivity across an expanded range of substrates, and should enable a new generation of labile iron probes with altered reaction kinetics and physiochemical properties.

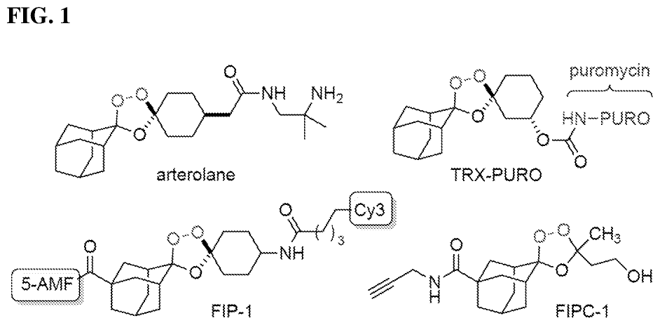

In the mid-1990s (1), Griesbaum and co-workers reported the co-ozonolysis of ketone and ketoxime reactants for the preparation of unsymmetrically substituted 1,2,4-trioxolanes. When cyclic ketone and oxime co-reactants are employed in this process, the resulting trioxolane adducts can be remarkably stable due to shielding of the endoperoxide bond by proximal axial C—H bonds of the surrounding carbocyclic ring systems. Vennerstrom and co-workers exploited this reaction and the shielding effect of the rigid adamantane ring system to develop the antimalarial agents arterolane (2) (FIG. 1) and artefenomel (3). The hindered endoperoxide embedded within their structures, like that in the 1,2,4-trioxoane ring of artemisinin derivatives, confers an antimalarial effect via initial Fenton-type reaction with unbound, or “labile”, ferrous iron sources in the parasite.

Increasing appreciation for the importance of labile iron as the bioavailable pool of iron in the cell has motivated the development of chemical probes capable of detecting iron with oxidation-state specificity (4). Detection of ferrous iron has been achieved almost exclusively through reactivity-based approaches (4c, 4d) in which ferrous iron promotes the reduction of N—O or O—O bonds to activate a fluorophore (e.g., SiRhoNox (5) and related analogs (6)), separate a FRET pair (e.g., TRX-FRET (7), FIP-1 (8)), release a tethered reporter payload (e.g., TRX-PURO (7), ICL-1 (9), HNG (10)), or covalently sequester a PET radionuclide in cells/tissues of animals (¹⁸F-TRX (11, 12)). Trioxane and trioxolane-based reagents have also been employed for chemoproteomic studies of the malaria parasite (13) and of mammalian cancer cells (FIPC-1 (14)). Whilst an arterolane-like pharmacophore has figured prominently in many of these first-generation probes, their further development and optimization is likely to require access to trioxolane systems exhibiting a broader range of iron reactivities and enhanced physiochemical properties for in vivo applications. We therefore reinvestigated the Griesbaum co-ozonolysis with the aim of enabling new structural architectures of potential utility for ferrous iron-reactive therapeutics and chemical probes.

The Griesbaum co-ozonolysis proceeds via [3+2]/retro-[3+2] reaction of the oxime reactant with ozone to afford a carbonyl oxide intermediate. This species then reacts with the ketone component in a final, stereochemistry determining [3+2] cycloaddition to afford 1,2,4-trioxolane adducts. The reaction of adamantane oximes with substituted cyclohexanones is known to proceed selectively via axial addition of carbonyl oxide to ketone, affording cis-4″ or trans-3″ adducts with useful (˜9:1 d.r.) diastereoselectivity (Scheme 1) (15). The seminal reports from Griesbaum described mostly undecorated alkyl and cycloalkyl substrates, whereas more recent work (7, 8, 14, 16) has focused on the adamantane oximes that were found to yield pharmacologically active products, though other groups have explored reactions of non-adamantane substrates (17, 18, 19). We previously reported (15) an optimized protocol to access 3′-hydroxy adducts useful for drug and reporter payload delivery in an iron(II)-dependent fashion. This protocol involved use of the ketone component as limiting reagent, and proceeds in good yields (˜70%) with adamantane oxime at 0° C. in CCl₄. The yields are lower when applied to substituted adamantanes, and particularly to non-adamantyl systems, with yields often in the range of 5-23% and in some cases failing altogether to afford the desired adducts.

Scheme 1. Griesbaum co-ozonolysis proceeds via conversion of the oxime to carbonyl oxide, followed by diastereoselective reaction with the ketone co-reactant, with axial addition favored, as shown.

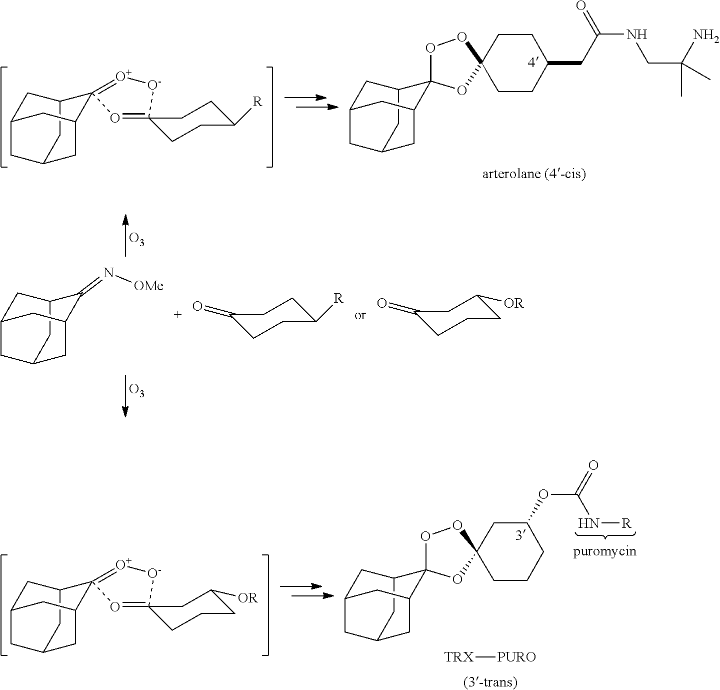

We hypothesized that side reactions of ozone and/or the highly reactive carbonyl oxide intermediate (20) may have contributed to lower yields with certain substrates. To explore this possibility, we evaluated the reaction of enantiopure ketone 2 (21) with a variety of substituted adamantanone oxime substrates under low temperature conditions reported previously (19) for a different substrate (Table 1). We were pleased to find that reactions of 2 with various oximes (3 equiv.) at −78° C. in hexanes, using an oxygen flow rate=1.1 liters per minute, or 6 g/hr O₃, afforded modest (50% for 3b) to excellent (77-94% for 3a, and 3c-3e) isolated yields of the desired adducts (Table 1). By contrast, our previous conditions afforded adduct 3a in acceptable but highly variable yields (48-71% here, previously (21) as high as 91%) while substituted adducts 3b-3e were obtained in poor yield (Table 1). Notably, the diastereofacial selectivity of the final [3+2] cycloaddition is further improved under the low temperature conditions. Thus, adducts 3a-3e were formed as a single trans diastereomer, as shown, whereas ˜10% of the cis diastereomer is formed under the original conditions. As was expected, little diastereofacial selectivity is observed with respect to unsymmetrical carbonyl oxides during the [3+2] cycloaddition (note that only one of the two diastereomers of 3b-3e is shown Table 1).

TABLE 1
Isolated yields of trioxolane adducts obtained under either conventional
reaction condition A (in CCl₄, 0° C.) or low temperature condition B (in hexane, −78° C.).

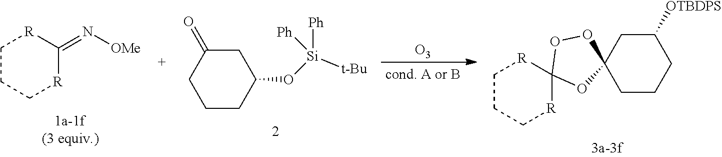

| Product | A: CCl₄, 0° C. | B: hexane, −78 ° C. |
|---|---|---|
| 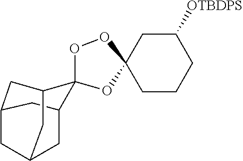 | 48-71% | 91% |
| 3a | | |
| 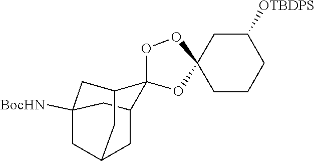 | 28%* | 50% |
| 3b | | |
| 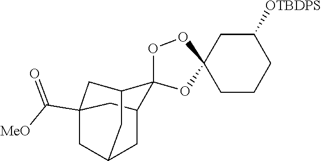 | 23% | 77% |
| 3c | | |
| 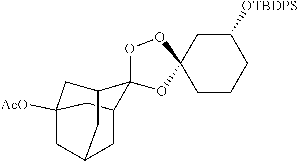 | 5% | 85% |
| 3d | | |
| 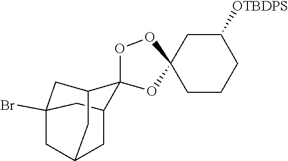 | 18% | 94% |
| 3e | | |
| 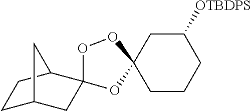 | 0% | |
| 3f | | |

**calculated yield based on ¹H NMR

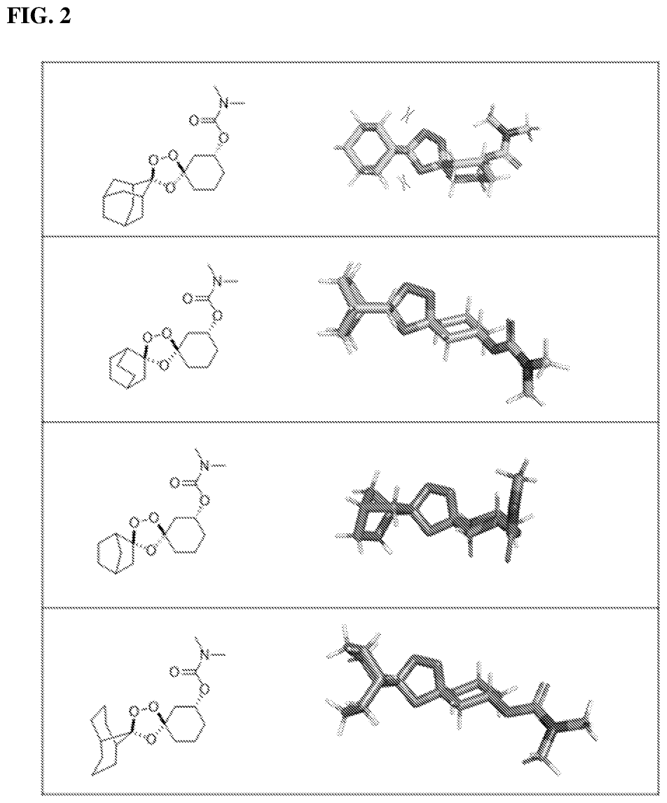

As noted above, the Fenton-type, iron(II)-specific reactivity of reporter-based probes like TRX-PURO and ICL-1 is modulated by the axial C—H bonds surrounding the endoperoxide function (22). To evaluate the potential of other aliphatic bicyclic ring systems to similarly shield the endoperoxide function, we computed minimized conformations of several potential adducts using MarvinSketch software (FIG. 2). From this analysis, we selected the bicyclo[2.2.1]heptane, bicyclo[2.2.2]octane, and bicyclo[3,3,1] nonane ring systems as likely to produce trioxolane adducts with desirable iron reactivity kinetics. Using low temperature reaction condition B, we were pleased to find that co-ozonolysis of 2 with bicyclo[2.2.1]heptan-2-one methyl oxime provided the desired adduct 3f in 81% isolated yield as a mixture of stereoisomers that were partially resolved by ¹H NMR (three distinct resonances in a 13:8:1 ratio). The same reaction failed to afford any isolable amount of 3f under condition A (Table 1).

Buoyed by the successful generation of 3f, we explored the co-ozonolysis of additional oxime substrates under low temperature conditions (Table 2). Thus, reaction of 2 with bicyclo[2.2.2]octan-2-one methyl oxime or bicycle[3.3.1]nonan-9-one methyl oxime afforded the desired adducts 3g and 3h in modest (26-44%) and excellent (80-87%) yields, respectively. The methyl oxime of camphor failed to afford adduct 3i, perhaps due to a more hindered steric environment around the oxime. Substituted cyclohexyl, cyclopentyl, and acyclic oximes were also investigated as substrates in the process. Cyclohexan-1-one oximes substituted at the 4-position afforded the desired adducts 3j (71%) and 31(75%) in good yields, while 2-bromocyclohexan-1-one methyl oxime afforded 3k in 53% yield. Unexpectedly, cyclopentanone oxime substrates failed to yield isolable quantities of the expected adducts 3m, 3n, and 3o. It is possible that the failure of these reactions reflects instability of the trioxolane adducts, or competing reactions of the carbonyl oxide (e.g., dimerization). Similarly, the methyl oximes of acetophenone and 4-methoxyphenylacetone failed to afford useful yields of the expected adducts 3p and 3q.

TABLE 2
Scope of Griesbaum co-ozonolysis involving structurally diverse oximes 1
and ketone 2 under low temperature conditions.

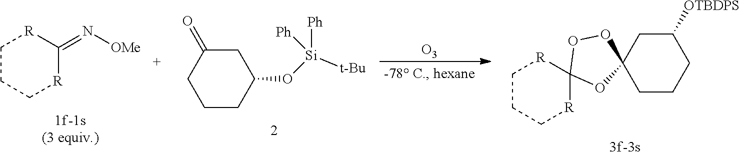

| | Isolated | | Isolated |
|---|---|---|---|
| Product | yield | Product | yield |
| 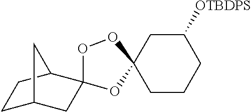 | 70-82% | 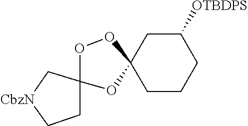 | 0% |
| 3f | | | |
| | | 3m | |
| 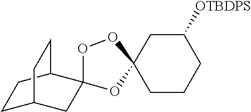 | 26-44% | 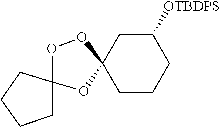 | 0% |
| 3g | | | |
| | | 3n | |
| 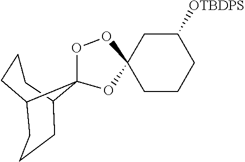 | 80-87% | 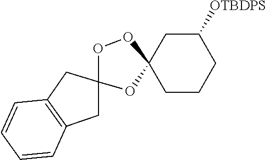 | 0% |
| 3h | | 3o | |
| 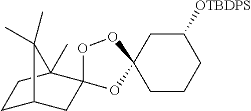 3i | 0% | 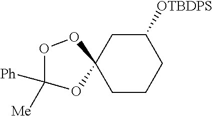 3p | 0% |
| 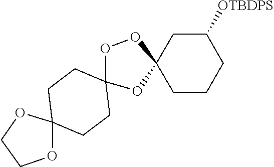 3j | 71% | 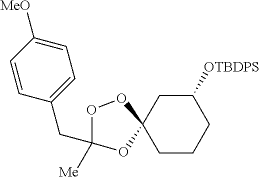 3q | 4% |
| 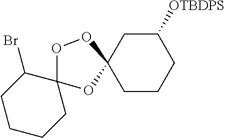 | 53% | 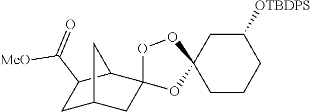 3r | 79% |
| 3k | | | |
| 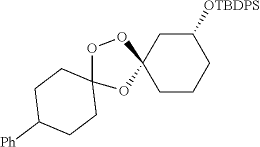 | 75% | 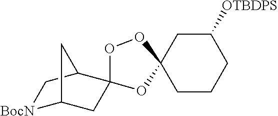 3s | 59% |
| 3l | | | |

Next, we sought to evaluate the Fe²⁺ reactivity of novel adducts like 3f, 3g, and 3h in the context of payload bearing trioxolane conjugates. Using conditions described previously by our group (23) for the conversion of 3a to the mefloquine conjugate TRX-MFQ, 3f-3h were similarly converted to iron(II)-sensitive conjugates of mefloquine (MFQ) and morpholine (Scheme 2). Hence, intermediates 3f-h were treated with TBAF in THF to afford the alcohols 4f-h, which were then converted immediately to the para-nitrophenyl carbonate intermediates 5f-h before a final coupling with either mefloquine to afford 6f-h or morpholine to afford 7f-h (Scheme 2).

Scheme 2. Synthesis of mefloquine and morpholine conjugates 6f-6h and 7f-7h from intermediates 3f-3h. MFQ=mefloquine, an antimalarial drug with a secondary amine function serving as the site of conjugation.

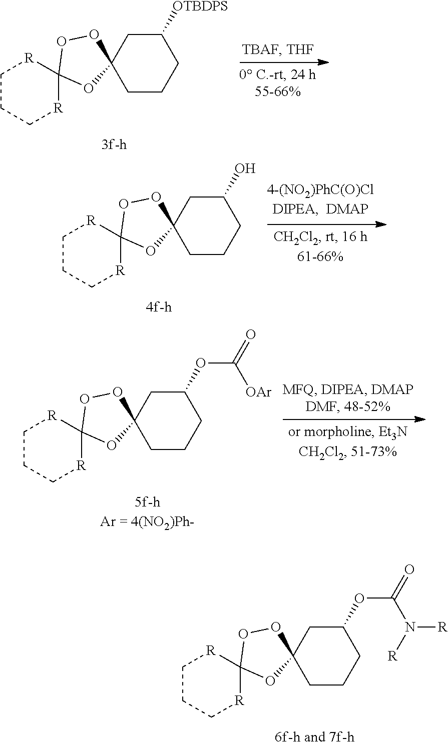

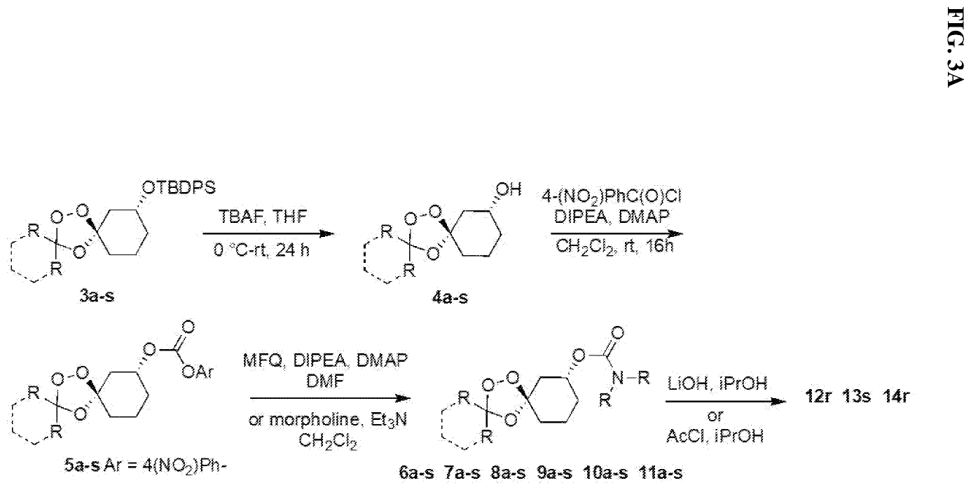

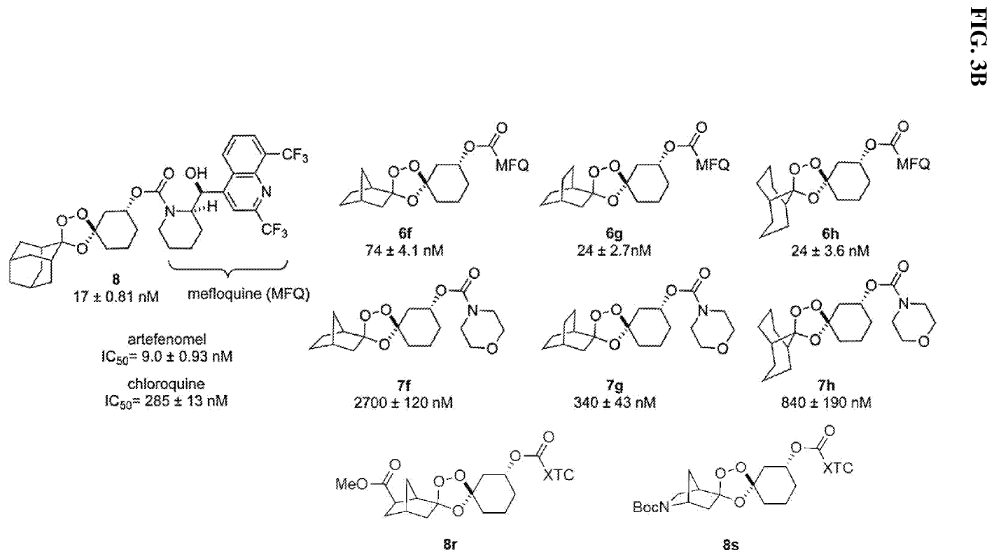

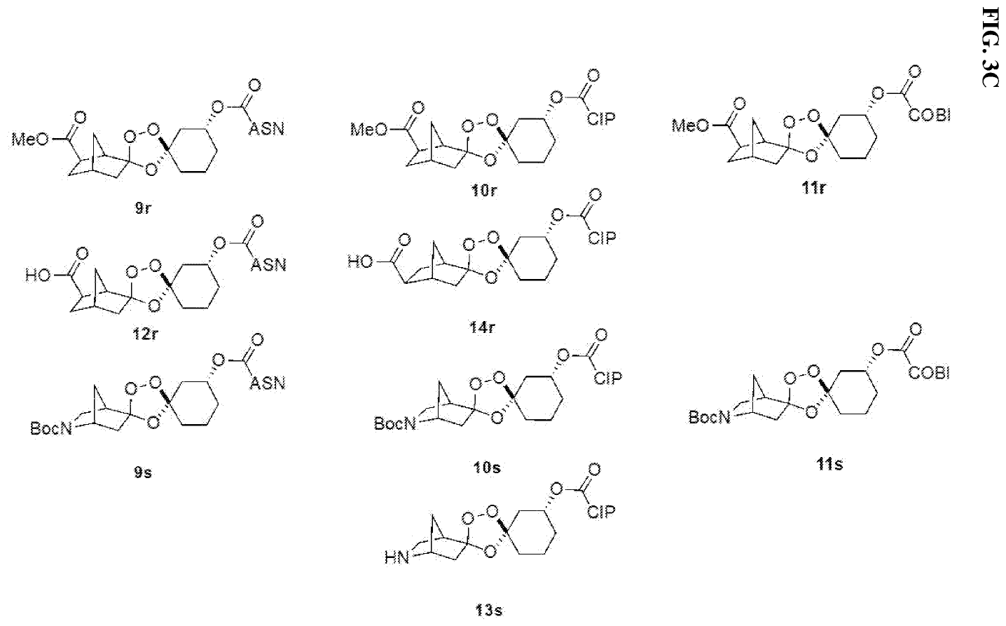

As a surrogate measure of Fe²⁺ reactivity under physiological conditions, and to evaluate their ability to undergo iron(II)-dependent payload release, we evaluated 6f-6h and 7f-7h along with TRX-MFQ (2₃) (6a) as positive control, for activity against cultured P. falciparum parasites (W2 strain) using a standard protocol (FIG. 3B) (24). Mefloquine bearing conjugates 6f-6h exhibited potent IC₅₀ values of 74 nM (for 6f) and 24 nM (6g and 6h), which were similar to that of the positive control 6a (IC₅₀=17 nM). By contrast, morpholine-bearing conjugates 7f-7h were markedly less potent, with IC₅₀ values between 340 and 2700 nM (FIG. 3B), which is 10-100 fold weaker than observed previously for congeneric adamantane-derived trioxolane comparators with a morpholine side chain (21). Accordingly, the potent anti-plasmodial activities of 6f-6h can be inferred to result from iron(II)-dependent activation and release of the mefloquine payload by the canonical mechanism (7, 25) of payload release from “TRX” conjugates.

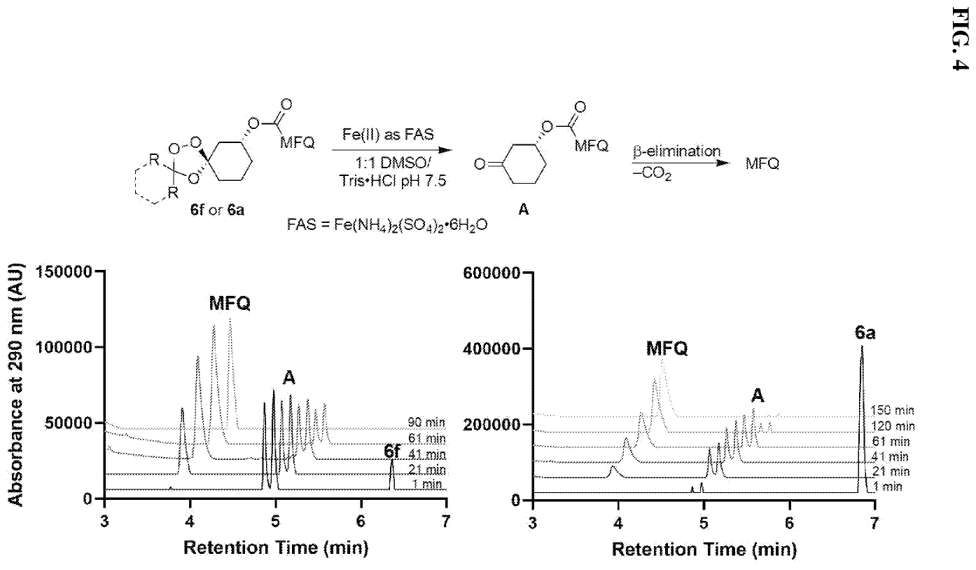

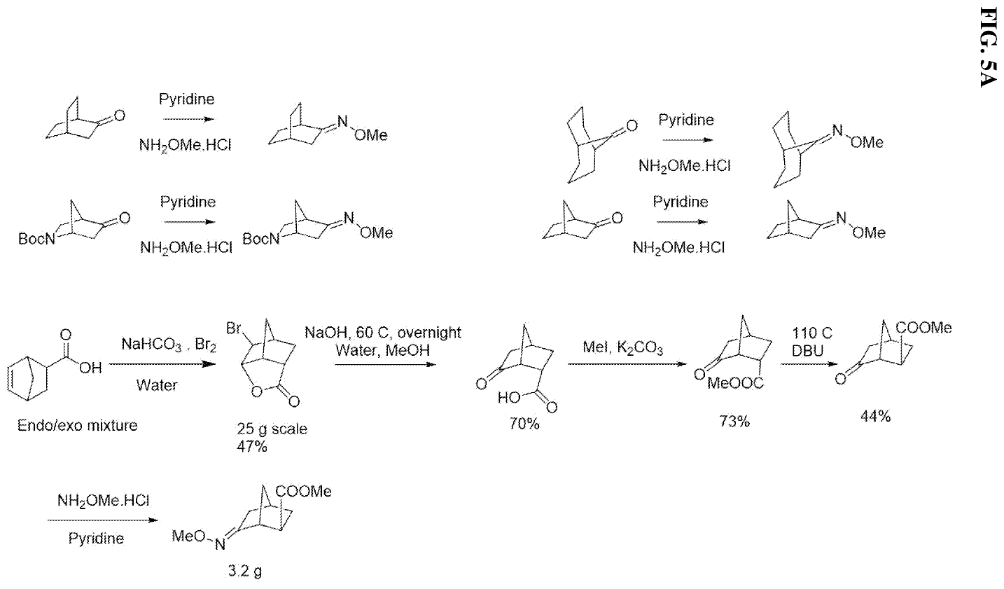

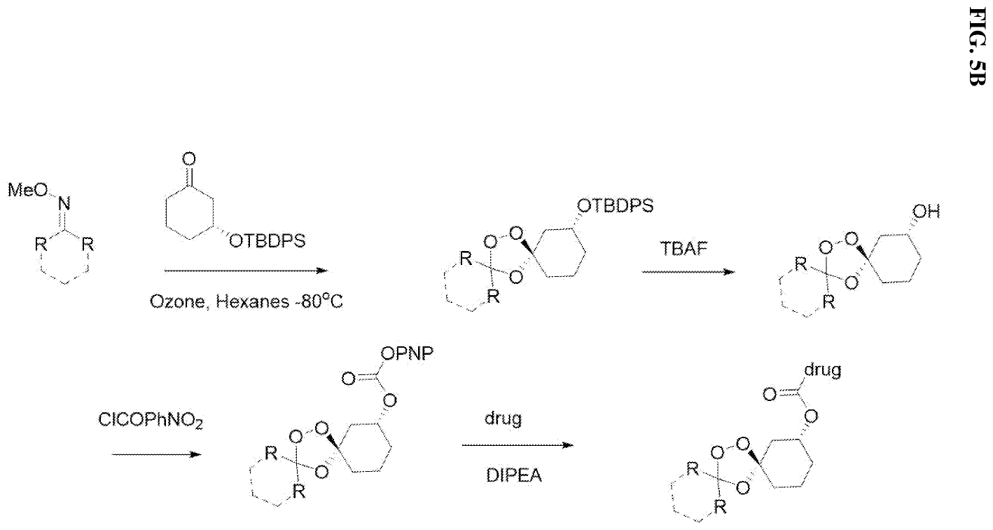

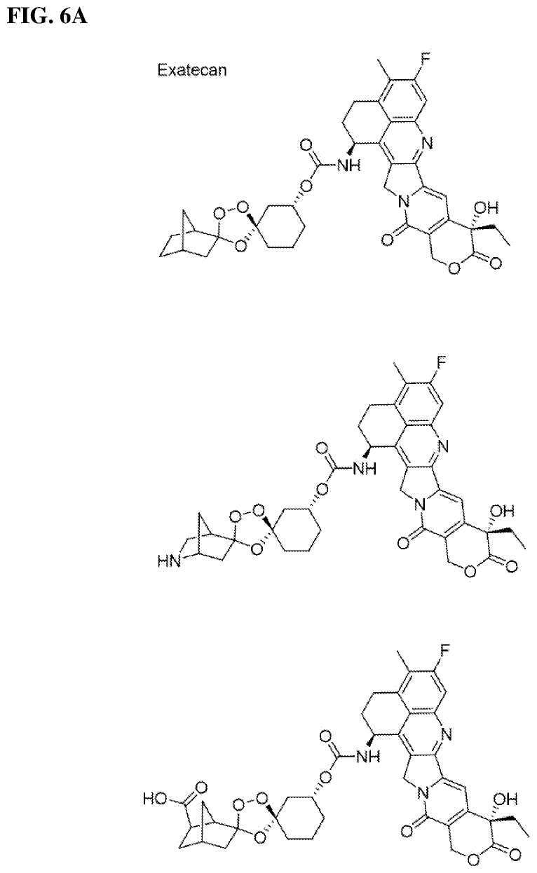

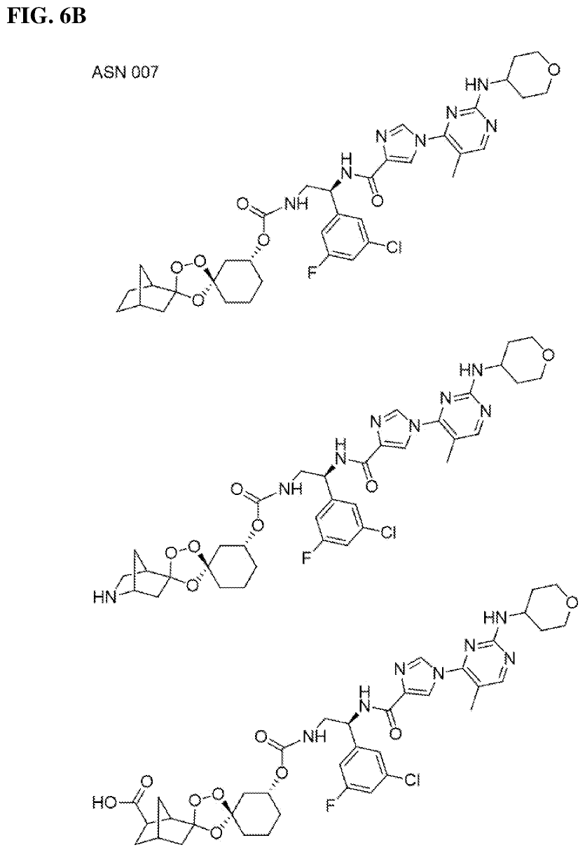

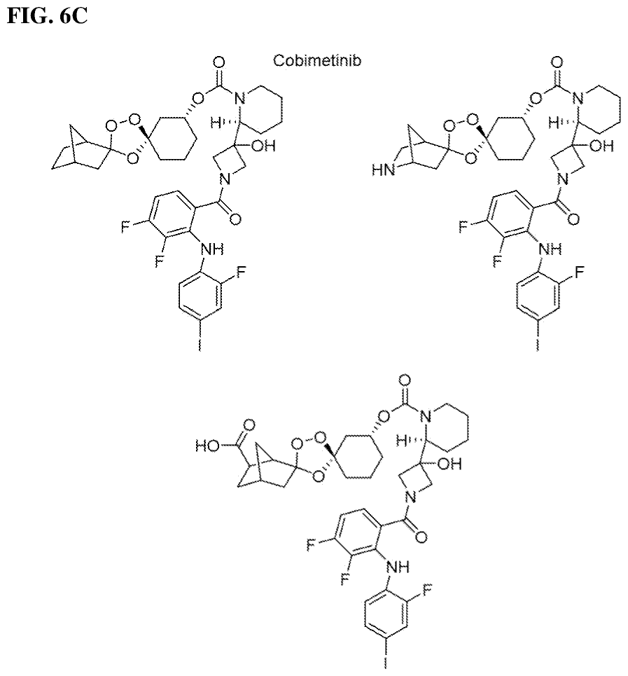

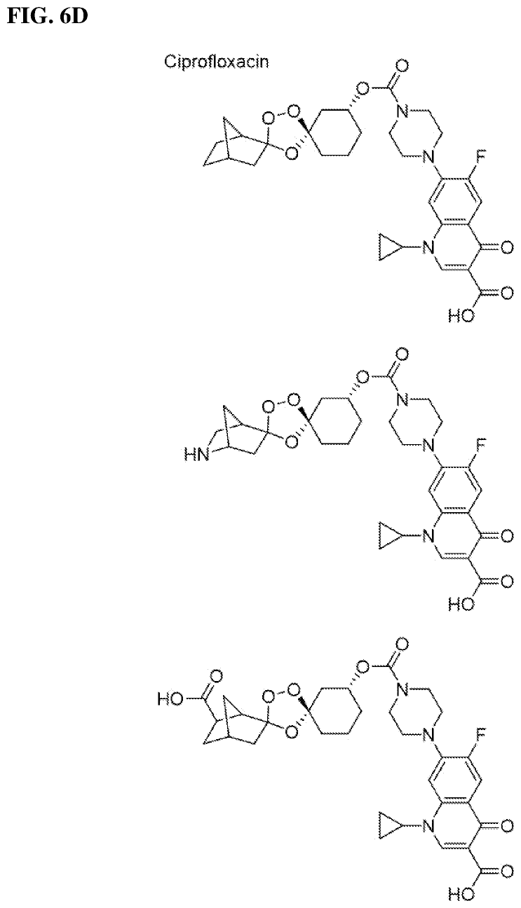

To further study the kinetics and regioselectivity of iron(II)-dependent activation, we followed the reaction of 6f and 6a with ferrous ammonium sulfate by UPLC/MS. As we have described previously for the progenitor TRX moiety (7, 25), efficient activation and payload release requires regioselective activation of the endoperoxide bond by Fe²⁺ such that the ketone intermediate A is produced preferentially over the alternative adamantan-2-one product (FIG. 4). In adamantane-based systems this regioselectivity is conferred by the steric effect of the adamantane ring, as noted previously (22). We predicted based on modeling that the bicyclo[2.2.1]heptane moiety of 6f should similarly shield the proximal oxygen atom from inner-sphere coordination with Fe²⁺ leading to regioselective peroxide scission. In the event, exposure of either 6f or 6a to Fe²⁺ (as ferrous-ammonium sulfate with pH 7.4 Tris buffer) led within minutes to clean conversion to the common cyclohexanone intermediate A (FIG. 4). No detectable quantity of the alternate bicyclo[2.2.1]heptan-2-one product was detected in the reactions of 6f, thus confirming that the process is highly regioselective. Conversion of 6f to A was moderately faster than for comparator 6a, with 6f fully consumed by the 11-minute time point. The kinetics of mefloquine release from intermediate A were comparable within experimental error. Taken together, the antiplasmodial and cell-free Fe²⁺ reactivity data indicate that efficient iron(II)-dependent uncaging and traceless release of payloads can be realized from non-adamantane based scaffolds such as 6f and likely as well from the other scaffolds described herein.

In summary, we explored the scope of Griesbaum co-ozonolysis under optimized low temperature reaction conditions. Overall, these conditions afford improved yields, substrate scope, and diastereoselectivity as compared to our previously described conditions. With the appropriate selection of ketoxime and ketone reactants, a variety of new adducts could be prepared using the reported conditions. Three of these new adducts were conjugated to drug or control payloads and shown in antiplasmodial assays and chemical Fe²⁺ reactivity studies to be competent sensors of ferrous iron, much like the parental TRX system.

These findings are significant insofar as they should enable iron(II)-activated chemistries to be applied to a broader range of drug or reporter payloads whilst maintaining physiochemical properties and iron(II)-dependent reactivity within a pharmacologically desirable range. As such, our findings have implications for antimalarial drug discovery, iron(II)-dependent drug delivery, and the continuing development of new chemical tools to study labile ferrous iron in biological settings.

### Example 2: Experimental Methods and Characterization Data

### Materials

All chemical reagents were obtained commercially and were used without further purification, unless otherwise stated. Anhydrous solvents were purchased from Sigma-Aldrich and were used without further purification. Solvents used for flash column chromatography and reaction workup procedures were purchased from either Sigma-Aldrich or Fisher Scientific. Column chromatography was performed on Silicycle Sili-prep cartridges using a Biotage Isolera Four automated flash chromatography system.

### Instrumentation

NMR spectra were recorded on either a Varian INOVA 400 MHz spectrometer (with 5 mm QuadNuclear Z-Grad Probe), calibrated to CH(D)Cl₃ as an internal reference (7.27 and 77.00 ppm for ¹H and ¹³C NMR spectra, respectively). Data for ¹H NMR spectra are reported in terms of chemical shift (δ, ppm), multiplicity, coupling constant (Hz), and integration. Data for ¹³C NMR spectra are reported in terms of chemical shift (δ, ppm), with multiplicity and coupling constants in the case of C—F coupling. The following abbreviations are used to denote the multiplicities: s=singlet, d=doublet, t=triplet, q=quartet, m=multiplet, br=broad, app=apparent, or combinations of these. UPLC-MS and compound purity were determined using a Waters Acquity QDa mass spectrometer equipped with FTN-H Sample Manager, Evaporative Light Scattering Detector and Photodiode Array Detector. Separations were carried out with Acquity UPLC® BEH C18, 1.7 mm, 2.1×50 mm column, at 25° C. using a mobile phase of water-acetonitrile containing a constant 0.05% formic acid.

Plasmodium falciparum EC₅₀ Determinations

The growth inhibition assay for P. falciparum was conducted as described previously with minor modifications. Briefly, P. falciparum strain W2 synchronized ring-stage parasites were cultured in human red blood cells in 96-well flat-bottom culture plates at 37° C., adjusted to 1% parasitemia and 2% hematocrit under an atmosphere of 3% O₂, 5% CO₂, and 91% N₂ in a final volume of 0.1 mL per well in RPMI-1640 media supplemented with 0.5% Albumax, 2 mM L-glutamine, and 100 mM hypoxanthine in the presence of various concentrations of inhibitors. Tested compounds were serially diluted 1:3 in the range 10 000-4.6 nM (or 1000-0.006 nM for more potent analogues), with a maximum DMSO concentration of 0.1%. Following 48 h of incubation, the cells were fixed by adding 0.1 mL of 2% formaldehyde in phosphate buffered saline, pH=7.4 (PBS). Parasite growth was evaluated by flow cytometry on a FACsort (Becton Dickinson) equipped with AMS-1 loader (Cytek Development) after staining with 1 nM of the DNA dye YOYO-1 (Molecular Probes) in 100 mM NH₄Cl, 0.1% Triton x-100 in 0.8% NaCl. Parasitemias were determined from dot plots (forward scatter vs fluorescence) using CELLQUEST software (Becton Dickinson). EC₅₀ values for growth inhibition were determined from plots of percentage control parasitemia over inhibitor concentration using GraphPad Prism software.

### Fe(II)-Fragmentation Assay

To 1 mL of trioxolane analog in DMSO (1 mM) at 37° C. was added 1 mL of Tris containing 50 mM ferrous ammonium sulfate. At various time points, 40 μL aliquots of the resulting mixture were removed and spun down in a mini centrifuge for 20 seconds, 20 μL of the supernatant was removed and 5 μL was injected into the UPLC. The concentration of the fragments was determined by UV from UPLC (290 nm). The resulting UV curves were plotted using GraphPad Prism software.

### Synthetic Procedures

General procedure A: original conditions for Griesbaum reaction. To an oven-dried 100 mL flask was charged with ketone 2 (1.0 equiv), oxime 1 (3.0 equiv) and CCl₄. The mixture was cooled to 0° C. and ozone was bubbled through the solution. O₂ flow=1 liter per minute, ozone gauge=3.5 (This setting amounts to ˜6 g/hour ozone production). The reaction mixture was stirred at −0° C. for 4 hours, at which point the reaction was judge complete based on UPLC-MS. The mixture was then bubbled with N₂ for 10 mins and concentrated. The resulting crude product was purified by flash column chromatography (column was pre-washed with 1% Et₃N in hexane) to yield desired compound.

General procedure B: improved, low temperature conditions for Griesbaum reaction. To an oven-dried 100 mL flask was charged with ketone 2 (1.0 equiv), oxime 1 (3.0 equiv) and hexane. The mixture was cooled to −78° C. and ozone was bubbled through the solution. O₂ flow=1 liter per minute, ozone gauge=3.5 (This setting amounts to ˜6 g/hour ozone production). For starting materials that are not soluble in hexane, a small amount of dichloromethane can be added to enhance solubility. The reaction flask was wrapped with aluminum foil (protected from light) and the reaction mixture stirred at −78° C. for 4 hours, at which point the reaction was judge complete based on UPLC-MS. The reaction mixture was then bubbled with N₂ for 10 mins and concentrated. The resulting crude product was purified by flash column chromatography (column was pre-washed with 1% Et₃N in hexane) to yield the desired compound.

General procedure C: Silyl ether deprotection. To a stirred solution of trioxolane 3 (1.0 equiv) in THF (20 mL) was added a solution of tetrabutylammonium fluoride (1.0 M in THF, 5.0 equiv) dropwise while stirring at 0° C. The reaction mixture was allowed to slowly warm to rt and was stirred for 12 h, at which point conversion was determined to be complete based on TLC and LC/MS analysis. The reaction was then diluted with brine (100 mL) and extracted with EtOAc (2×100 mL). The organic layer was then dried (MgSO₄), filtered, and concentrated under reduced pressure to afford a yellow oil. The crude material was purified using flash column chromatography ((column was washed with 1% Et₃N in hexane first, 0-50% EtOAc-Hexanes) to yield the desired product 4.

General procedure D: para-nitrophenyl carbonate formation. To an oven-dried round-bottom flask containing a magnetic stir bar under an Ar (g) atmosphere was added alcohol 4 (1.0 equiv), dichloromethane, N,N-diisopropylethylamine (3.0 equiv), and 4-dimethylaminopyridine (1.0 equiv). The mixture was cooled to 0° C. while 4-nitrophenyl chloroformate (3.0 equiv) was added as a solid in two portions. The reaction mixture was allowed to warm to rt and was stirred for 3 h. The reaction was diluted with DI H₂O (100 mL) and extracted with EtOAc (1×100 mL). The organic layer was washed repeatedly with 1 M aq. K₂CO₃ solution until the aqueous layer was colorless and no longer yellow (indicating that most of the p-nitrophenol had been successfully removed from the organic layer). The organic layer was then dried (MgSO₄), filtered, and concentrated under reduced pressure to yield a viscous yellow oil. The crude material was purified using flash column chromatography (0-25% EtOAc-hexanes; column was pre-washed with 1% Et₃N in hexane) to yield the desired product 5.

General procedure E: coupling to mefloquine. To a solution of 5 (1.0 equiv) in DMF was added N,N-diisopropylethylamine (5.0 equiv), dimethylaminopyridine (0.5 equiv) followed by, mefloquine (1.2 equiv) at rt. The bright yellow mixture was allowed to stir at rt for 16 h. The reaction was quenched with 1 M aq NaOH (20 mL) and diluted with EtOAc (30 mL). The organic phase was separated and washed with additional 1 M aq NaOH (4×30 mL) until the aqueous layer was colorless (indicating that p-nitrophenol had been successfully removed from the organic layer). The combined aqueous layers were then back extracted with EtOAc (1×30 mL). The combined organic layers were then washed with brine (20 mL), dried (MgSO₄), filtered, and concentrated under reduced pressure. The crude residue was purified using flash column chromatography (0-80% EtOAc-hexanes; column was pre-washed with 1% Et₃N in hexane) to give the desired product 6.

General procedure F: coupling to morpholine. To a solution of 5 (1.0 equiv) in dichloromethane was added Et₃N (3.0 equiv), followed by, morpholine HCl salt (1.4 equiv) at rt. The bright yellow mixture was allowed to stir at rt for 3 h. The reaction was quenched with 1 M aq NaOH (20 mL) and diluted with EtOAc (30 mL). The organic phase was separated and washed with additional 1 M aq NaOH (4×30 mL) until the aqueous layer was colorless (indicating that p-nitrophenol had been successfully removed from the organic layer). The combined aqueous layers were then back extracted with EtOAc (1×30 mL). The combined organic layers were then washed with brine (20 mL), dried (MgSO₄), filtered, and concentrated under reduced pressure. The crude residue was purified using flash column chromatography (0-80% EtOAc-hexanes; column was pre-washed with 1% Et₃N in hexane) to give the desired product 7.

General procedure G: oxime synthesis. To a pressure vessel containing a magnetic stir bar was added ketone (1.0 equiv) followed by MeOH, pyridine (1.5 equiv) and methoxylamine hydrochloride (1.5 equiv). The reaction vessel was then sealed with a teflon screw cap and heated to 90° C. behind a blast shield for 3 h. The mixture was then cooled to rt and the cap was carefully unscrewed. The reaction mixture was then transferred to a flask and concentrated under reduced pressure to a crude semi-solid. The crude residue was diluted with 10% aq KHSO₄ solution (115 mL) and extracted with EtOAc (1×200 mL). The organic phase was washed with additional 10% aq KHSO₄ solution (3×60 mL) and the aqueous layers back-extracted with EtOAc (1×150 mL). The combined organic phases were washed with brine (1×150 mL), dried (MgSO₄), filtered and concentrated to the desired product 1, which solidified under high vacuum to give oxime, which was sufficiently pure to be carried onto the next step without further purification.

General procedure H. To a pressure vessel containing a magnetic stir bar was added ketone (1.0 equiv) followed by MeOH, pyridine (1.5 equiv) and methoxylamine hydrochloride (1.5 equiv). The reaction vessel was then sealed with a teflon screw cap and heated to 50° C. overnight. The mixture was then cooled to room temperature and the cap was carefully unscrewed. The reaction mixture was then transferred to a flask and concentrated under reduced pressure to a crude semi-solid. The crude residue was diluted with 10% aq KHSO₄ solution and extracted with EtOAc (1×200 mL). The organic phase was washed with additional 10% aq KHSO₄ solution (3×60 mL) and the aqueous layers back-extracted with EtOAc (1×150 mL). The combined organic phases were washed with brine (1×150 mL), dried (MgSO₄), filtered and concentrated to a colorless oil, which solidified under high vacuum to give oxime, which was sufficiently pure to be carried onto the next step without further purification.

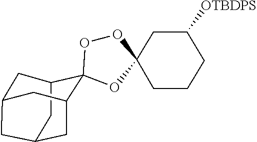

Prepared according to general procedure A using 20 mL CCl₄, ketone 2 (330.0 mg, 0.94 mmol, 1.0 equiv), and oxime 1a (520.6 mg, 2.88 mmol, 2.5 equiv). Chromatography (0-20% EtOAc/hexanes gradient elution) to afforded 360.0 mg (71%) of 3a as a white solid (cis:trans=1:10).

Prepared according to general procedure B using 20 mL hexane, ketone 2 (50.0 mg, 0.14 mmol, 1.0 equiv), and oxime 1a (76.3 mg, 0.43 mmol, 3 equiv). Chromatography (0-20% EtOAc/hexanes gradient elution) to afforded 67.5 mg (91%) of 3a as a white solid (dr >20:1). ¹H NMR (400 MHz, CDCl₃): δ 7.69 (td, J=7.7, 1.5 Hz, 4H), 7.36-7.46 (m, 6H), 3.78-3.85 (m, 1H), 1.47-2.05 (m, 20H), 1.19-1.36 (m, 2H), 1.08 (s, 9H). ¹³C NMR (100 MHz, CDCl₃): δ 135.7 (two peaks), 134.5, 134.4, 129.5 (two peaks), 127.6, 127.5, 111.2, 109.2, 69.8, 43.7, 36.8, 36.3, 36.2, 34.9, 34.8, 34.7, 34.6, 34.4, 33.8, 33.2, 27.0, 26.9, 26.5, 19.9, 19.2. LRMS (ESI) caled for C₃₂H₄₂NaO₄Si [M+Na]⁺ m/z 541.28, found 541.56.

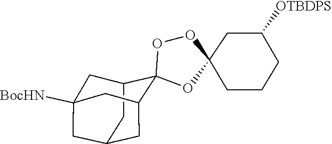

Prepared according to general procedure A using 40 mL CCl₄, ketone 2 (1.00 g, 2.84 mmol, 1.0 equiv, contaminated with 39% TBDPSOH), and oxime 1e (1.67 g, 5.68 mmol, 2.00 equiv). Chromatography (0-15% EtOAc/hexanes gradient elution) afforded 718.9 mg (contaminated with 57% TBDPSOH by ¹HNMR integration, calculated yield 28%) of 3e as colorless solid.

Prepared according to general procedure B using 30 mL hexane, ketone 2 (100 mg, 0.28 mmol, 1.0 equiv), and oxime 1b (250 mg, 0.85 mmol, 3.0 equiv). Chromatography (0-20% EtOAc/hexanes gradient elution) to afforded 89.1 mg (50%) of 3b as colorless solid. ¹H NMR (400 MHz, CDCl₃) δ 7.64-7.71 (m, 4H), 7.34-7.46 (m, 6H), 4.63 (br s, 1H), 3.73-3.83 (m, 1H), 2.04-2.15 (m, 2H), 1.90-2.02 (m, 8H), 1.68-1.84 (m, 6H), 1.54-1.63 (m, 4H), 1.44 (s, 9H), 1.20-1.29 (m, 2H), 1.08 (s, 9H); ¹³C NMR (100 MHz, CDCl₃) δ 184.4, 135.7 (two peaks), 134.4 (two peaks), 129.5 (two peaks), 127.6 (two peaks), 110.0 (two peaks), 109.5 (two peaks), 69.7 (two peaks), 49.5, 49.2, 43.6 (two peaks), 40.0, 39.9 (two peaks), 38.7, 37.1 (two peaks), 36.8 (two peaks), 34.3, 33.7, 33.5 (two peaks), 28.4, 27.9, 27.5, 27.0, 19.8, 19.1 several minor diastereomer peaks overlapping or not observed; HRMS(ESI) calculated for C₃₇H₅₂O₆NSiNa [M+Na]⁺ m/z 656.3378, found 656.3375.

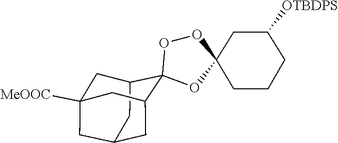

Prepared according to general procedure A using 20 mL CCl₄, ketone 2 (50 mg, 0.14 mmol, 1.0 equiv), and oxime 1c (101 mg, 0.43 mmol, 3.0 equiv). Chromatography (0-20% EtOAc/hexanes gradient elution) to afforded 19.3 mg (23%) of 3c as oil (cis:trans=1:10).

Prepared according to general procedure B using 30 mL hexane, ketone 2 (400 mg, 1.13 mmol, 1.0 equiv), and oxime 1c (0.81 g, 3.41 mmol, 3.0 equiv). Chromatography (0-20% EtOAc/hexanes gradient elution) to afforded 501.3 mg (77%) of 3c as oil (dr >20:1). ¹H NMR (400 MHz, CDCl₃) δ 7.62-7.71 (m, 4H), 7.34-7.46 (m, 6H), 3.73-3.83 (m, 1H), 3.64 (app d, 3H), 2.08-2.20 (m, 2H), 1.56-1.98 (m, 17H), 1.20-1.30 (m, 2H), 1.08 (s, 9H); ¹³C NMR (100 MHz, CDCl₃) δ 177.2, 135.7 (two peaks), 134.3 (two peaks), 129.5 (two peaks), 127.5 (two peaks), 110.0, 109.5 (two peaks), 69.7, 51.7, 43.6, 39.8, 38.1, 36.3 (two peaks), 36.0, 35.8, 35.7, 34.3, 33.7 (two peaks), 33.5, 27.0, 26.5, 26.1, 24.9, 19.8, 19.1 several minor diastereomer peaks overlapping or not observed; HRMS(ESI) calculated for C₃₄H₄₄O₆SiNa [M+Na]⁺ m/z 599.2799, found 599.2806.

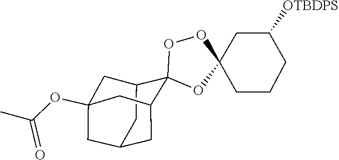

Prepared according to general procedure A using 30 mL CCl₄, ketone 2 (50 mg, 0.14 mmol, 1.0 equiv), and oxime 1d (101 mg, 0.43 mmol, 3.0 equiv). Chromatography (0-5% EtOAc/hexanes gradient elution) to afforded 4.2 mg (5.1%) of 3d as colorless solid.

Prepared according to general procedure B using 30 mL hexane, ketone 2 (50 mg, 0.14 mmol, 1.0 equiv), and oxime 1d (101 mg, 0.43 mmol, 3.0 equiv). Chromatography (0-5% EtOAc/hexanes gradient elution) to afforded 69.3 mg (84%) of 3d as colorless solid. ¹H NMR (400 MHz, CDCl₃) δ 7.60-7.79 (m, 4H), 7.35-7.48 (m, 6H), 3.74-3.90 (m, 1H), 2.19-2.38 (m, 2H), 2.05-2.19 (m, 7H), 1.94-2.02 (m, 5H), 1.46-1.92 (m, 11H), 1.19-1.35 (m, 3H), 1.09 (s, 9H); ¹³C NMR (100 MHz, CDCl₃) δ 170.2, 135.8 (2 peaks), 134.4 (2 peaks), 129.6 (3 peaks), 127.6 (2 peaks), 109.6 (4 peaks), 78.6, 78.2, 69.7, 43.6 (2 peaks), 40.0 (2 peaks), 38.4, 38.2, 38.0 (2 peaks), 34.3, 33.7 (2 peaks), 33.5 (2 peaks), 33.2 (2 peaks), 29.1, 28.8, 27.0, 22.6 (2 peaks), 19.9, 19.1; HRMS(ESI) calculated for C₃₄H₄₄O₆SiNa [M+Na]⁺ 599.2799 found m/z: 599.2799.

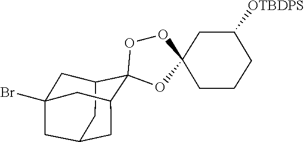

Prepared according to general procedure A using 30 mL CCl₄, ketone 2 (50 mg, 0.14 mmol, 1.0 equiv), and oxime 1e (101 mg, 0.43 mmol, 3.0 equiv). Chromatography (0-20% EtOAc/hexanes gradient elution) to afforded 15.0 mg (18%) of 3e as colorless solid.

Prepared according to general procedure B using 30 mL hexane, ketone 2 (50 mg, 0.14 mmol, 1.0 equiv), and oxime 1e (110 mg, 0.43 mmol, 3.0 equiv). Chromatography (0-20% EtOAc/hexanes gradient elution) to afforded 80.1 mg (94%) of 3e as colorless solid. ¹H NMR (400 MHz, CDCl₃) δ 7.64-7.71 (m, 4H), 7.34-7.47 (m, 6H), 3.77-3.83 (m, 1H), 2.46-2.65 (m, 2H), 2.16-2.37 (m, 4H), 1.46-2.03 (m, 13H), 1.17-1.34 (m, 2H), 1.08 (s, 9H); ¹³C NMR (100 MHz, CDCl₃) δ 135.7 (two peaks), 134.3 (two peaks), 129.5 (two peaks), 127.5 (two peaks), 109.7 (two peaks), 108.9 (minor diastereomer), 108.8 (major diastereomer), 69.6, 62.5 (minor diastereomer), 62.1 (major diastereomer), 48.4 (two peaks), 45.9, 45.6, 43.5, 45.4, 39.7 (two peaks), 39.5 (two peaks), 34.2, 33.6 (two peaks), 32.9, 32.8, 32.6 (two peaks), 30.8, 30.4, 27.0, 19.7, 19.1 (two peaks) several minor diastereomer peaks overlapping or not observed; HRMS(ESI) calculated for C₃₂H₄₁O₄BrSiNa [M+Na]⁺ m/z 619.1850, found 619.1854.

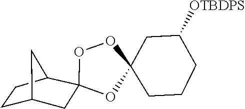

Prepared according to general procedure B using 30 mL hexane, ketone 2 (150 mg, 0.43 mmol, 1.0 equiv), and oxime if (178 mg, 1.28 mmol, 3.0 equiv). Chromatography (0-20% EtOAc/hexanes gradient elution) to afforded 166.2 mg (82%) of 3f as oil (13:8:1 dr). ¹H NMR (400 MHz, CDCl₃) δ 7.65-7.72 (m, 4H), 7.35-7.47 (m, 6H), 3.80-3.90 (m, 1H, minor diastereomer), 3.70-3.80 (m, 1H, major diastereomer), 2.22-2.30 (m, 1H), 2.10-2.17 (m, 1H), 1.71-2.02 (m, 4H), 1.12-1.65 (m, 12H), 1.08 (s, 9H); ¹³C NMR (100 MHz, CDCl₃) δ 135.8 (multiple peaks), 134.4 (multiple peaks), 129.6 (multiple peaks), 127.5 (multiple peaks), 115.9, 109.2 (two peaks), 69.7 (two peaks), 45.0, 44.9, 43.8, 43.1, 42.1, 41.0, 37.6, 37.5, 35.4, 35.2, 34.3 (two peaks), 33.8, 33.1, 27.8, 27.7, 27.0 (two peaks), 21.7, 21.6, 19.9, 19.7, 19.1 (two peaks) several minor diastereomer peaks overlapping or not observed; HRMS(ESI) calculated for C₂₉H₃₈O₄SiNa [M+Na]⁺ m/z 501.2432, found 501.2436.

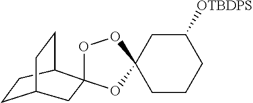

Prepared according to general procedure B using 30 mL hexane, ketone 2 (50 mg, 0.14 mmol, 1.0 equiv), and oxime 1g (65 mg, 0.43 mmol, 3.0 equiv). Chromatography (0-20% EtOAc/hexanes gradient elution) to afforded 31 mg (44%) of 3g as oil (4:1 dr). ¹H NMR (400 MHz, CDCl₃) δ 7.62-7.70 (m, 4H), 7.32-7.46 (m, 6H), 3.80-3.89 (m, 1H, minor diastereomer), 3.69-3.80 (m, 1H, major diastereomer), 1.68-1.88 (m, 9H), 1.57-1.65 (m, 2H), 1.34-1.50 (m, 7H), 1.21-1.29 (m, 2H), 1.08 (app d, 9H); ¹³C NMR (100 MHz, CDCl₃) δ 135.8 (two peaks), 134.4 (two peaks), 129.5 (two peaks), 127.5 (two peaks), 110.1, 109.1, 69.7, 43.4, 37.7, 34.4, 33.9, 32.7, 27.0, 25.9, 24.2, 21.0, 20.9, 19.8, 19.1 several minor diastereomer peaks overlapping or not observed; HRMS(ESI) calculated for C₃₀H₄₀O₄SiNa [M+Na]⁺ m/z 515.2588, found 515.2594.

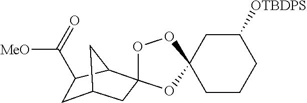

Prepared according to general procedure B using 30 mL hexane, ketone 2 (50 mg, 0.15 mmol, 1.0 equiv), and oxime 1r (84 mg, 0.43 mmol, 3.0 equiv). Chromatography (0-20% EtOAc/hexanes gradient elution) to afforded 60 mg (79%) of 3r as oil. ¹H NMR (400 MHz, Chloroform-d) δ 7.77-7.62 (m, 4H), 7.47-7.32 (m, 6H), 3.91-3.60 (m, 4H), 2.94-2.75 (m, 1H), 2.58-2.40 (m, 1H), 2.39-2.28 (m, 1H), 1.97-1.23 (m, 14H), 1.12-0.99 (m, 9H); LRMS(ESI) calculated for C₃₁H₄₀O₆SiNa [M+Na]⁺ m/z 559.25, found 559.21.

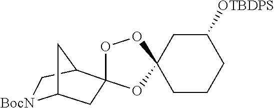

Prepared according to general procedure B using 30 mL hexane, ketone 2 (75 mg, 0.21 mmol, 1.0 equiv), and oxime is (154 mg, 0.64 mmol, 3.0 equiv). Chromatography (0-20% EtOAc/hexanes gradient elution) to afforded 73 mg (59%) of 3s as oil. ¹H NMR (400 MHz, Chloroform-d) δ 7.80-7.59 (m, 4H), 7.49-7.29 (m, 6H), 4.30-4.00 (m, 1H), 3.89-3.69 (m, 1H), 3.46-3.30 (m, 1H), 3.21-2.98 (m, 1H), 2.54-2.31 (m, 1H), 2.19-1.52 (m, 11H), 1.51-1.38 (m, 9H), 1.35-1.22 (m, 2H), 1.07 (s, 9H, major), 1.06 (s, 9H, minor); LRMS(ESI) calculated for C₃₃H₄₅O₆SiNNa [M+Na]⁺ m/z 602.29, found 602.21.

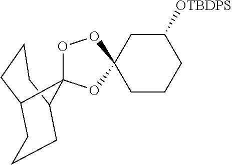

Prepared according to general procedure B using 30 mL hexane, ketone 2 (200 mg, 0.57 mmol, 1.0 equiv), and oxime 1h (285 mg, 1.70 mmol, 3.0 equiv). Chromatography (0-20% EtOAc/hexanes gradient elution) to afforded 250.1 mg (87%) of 3h as oil. ¹H NMR (400 MHz, CDCl₃) δ 7.62-7.72 (m, 4H), 7.34-7.46 (m, 6H), 3.76-3.83 (m, 1H), 2.08-2.20 (m, 2H), 1.56-1.98 (m, 17H), 1.20-1.30 (m, 2H), 1.08 (s, 9H); ¹³C NMR (100 MHz, CDCl₃) δ 135.7 (two peaks), 134.4 (two peaks), 129.5 (two peaks), 127.5 (two peaks), 110.1, 109.1, 69.8, 43.8, 36.1, 36.0, 34.4, 33.9, 33.4, 33.8, 29.7, 29.4, 29.3, 29.2, 27.9, 27.0, 20.8, 20.4, 19.9, 19.1 several minor diastereomer peaks overlapping or not observed; HRMS(ESI) calculated for C₃₁H₄₂O₄SiNa [M+Na]⁺ m/z 529.2745, found 529.2751.

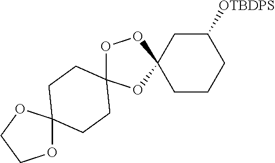

Prepared according to general procedure B using 30 mL hexane, ketone 2 (100 mg, 0.28 mmol, 1.0 equiv), and oxime 1j (158 mg, 0.85 mmol, 3.0 equiv). Chromatography (0-20% EtOAc/hexanes gradient elution) to afforded 105.3 mg (71%) of 3j as oil. ¹H NMR (400 MHz, CDCl₃) δ 7.63-7.70 (m, 4H), 7.35-7.47 (m, 6H), 4.17-4.22 (m, 1H, minor diastereomer), 3.90-4.00 (m, 4H), 3.75-3.84 (m, 1H, major diastereomer), 1.94-2.02 (m, 1H), 1.70-1.88 (m, 10H), 1.55-1.62 (m, 2H), 1.44-1.51 (m, 1H), 1.20-1.31 (m, 2H), 1.07 (s, 9H); ¹³C NMR (100 MHz, CDCl₃) δ 135.8 (two peaks), 134.4 (two peaks), 129.6 (two peaks), 127.6 (two peaks), 109.5, 107.9, 107.7 (minor diastereomer), 69.7, 64.4 (two peaks), 43.5, 34.3, 33.5, 32.1, 31.9, 31.5, 31.3, 27.0, 19.9, 19.1 several minor diastereomer peaks overlapping or not observed; HRMS(ESI) calculated for C₃₀H₄₀O₆SiNa [M+Na]⁺ m/z 547.2486, found 547.2492.

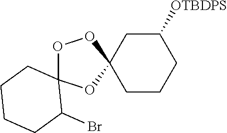

Prepared according to general procedure B using 30 mL hexane, ketone 2 (50 mg, 0.14 mmol, 1.0 equiv), and oxime 1k (88 mg, 0.43 mmol, 3.0 equiv). Chromatography (0-20% EtOAc/hexanes gradient elution) to afforded 42.3 mg (53%) of 3k as oil (6:4 dr). ¹H NMR (400 MHz, CDCl₃) δ 7.62-7.70 (m, 4H), 7.34-7.50 (m, 6H), 4.23-4.25 (m, 1H, minor diastereomer), 4.03-4.10 (m, 1H, major diastereomer), 3.77-3.88 (m, 1H), 1.96-2.22 (m, 4H), 1.71-1.83 (m, 4H), 1.46-1.66 (m, 6H), 1.23-1.34 (m, 2H), 1.08 (app d, 9H); ¹³C NMR (100 MHz, CDCl₃) δ 135.7 (multiple peaks), 134.3 (multiple peaks), 129.7 (multiple peaks), 127.6 (multiple peaks), 110.4 (two peaks), 107.3 (minor diastereomer), 107.2 (major diastereomer), 69.6, 53.0, 52.7, 43.6, 43.0, 34.2, 33.6, 33.2, 32.6, 31.9, 27.0, 23.2, 23.0, 19.9, 19.6, 19.1 (two peaks) several minor diastereomer peaks overlapping or not observed; HRMS(ESI) calculated for C₂₈H₃₇O₄BrSiNa [M+Na]⁺ m/z 567.1537, found 567.1543.

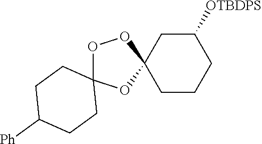

Prepared according to general procedure B using 30 mL hexane, ketone 2 (100 mg, 0.28 mmol, 1.0 equiv), and oxime 11 (173 mg, 0.85 mmol, 3.0 equiv). Chromatography (0-20% EtOAc/hexanes gradient elution) to afforded 115.5 mg (75%) of 31 as oil. ¹H NMR (400 MHz, CDCl₃) δ 7.74-7.80 (m, 4H), 7.43-7.52 (m, 6H), 7.35-7.40 (m, 2H), 7.25-7.31 (m, 3H), 3.98-4.10 (m, 1H, minor diastereomer), 3.85-3.96 (m, 1H, major diastereomer), 2.55-2.64 (m, 1H), 2.07-2.23 (m, 2H), 1.83-2.01 (m, 11H), 1.68-1.74 (m, 1H), 1.55-1.64 (m, 1H), 1.32-1.42 (m, 2H), 1.17 (s, 9H); ¹³C NMR (100 MHz, CDCl₃) δ 146.1, 135.8 (two peaks), 134.4 (two peaks), 129.6 (two peaks), 127.6 (two peaks), 126.9, 126.7, 126.2 (two peaks), 109.6, 109.2 (minor diastereomer), 108.2 (two peaks), 69.7, 43.5, 43.2, 34.6, 34.3, 34.0, 33.6, 31.2, 31.0, 29.7, 27.0, 19.8, 19.1 several minor diastereomer peaks overlapping or not observed; HRMS(ESI) calculated for C₃₄H₄₂O₄SiNa [M+Na]⁺ m/z 565.2745, found 565.2747.

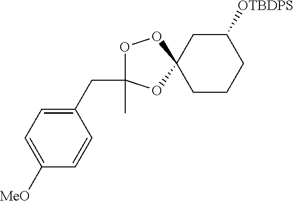

Prepared according to general procedure B using 30 mL hexane, ketone 2 (50 mg, 0.14 mmol, 1.0 equiv), and oxime 1q (82.0 mg, 0.43 mmol). Chromatography (0-20% EtOAc/hexanes gradient elution) to afforded 3 mg (4%) of 3q as oil (1.4:1 dr). ¹H NMR (400 MHz, CDCl₃) δ 7.62-7.70 (m, 4H), 7.32-7.44 (m, 6H), 7.11 (dd, 2H, J=8.8, 13.8 Hz), 6.82 (dd, 2H, J=5.6, 8.5 Hz), 3.69-3.86 (m, 4H), 2.89 (ABq, 1H, J=13.9, 32.9 Hz, minor diastereomer), 2.80 (ABq, 1H, J=14.1, 43.8 Hz, major diastereomer), 1.57-2.04 (m, 8H), 1.21-1.30 (m, 5H), 1.08 (app d, 9H); ¹³C NMR (100 MHz, CDCl₃) δ 158.44, 135.8 (two peaks), 134.3 (two peaks), 131.4 (two peaks), 129.5 (three peaks), 128.0, 127.5 (three peaks), 113.5, 109.9 (two peaks), 109.2, 69.7 (two peaks), 52.2 (two peaks), 43.4, 43.2, 42.9, 34.3, 33.6, 33.2, 27.0, 22.7, 21.8, 19.8, 19.1 several minor diastereomer peaks overlapping or not observed; HRMS(ESI) calculated for C₃₂H₄₀O₅SiNa [M+Na]⁺ m/z 555.2537, found 555.2537.

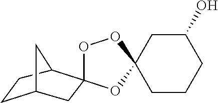

Prepared according to general procedure C using 10 mL THF, trioxloane 3f (87 mg, 0.18 mmol, 1.0 equiv), and TBAF (1M in THF, 0.91 mL, 0.91 mmol, 5.0 equiv). Chromatography (0-50% EtOAc/hexanes gradient elution) to afforded 25.0 mg (57%) of 4f as oil. ¹H NMR (400 MHz, CDCl₃) δ 3.86-4.04 (m, 1H), 2.25-2.40 (m, 3H), 2.02-2.11 (m, 1H), 1.91-1.99 (m, 1H) 1.60-1.85 (m, 7H), 1.38-1.57 (m, 5H), 1.19-1.35 (m, 2H); ¹³C NMR (100 MHz, CDCl₃) δ 116.5 (two peaks), 108.9 (two peaks), 68.0, 44.9 (two peaks), 42.2, 41.5, 41.4, 41.2, 37.6, 35.3 (two peaks), 34.0, 33.2, 33.1, 32.8, 27.7, 21.6, 19.3, 19.0 several minor diastereomer peaks overlapping or not observed; LRMS(ESI) calculated for C₁₃H₂₀O₄NNa [M+Na]⁺ m/z 263.13, found 263.09.

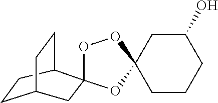

Prepared according to general procedure C using 10 mL THF, trioxolane 3g (70 mg, 0.14 mmol, 1.0 equiv), and TBAF (1 M in THF, 0.71 mL, 0.71 mmol, 5.0 equiv). Chromatography (0-50% EtOAc/hexanes gradient elution) to afforded 20.0 mg (55%) of 4g as oil. ¹H NMR (400 MHz, CDCl₃) δ 3.86-4.03 (m, 1H), 2.38 (br, 1H), 1.39-2.14 (m, 20H); ¹³C NMR (100 MHz, CDCl₃) δ 111.6, 108.9 (two peaks), 68.0, 42.2, 41.4, 38.1 (two peaks), 34.2, 33.3, 33.1 (two peaks), 32.8 (two peaks), 25.9, 24.4, 24.1, 21.0 (two peaks), 19.3, 19.0 several minor diastereomer peaks overlapping or not observed; LRMS(ESI) calculated for C₁₄H₂₂O₄NNa [M+Na]⁺ m/z 277.14, found 277.09.

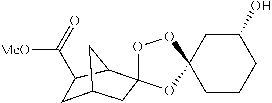

Prepared according to general procedure C using 10 mL THF, intermediate 3r (300 mg, 0.56 mmol, 1.0 equiv), and TBAF (1M in THF, 2.79 mL, 2.79 mmol, 5.0 equiv). Chromatography (0-50% EtOAc/hexanes gradient elution) to afforded 95.0 mg (58%) of 4r as oil. ¹H NMR (400 MHz, Chloroform-d) δ 3.95-3.77 (m, 1H), 3.66 (s, 3H, minor), 3.65 (s, 3H, major), 2.86 (dd, J=9.0, 5.2 Hz, 1H), 2.64-2.54 (m, 1H), 2.35 (br, 1H), 2.12-1.29 (m, 14H); LRMS(ESI) calculated for C₁₅H₂₂O₆Na [M+Na]⁺ m/z 321.13, found 321.08.

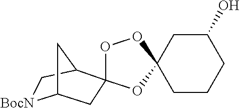

Prepared according to general procedure C using 10 mL THF, intermediate 3s (260 mg, 0.45 mmol, 1.0 equiv), and TBAF (1M in THF, 2.24 mL, 2.24 mmol, 5.0 equiv). Chromatography (0-50% EtOAc/hexanes gradient elution) to afford 120.0 mg (78%) of 4s as oil. ¹H NMR (400 MHz, Chloroform-d) δ 4.24-4.10 (m, 1H), 3.87-3.66 (m, 1H), 3.45-3.32 (m, 1H), 3.16-3.00 (m, 1H), 2.83 (s, 1H), 2.55 (d, J=10.0 Hz, 1H), 2.18-1.26 (m, 21H); LRMS(ESI) calculated for C₁₇H₂₇O₆Na [M+Na]⁺ m/z 364.17, found 364.18.

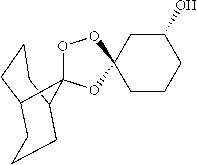

Prepared according to general procedure C using 10 mL THF, trioxolane 3h (250 mg, 0.49 mmol, 1.0 equiv), and TBAF (1M in THF, 2.47 mL, 2.47 mmol, 5.0 equiv). Chromatography (0-50% EtOAc/hexanes gradient elution) to afforded 132.0 mg (66%) of 4h as oil. ¹H NMR (400 MHz, CDCl₃) δ 3.86-4.03 (m, 1H), 2.69 (br, 1H), 1.35-2.10 (m, 22H); ¹³C NMR (100 MHz, CDCl₃) δ 111.8, 108.9, 67.9, 41.9, 36.0 (two peaks), 33.8, 33.1, 29.6 (two peaks), 29.3, 20.7, 20.3, 19.1 several minor diastereomer peaks overlapping or not observed; LRMS(ESI) calculated for C₁₅H₂₄O₄NNa [M+Na]⁺ m/z 291.16, found 291.09.

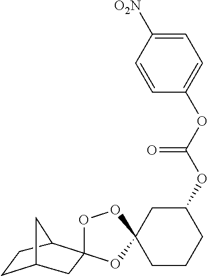

Prepared according to general procedure D using 5 mL DCM, alcohol 4f (50.0 mg, 0.21 mmol, 1.0 equiv), diisopropylethylamine (80.7 mg, 0.62 mmol, 3.0 equiv), 4-dimethylaminopyridine (25.4 mg, 0.21 mmol, 1.0 equiv) and 4-nitrophenyl chloroformate (126.0 mg, 0.62 mmol, 3.0 equiv). Chromatography (0-50% EtOAc/hexanes gradient elution) to afforded 56.0 mg (66%) of 5f as oil. ¹H NMR (400 MHz, CDCl₃) δ 8.29 (d, 2H, J=9.3 Hz), 7.39 (d, 2H, J=9.3 Hz), 4.76-4.90 (m, 1H), 2.29-2.51 (m, 3H), 2.08-2.18 (m, 1H), 1.31-2.00 (m, 14H); ¹³C NMR (100 MHz, CDCl₃) δ 155.3, 151.3, 145.3, 125.3, 121.7, 116.6 (two peaks), 108.2 (two peaks), 76.1, 45.1, 45.0, 41.6, 41.2, 39.8, 38.9, 37.5 (two peaks), 36.3 (two peaks), 33.6, 32.8, 30.0, 27.7, 21.5, 19.7, 19.4 several minor diastereomer peaks overlapping or not observed; LRMS(ESI) calculated for C₂₀H₂₃O₈NNa [M+Na]⁺ m/z 428.13, found 428.14.

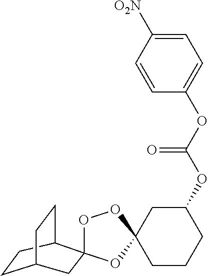

Prepared according to general procedure D using 5 mL DCM, alcohol 4g (12.0 mg, 0.05 mmol, 1.0 equiv), diisopropylethylamine (18.0 mg, 0.14 mmol, 3.0 equiv), 4-dimethylaminopyridine (5.8 mg, 0.05 mmol, 1.0 equiv) and 4-nitrophenyl chloroformate (29.0 mg, 0.14 mmol, 3.0 equiv). Chromatography (0-50% EtOAc/hexanes gradient elution) to afforded 10.0 mg (51%) of 5g as oil. ¹H NMR (400 MHz, CDCl₃) δ 8.28 (d, 2H, J=9.3 Hz), 7.38 (d, 2H, J=9.3 Hz), 4.77-4.94 (m, 1H), 2.21-2.58 (m, 1H), 1.77-2.02 (m, 9H), 1.39-1.59 (m, 10H); ¹³C NMR (100 MHz, CDCl₃) δ 155.5, 151.5, 145.3, 125.3, 121.7, 111.8 (two peaks), 108.2 (two peaks), 76.1, 39.8, 39.2, 38.2, 38.0, 33.8, 33.2 (two peaks), 30.1, 25.9, 24.4, 24.1, 21.1 (two peaks), 19.3 several minor diastereomer peaks overlapping or not observed; LRMS(ESI) calculated for C₂₁H₃₅O₈NNa [M+Na]⁺ m/z 442.15, found 442.29.

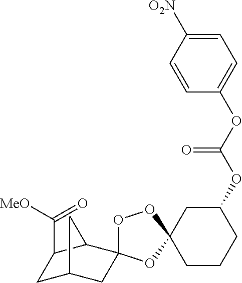

Prepared according to general procedure D using 5 mL DCM, alcohol 4r (90.0 mg, 0.30 mmol, 1.0 equiv), diisopropylethylamine (117.0 mg, 0.91 mmol, 3.0 equiv), 4-dimethylaminopyridine (36.9 mg, 0.30 mmol, 1.0 equiv) and 4-nitrophenyl chloroformate (182.0 mg, 0.91 mmol, 3.0 equiv). Chromatography (0-50% EtOAc/hexanes gradient elution) to afford 91.0 mg (65%) of intermediate 5r as oil. ¹H NMR (400 MHz, Chloroform-d) δ 8.27 (d, J=9.2 Hz, 2H), 7.38 (d, J=9.2 Hz, 2H), 4.96-4.72 (m, 1H), 3.68 (s, 3H), 2.88 (dd, J=9.3, 5.2 Hz, 1H), 2.58 (s, 1H), 2.48 (ddt, J=13.0, 4.4, 2.0 Hz, 1H), 2.36 (s, 1H), 2.18-2.10 (m, 1H), 1.99-1.79 (m, 6H), 1.68-1.45 (m, 6H); LRMS(ESI) calculated for C₂₂H₂₅O₁₀NNa [M+Na]⁺ m/z 486.13, found 486.17.

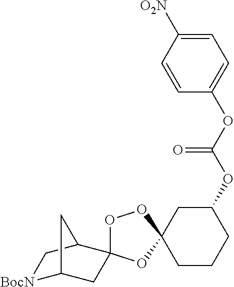

Prepared according to general procedure D using 5 mL DCM, alcohol 4s (120.0 mg, 0.35 mmol, 1.0 equiv), diisopropylethylamine (136.0 mg, 1.05 mmol, 3.0 equiv), 4-dimethylaminopyridine (43.0 mg, 0.35 mmol, 1.0 equiv) and 4-nitrophenyl chloroformate (212.0 mg, 1.05 mmol, 3.0 equiv). Chromatography (0-50% EtOAc/hexanes gradient elution) to afford 105.0 mg (59%) of intermediate 5s as oil. ¹H NMR (400 MHz, Chloroform-d) δ 8.25 (d, J=9.2 Hz, 2H), 7.36 (d, J=9.2 Hz, 2H), 4.92-4.70 (m, 1H), 4.27-4.09 (m, 1H), 3.41 (t, J=10.8 Hz, 1H), 3.10 (t, J=11.0 Hz, 1H), 2.57 (s, 1H), 2.51-2.25 (m, 1H), 2.18-1.73 (m, 9H), 1.43 (s, 11H); LRMS(ESI) calculated for C₂₄H₃₀O₁₀N₂Na [M+Na]⁺ m/z 529.17, found 529.19.

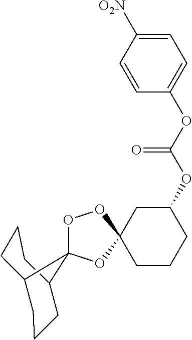

Prepared according to general procedure D using 5 mL DCM, alcohol 4h (85.0 mg, 0.32 mmol, 1.0 equiv), diisopropylethylamine (123.0 mg, 0.95 mmol, 3.0 equiv), 4-dimethylaminopyridine (38.7 mg, 0.32 mmol, 1.0 equiv) and 4-nitrophenyl chloroformate (192.0 mg, 0.95 mmol, 3.0 equiv). Chromatography (0-50% EtOAc/hexanes gradient elution) to afforded 83.8 mg (61%) of 5h as oil. ¹H NMR (400 MHz, CDCl₃) δ 8.28 (d, 2H, J=9.3 Hz), 7.38 (d, 2H, J=9.3 Hz), 4.77-5.04 (m, 1H), 2.34-2.45 (m, 1H), 1.41-2.10 (m, 21H); ¹³C NMR (100 MHz, CDCl₃) δ 155.5, 151.5, 145.3, 125.2, 121.7, 111.9, 108.1, 76.2, 39.6, 36.2, 36.1, 33.5, 30.0, 29.6, 29.4, 29.3 (two peaks), 20.8, 20.4, 19.5 several minor diastereomer peaks overlapping or not observed; LRMS(ESI) calculated for C₂₂H₂₇O₈NNa [M+Na]⁺ m/z 456.16, found 456.01.

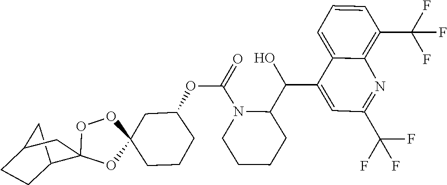

Prepared according to general procedure E using 5 mL DMF, intermediate 5f (10.0 mg, 0.03 mmol, 1.0 equiv), diisopropylethylamine (16.0 mg, 0.12 mmol, 5.0 equiv), 4-dimethylaminopyridine (1.5 mg, 0.01 mmol, 0.5 equiv) and mefloquine (12.0 mg, 0.03 mmol, 1.2 equiv). Chromatography (0-80% EtOAc/hexanes gradient elution) to afforded 8.3 mg (52%) of 6f as oil. ¹H NMR (400 MHz, CDCl₃) δ 8.70 (dd, 1H, J=3.4, 8.0 Hz), 8.19 (d, 1H, J=7.1 Hz), 8.09 (s, 1H), 7.79 (t, 1H, J=7.1 Hz), 5.93 (br, 1H), 4.69-4.85 (m, 1H), 4.21-4.36 (m, 1H), 3.86-4.02 (m, 1H), 3.29-3.44 (m, 1H), 2.99 (br, 1H), 2.29 (s, 3H), 1.45-1.89 (m, 21H); ¹⁹F NMR (376 MHz, CDCl₃) δ −60.3, −67.9; ¹³C NMR (100 MHz, CDCl₃) δ 155.6, 150.9, 150.7, 143.7, 129.5, 128.9 (multiple peaks), 128.2 (two peaks), 127.3 (two peaks), 126.7, 122.6, 122.2, 116.5, 116.4, 116.3, 115.4 (two peaks), 109.0 (two peaks), 108.5 (two peaks), 72.2 (two peaks), 71.6, 68.0 (two peaks), 56.9 (multiple peaks), 44.9, 42.2, 41.1-41.6 (multiple peaks), 40.3 (two peaks), 39.6 (two peaks), 37.5-37.6 (multiple peaks), 35.3 (two peaks), 34.0, 33.9 (two peaks), 33.0-33.2 (multiple peaks), 30.6, 29.7 (multiple peaks), 27.7, 24.0 (two peaks), 22.1, 21.6 (two peaks), 19.8, 19.4, 19.0 several minor diastereomer peaks overlapping or not observed; HRMS(ESI) calculated for C₃₁H₃₄O₆N₂F₆Na [M+Na]⁺ m/z 667.2213, found 667.2220.

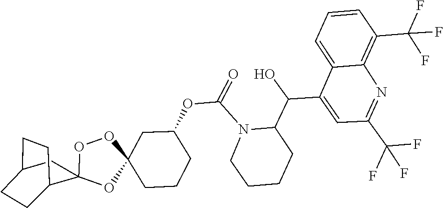

Prepared according to general procedure E using 5 mL DMF, intermediate 5g (10.0 mg, 0.02 mmol, 1.0 equiv), diisopropylethylamine (15.0 mg, 0.12 mmol, 5.0 equiv), 4-dimethylaminopyridine (1.5 mg, 0.01 mmol, 0.5 equiv) and mefloquine (12.0 mg, 0.03 mmol, 1.2 equiv). Chromatography (0-80% EtOAc/hexanes gradient elution) to afforded 7.6 mg (48%) of 6g as oil. ¹H NMR (400 MHz, CDCl₃) δ 8.71 (d, 1H, J=8.7 Hz), 8.19 (d, 1H, J=7.3 Hz), 8.11 (s, 1H), 7.79 (app t, 1H, J=7.8 Hz), 5.94 (br, 1H), 4.71-4.88 (m, 1H), 4.23-4.35 (m, 1H), 3.87-4.00 (m, 1H), 3.35-3.47 (m, 1H), 2.80-3.12 (br, 1H), 2.06-2.16 (m, 1H), 1.45-1.94 (m, 25H); ¹⁹F NMR (376 MHz, CDCl₃) δ −60.3, −67.9; ¹³C NMR (100 MHz, CDCl₃) δ 155.7, 150.8, 150.7, 143.7, 128.9, 128.8, 128.2, 127.3, 126.7, 122.6, 115.4, 111.7, 111.5, 108.6, 108.5, 72.2, 71.6, 68.0, 57.0, 42.2, 41.3, 39.6, 39.5, 38.0-38.2 (multiple peaks), 34.1 (two peaks), 33.2, 32.8 (multiple peaks), 30.6, 25.9 (two peaks), 24.4, 24.1, 24.0, 21.1, 21.0 (two peaks), 19.9, 19.4, 19.0 several minor diastereomer peaks overlapping or not observed; HRMS(ESI) calculated for C₃₂H₃₆O₆N₂F₆Na [M+Na]⁺ m/z 681.2370, found 681.2386.

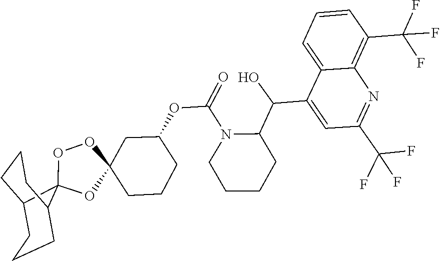

Prepared according to general procedure E using 5 mL DMF, intermediate 5h (10.0 mg, 0.02 mmol, 1.0 equiv), diisopropylethylamine (15.0 mg, 0.12 mmol, 5.0 equiv), 4-dimethylaminopyridine (1.4 mg, 0.01 mmol, 0.5 equiv) and mefloquine (11.5 mg, 0.03 mmol, 1.2 equiv). Chromatography (0-80% EtOAc/hexanes gradient elution) to afford 7.0 mg (50%) of 6h as oil. ¹H NMR (400 MHz, CDCl₃) δ 8.73 (d, 1H, J=8.7 Hz), 8.20 (d, 1H, J=7.1 Hz), 8.11 (app d, 1H, J=3.7 Hz), 7.81 (app t, 1H, J=7.8 Hz), 5.95 (br, 1H), 4.77-4.88 (m, 1H), 4.25-4.39 (m, 1H), 3.91-4.05 (m, 1H), 3.35-3.47 (m, 1H), 2.19-2.28 (m, 1H), 1.45-2.07 (m, 28H); ¹⁹F NMR (376 MHz, CDCl₃) δ −60.3, −67.9; ¹³C NMR (100 MHz, CDCl₃) δ 155.6, 151.0, 150.8, 143.7, 128.9, 128.8, 128.2 (two peaks), 127.3, 126.8, 122.6, 122.2, 115.4, 111.9, 111.7, 108.9, 108.5, 72.2, 71.6, 68.0, 56.9, 42.2, 41.7, 40.9, 40.0, 36.0-36.2 (multiple peaks), 34.1 (two peaks), 33.8, 33.1, 30.6, 29.3-29.7 (multiple peaks), 24.0 (two peaks), 20.9, 20.8, 19.9, 19.5, 19.1 several minor diastereomer peaks overlapping or not observed; HRMS(ESI) calculated for C₃₃H₃₈O₆N₂F₆Na [M+Na]⁺ m/z 695.2526, found 695.2538.

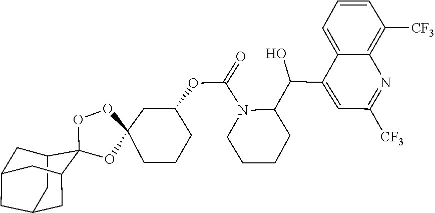

Prepared according to general procedure E using 5 mL DMF, intermediate 5a (100.0 mg, 0.22 mmol, 1.0 equiv), diisopropylethylamine (145.0 mg, 1.12 mmol, 5.0 equiv), 4-dimethylaminopyridine (13.7 mg, 0.11 mmol, 0.5 equiv) and mefloquine (112.0 mg, 0.27 mmol, 1.2 equiv). Chromatography (0-80% EtOAc/hexanes gradient elution) to afford 91.6 mg (60%) of 6a as a colorless solid. ¹H NMR (400 MHz, CDCl₃) δ 8.67 (br t, J=8.4 Hz, 1H), 8.17 (t, J=6.5 Hz, 1H), 8.10 (d, J=5.8 Hz, 1H), 7.76 (dt, J=11.8, 8.1 Hz, 1H), 5.90 (br d, J=3.4 Hz, 1H), 4.69-4.99 (m, 1H), 4.69-4.88 (m, 1H), 4.18-4.36 (m, 1H), 3.95 (br t, J=14.9 Hz, 1H), 3.23-3.49 (m, 1H), 3.12 (br s, 1H), 2.17-2.28 (m, 1H), 1.61-2.14 (m, 24H), 1.73-1.88 (m, 4H), 1.40-1.63 (m, 2H); ¹³C NMR (100 MHz, CDCl₃) δ 171.3, 155.6 (several peaks), 151.1, 150.9, 148.4, 148.3, 143.7, 129.4, 129.1, 128.8 (several peaks), 128.2, 128.1, 127.3, 126.8 (2 peaks), 125.4, 124.9 (2 peaks), 123.8, 122.7, 122.2, 121.7, 119.9, 115.4 (several peaks), 111.7, 108.6, 108.6, 77.2, 72.2, 72.0, 71.7, 60.5, 56.8 (several peaks), 42.1 (several peaks), 40.1, 39.9, 36.8, 36.6, 36.3 (3 peaks), 34.9, 34.8 (several peaks), 34.7, 34.0, 33.9, 30.6, 26.9, 26.4, 24.7, 24.0, 22.2, 21.1, 19.8 (several peaks), 19.6 (2 peaks); ¹⁹F NMR (376 MHz, CDCl₃) δ −60.30, −60.33, −67.87; HRMS (ESI): m/z [M]+Na⁺ calcd for: C₃₄H₃₈F₆N₂O₆Na: 707.2526, found m/z: 707.2529; LRMS (ESI): m/z [M+Na]⁺ calcd for: C₃₄H₃₈F₆N₂O₆Na: 707.25, found m/z: 707.25, retention time (diode array 290 nm)=6.51 mins.

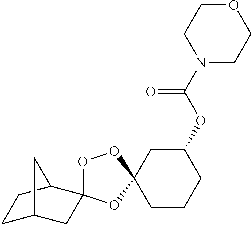

Prepared according to general procedure F using 5 mL DCM, intermediate 5f (18.0 mg, 0.04 mmol, 1.0 equiv), triethylamine (13.0 mg, 0.13 mmol, 3.0 equiv), and morpholine HCl salt (7.7 mg, 0.06 mmol, 1.4 equiv). Chromatography (0-50% EtOAc/hexanes gradient elution) to afforded 8.0 mg (51%) of 7f as oil. ¹H NMR (400 MHz, CDCl₃) δ 4.76-4.87 (m, 1H), 3.61-3.70 (m, 4H), 3.42-3.51 (m, 4H), 2.15-2.30 (m, 3H), 1.65-2.00 (m, 7H), 1.24-1.58 (m, 8H); ¹³C NMR (100 MHz, CDCl₃) δ 154.6, 116.3 (two peaks), 108.5 (two peaks), 71.4, 71.3, 66.6 (br), 45.0 (two peaks), 41.6, 40.1, 39.3, 37.6 (two peaks), 35.3 (two peaks), 34.0, 33.0, 30.6, 27.7 (two peaks), 21.6, 19.7, 19.4 several minor diastereomer peaks overlapping or not observed; HRMS(ESI) calculated for C₁₈H₂₇O₆NNa [M+Na]⁺ m/z 376.1731, found 376.1733.

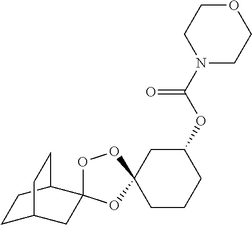

Prepared according to general procedure F using 5 mL DCM, intermediate 5g (12.0 mg, 0.03 mmol, 1.0 equiv), triethylamine (8.7 mg, 0.09 mmol, 3.0 equiv), and morpholine HCl salt (4.9 mg, 0.04 mmol, 1.4 equiv). Chromatography (0-50% EtOAc/hexanes gradient elution) to afforded 6.0 mg (60%) of 7g as oil. ¹H NMR (400 MHz, CDCl₃) δ 4.76-5.03 (m, 1H), 3.61-3.73 (m, 4H), 3.44-3.51 (m, 4H), 2.09-2.35 (m, 1H), 1.40-1.97 (m, 19H); ¹³C NMR (100 MHz, CDCl₃) δ 154.6, 111.4, 108.5 (two peaks), 71.5, 71.3, 66.6 (two peaks), 40.2, 39.4, 38.3, 38.0, 34.3, 33.4, 32.8 (two peaks), 30.6 (two peaks), 25.9, 24.5, 24.1, 21.0 (three peaks), 19.7, 19.4 several minor diastereomer peaks overlapping or not observed; HRMS(ESI) calculated for C₁₉H₂₉O₆NNa [M+Na]⁺ m/z 390.1889, found 390.1887.

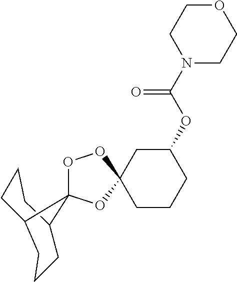

Prepared according to general procedure F using 5 mL DCM, intermediate 5h (25.0 mg, 0.06 mmol, 1.0 equiv), triethylamine (18.0 mg, 0.17 mmol, 3.0 equiv), and morpholine HCl salt (10 mg, 0.08 mmol, 1.4 equiv). Chromatography (0-50% EtOAc/hexanes gradient elution) to afforded 16.0 mg (73%) of 7h as oil. ¹H NMR (400 MHz, CDCl₃) δ 4.78-4.97 (m, 1H), 3.61-3.70 (m, 4H), 3.42-3.51 (m, 4H), 2.20-2.28 (m, 1H), 1.46-2.05 (m, 21H); ¹³C NMR (100 MHz, CDCl₃) δ 154.7, 111.7, 108.5, 71.4, 66.6 (br), 39.8, 36.2 (two peaks), 34.2, 30.6, 29.7, 29.4, 29.2, 24.7, 20.9, 20.4, 19.4 several minor diastereomer peaks overlapping or not observed; HRMS(ESI) calculated for C₂₀H₃₁O₆NNa [M+Na]⁺ m/z 404.2044, found 404.2049.

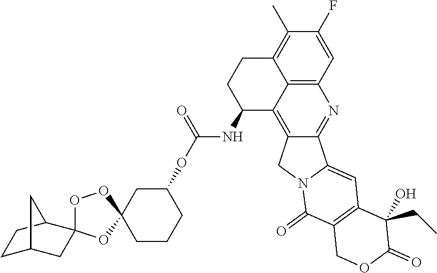

To a solution of intermediate 5f (25.0 mg, 0.062 mmol, 1.0 equiv) in 5 mL DMF was added N,N-diisopropylethylamine (24.0 mg, 0.185 mmol, 3.0 equiv), dimethylaminopyridine (9.0 mg, 0.074 mmol, 1.2 equiv) followed by exatecan mesylate (32.8 mg, 0.062 mmol, 1.0 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude residue was purified using reverse phase C18 column chromatography (0-100% water-MeCN) to give 23.1 mg of 8f (53.4%). ¹H NMR (400 MHz, Chloroform-d) δ 7.59-7.34 (m, 2H), 5.69-5.57 (m, 1H), 5.38-4.78 (m, 5H), 3.96 (d, J=6.5 Hz, 1H), 3.20-2.98 (m, 2H), 2.58-2.12 (m, 8H), 1.98-1.03 (m, 21H); LRMS(ESI) calculated for C₃₈H₄₁FN₃O₉[M+H]⁺ m/z 702.28, found 702.30.

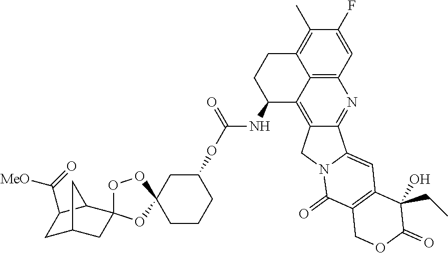

To a solution of intermediate 5r (25.0 mg, 0.054 mmol, 1.0 equiv) in 5 mL DMF was added N,N-diisopropylethylamine (21.0 mg, 0.162 mmol, 3.0 equiv), dimethylaminopyridine (8.0 mg, 0.065, 1.2 equiv) followed by, exatecan mesylate (28.7 mg, 0.054 mmol, 1.0 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude residue was purified using reverse phase C18 column chromatography (0-100% water-MeCN) to give 15.6 mg of 8r (38%). ¹H NMR (400 MHz, Chloroform-d) δ 7.63 (d, J=10.6 Hz, 1H), 7.59 (s, 1H), 5.72 (d, J=16.3 Hz, 1H), 5.39-4.84 (m, 6H), 3.77-3.64 (m, 3H), 3.16 (s, 2H), 2.98-2.89 (m, 1H), 2.68-2.54 (m, 1H), 2.47-2.20 (m, 8H), 1.99-1.39 (m, 14H), 1.12-1.04 (m, 3H); LRMS(ESI) calculated for C₄₀H₄₃FN₃O₁₁ [M+H]⁺ m/z 760.28, found 760.27.

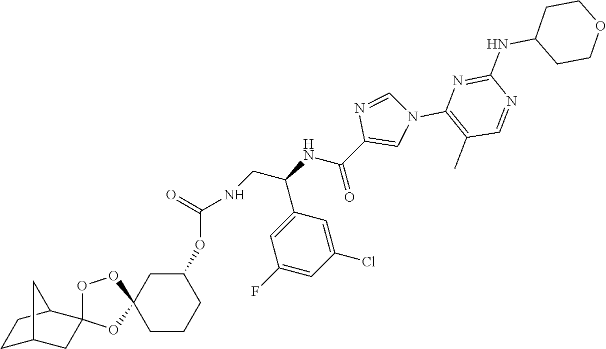

To a solution of intermediate 5f (25.0 mg, 0.062 mmol, 1.0 equiv) in 5 mL DMF was added N,N-diisopropylethylamine (24.0 mg, 0.185 mmol, 3.0 equiv), dimethylaminopyridine (9.0 mg, 0.074 mmol, 1.2 equiv) followed by, ASN007 (29.2 mg, 0.062 mmol, 1.0 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude residue was purified using fresh column chromatography (0-30% DCM-MeOH) to give 26.4 mg of 9f (57.8%). ¹H NMR (400 MHz, Chloroform-d) δ 8.24 (s, 1H), 8.18-7.99 (m, 2H), 7.16 (s, 1H), 7.11-6.91 (m, 2H), 5.36-5.07 (m, 3H), 4.87-4.63 (m, 1H), 4.05-3.90 (m, 3H), 3.70-3.35 (m, 4H), 2.34-2.21 (m, 5H), 2.05-1.20 (m, 20H); LRMS(ESI) calculated for C₃₆H₄₄FClN₇O₇[M+H]⁺ m/z 740.30, found 740.32.

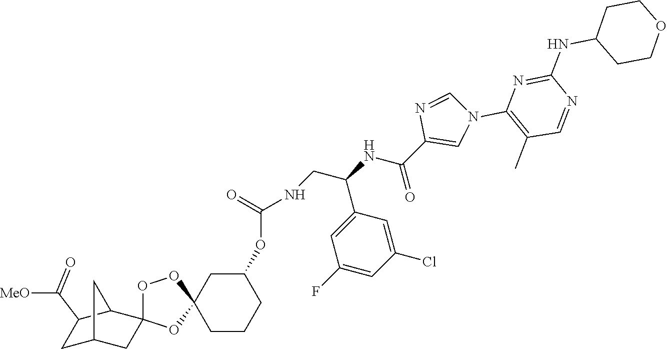

To a solution of intermediate 5r (25.0 mg, 0.054 mmol, 1.0 equiv) in 5 mL DMF was added N,N-diisopropylethylamine (21.0 mg, 0.162 mmol, 3.0 equiv), dimethylaminopyridine (8.0 mg, 0.065, 1.2 equiv) followed by, ASN007 (28.7 mg, 0.054 mmol, 1.0 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude residue was purified using fresh column chromatography (0-30% DCM-MeOH) to give 15.3 mg of 9r (35.5%). ¹H NMR (400 MHz, Chloroform-d) δ 8.25 (s, 1H), 8.18-8.00 (m, 2H), 7.21-7.10 (m, 1H), 7.06-6.95 (m, 2H), 5.34-4.91 (m, 3H), 4.85-4.63 (m, 1H), 3.99 (dt, J=11.9, 3.7 Hz, 3H), 3.78-3.47 (m, 6H), 2.92-2.79 (m, 1H), 2.41-2.12 (m, 5H), 2.05-1.17 (m, 20H); LRMS(ESI) calculated for C₃₈H₄₆FClN₇O₉N [M+H]⁺ m/z 798.30, found 789.29.

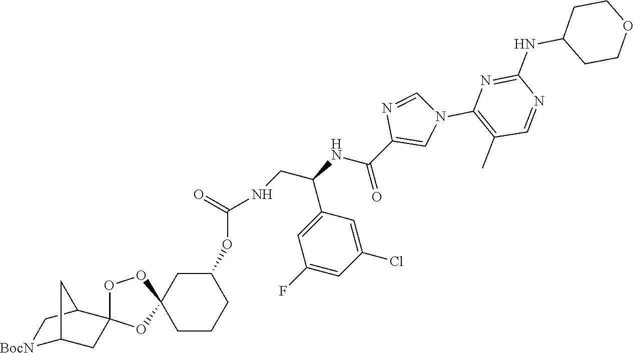

To a solution of intermediate 5s (28.0 mg, 0.055 mmol, 1.0 equiv) in 5 mL DMF was added N,N-diisopropylethylamine (21.4 mg, 0.17 mmol, 3.25 equiv), followed by, ASN007 (28.8 mg, 0.061 mmol, 1.1 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude residue was purified using fresh column chromatography (0-30% DCM-MeOH) to give 29 mg of 9s (70%). ¹H NMR (400 MHz, Chloroform-d) δ 8.25 (s, 1H), 8.17-8.00 (m, 3H), 7.16 (s, 1H), 7.00 (d, J=8.6 Hz, 2H), 5.30-5.03 (m, 3H), 4.81-4.60 (m, 1H), 4.27-4.05 (m, 1H), 3.99 (dt, J=11.7, 3.6 Hz, 3H), 3.68-3.36 (m, 5H), 3.21-3.01 (m, 1H), 2.63-2.47 (m, 1H), 2.28 (s, 3H), 2.24-1.64 (m, 13H), 1.63-1.50 (m, 3H), 1.43 (s, 9H); LRMS(ESI) calculated for C₄₀H₅₁FClN₈O₉ [M+H]⁺ m/z 841.35, found 841.46.

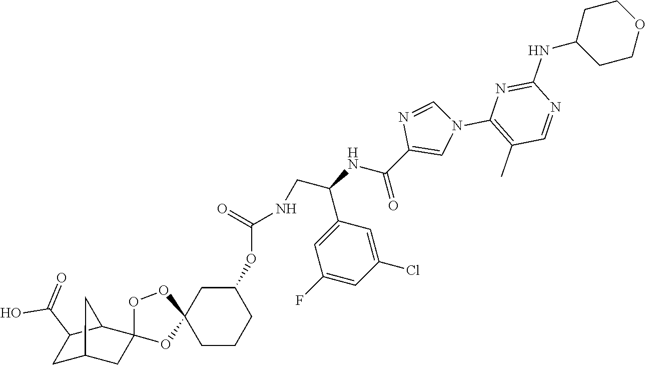

To a solution of 9r (3.2 mg, 0.004 mmol, 1 equiv) in 5 mL IPA was added 1 M LiOH (32 μL, 0.032 mmol, 8 equiv). The reaction was stirred at 40° C. for 16 hours. The reaction mixture was concentrated under reduced pressure. The crude was dissolved in DMSO and was purified using reverse phase C18 column chromatography (0-100% water-MeCN) to give 2.3 mg of 12r (73%). ¹H NMR (400 MHz, Chloroform-d) δ 8.32-8.18 (m, 1H), 8.19-7.94 (m, 2H), 7.17 (s, 1H), 7.09-6.91 (m, 2H), 5.31-4.48 (m, 4H), 4.06-3.82 (m, 3H), 3.71-3.40 (m, 3H), 3.36-3.09 (m, 1H), 2.35-2.03 (m, 5H), 2.00-1.15 (m, 20H); LRMS(ESI) calculated for C₃₇H₄₄ClFN₇O₉ [M+H]⁺ m/z 784.28, found 783.29.

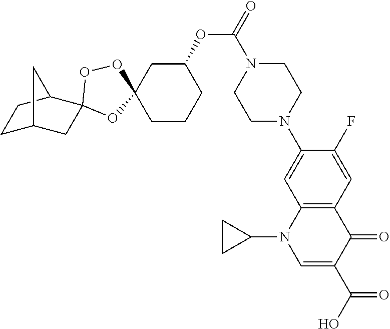

To a solution of intermediate 5f (25.0 mg, 0.062 mmol, 1.0 equiv) in 5 mL DMF was added N,N-diisopropylethylamine (24.0 mg, 0.185 mmol, 3.0 equiv), followed by, ciprofloxacin (51.0 mg, 0.15 mmol, 2.5 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude was dissolved in DMSO and was purified using reverse phase C18 column chromatography (0-100% water-MeCN) to give 20.4 mg of 10f (55%). ¹H NMR (400 MHz, Methanol-d₄/Dichloromethane-d₂=1:0.5) δ 8.91 (br, 1H), 8.04 (br, 1H), 7.48 (br, 1H), 4.78 (s, 1H), 3.88-3.49 (m, 6H), 3.25-3.42 (m, 4H, overlap MeOD peak), 2.41-2.14 (m, 4H), 2.01-1.29 (m, 21H); LRMS(ESI) calculated for C₃₁H₃₇FN₃O₈[M+H]⁺ m/z 598.26, found 598.26.

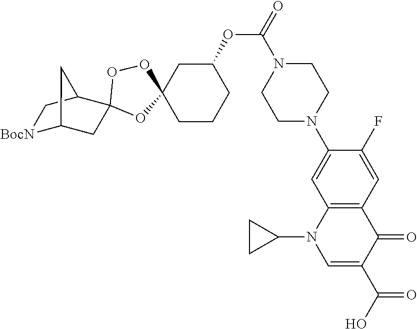

To a solution of intermediate 5s (30.0 mg, 0.059 mmol, 1.0 equiv) in 5 mL DMF was added N,N-diisopropylethylamine (25.0 mg, 0.19 mmol, 3.25 equiv), followed by, ciprofloxacin (49.0 mg, 0.15 mmol, 2.5 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude was dissolved in DMSO and was purified using reverse phase C18 column chromatography (0-100% water-MeCN) to give 29 mg of 10s (70%). ¹H NMR (400 MHz, Chloroform-d) δ 8.73 (s, 1H), 7.99 (d, J=12.9 Hz, 1H), 7.35 (d, J=7.0 Hz, 1H), 4.87-4.70 (m, 1H), 4.35-4.07 (m, 1H), 3.82-3.61 (m, 4H), 3.55 (s, 1H), 3.43 (t, J=11.0 Hz, 1H), 3.38-3.20 (m, 4H), 3.11 (t, J=10.9 Hz, 1H), 2.60 (s, 1H), 2.37-2.20 (m, 1H), 2.07-1.68 (m, 9H), 1.58-1.16 (m, 15H); LRMS(ESI) calculated for C₃₅H₄₄FN₄O₁₀ [M+H]⁺ m/z 699.30, found 699.35.

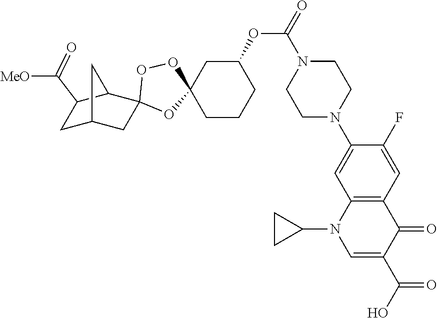

To a solution of intermediate 5r (40.0 mg, 0.086 mmol, 1.0 equiv) in 5 mL DMF was added N,N-diisopropylethylamine (36.0 mg, 0.28 mmol, 3.25 equiv), followed by, ciprofloxacin (71.0 mg, 0.22 mmol, 2.5 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude was dissolved in DMSO and was purified using reverse phase C18 column chromatography (0-100% water-MeCN) to give 20 mg of 10r (35%). ¹H NMR (400 MHz, Chloroform-d) δ 14.92 (s, 1H), 8.75 (d, J=1.5 Hz, 1H), 8.02 (dd, J=12.8, 1.7 Hz, 1H), 7.36 (dd, J=7.0, 4.1 Hz, 1H), 4.94-4.64 (m, 1H), 3.95-3.68 (m, 4H), 3.67 (s, 3H), 3.45-3.22 (m, 4H), 2.94-2.82 (m, 1H), 2.58 (s, 1H), 2.43-2.21 (m, 2H), 2.02-1.35 (m, 16H), 1.27-1.20 (m, 4H); LRMS(ESI) calculated for C₃₃H₃₉FN₃O₁₀ [M+H]⁺ m/z 656.26, found 656.23.

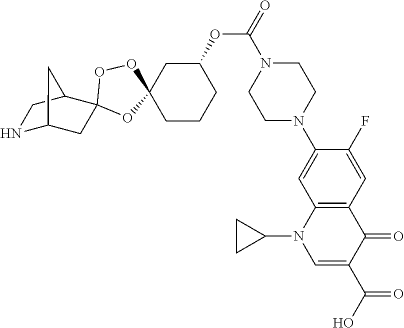

To a solution of 10s (29 mg, 0.042 mmol, 1 equiv) in 5 mL of IPA was added acetyl chloride (89 μL, 1.2 mmol, 30 equiv). The reaction was stirred at RT overnight. The reaction mixture was concentrated under reduced pressure to remove solvent. The crude was dissolved in DMSO and was purified using reverse phase C18 column chromatography (0-100% water-MeCN) to give 7.2 mg of 13s (29%). ¹H NMR (400 MHz, DMSO-d₆) δ 8.67 (s, 1H), 7.93 (dd, J=13.2, 4.8 Hz, 1H), 7.67-7.53 (m, 1H), 6.55 (s, 1H), 4.64 (s, 1H), 4.04 (s, 1H), 3.86-3.66 (m, 2H), 3.70-3.50 (m, 4H), 3.14-2.95 (m, 4H), 2.71 (s, 1H), 2.34-1.15 (m, 17H); LRMS(ESI) calculated for C₃₀H₃₆FN₄O₈[M+H]⁺ m/z 599.25, found 599.31.

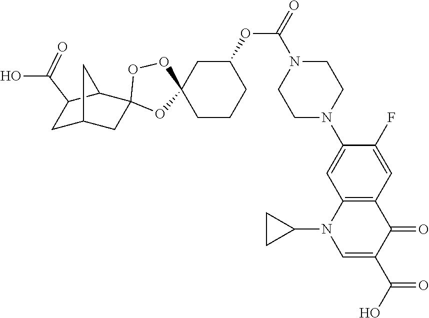

To a solution of 10r (20 mg, 0.031 mmol, 1 equiv) in 5 mL IPA was added 1 M LiOH (210 μL, 0.21 mmol, 7 equiv). The reaction was stirred at 40° C. for 16 hours. The reaction mixture was concentrated under reduced pressure. The crude was dissolved in DMSO and was purified using reverse phase C18 column chromatography (0-100% water-MeCN) to give 7.1 mg of 14r (36%). ¹H NMR (400 MHz, DMSO-d₆) δ 8.69 (d, J=2.6 Hz, 1H), 7.85 (dd, J=13.4, 5.2 Hz, 1H), 7.49 (d, J=7.1 Hz, 1H), 4.65 (s, 1H), 4.12-4.02 (m, 1H), 3.81-3.51 (m, 4H), 3.27-3.14 (m, 4H), 2.45-2.13 (m, 4H), 1.96-1.66 (m, 6H), 1.49-1.00 (m, 11H); LRMS(ESI) calculated for C₃₂H₃₇FN₃O₁₀ [M+H]⁺ m/z 642.24, found 642.33.

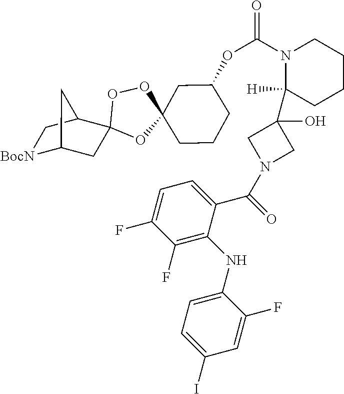

To a solution of 5s (30.0 mg, 0.059 mmol, 1.0 equiv) in 5 mL DMF was added N,N-diisopropylethylamine (61.0 mg, 0.47 mmol, 8 equiv), followed by dimethylaminopyridine (8.7 mg, 0.087, 1.2 equiv) and cobimetnib (94.4 mg, 0.178 mmol, 3 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude residue was purified using fresh column chromatography (0-30% DCM-MeOH) to give 27 mg of 11s (51%). ¹H NMR (400 MHz, Chloroform-d) δ 8.54 (br, 1H), 8.38 (br, 1H), 7.38 (dd, J=10.3, 1.9 Hz, 1H), 7.31 (dt, J=8.4, 1.5 Hz, 1H), 7.12 (br, 1H), 6.81 (d, J=8.1 Hz, 1H), 6.73-6.51 (m, 1H), 5.32 (br, 1H), 4.82-4.60 (m, 1H), 4.28-3.81 (m, 7H), 3.47-3.34 (m, 2H), 3.10 (t, J=13.1 Hz, 1H), 2.91 (s, 1H), 2.57 (s, 1H), 1.99-1.27 (m, 26H); LRMS(ESI) calculated for C₃₉H₄₆F₃IN₄O₉[M+H]⁺ m/z 899.23, found 899.18.

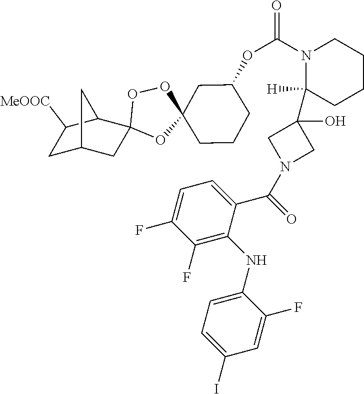

To a solution of 5r (25.0 mg, 0.054 mmol, 1.0 equiv) in 5 mL DMF was added N,N-diisopropylethylamine (2.7 mg, 0.175 mmol, 3.25 equiv), followed by dimethylaminopyridine (7.9 mg, 0.065, 1.2 equiv) and cobimetnib (86.0 mg, 0.162 mmol, 3 equiv) at rt. The bright yellow mixture was allowed to stir at rt for 16 h. The reaction was quenched with water (20 mL) and diluted with EtOAc (30 mL). The organic phase was separated and washed with additional water (4×30 mL). The combined aqueous layers were then back extracted with EtOAc (1×30 mL). The combined organic layers were then washed with brine (20 mL), dried (MgSO₄), filtered, and concentrated under reduced pressure. The crude residue was purified using fresh column chromatography (0-30% DCM-MeOH) to give 18 mg of 11r (39%). ¹H NMR (400 MHz, Chloroform-d) δ 8.55 (br, 1H, major), 8.40 (br, 1H, minor), 7.39 (dd, J=10.3, 1.9 Hz, 1H), 7.32 (d, J=8.5 Hz, 1H), 7.14 (s, 1H), 6.91-6.69 (m, 1H), 6.60 (q, J=7.6 Hz, 1H), 5.38 (br, 1H), 4.75 (s, 1H), 4.29-4.12 (m, 2H), 4.09-3.84 (m, 3H), 3.68 (s, 3H, minor), 3.67 (s, 3H, major), 3.37 (s, 1H), 2.88 (dt, J=9.7, 5.4 Hz, 2H), 2.57 (s, 1H), 2.41-2.28 (m, 1H), 2.26-1.28 (m, 19H); LRMS(ESI) calculated for C₃₇H₄₂F₃IN₃O₉ [M+H]⁺ m/z 856.19, found 856.11.

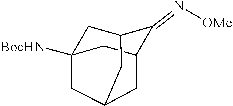 [SMILES: CON=C1C2CC3CC1CC(NC(=O)OC(C)(C)C)(C3)C2]

Prepared according to general procedure G using 35 mL MeOH, tert-butyl (4-(oxo)adamantan-1-yl)carbamate (4.61 g, 17.4 mmol, 1.0 equiv), pyridine (2.76 g, 34.8 mmol, 2.0 equiv), and O-methylhydroxylamine hydrochloride (2.23 g, 26.7 mmol, 1.5 equiv). Crude oxime 1b (5.0 g, 98%) was obtained as solid. ¹H NMR (400 MHz, CDCl₃) δ 4.43 (br s, 1H), 3.79 (s, 3H), 3.55 (br s, 1H), 2.63 (br s, 1H), 2.14-2.21 (m, 1H), 2.00-2.14 (m, 5H), 1.92-2.00 (m, 2H), 1.83-1.89 (m, 1H), 1.67-1.83 (m, 3H), 1.41 (s, 9H); ¹³C NMR (100 MHz, CDCl₃) δ 164.2, 61.0, 50.1, 42.4, 41.1, 37.9, 36.6, 36.4, 29.7, 29.0, 28.4; LRMS(ESI) calculated for C₁₆H₂₇N₂O₃ [M+H]⁺ m/z 295.20, found 295.29.

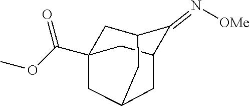 [SMILES: CON=C1C2CC3CC1CC(C(=O)OC)(C3)C2]

Prepared according to general procedure G using 30 mL MeOH, benzyl 3-oxopyrrolidine-1-carboxylate (1.0 g, 4.4 mmol, 1.0 equiv), pyridine (0.52 g, 6.6 mmol, 1.5 equiv), and O-methylhydroxylamine hydrochloride (0.55 g, 6.6 mmol, 1.5 equiv). Crude oxime 1c (0.84 g, 82%) was obtained as solid. ¹H NMR (400 MHz, CDCl₃) δ3.78 (s, 3H), 3.63 (s, 3H), 3.51-3.53 (m, 1H), 2.56-2.63 (m), 2.09-2.14 (m, 1H), 2.03-2.09 (m, 1H), 2.00-2.03 (m, 1H), 1.94-2.00 (m, 4H), 1.87-1.94 (m, 1H), 1.79-1.85 (m, 2H), 1.72-1.79 (m, 1H); ¹³C NMR (100 MHz, CDCl₃) δ 176.7, 164.4, 61.0, 51.8, 40.7, 40.0, 38.6, 38.0, 36.5, 35.5, 28.7, 27.6; LRMS(ESI) calculated for C₁₃H₂₀NO₃ [M+H]⁺ m/z 238.14, found 237.96.

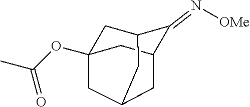 [SMILES: CON=C1C2CC3CC1CC(OC(C)=O)(C3)C2]

To a 500 mL pressure vessel containing a magnetic stir bar was added 4-oxoadamantan-1-yl acetate (1.18 g, 5.67 mmol, 1.0 equiv) followed by MeOH (17.5 mL), pyridine (917 uL, 11.3 mmol, 2.0 equiv) and methoxylamine hydrochloride (729 mg, 8.73 mmol, 1.5 equiv). The vessel was sealed with a teflon screw cap and heated to 90° C. behind a blast shield for 1.5 h. The mixture was then cooled to rt and the cap carefully unscrewed to allow for slow venting. The reaction mixture was transferred to an rb flask and concentrated under reduced pressure to a crude semi-solid. The crude residue was diluted with 10% aq KHSO₄ solution (60 mL) and extracted with EtOAc (1×200 mL). The organic phase was washed with additional 10% aq KHSO₄ solution (3×60 mL) and the aqueous layers back-extracted with EtOAc (1×150 mL). The combined organic phases were washed with brine (1×150 mL), dried (MgSO₄), filtered and concentrated to a colorless oil which solidified under high vacuum to give the desired oxime 1d (1.34 g, 5.65 mmol, 99%), which was sufficiently pure to be carried onto the next step without further purification. ¹H NMR (400 MHz, CDCl₃) δ 3.83 (s, 3H), 3.60-3.69 (m, 1H), 3.19-3.20 (m, 1H), 2.54-2.81 (m, 1H), 2.22-2.35 (m, 6H), 1.97-2.19 (m, 5H), 1.98 (s, 3H major diastereomer), 1.97 (s, 3H minor diasteromer), 1.88-1.94 (m, 1H), 1.53-1.87 (m, 4H); ¹³C NMR (100 MHz, CDCl₃) δ 170.2, 163.6, 79.4, 78.7, 77.2, 61.1, 41.7, 40.3 (2 peaks), 37.8, 37.5, 37.3, 36.4, 35.0, 32.7, 30.5, 30.3, 29.5, 22.6, 22.5; LRMS (ESI): m/z [M]+H⁺ calcd for: C₁₃H₂₀NO₃: 238.14 found m/z: 238.20.

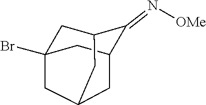 [SMILES: CON=C1C2CC3CC1CC(Br)(C3)C2]

Prepared according to general procedure G using 30 mL MeOH, benzyl 3-oxopyrrolidine-1-carboxylate (1.0 g, 4.4 mmol, 1.0 equiv), pyridine (0.52 g, 6.6 mmol, 1.5 equiv), and O-methylhydroxylamine hydrochloride (0.55 g, 6.6 mmol, 1.5 equiv). Crude oxime 1e (0.84 g, 82%) was obtained as oil. ¹H NMR (400 MHz, CDCl₃) δ 3.81 (s, 3H), 2.66 (br, 1H), 2.38-2.55 (m, 4H), 2.12-2.31 (m, 3H), 1.76-2.00 (m, 4H); ¹³C NMR (100 MHz, CDCl₃) δ 162.4, 64.3, 62.2, 61.1, 49.5, 49.0, 48.1, 45.0, 39.3, 37.1, 36.8, 35.7, 32.4, 31.8, 31.3; LRMS(ESI) calculated for C₁₁H₁₇BrON [M+H]⁺ m/z 258.05, found 257.99.

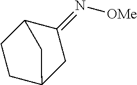

Prepared according to general procedure G using 300 mL MeOH, bicyclo[2.2.1]heptan-2-one (10.0 g, 90.8 mmol, 1.0 equiv), pyridine (10.8 g, 136.2 mmol, 1.5 equiv), and O-methylhydroxylamine hydrochloride (11.4 g, 136.2 mmol, 1.5 equiv). Crude oxime if (9.8 g, 78%) was obtained as oil. ¹H NMR (400 MHz, CDCl₃) δ 3.79 (S, 3H), 2.79-2.84 (m, 1H), 2.43-2.49 (m, 1H), 2.16-2.25 (m, 1H), 1.96-2.05 (m, 1H), 1.57-1.76 (m, 2H), 1.23-1.51 (m, 4H); ¹³C NMR (100 MHz, CDCl₃) δ 166.9, 61.2, 61.0, 42.3, 38.9, 38.3, 37.3, 35.2, 35.3, 35.1, 27.6, 27.3, 27.1, 26.1; LRMS(ESI) calculated for C₈H₁₄ON [M+H]⁺ m/z 140.11, found 140.13.

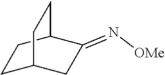 [SMILES: CO/N=C1\CC2CCC1CC2]

Prepared according to general procedure G using 30 mL MeOH, bicyclo[2.2.2]octan-2-one (1.0 g, 8.1 mmol, 1.0 equiv), pyridine (0.96 g, 12.1 mmol, 1.5 equiv), and O-methylhydroxylamine hydrochloride (1.0 g, 12.1 mmol, 1.5 equiv). Crude oxime 1g (0.84 g, 68%) was obtained as oil. ¹H NMR (400 MHz, CDCl₃) δ 3.84 (s, 3H), 2.28-2.38 (m, 3H), 1.90-1.95 (m, 1H), 1.44-1.75 (m, 9H); ¹³C NMR (100 MHz, CDCl₃) δ 164.6, 61.0, 31.8, 31.6, 25.4, 25.1, 24.9; LRMS(ESI) calculated for C₉H₁₆ON [M+H]⁺ m/z 154.12, found 154.13.

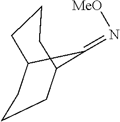 [SMILES: CON=C1C2CCCC1CCC2]

Prepared according to general procedure G using 60 mL MeOH, bicyclo[3.3.1]nonan-9-one (2.0 g, 14.5 mmol, 1.0 equiv), pyridine (1.72 g, 21.7 mmol, 1.5 equiv), and O-methylhydroxylamine hydrochloride (1.81 g, 21.7 mmol, 1.5 equiv). Crude oxime 1h (2.1 g, 87%) was obtained as oil. ¹H NMR (400 MHz, CDCl₃) δ 3.76-3.84 (m, 3H), 3.38 (br, 1H), 2.45 (br, 1H), 1.74-2.07 (m, 10H), 1.44-1.58 (m, 2H); ¹³C NMR (100 MHz, CDCl₃) δ 167.1, 60.8, 36.0, 33.4, 32.0, 29.3, 21.2; LRMS(ESI) calculated for C₁₃H₁₈ON [M+H]⁺ m/z 168.14, found 168.21.

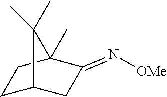

Prepared according to general procedure G using 100 mL MeOH, camphor (5.0 g, 32.8 mmol, 1.0 equiv), pyridine (4.11 g, 49.3 mmol, 1.5 equiv), and O-methylhydroxylamine hydrochloride (3.9 g, 49.3 mmol, 1.5 equiv). Crude oxime 1i (5.14 g, 86%) was obtained as oil. ¹H NMR (400 MHz, CDCl₃) δ 3.82 (s, 3H), 2.47 (dt, 1H, J=4.1, 17.8 Hz), 1.97 (d, 1H, J=17.8 Hz), 1.78-1.88 (m, 2H), 1.70 (td, 1H, J=4.1, 12.4 Hz), 1.44 (ddd, 1H, J=4.4, 9.3, 13.4 Hz), 1.21 (ddd, 1H, J=3.9, 9.0, 13.2 Hz), 1.01 (s, 3H), 0.90 (s, 3H), 0.79 (s, 3H); ¹³C NMR (100 MHz, CDCl₃) δ 169.2, 61.2, 51.6, 48.1, 43.7, 33.6, 32.9, 27.3, 19.4, 18.5, 11.2; LRMS(ESI) calculated for C₁₃H₁₈ON [M+H]⁺ m/z 186.15, found 186.16.

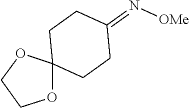 [SMILES: CON=C1CCC2(CC1)OCCO2]

Prepared according to general procedure G using 30 mL MeOH, 1,4-dioxaspiro[4.5]decan-8-one (1.0 g, 6.4 mmol, 1.0 equiv), pyridine (0.76 g, 9.6 mmol, 1.5 equiv), and O-methylhydroxylamine hydrochloride (0.8 g, 9.6 mmol, 1.5 equiv). Crude oxime 1j (1.0 g, 84%) was obtained as oil. ¹H NMR (400 MHz, CDCl₃) δ 3.79-3.99 (m, 4H), 3.20 (s, 3H, minor E/Z isomer), 3.17 (s, 3H, major E/Z isomer), 2.23-2.64 (m, 2H), 1.61-1.90 (m, 6H); ¹³C NMR (100 MHz, CDCl₃) δ 157.7, 108.5, 108.0, 99.2, 64.4, 64.2, 61.0 (two peaks), 47.8, 34.4, 34.2, 31.2, 29.7, 28.9, 28.7, 21.7; LRMS(ESI) calculated for C₉H₁₆O₃N [M+H]⁺ m/z 186.11, found 186.21.

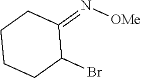

To a pressure vessel containing a magnetic stir bar was added 2-bromocyclohexan-1-one (0.5 g, 2.8 mmol, 1.0 equiv) followed by 20 mL MeOH, pyridine (0.34 g, 4.3 mmol, 1.5 equiv) and methoxylamine hydrochloride (0.35 g, 4.3 mmol, 1.5 equiv). The reaction vessel was then sealed with a teflon screw cap and stirred for 16 h at room temperature. The reaction mixture was then transferred to a flask and concentrated under reduced pressure to a crude oil. The crude residue was diluted with 10% aq KHSO₄ solution (115 mL) and extracted with EtOAc (1×200 mL). The organic phase was washed with additional 10% aq KHSO₄ solution (3×60 mL) and the aqueous layers back-extracted with EtOAc (1×150 mL). The combined organic phases were washed with brine (1×150 mL), dried (MgSO₄), filtered and concentrated to a colorless oil, which solidified under high vacuum to give oxime 1k (0.50 g, 85%), which was sufficiently pure to be carried onto the next step without further purification. ¹H NMR (400 MHz, CDCl₃) δ 5.57 (br, 1H, minor E/Z isomer), 4.91 (br, 1H, major E/Z isomer), 3.88 (s, 3H, minor E/Z isomer), 3.85 (s, 3H, major E/Z isomer), 3.10 (d, 1H, J=14.9 Hz major E/Z isomer), 2.66 (td, 1H, J=4.6, 14.1 Hz minor E/Z isomer), 1.60-2.35 (m, 5H), 1.29-1.49 (m, 1H); ¹³C NMR (100 MHz, CDCl₃) δ 157.8, 156.3, 61.6, 51.8, 39.2, 35.9, 34.2, 27.4, 26.0, 24.9, 20.6 (three peaks); LRMS(ESI) calculated for C₇H₁₃ON [M+H]⁺ m/z 206.02, found 206.01.

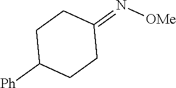 [SMILES: CON=C1CCC(c2ccccc2)CC1]

Prepared according to general procedure G using 60 mL MeOH, 4-phenylcyclohexan-1-one (2.0 g, 11.5 mmol, 1.0 equiv), pyridine (1.36 g, 17.2 mmol, 1.5 equiv), and O-methylhydroxylamine hydrochloride (1.44 g, 17.2 mmol, 1.5 equiv). Crude oxime 11 (1.7 g, 73%) was obtained as oil. ¹H NMR (400 MHz, CDCl₃) δ 7.29-7.34 (m, 2H), 7.20-7.25 (m, 3H), 3.88 (s, 3H), 3.40 (ddt, 1H, J=2.2, 4.4, 14.4 Hz), 2.78 (tt, 1H, J=3.4, 15.6 Hz), 2.55 (ddt, 1H, J=2.4, 4.4, 14.1 Hz), 2.26 (td, 1H, J=4.9, 13.9 Hz), 2.01-2.14 (m, 2H), 1.89 (td, 1H, J=4.9, 13.9 Hz), 1.59-1.78 (m, 2H); ¹³C NMR (100 MHz, CDCl₃) δ 158.9, 145.7, 128.4, 126.4, 126.2, 61.0, 43.6, 34.0, 32.9, 31.9, 24.8; LRMS(ESI) calculated for C₁₃H₁₈ON [M+H]⁺ m/z 204.14, found 204.21.

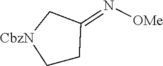 [SMILES: CO/N=C1\CCN(C(=O)OCc2ccccc2)C1]

Prepared according to general procedure G using 30 mL MeOH, benzyl 3-oxopyrrolidine-1-carboxylate (0.9 g, 4.1 mmol, 1.0 equiv), pyridine (0.48 g, 6.1 mmol, 1.5 equiv), and O-methylhydroxylamine hydrochloride (0.51 g, 6.1 mmol, 1.5 equiv). Crude oxime 1m (0.84 g, 82%) was obtained as oil. ¹H NMR (400 MHz, CDCl₃) δ 7.29-7.40 (m, 5H), 5.16 (d, 2H, J=2.4 Hz), 4.13 (br, 2H), 3.88 (s, 3H, minor E/Z isomer), 3.87 (s, 3H, major E/Z isomer), 3.67 (br, 1H), 2.68-2.79 (m, 2H); ¹³C NMR (100 MHz, CDCl₃) δ 158.7, 157.9, 154.6, 154.5, 136.4, 128.3, 130.0 (multiple peaks), 67.0, 61.8 (two peaks), [47.7, 47.4, 46.0, 45.6 (E/Z isomers & and conformers)], [44.5, 44.2, 44.0, 43.8 (E/Z isomers & and conformers)], [28.7, 28.0, 26.1, 25.3 (E/Z isomers & and conformers)]; LRMS(ESI) calculated for C₁₃H₁₇O₃N₂ [M+H]⁺ m/z 249.12, found 249.15.

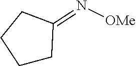

To a pressure vessel containing a magnetic stir bar was added cyclopentanone (2.0 g, 23.8 mmol, 1.0 equiv) followed by 60 mL MeOH, pyridine (2.8 g, 35.7 mmol, 1.5 equiv) and methoxylamine hydrochloride (2.8 g, 35.7 mmol, 1.5 equiv). The reaction vessel was then sealed with a teflon screw cap and stirred for 16 h. The reaction mixture was then transferred to a flask and concentrated under reduced pressure to a crude oil. The crude residue was diluted with 10% aq KHSO₄ solution (115 mL) and extracted with EtOAc (1×200 mL). The organic phase was washed with additional 10% aq KHSO₄ solution (3×60 mL) and the aqueous layers back-extracted with EtOAc (1×150 mL). The combined organic phases were washed with brine (1×150 mL), dried (MgSO₄), filtered and concentrated to a colorless oil, which solidified under high vacuum to give oxime in (0.45 g, 17%), which was sufficiently pure to be carried onto the next step without further purification. ¹H NMR (400 MHz, CDCl₃) δ 3.83 (s, 3H), 2.29-2.46 (m, 4H), 1.68-1.78 (m, 4H); ¹³C NMR (100 MHz, CDCl₃) δ 166.3, 61.3, 31.0, 27.5, 25.1, 24.7; LRMS(ESI) calculated for C₆H₁₂ON [M+H]⁺ m/z 204.14, found 204.21.

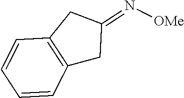 [SMILES: CON=C1Cc2ccccc2C1]

Prepared according to general procedure G using 30 mL MeOH, 1,3-dihydro-2H-inden-2-one (1.0 g, 7.6 mmol, 1.0 equiv), pyridine (0.90 g, 11.3 mmol, 1.5 equiv), and O-methylhydroxylamine hydrochloride (0.95 g, 11.3 mmol, 1.5 equiv). Crude oxime to (1.11 g, 91%) was obtained as oil. ¹H NMR (400 MHz, CDCl₃) δ 7.22-7.35 (m, 4H), 3.96 (s, 3H), 3.79-3.84 (m, 4H); ¹³C NMR (100 MHz, CDCl₃) δ 161.6, 139.1, 139.0, 127.1, 127.0, 125.1, 124.7, 61.7, 36.5, 34.5; LRMS(ESI) calculated for C₁₀H₁₂ON [M+H]⁺ m/z 162.09, found 162.07.

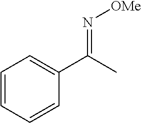

Prepared according to general procedure G using 30 mL MeOH, acetophenone (1.0 g, 8.3 mmol, 1.0 equiv), pyridine (0.99 g, 12.5 mmol, 1.5 equiv), and O-methylhydroxylamine hydrochloride (1.0 g, 12.5 mmol, 1.5 equiv). Crude oxime 1p (1.14 g, 92%) was obtained as oil. ¹H NMR (400 MHz, CDCl₃) δ 7.65-7.70 (m, 2H), 7.35-7.41 (m, 3H), 4.02 (s, 3H), 2.25 (s, 3H); ¹³C NMR (100 MHz, CDCl₃) δ 154.6, 136.6, 129.0, 128.4, 126.0, 61.9, 12.6; LRMS(ESI) calculated for C₉H₁₂ON [M+H]⁺ m/z 150.09, found 150.08.

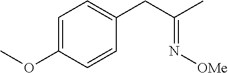

Prepared according to general procedure G using 30 mL MeOH, 4-(4-methoxyphenyl)butan-2-one (1.0 g, 6.1 mmol, 1.0 equiv), pyridine (723.0 mg, 9.1 mmol, 1.5 equiv), and O-methylhydroxylamine hydrochloride (763 mg, 9.1 mmol, 1.5 equiv). Crude oxime 1q (1.1 g, 94%) was obtained as oil. ¹H NMR (400 MHz, CDCl₃) δ 7.09-7.17 (m, 2H), 6.82-6.88 (m, 2H), 3.89 (app d, 3H), 3.80 (s, 3H), 3.62 (s, 2H, minor E/Z isomer), 3.41 (s, 2H, major E/Z isomer), 1.78 (s, 3H, minor E/Z isomer), 1.73 (s, 3H, major E/Z isomer); ¹³C NMR (100 MHz, CDCl₃) δ 158.5, 156.8, 130.0, 129.9, 128.9, 114.0, 61.2, 61.1, 55.2, 41.2, 34.4, 19.6, 13.5; LRMS(ESI) calculated for C₁₁H₁₆O₂N [M+H]⁺ m/z 194.12, found 194.11.

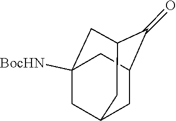 [SMILES: CC(C)(C)OC(=O)NC12CC3CC(C1)C(=O)C(C3)C2]

To an oven-dried rb flask equipped with a reflux condenser and magnetic stir bar under an Ar(g) atmosphere was added 4-oxoadamantane-1-carboxylic acid (2.00 g, 10.3 mmol, 1.0 equiv) followed by anhydrous toluene (38 mL) and NEt₃ (1.95 mL, 14.0 mmol, 1.36 equiv). The reaction mixture was cooled to 0° C. and placed behind a blast shield before diphenylphosphoryl azide (2.21 mL, 10.3 mmol, 1.00 equiv) was added dropwise over 5 mins. The reaction mixture was stirred at 0° C. for 5 mins and allowed to warm to rt over 10 mins. The reaction was then gradually heated to 90° C. for 2 h until TLC indicated the reaction was complete. The mixture was then cooled to rt and concentrated under reduced pressure to a viscous pale yellow semi-solid. tert-Butanol (42.0 mL, 0.44 mol, 42.6 equiv) was added and the mixture was transferred to a sealed tube with a threaded screw cap and heated to 120° C. behind a blast shield for 16 h. The reaction was then cooled to rt, diluted with 1 M aq Na₂CO₃ solution (150 mL) and extracted with EtOAc (1×150 mL). The aqueous layer was back-extracted with EtOAc (3×150 mL) and the combined organic layers washed with brine (1×250 mL), dried (MgSO₄), filtered and concentrated to a crude residue. Purification via flash column chromatography (0-30% EtOAc-Hexanes) gave tert-butyl (4-(oxo)adamantan-1-yl)carbamate (1.91 g, 7.20 mmol, 70%) as a colorless solid. ¹H NMR (400 MHz, CDCl₃) δ 4.52 (br s, 1H), 2.54 (br s, 2H), 2.06-2.22 (m, 7H), 1.97 (br d, J=11.7 Hz, 2H), 1.89 (br d, J=12.4 Hz, 2H), 1.34-1.53 (m, 9H); ¹³C NMR (100 MHz, CDCl₃) δ 216.5, 49.5, 46.4, 42.1, 38.2, 28.6, 28.4; LRMS (ESI): m/z [M]−OtBu+H⁺ calcd for C₁₁H₁₆NO₃: 210.11, found m/z: 210.15.

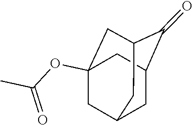

To a round bottom flask containing a magnetic stir bar under an Ar(g) atmosphere was added 2-adamtanone-5-carboxylic acid (7.50 g, 38.62 mmol, 1.00 equiv), anhydrous DMF (64 mL), K₂CO₃ (8.01 g, 57.92, 1.50 equiv) and iodomethane (3.61 mL, 57.92 mmol, 1.50 equiv). The reaction stirred for 16 h at rt, after which the reaction was judged complete by TLC and was diluted with DI H₂O (100 mL) and extracted with EtOAc (1×300 mL). The organic layer was washed with water (6×100 mL) and the combined aqueous layers were back-extracted with EtOAc (1×200 mL). The combined organic phases were washed with brine (1×400 mL), dried (MgSO₄), filtered and concentrated under reduced pressure to a crude brown oil that solidified on cooling to rt. Purification via flash column chromatography (0-50% EtOAc-Hexanes) afforded methyl 2-adamtanone-5-carboxylate (7.12 g, 34.19 mmol, 89%) as a colorless solid. ¹H NMR (400 MHz, CDCl₃) δ 3.67 (s, 3H), 2.58 (br s, 2H), 2.16-2.21 (m, 5H), 2.10 (d, J=2.7 Hz, 2H), 1.95-2.06 (m, 4H); ¹³C NMR (100 MHz, CDCl₃) δ 216.5, 176.2, 52.0, 45.8, 40.3, 40.1, 38.3, 37.8, 27.3; LRMS (ESI): m/z [M]+H⁺ calcd for C₁₂H₁₇O₃: 209.12, found m/z: 208.96.

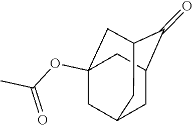

A solution of 5-hydroxy-2-adamantanone (1.00 g, 6.02 mmol, 1.00 equiv) in CH₂Cl₂ (20 mL) was treated with dimethylaminopyridine (0.80 g, 6.55 mmol, 1.09 equiv) and acetic anhydride (0.80 mL, 0.82 mmol, 1.47 equiv) and the reaction stirred overnight at 50° C. The solvent was removed under reduced pressure and the residue partitioned between DI water (150 mL) and EtOAc (150 mL). The aqueous layer was extracted with EtOAc (2×100 mL) and the combined organic layers were washed with satd. aq NaCl₂ solution (1×200 mL), dried (MgSO₄), filtered and concentrated under reduced pressure to a crude residue. Purification via flash column chromatography (0-20% EtOAc-Hexanes) afforded 5-acetoxy-2-adamantanone (1.20 g, 5.76 mmol, 96%) as an off-white solid. ¹H NMR (400 MHz, CDCl₃) δ 2.65 (br s, 2H), 2.30-2.53 (m, 7H), 1.92-2.06 (m, 7H); ¹³C NMR (100 MHz, CDCl₃) δ 215.6, 170.2, 77.6, 47.0, 41.3, 39.8, 38.2, 29.8, 22.4; LRMS (ESI): m/z [M]+H⁺ calcd for C₁₂H₁₇O₃: 209.12, found m/z: 209.16.

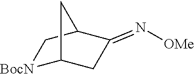 [SMILES: CO/N=C1\CC2CC1CN2C(=O)OC(C)(C)C]

Prepared according to general procedure H using 60 mL MeOH, tert-butyl 5-oxo-2-azabicyclo[2.2.1]heptane-2-carboxylate (1.0 g, 4.7 mmol, 1.0 equiv), pyridine (0.75 g, 9.5 mmol, 2 equiv), and O-methylhydroxylamine hydrochloride (0.59 g, 7.1 mmol, 1.5 equiv). Crude oxime 1r (0.93 g, 82%) was obtained as orange oil. ¹H NMR (400 MHz, Chloroform-d) δ 4.49 (br, 1H, minor), 4.35 (br, 1H, major), 3.84 (s, 3H), 3.43-3.18 (m, 2H), 3.14 (s, 1H), 2.58-2.28 (m, 2H), 1.86 (t, J=13.3 Hz, 1H), 1.68 (d, J=9.9 Hz, 1H), 1.45 (s, 9H); LRMS(ESI) calculated for C₁₂H₂₁O₃N₂ [M+H]⁺ m/z 241.16, found 241.27.

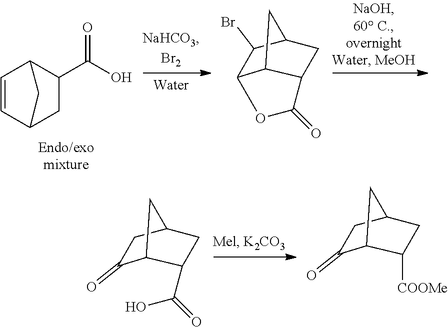 [SMILES: O=C(O)C1CC2C=CC1C2 ; O=C1OC2C(Br)C3CC1C2C3 ; O=C(O)C1CC2CC(=O)C1C2 ; COC(=O)C1CC2CC(=O)C1C2]

To a solution of 5-norbornene-2-carboxylic acid (25.0 g, 181 mmol, 1 equiv) in water (400 mL) was added sodium bicarbonate (76.0 g, 905 mmol, 5 equiv). The resulting solution was stirred for 5 min to give a clear solution. Neat bromine (72.3 g, 452 mmol, 2.5 equiv) was added to the mixture slowly at 0° C. The reaction was then warmed up to room temperature and stirred overnight. The crude mixture was extracted with ether (4×200 mL). The organic layers were dried over MgSO₄, filtered, and concentrated to an orange oil. The oil was used without purification. The crude bromo-lactone was dissolved in MeOH (150 mL) and an ice-cold solution of NaOH (28.9 g, 722.4 mmol, 4 equiv) in water (50 mL). The NaOH solution was added in 5 portions with vigorous stirring overnight at 60° C. The aqueous residue was carefully neutralized with concentrated HCl and acidified to pH 1. The acidified solution was extracted with ethyl acetate (4×200 mL). The organic layers were dried over MgSO₄, filtered, and concentrated to yield endo-6-oxobicyclo[2.2.1]heptane-2-carboxylic acid as a yellow oil (16.5 g). The crude acid (16.5 g, 107 mmol, 1 equiv) was dissolved in DMF (50 mL) and treated with solid K₂CO₃ (22.2 g, 161 mmol, 1.5 equiv) and Mel (22.8 g, 161 mmol, 1.5 equiv). The reaction mixture was stirred at room temperature overnight. The reaction solution was quenched with water, extracted with ethyl acetate (100 mL×2). The organic layers were washed with water (100 mL×5), dried over MgSO₄, filtered, and concentrated to yield a pale-yellow oil. The crude oil was purified by flash column chromatography to afford endo-methyl 6-oxobicyclo[2.2.1]heptane-2-carboxylate as a pale-yellow oil (8.5 g, 51 mmol, 28.2% over 3 steps). ¹H NMR (400 MHz, Chloroform-d) δ 3.66 (s, 2H), 3.01 (dt, J=11.1, 4.7 Hz, 1H), 2.85-2.80 (m, 1H), 2.73-2.66 (m, 1H), 2.15-1.94 (m, 4H), 1.85 (ddd, J=12.6, 4.7, 2.4 Hz, 1H), 1.81-1.74 (m, 1H), 1.74-1.65 (m, 1H); LRMS(ESI) calculated for C₉H₁₃O₃[M+H]⁺ m/z 169.09, found 169.11.

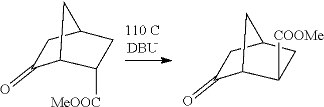 [SMILES: COC(=O)C1CC2CC(=O)C1C2 ; COC(=O)C1CC2CC(=O)C1C2]

To a solution of endo-methyl-6-oxobicyclo[2.2.1]heptane-2-carboxylate (3.2 g, 19.0 mmol, 1 equiv) in toluene (10 mL) was added DBU (5.8 g, 38.0 mmol, 2 equiv). The mixture was heated to 110° C. overnight. The reaction was cooled to room temperature and quenched with 1M HCl (50 mL). The resulting solution was extracted with ethyl acetate (50 mL×2), dried over MgSO₄ and concentrated to yield a pale-yellow oil. The crude oil was purified by flash column chromatography to afford exo-methyl-6-oxobicyclo[2.2.1]heptane-2-carboxylate as a pale yellow oil (1.4 g, 8.3 mmol, 44%). ¹H NMR (400 MHz, Chloroform-d) δ 3.72 (s, 3H), 2.88 (s, 1H), 2.77 (br, 1H), 2.72-2.62 (m, 1H), 2.22-2.05 (m, 2H), 1.94-1.83 (m, 2H), 1.82-1.65 (m, 2H); LRMS(ESI) calculated for C₉H₁₃O₃[M+H]⁺ m/z 169.09, found 169.12.

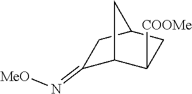 [SMILES: CO/N=C1\CC2CC(C(=O)OC)C1C2]

Prepared according to general procedure H using 60 mL MeOH, exo-methyl-6-oxobicyclo[2.2.1]heptane-2-carboxylate (0.5 g, 3.2 mmol, 1.0 equiv), pyridine (0.35 g, 4.5 mmol, 1.5 equiv), and O-methylhydroxylamine hydrochloride (0.37 g, 4.5 mmol, 1.5 equiv). Crude oxime is (0.50 g, 85%) was obtained as yellow oil. ¹H NMR (400 MHz, Chloroform-d) δ 3.81 (s, 3H, minor), 3.80 (s, 3H, major), 3.67 (s, 3H, minor), 3.66 (s, 3H), 3.11 (s, 1H), 2.67-2.55 (m, 1H), 2.58-2.46 (m, 1H), 2.30-2.16 (m, 1H), 2.05-1.92 (m, 2H), 1.71-1.56 (m, 2H), 1.48-1.38 (m, 1H); (ESI) calculated for C₁₀H₁₆NO₃ [M+H]⁺ m/z 198.11, found 198.21.

### Example 3: Additional Experimental Methods and Characterization Data

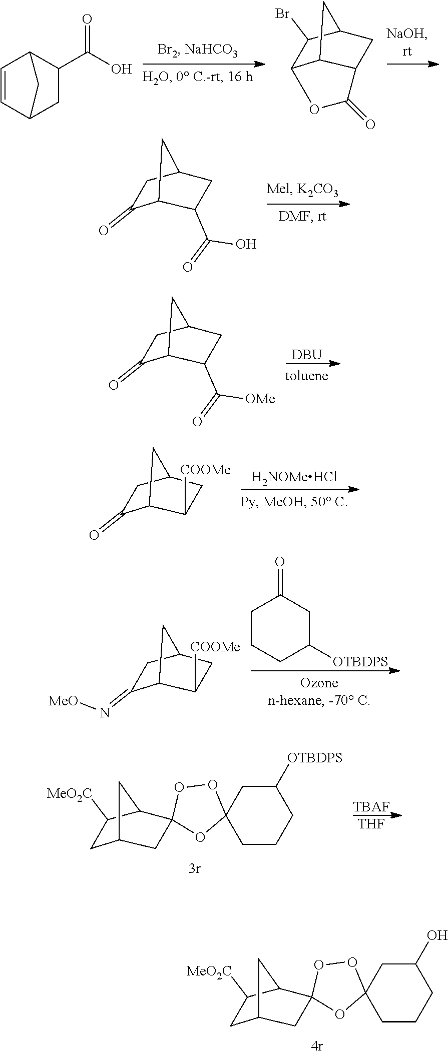

### 6-bromohexahydro-2H-3,5-methanocyclopenta[b]furan-2-one

To a solution of bicyclo[2.2.1]hept-5-ene-2-carboxylic acid (800 g, 5.8 mol) in water (8 L) was added sodium bicarbonate (1461.6 g, 17.4 mol), and the mixture was stirred at room temperature for 5 min. Bromine (928 g, 5.8 mol) was slowly added to the reaction mixture at 0° C. The resulting mixture was warmed up to room temperature and stirred overnight. The mixture was extracted with ether (500 mL×3). The combined organic layers were dried over Na₂SO₄, filtered and concentrated to afford orange oil (680 g, 54.3% yield), which was directly used for the next step without further purification.

### (1R,2S,4S)-6-oxobicyclo[2.2.1]heptane-2-carboxylic acid)

To a solution of 6-bromohexahydro-2H-3,5-methanocyclopenta[b]furan-2-one (680 g, 3.12 mol) in water (14.4 L) was added NaOH (504 g, 12.48 mol), and the mixture was stirred at room temperature overnight. When it was completed, the mixture was carefully acidified to pH=1 with concentrated HCl. The acidified solution was extracted with DCM (2 L×3), dried over Na₂SO₄, filtered and concentrated in vacuo to afford brown oil (432.0 g, 89.3% yield), which was directly used for the next step without further purification.

### Methyl (1R,2S,4S)-6-oxobicyclo[2.2.1]heptane-2-carboxylate

To a solution of (1R,2S,4S)-6-oxobicyclo[2.2.1]heptane-2-carboxylic acid (432.0 g, 2.83 mol) in DMF (2.0 L) was added K₂CO₃ (1171.6 g, 8.49 mol), and the mixture was stirred for 10 min before iodomethane (602.8 g, 4.245 mol) was slowly injected to the reaction mixture. The reaction was stirred at room temperature for 16 h. The mixture was diluted with water (3.0 L) and extracted by DCM (1.0 L×3). The combined organic layers were washed with brine, dried over Na₂SO₄, filtered and concentrated under reduced pressure. The residue was purified by column chromatography (EA/PE=1/4) to give methyl (1R,2S,4S)-6-oxobicyclo[2.2.1]heptane-2-carboxylate (284.7 g, 59.8% yield) as a yellow oil.

### Methyl (1R,2R,4S)-6-oxobicyclo[2.2.1]heptane-2-carboxylate

To a solution of methyl (1R,2S,4S)-6-oxobicyclo[2.2.1]heptane-2-carboxylate (284.7 g, 1.69 mol) in toluene (2.0 L) was added DBU (412.6 g, 2.70 mol), and the mixture was heated to 110° C. for 16 h. When it was completed, the mixture was concentrated. The residue was purified by column chromatography (EA/PE=1/4) to give methyl (1R,2R,4S)-6-oxobicyclo[2.2.1]heptane-2-carboxylate (121.0 g, 42.5% yield) as a yellow oil.

### Methyl (1R,2R,4S,E)-6-(methoxyimino)bicyclo[2.2.1]heptane-2-carboxylate

Prepared based on general procedure H. To a solution of methyl (1R,2R,4S)-6-oxobicyclo[2.2.1]heptane-2-carboxylate (100.0 g, 0.595 mol) and methoxylamine hydrochloride (75.0 g, 0.893 mol) in MeOH (1.0 L) was added pyridine (70.5 g, 0.893 mol), and the mixture was stirred at 50° C. for 3 h. When the reaction was completed, the mixture was concentrated. The reaidue was purified by column chromatography (PE/EA=5/1) to give methyl (1R,2R,4S,E)-6-(methoxyimino)bicyclo[2.2.1]heptane-2-carboxylate (75.5 g, 64.4% yield) as a colorless oil.

### Methyl (1R,2S,4S,6R)-3″-((tert-butyldiphenylsilyl)oxy)dispiro[bicyclo[2.2.1]heptane-2,3′-[1,2,4] trioxolane-5′,1″-cyclohexane]-6-carboxylate

Prepared based on general procedure B. The solution of methyl (1R,2R,4S,E)-6-(methoxyimino)bicyclo[2.2.1]heptane-2-carboxylate (75.5 g, 0.383 mol) and 3-((tert-butyldiphenylsilyl)oxy)cyclohexan-1-one (90.0 g, 0.255 mol) in n-hexane (7.5 L) was cooled to −78° C. The reaction mixture stirred at −70° C. for 2.5 h bubbled with ozone. When the reaction was completed, nitrogen was purged into the reaction for 1 h to remove excess ozone. The reaction was concentrated. The residue was purified by column chromatography (EA/PE=14/1) to give methyl (1R,2S,4S,6R)-3″-((tert-butyldiphenylsilyl)oxy)dispiro[bicyclo[2.2.1]heptane-2,3′-[1,2,4]trioxolane-5′,1″-cyclohexane]-6-carboxylate (3r; 60.0 g, 43.8% yield) as a colorless oil.

### Methyl (1R,2S,4S,6R)-3″-hydroxydispiro[bicyclo[2.2.1]heptane-2,3′-[1,2,4]trioxolane-5′,1″-cyclohexane]-6-carboxylate

Prepared based on general procedure C. To a stirred solution of methyl (1R,2S,4S,6R)-3″-((tert-butyldiphenylsilyl)oxy)dispiro[bicyclo[2.2.1]heptane-2,3′-[1,2,4]trioxolane-5′,1″-cyclohexane]-6-carboxylate (60.0 g, 0.112 mol, 3r) in THF (1.2 L) was added tetrabutylammonium fluoride (146.4 g, 0.56 mol) at 0° C., and the reaction mixture was stirred for 2 h at room temperature. When the reaction was completed, the mixture was concentrated. The residue was diluted with water (500 mL) and extracted with DCM (500 mL×3). The combined organic layers were washed with brine, dried over Na₂SO₄, filtered and concentrated under reduced pressure. The residue was purified by column chromatography (PE/EA=3/1) to afford methyl (1R,2S,4S,6R)-3″-hydroxydispiro[bicyclo[2.2.1]heptane-2,3′-[1,2,4]trioxolane-5′,1″-cyclohexane]-6-carboxylate (4r; 26.8 g, 80.4% yield) as colorless oil. ¹H NMR (400 MHz, CDCl₃) δ 3.97-3.87 (m, 1H), 3.68 (d, J=2.0 Hz, 3H), 2.90-2.86 (m, 1H), 2.62-2.59 (m, 1H), 2.42-2.29 (m, 1H), 2.15-1.71 (m, 9H), 1.65-1.51 (m, 6H).

### Tert-butyl((11-(tert-butyl)-7,14,15-trioxadispiro[5.1.5⁸.2⁶]pentadecan-2-yl)oxy)diphenylsilane

Prepared based on general procedure B. The solution of 4-(tert-butyl)cyclohexan-1-one O-methyl oxime (16.9 g, 92.3 mmol) and 3-((tert-butyldiphenylsilyl)oxy)cyclohexan-1-one (16.2 g, 46.0 mmol) in hexane (800 mL) was cooled to −78° C. Ozone was then bubbled and the reaction was stirred at −78° C. for 7 h. The reaction was purged with nitrogen for 30 min to remove ozone. The mixture was concentrated and the residue was purified by column chromatography (EA/PE=1/50) to afford tert-butyl((11-(tert-butyl)-7,14,15-trioxadispiro[5.1.5⁸.2⁶]pentadecan-2-yl)oxy)diphenylsilane as a colorless oil (10.5 g, 43.8% yield).

### 11-(tert-butyl)-7,14,15-trioxadispiro[5.1.5⁸.2⁶]pentadecan-2-ol

Prepared based on general procedure C. To a solution of tert-butyl((11-(tert-butyl)-7,14,15-trioxadispiro[5.1.5⁸.2⁶]pentadecan-2-yl)oxy)diphenylsilane (33.7 g, 64.5 mmol) in THF (150 mL) was added TBAF (101.8 g, 322.7 mmol) at 0° C., and the reaction mixture was stirred at room temperature overnight. The reaction mixture was diluted with water and extracted with EA (100 mL×2). The combined organic layers were washed with brine, dried over MgSO₄, filtered and concentrated under reduced pressure. The residue was purified by column chromatography (EA/PE=1/20) to afford 11-(tert-butyl)-7,14,15-trioxadispiro[5.1.5⁸.2⁶]pentadecan-2-ol (10.1 g, 55.1% yield) as a white solid. ¹H NMR (400 MHz, CDCl₃) δ 3.95-3.93 (m, 1H), 2.13-1.57 (m, 12H), 1.55-1.41 (m, 2H), 1.36-1.17 (m, 2H), 1.09-1.02 (m, 1H), 0.86 (d, J=8.0 Hz, 9H).

### Methyl-4-(methoxyimino)adamantane-1-carboxylate

To a solution of 4-oxoadamantane-1-carboxylate (100.6 g, 483.6 mmol) in methanol (1000 mL) were added pyridine (76.4 g, 967.3 mmol) and methoxyamine hyhdrochloride (44.2 g, 532.0 mol), and the reaction was stirred at room temperature overnight. The reaction mixture was concentrated and the resulting residue was triturated with water (3 L). The resulting solid was filtered and dried to afford methyl-4-(methoxyimino)adamantane-1-carboxylate (106.6 g, 93% yield) as a white solid.

### Methyl-3″-((tert-butyldiphenylsilyl)oxy)dispiro[adamantane-2,3′-[1,2,4]trioxolane-5′,1″-cyclohexane]-5-carboxylate

Prepared based on general procedure B. The solution of methyl-4-(methoxyimino)adamantane-1-carboxylate (30.0 g, 126.58 mmol) and 3-((tert-butyldiphenylsilyl)oxy)cyclohexan-1-one (17.8 g, 50.63 mmol) in n-hexane (2.2 L) was cooled to −78° C. Ozone was charged into the mixture at −70° C. for 3 h. Nitrogen was purged into the mixture for 30 min to remove ozone. The reaction was concentrated. The residue was purified by column chromatography (PE/EA=20/1) to afford methyl-3″-((tert-butyldiphenylsilyl)oxy)dispiro[adamantane-2,3′-[1,2,4]trioxolane-5′,1″-cyclohexane]-5-carboxylate (26.4 g, 89% yield) as a colorless oil.

### Methyl-3″-hydroxydispiro[adamantane-2,3′-[1,2,4]trioxolane-5′,1″-cyclohexane]-5-carboxylate

To a solution of methyl-3″-((tert-butyldiphenylsilyl)oxy)dispiro[adamantane-2,3′-[1,2,4]trioxolane-5′,1″-cyclohexane]-5-carboxylate (56.0 g, 45.83 mmol) in THF (600 mL) was added TBAF (135.6 g, 486.11 mmol), and the reaction mixture was stirred at 0° C. for 3 h. The reaction mixture was diluted with H₂O (1.5 L) and extracted with EA (300 mL×3). The combined organic layers were washed with brine, dried over Na₂SO₄, filtered and concentrated under reduced pressure. The residue was purified by column chromatography (PE/EA=5/1) to afford methyl -3″-hydroxydispiro[adamantane-2,3′-[1,2,4]trioxolane-5′,1″-cyclohexane]-5-carboxylate (25.06 g, 76% yield) as a colorless oil. ¹H NMR (400 MHz, CDCl₃) δ 3.90-3.81 (m, 1H), 3.58 (d, J=4.8 Hz, 3H), 2.13-2.03 (m, 1H), 2.03-1.99 (m, 5H), 1.99-1.52 (m, 14H), 1.52-1.35 (m, 2H).

### Methyl 10-((tert-butyldiphenylsilyl)oxy)-7,14,15-trioxadispiro[5.1.5⁸.2⁶]pentadecane-3-carboxylate

Prepared based on general procedure B. The solution of methyl 4-(methoxyimino)cyclohexane-1-carboxylate (87.0 g, 468 mmol) and 3-((tert-butyldiphenylsilyl)oxy)cyclohexan-1-one (60 g, 187 mmol) in n-hexane (2 L) was cooled to 0° C. Ozone was charged into the mixture at 0° C. for 3 h. When it was completed, nitrogen was purged into the mixture for 30 min to remove ozone. The mixture was concentrated. The residue was purified by column chromatography (PE/EA=10/1) to afford methyl 10-((tert-butyldiphenylsilyl)oxy)-7,14,15-trioxadispiro[5.1.5⁸.2⁶]pentadecane-3-carboxylate (89.0 g, 90.5% yield) as a colorless oil.

### Methyl 10-hydroxy-7,14,15-trioxadispiro[5.1.5⁸.2⁶]pentadecane-3-carboxylate

Prepared based on general procedure C. To a solution of methyl 10-((tert-butyldiphenylsilyl)oxy)-7,14,15-trioxadispiro[5.1.5⁸.2⁶]pentadecane-3-carboxylate (89.0 g, 170 mmol) in THF (1.5 L) was added TBAF (221.9 g, 849 mmol), and the reaction mixture was stirred at room temperature for 3 h. The reaction mixture was diluted with H₂O (1.5 L) and extracted with EA (1.5 L×2). The combined organic layers were washed with brine, dried over Na₂SO₄, filtered and concentrated under reduced pressure. The residue was purified by column chromatography (PE/EA=3/1) to afford methyl 10-hydroxy-7,14,15-trioxadispiro[5.1.5⁸.2⁶]pentadecane-3-carboxylate (30.0 g, 61% yield) as pale yellow oil. ¹H NMR (400 MHz, CDCl₃) δ 3.93-3.90 (m, 1H), 3.69-3.67 (d, J=8.0 Hz, 3H), 2.41-2.33 (m, 1H), 2.09-1.95 (m, 7H), 1.95-1.66 (m, 8H), 1.55-1.43 (m, 2H).

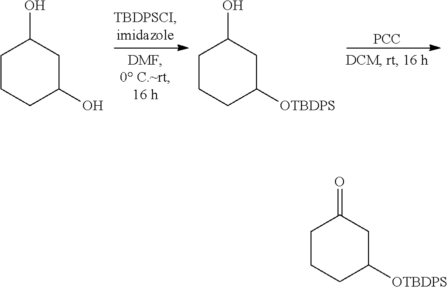

### 3-((tert-butyldiphenylsilyl)oxy)cyclohexan-1-ol

To a solution of 1,3-cyclohexanediol (100 g, 0.86 mo) and imidazole (87.9 g, 1.29 mol) in DMF (1000 mL) was added TBDPSCl (227.6 g, 0.83 mol), and the reaction mixture was stirred at room temperature for 16 h. The reaction mixture was diluted with water (800 mL) and extracted with EA (500 mL×3). The combined organic layers were washed with brine, dried over Na₂SO₄, filtered and concentrated under reduced pressure. The residue was purified by column chromatography (PE/EA=6/1) to afford the desired product as a colorless oil (110 g, 36% yield). ¹H NMR (400 MHz, CDCl₃) δ 7.68-7.70 (m, 4H), 7.38-7.46 (m, 6H), 4.14-4.16 (m, 1H), 3.88-3.89 (m, 1H), 3.71 (s, 1H), 1.83-1.90 (m, 2H), 1.35-1.54 (m, 5H), 1.09 (s, 9H).

### 3-((tert-butyldiphenylsilyl)oxy)cyclohexan-1-one

To a solution of 3-((tert-butyldiphenylsilyl)oxy)cyclohexan-1-ol (110 g, 0.310 mol) in DCM (1 L) was added PCC (133.6 g, 0.620 mol,), and the reaction mixture was stirred at room temperature for 16 h. The reaction mixture was diluted with H₂O (1.2 L) and extracted with DCM (800 mL×3). The combined organic layers were washed with brine, dried over Na₂SO₄, filtered and concentrated under reduced pressure. The residue was purified by column chromatography (PE/EA=10/1) to afford the desired product (101 g, 92% yield) as a colorless oil. ¹H NMR (400 MHz, CDCl₃) δ 7.66-7.63 (m, 4H), 7.45-7.25 (m, 6H), 4.20-4.17 (m, 1H), 2.42 (d, J=4.0 Hz, 2H), 2.39-2.30 (m, 1H), 2.28-2.20 (m, 1H), 2.19-2.11 (m, 1H), 1.78-1.75 (m, 2H), 1.74-1.63 (m, 1H), 1.05 (s, 9H).

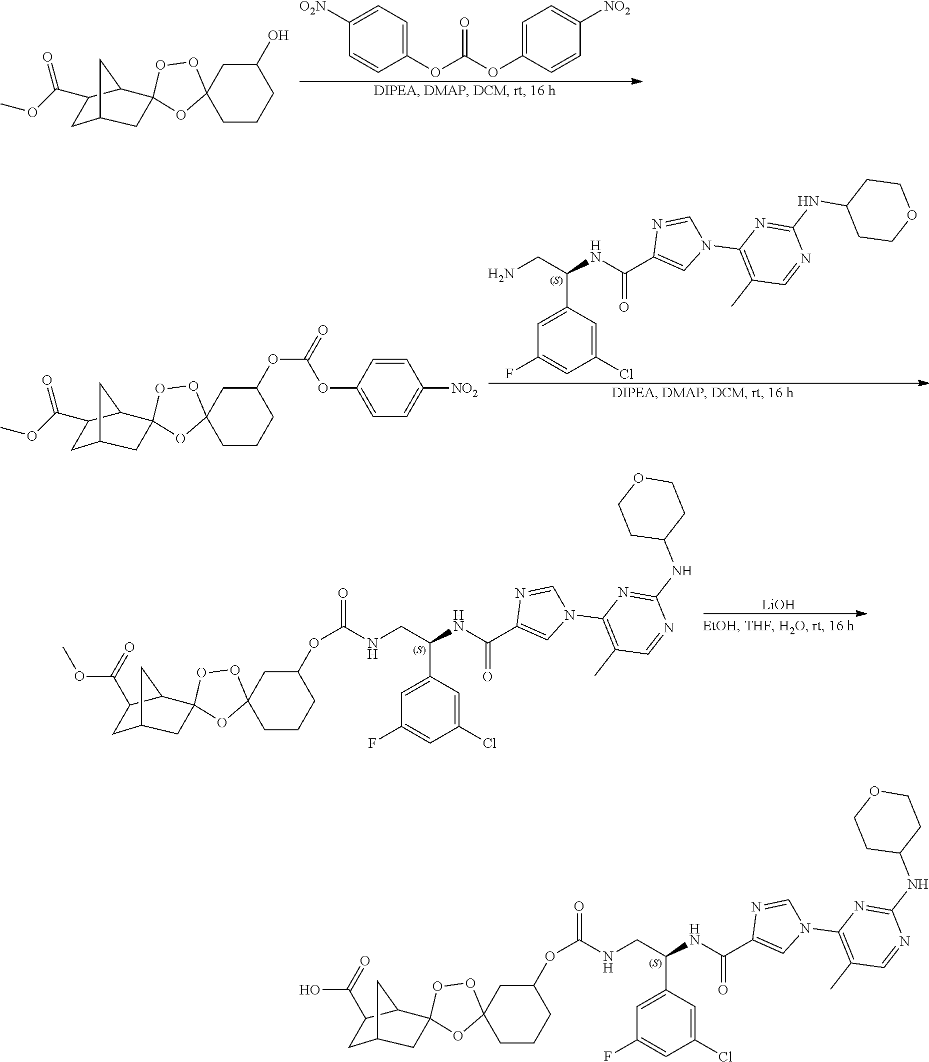

### Methyl (1R,2S,4S,6R)-3″-(((4-nitrophenoxy)carbonyl)oxy)dispiro[bicyclo[2.2.1]heptane-2,3′-[1,2,4]trioxolane-5′,1″-cyclohexane]-6-carboxylate

To a solution of methyl (1R,2S,4S,6R)-3″-Hydroxydispiro[bicyclo[2.2.1]heptane-2,3′-[1,2,4]trioxolane-5′,1″-cyclohexane]-6-carboxylate (2.0 g, 6.7 mmol; 4r, obtained from 3r via deprotection using General Procedure C), DIPEA (3.5 g, 26.8 mmol) and DMAP (81.8 mg, 0.67 mmol) in DCM (60 mL) was added bis(4-nitrophenyl) carbonate (4.08 g, 13.4 mmol). The reaction was stirred at room temperature overnight. The solvent was removed by vacuum. The residue was purified by column chromatography (PE/EA=20/1) to give methyl (1R,2S,4S,6R)-3″-(((4-nitrophenoxy)carbonyl)oxy)dispiro[bicyclo[2.2.1]heptane-2,3′-[1,2,4]trioxolane-5′,1″-cyclohexane]-6-carboxylate (2.0 g, 64.5% yield) as a light yellow solid. ¹H NMR (400 MHz, CDCl₃) δ 8.29 (d, J=8.4 Hz, 2H), 7.39 (d, J=8.4 Hz, 2H), 4.81 (s, 1H), 3.67 (d, J=12.0 Hz, 3H), 2.88 (s, 1H), 2.62 (d, J=18.0 Hz, 1H), 2.37 (m, 2H), 2.15-1.79 (m, 6H), 1.59 (m, 7H).

### Methyl (1R,2S,4S,6R)-3″-((((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamoyl)oxy)dispiro[bicyclo[2.2.1]heptane-2,3′-[1,2,4]trioxolane-5′,1″-cyclohexane]-6-carboxylate

Prepared based on general procedure E. To a stirred solution of (S)—N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide (200 mg, 0.43 mmol), DIPEA (164 mg, 1.30 mmol) and DMAP (6 mg, 0.04 mmol) in DMF (7 mL) was added methyl (1R,2S,4S,6R)-3″-(((4-nitrophenoxy)carbonyl)oxy)dispiro[bicyclo[2.2.1]heptane-2,3′-[1,2,4]trioxolane-5′,1″-cyclohexane]-6-carboxylate (200 mg, 0.43 mmol). The mixture was stirred at room temperature overnight. The reaction mixture was diluted with water (10 mL) and extracted by DCM (10 mL×3). The combined organic layers were washed by brine, dried over anhydrous Na₂SO₄, filtered and concentrated. The residue was purified by column chromatography (DCM/MeOH=20/1) to give methyl (1R,2S,4S,6R)-3″-((((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamoyl)oxy)dispiro[bicyclo[2.2.1]heptane-2,3′-[1,2,4]trioxolane-5′,1″-cyclohexane]-6-carboxylate (300 mg, 89.0% yield) as a light yellow solid. LCMS: Calculated Exact Mass=797.3, Found [M+H]⁺ (ESI+)=798.4.

### (1R,2S,4S,6R)-3″-((((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamoyl)oxy)dispiro[bicyclo[2.2.1]heptane-2,3′-[1,2,4]trioxolane-5′,1″-cyclohexane]-6-carboxylic acid

To a stirred solution of methyl (1R,2S,4S,6R)-3″-((((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamoyl)oxy)dispiro[bicyclo[2.2.1]heptane-2,3′-[1,2,4]trioxolane-5′,1″-cyclohexane]-6-carboxylate (300 mg, 0.38 mmol) in THF (30 mL) and EtOH (2 mL) was added a solution of LiOH (72 mg, 3.0 mmol, dissolved in 15 mL H₂O). The mixture was stirred at room temperature overnight. When the reaction was completed, the organic solvent was removed in vacuum. The resulted mixture was adjusted to pH=5-6 by 1N HCl. The mixture was extracted by DCM (30 mL×3). The combined organic layers were washed by brine, dried over anhydrous Na₂SO₄, filtered and concentrated. The residue was purified by column chromatography (DCM/MeOH=20/1) to give (1R,2S,4S,6R)-3″-((((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamoyl)oxy)dispiro[bicyclo[2.2.1]heptane-2,3′-[1,2,4]trioxolane-5′,1″-cyclohexane]-6-carboxylic acid (141 mg, 48.0% yield) as a white solid. LCMS: Calculated Exact Mass=783.2, Found [M+H]⁺ (ESI+)=784.5. ¹H NMR (400 MHz, DMSO-d₆) δ 12.22 (br. s, 1H), 8.79-8.77 (m, 1H), 8.36 (s, 1H), 8.30 (s, 1H), 8.11 (s, 1H), 7.41-7.20 (m, 5H), 5.14 (d, J=8.0 Hz, 1H), 4.49-4.38 (m, 1H), 3.87-3.84 (m, 3H), 3.46-3.36 (m, 4H), 2.65-2.56 (m, 1H), 2.46-2.44 (m, 1H), 2.29 (s, 1H), 2.19 (s, 3H), 1.93-1.66 (m, 8H), 1.58-1.16 (m, 10H).

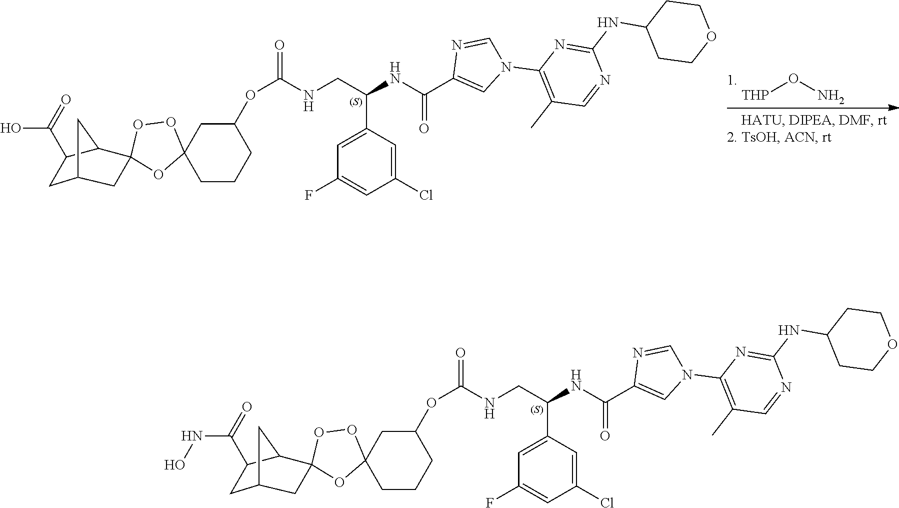

### (1R,2S,4S,6R)-6-(((tetrahydro-2H-pyran-2-yl)oxy)carbamoyl)dispiro[bicyclo[2.2.1]heptane-2,3′-[1,2,4]trioxolane-5′,1″-cyclohexan]-3″-yl ((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamate

To a solution of (1R,2S,4S,6R)-3″-((((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamoyl)oxy)dispiro[bicyclo[2.2.1]heptane-2,3′-[1,2,4]trioxolane-5′,1″-cyclohexane]-6-carboxylic acid (50 mg, 0.064 mmol) and O-(tetrahydro-2H-pyran-2-yl)hydroxylamine (22.4 mg, 0.19 mmol) in DMF (3 mL) were added HATU (36.3 mg, 0.096 mmol) and DIPEA (41 mg, 0.32 mmol). The reaction was stirred at room temperature overnight. After completion, the reaction mixture was diluted with water and extracted by DCM (5 mL×3). The combined organic layers were washed by brine, dried over anhydrous Na₂SO₄, filtered and concentrated under reduced pressure. The residue was purified by column chromatography (DCM/MeOH=20/1) to give (1R,2S,4S,6R)-6-(((tetrahydro-2H-pyran-2-yl)oxy)carbamoyl)dispiro[bicyclo[2.2.1]heptane-2,3′-[1,2,4]trioxolane-5′,1″-cyclohexan]-3″-yl ((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamate (52.0 mg, 91.2% yield) as a white solid. LCMS: Calculated Exact Mass=882.3, Found [M+H]⁺ (ESI+)=883.5

### (1R,2S,4S,6R)-6-(hydroxycarbamoyl)dispiro[bicyclo[2.2.1]heptane-2,3′-[1,2,4]trioxolane-5′,1″-cyclohexan]-3″-yl ((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamate

To a solution of (1R,2S,4S,6R)-6-(((tetrahydro-2H-pyran-2-yl)oxy)carbamoyl)dispiro[bicyclo[2.2.1]heptane-2,3′-[1,2,4]trioxolane-5′,1″-cyclohexan]-3″-yl ((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamate (52 mg, 0.059 mmol) in acetonitrile (5 mL) was added p-toluenesulfonic acid (101.5 mg, 0.59 mmol). The mixture was stirred at room temperature for 2 hours. After completion, the reaction mixture was diluted with water and extracted by DCM (5 mL×3). The combined organic phase was washed by brine, dried over anhydrous Na₂SO₄, filtered and concentrated under reduced pressure. The residue was purified by Prep-HPLC to give (1R,2S,4S,6R)-6-(hydroxycarbamoyl)dispiro[bicyclo[2.2.1]heptane-2,3′-[1,2,4]trioxolane-5′,1″-cyclohexan]-3″-yl ((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamate (11.5 mg, 24.9% yield) as a white solid. LCMS: Calculated Exact Mass=798.3, Found [M+H]⁺ (ESI+)=799.5. ¹H NMR (400 MHz, DMSO-d₆) δ 10.52-10.50 (m, 1H), 8.79-8.65 (m, 2H), 8.36-8.30 (m, 2H), 8.11 (s, 1H), 7.41-7.28 (m, 4H), 7.22-7.20 (m, 1H), 5.15-5.13 (m, 1H), 4.45-4.41 (m, 1H), 3.91-3.84 (m, 3H), 3.41-3.32 (m, 4H), 2.49-2.45 (m, 1H), 2.25-2.20 (m, 2H), 2.19 (s, 3H), 2.13-2.00 (m, 1H), 1.84-1.66 (m, 9H), 1.55-1.42 (m, 6H), 1.31-1.24 (m, 2H).

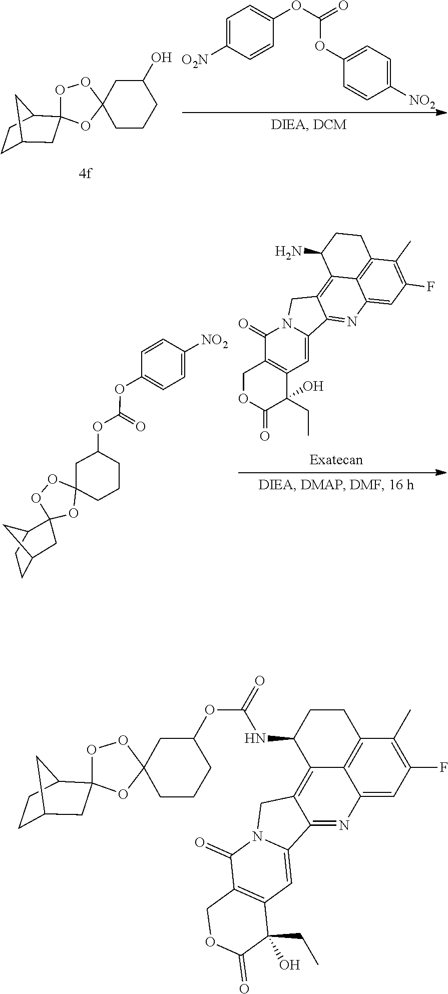 [SMILES: OC1CCCC2(C1)OOC1(CC3CCC1C3)O2 ; O=C(Oc1ccc([N+](=O)[O-])cc1)Oc1ccc([N+](=O)[O-])cc1 ; O=C(Oc1ccc([N+](=O)[O-])cc1)OC1CCCC2(C1)OOC1(CC3CCC1C3)O2 ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)OC1CCCC2(C1)OOC1(CC4CCC1C4)O2)CC3]

### (1S,2S,4R)-dispiro[bicyclo[2.2.1]heptane-2,3′-[1,2,4]trioxolane-5′,1″-cyclohexan]-3″-yl (4-nitrophenyl) carbonate

To a solution of (1S,2S,4R)-dispiro[bicyclo[2.2.1]heptane-2,3′-[1,2,4]trioxolane-5′,1″-cyclohexan]-3″-ol (800 mg, 2.80 mol; 4f obtained from 3f via deprotection using General Procedure C) and DIPEA (1.07 g, 8.40 mmol) in DCM (10 mL) was added bis(4-nitrophenyl) carbonate (1.28 g, 4.20 mmol). The reaction mixture was stirred at room temperature overnight. After completion, the reaction mixture was concentrated under reduced pressure. The residue was purified by column chromatography (PE/EA=5/1) to afford the desired product as a white solid (755.9 mg, 67% yield). LCMS: Calculated Exact Mass=405.1, Found [M+H] (ESI+)=406.2.

### (1S,2S,4R)-dispiro[bicyclo[2.2.1]heptane-2,3′-[1,2,4]trioxolane-5′,1″-cyclohexan]-3″-yl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate

Prepared based on general procedure E. To a solution of (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (210 mg, 0.48 mmol), DIPEA (208 mg, 1.61 mmol) and DMAP (13 mg, 0.11 mmol) in DMF (5 mL) was added (1S,2S,4R)-dispiro[bicyclo[2.2.1]heptane-2,3′-[1,2,4]trioxolane-5′,1″-cyclohexan]-3″-yl (4-nitrophenyl) carbonate (217 mg, 0.54 mmol). The mixture was stirred at room temperature for 16 hours. After completion, the reaction mixture was diluted with water (10 mL) and extracted with DCM (5 mL×3). The combined organic layers were washed by brine, dried over anhydrous Na₂SO₄, filtered and concentrated under reduced pressure. The residue was purified by column chromatography (DCM/MeOH=5/1) to afford the desired product as a white solid (132.6 mg, 35% yield). LCMS: Calculated Exact Mass=701.3, Found [M+H](ESI+)=702.6. ¹H NMR (400 MHz, DMSO) δ 8.08-7.90 (m, 1H), 7.73 (d, J=10.8 Hz, 1H), 7.28 (s, 1H), 6.50 (s, 1H), 5.40 (s, 2H), 5.30-5.04 (m, 3H), 4.68 (s, 1H), 3.31-3.22 (m, 1H), 3.15-3.02 (m, 1H), 2.33 (s, 3H), 2.31-2.03 (m, 5H), 1.93-1.79 (m, 4H), 1.78-1.64 (m, 2H), 1.58-1.03 (m, 11H), 0.86 (t, J=7.2 Hz, 3H).

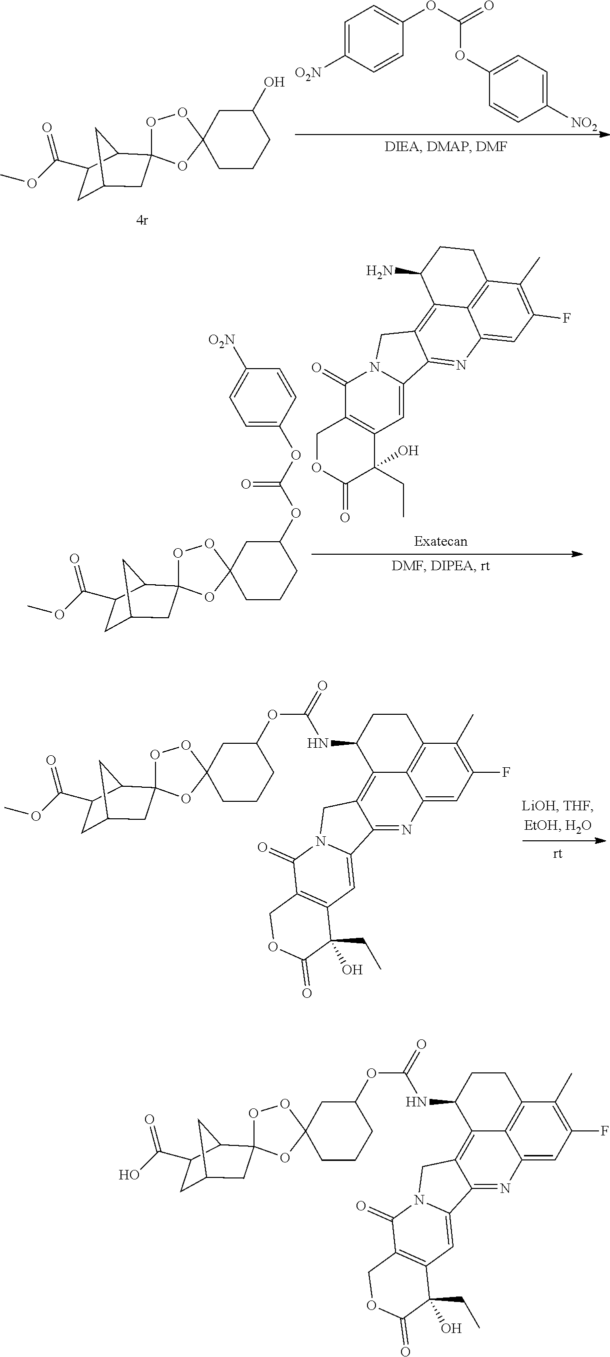

### Methyl (1R,2S,4S,6R)-3″-(((4-nitrophenoxy)carbonyl)oxy)dispiro[bicyclo[2.2.1]heptane-2,3′-[1,2,4]trioxolane-5′,1″-cyclohexane]-6-carboxylate

To a solution of methyl (1R,2S,4S,6R)-3″-hydroxydispiro[bicyclo[2.2.1]heptane-2,3′-[1,2,4]trioxolane-5′,1″-cyclohexane]-6-carboxylate (2.0 g, 6.7 mmol; 4r, obtained via deprotection of 3r using General Procedure C), DMAP (82 mg, 0.67 mmol) and DIEA (3.5 g, 26.8 mmol) in DMF (60 mL) was added bis(4-nitrophenyl) carbonate (4.08 g, 13.4 mmol). The reaction was stirred at room temperature overnight. After completion, the mixture was concentrated under reduced pressure. The residue was purified by column chromatography (PE/EA=20/1) to give the product as a yellow solid (2.0 g, 64.5% yield). LCMS: Calculated Exact Mass=463.15, Found [M+H]⁺ (ESI+)=464.2. ¹H NMR (400 MHz, CDCl₃) δ 8.29 (d, J=8.4 Hz, 2H), 7.39 (d, J=8.4 Hz, 2H), 4.81 (s, 1H), 3.67 (m, 3H), 2.90 (m, 1H), 2.62 (m, 1H), 2.32 (d, J=41.8 Hz, 2H), 2.21-1.73 (m, 6H), 1.75-1.39 (m, 7H).

### Methyl (1R,2S,4S,6R)-3″-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)dispiro[bicyclo[2.2.1]heptane-2,3′-[1,2,4]trioxolane-5′,1″-cyclohexane]-6-carboxylate

Prepared based on general procedure E. To a solution of (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (180 mg, 0.44 mmol) and DIEA (170 mg, 1.31 mmol) in DMF (5 mL) was added methyl (1R,2S,4S,6R)-3″-(((4-nitrophenoxy)carbonyl)oxy)dispiro[bicyclo[2.2.1]heptane-2,3′-[1,2,4]trioxolane-5′,1″-cyclohexane]-6-carboxylate (200 mg, 0.46 mmol). The reaction mixture was stirred at room temperature overnight. After completion, the reaction mixture was concentrated under reduced pressure. The residue was purified by column chromatography (DCM/MeOH=20/1) to give the product as a yellow solid (130 mg, 41.4% yield). LCMS: Calculated Exact Mass=759.3, Found [M+H]⁺ (ESI+)=760.2.

### (1R,2S,4S,6R)-3″-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)dispiro[bicyclo[2.2.1]heptane-2,3′-[1,2,4]trioxolane-5′,1″-cyclohexane]-6-carboxylic acid

To a solution of methyl (1R,2S,4S,6R)-3″-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)dispiro[bicyclo[2.2.1]heptane-2,3′-[1,2,4]trioxolane-5′,1″-cyclohexane]-6-carboxylate (120 mg, 0.16 mmol) in EtOH (6 mL) and H₂O (6 ml) was added LiOH (11.4 mg, 0.48 mmol). The reaction mixture was stirred at room temperature for 16 h. The reaction mixture was adjusted to pH=5-6 with 1 N HCl and extracted with EA (5 mL×3). The combined organic layers were washed with brine, dried over anhydrous Na₂SO₄, filtered and concentrated under reduced pressure. The residue was purified by Prep-TLC (DCM/MeOH=20/1) to afford the desired product as a white solid (35.6 mg, 30.2% yield). LCMS: Calculated Exact Mass=745.2, Found [M+H]⁺ (ESI+)=746.5. ¹H NMR (400 MHz, DMSO-d₆) δ 8.00-7.98 (m, 1H), 7.76 (d, J=8.0 Hz, 1H), 7.30 (d, J=4.0 Hz, 1H), 6.51 (s, 1H), 5.43 (s, 2H), 5.23-5.10 (m, 3H), 4.68 (s, 1H), 3.23-3.10 (m, 2H), 2.65-2.52 (m, 1H), 2.25 (s, 3H), 2.25-2.23 (m, 2H), 2.16-2.02 (m, 2H), 1.97-1.68 (m, 8H), 1.53-1.36 (m, 7H), 1.26-1.24 (m, 1H), 0.90-0.85 (m, 3H).

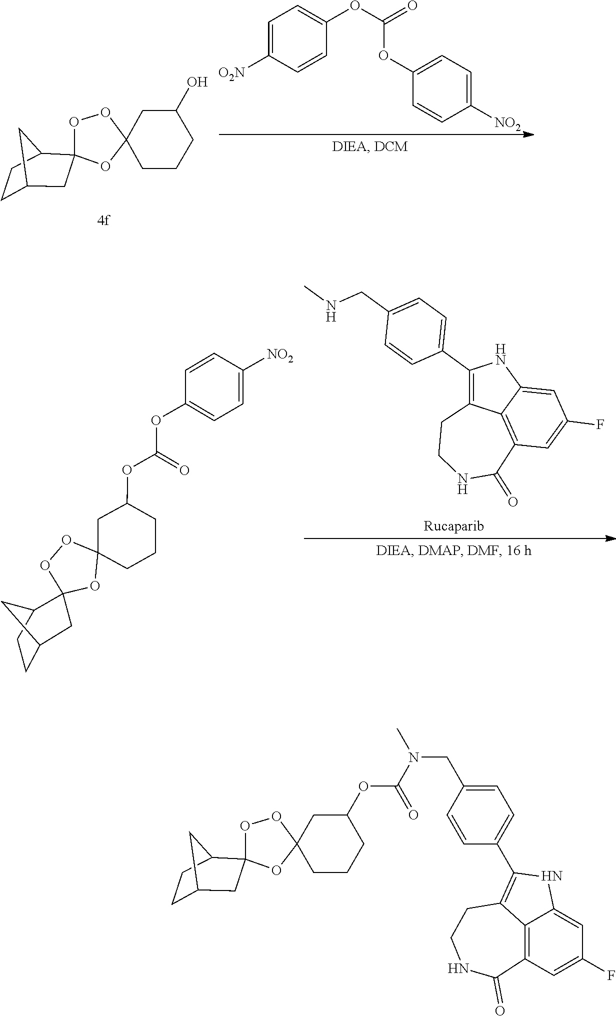 [SMILES: OC1CCCC2(C1)OOC1(CC3CCC1C3)O2 ; O=C(Oc1ccc([N+](=O)[O-])cc1)Oc1ccc([N+](=O)[O-])cc1 ; O=C(Oc1ccc([N+](=O)[O-])cc1)OC1CCCC2(C1)OOC1(CC3CCC1C3)O2 ; CNCc1ccc(-c2[nH]c3cc(F)cc4c3c2CCNC4=O)cc1 ; CN(Cc1ccc(-c2[nH]c3cc(F)cc4c3c2CCNC4=O)cc1)C(=O)OC1CCCC2(C1)OOC1(CC3CCC1C3)O2]

### (1S,2S,4R)-dispiro[bicyclo[2.2.1]heptane-2,3′-[1,2,4]trioxolane-5′,1″-cyclohexan]-3″-yl (4-nitrophenyl) carbonate

To a solution of (1S,2S,4R)-dispiro[bicyclo[2.2.1]heptane-2,3′-[1,2,4]trioxolane-5′,1″-cyclohexan]-3″-ol (800 mg, 2.80 mol; 4f obtained from 3f via deprotection using General Procedure C) and DIPEA (1.07 g, 8.40 mmol) in DCM (10 mL) was added bis(4-nitrophenyl) carbonate (1.28 g, 4.20 mmol). The reaction mixture was stirred at room temperature overnight. After completion, the reaction mixture was concentrated under reduced pressure. The residue was purified by column chromatography (PE/EA=5/1) to afford the desired product as a white solid (755.9 mg, 67% yield). LCMS: Calculated Exact Mass=405.1, Found [M+H](ESI+)=406.2.

### (1S,2S,4R)-dispiro[bicyclo[2.2.1]heptane-2,3′-[1,2,4]trioxolane-5′,1″-cyclohexan]-3″-yl (4-(8-fluoro-1-oxo-2,3,4,6-tetrahydro-1H-azepino[5,4,3-cd]indol-5-yl)benzyl)(methyl)carbamate

Prepared based on general procedure E. To a solution of 8-fluoro-5-(4-((methylamino)methyl)phenyl)-2,3,4,6-tetrahydro-1H-azepino[5,4,3-cd]indol-1-one (112 mg, 0.35 mmol), DIPEA (156 mg, 1.13 mmol) and DMAP (9 mg, 0.07 mmol) in DMF (5 mL) was added (1S,2S,4R)-dispiro[bicyclo[2.2.1]heptane-2,3′-[1,2,4]trioxolane-5′,1″-cyclohexan]-3″-yl (4-nitrophenyl) carbonate (153 mg, 0.38 mmol). The reaction was stirred at room temperature for 16 hours. After completion, the reaction mixture was diluted with water (10 mL) and extracted by DCM (5 mL×3). The combined organic layers were washed by brine, dried over anhydrous Na₂SO₄, filtered and concentrated under reduced pressure. The residue was purified by column chromatography (DCM/MeOH=5/1) to afford the desired product as a white solid (132.6 mg, 59% yield). LCMS: Calculated Exact Mass=589.3, Found [M+H](ESI+)=590.6. ¹H NMR (400 MHz, DMSO-d₆) δ 11.68 (s, 1H), 8.25 (t, J=6.0 Hz, 1H), 7.63-7.61 (m, 2H), 7.44-7.38 (m, 3H), 7.32 (dd, J=10.4, 2.4 Hz, 1H), 4.73-4.61 (m, 1H), 4.56-4.39 (m, 2H), 3.40-3.38 (m, 2H), 3.04 (s, 2H), 2.84 (s, 3H), 2.23 (s, 2H), 2.15-1.98 (m, 1H), 1.93-1.63 (m, 6H), 1.50-1.31 (m, 7H), 1.29-1.15 (m, 2H).

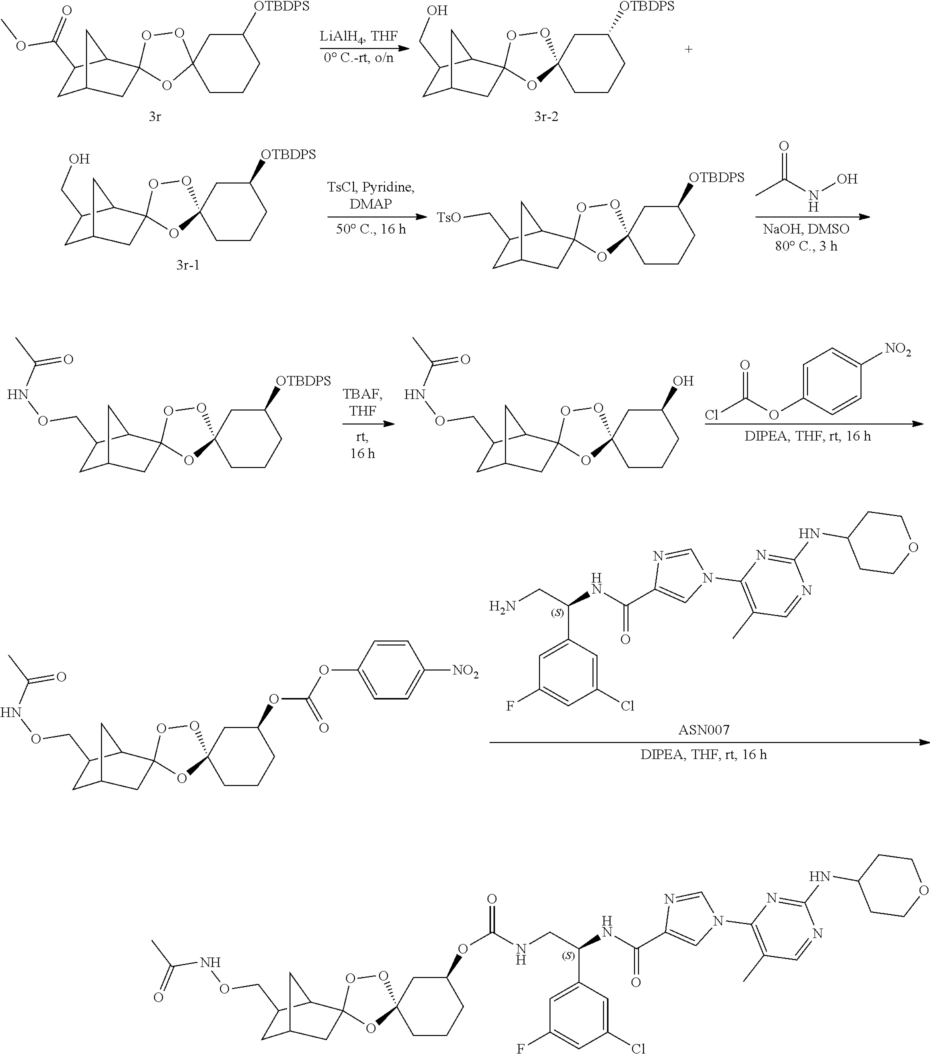

### ((1R,2S,4R,6R)-3″-((tert-butyldiphenylsilyl)oxy)dispiro[bicyclo[2.2.1]heptane-2,3′-[1,2,4]trioxolane-5′,1″-cyclohexan]-6-yl)methanol

To a solution of methyl (1R,2S,4S,6R)-3″-((tert-butyldiphenylsilyl)oxy)dispiro[bicyclo[2.2.1]heptane-2,3′-[1,2,4]trioxolane-5′,1″-cyclohexane]-6-carboxylate (3r; 5.8 g, 10.82 mmol) in THF (100 ml) was slowly added lithium aluminum hydride (617 mg, 16.23 mmol) at 0° C., then the mixture was slowly warmed to room temperature and stirred overnight. When the reaction was completed by TLC, sodium sulfate decahydrate (10 g) was added. And the mixture was stirred at room temperature for 1 h. Then the mixture was filtered, the filtrate was concentrated. The residue was purified by column chromatography (PE/EA=8/1) to give the two diastereomers. Less polar isomer (3r-1): 1.8 g as a colorless oil. ¹H NMR (400 MHz, CDCl₃) δ 7.64-7.54 (m, 4H), 7.39-7.25 (m, 6H), 3.74-3.59 (m, 1H), 3.43-3.27 (m, 2H), 2.20 (s, 1H), 2.09-1.98 (m, 2H), 1.92-1.80 (m, 2H), 1.78-1.65 (m, 3H), 1.55-1.44 (m, 2H), 1.40-1.25 (m, 5H), 1.23-1.13 (m, 3H), 0.98 (m, 9H). More polar isomer (3r-2): 2.1 g as a colorless oil: ¹H NMR (400 MHz, CDCl₃) δ 7.65-7.52 (m, 4H), 7.40-7.22 (m, 6H), 3.84-3.67 (m, 1H), 3.39-3.26 (m, 2H), 2.24-2.09 (m, 2H), 2.02 (dd, J=12.7, 8.4 Hz, 2H), 1.78 (ddd, J=22.0, 11.8, 8.8 Hz, 1H), 1.71-1.61 (m, 2H), 1.59-1.08 (m, 11H), 1.00 (m, 9H).

### ((1R,2S,3″S,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methyl 4-methylbenzenesulfonate

To a solution of ((1R,2S,3″S,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methanol (3r-1; 400 mg, 0.78 mmol) in pyridine (10 mL) was added tosyl chloride (224 mg, 1.18 mmol) and DMAP (8 mg, 0.07 mmol), and the reaction mixture was stirred at 50° C. for 16 h. The reaction mixture was diluted with water (5 mL) and extracted with EA (5 mL×3). The combined organic phase was washed with brine, dried over Na₂SO₄, filtered and concentrated. The residue was purified by column chromatography (PE/EA=20/1) to afford the desired product as a colorless oil (260 mg, 51% yield). LCMS: Calculated Exact Mass=662.9, Found [M+H]⁺ (ESI+)=663.7.

### N-(((1R,2S,3″S,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methoxy)acetamide

To a solution of ((1R,2S,3″S,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methyl 4-methylbenzenesulfonate (280 mg, 0.42 mmol) in DMSO (5 mL) was added NaOH (67 mg, 1.68 mmol) and N-hydroxyacetamide (158 mg, 2.10 mmol). The reaction mixture was stirred at 80° C. for 3 h. After completion, the reaction mixture was cooled to room temperature, diluted with H₂O (5 mL) and extracted with EA (10 mL×3). The combined organic layers were washed with brine, dried over anhydrous Na₂SO₄, filtered and concentrated. The residue was purified by column chromatography (PE/EA=2/1) to afford the desired product (80 mg, 33% yield) as a white solid. LCMS: Calculated Exact Mass=565.3, Found [M+Na]⁺ (ESI+)=588.5.

### N-(((1R,2S,3″S,4R,5′S,6R)-3″-hydroxydispiro[bicyclo[2.2.1]heptane-2,3′-[1,2,4]trioxolane-5′,1″-cyclohexan]-6-yl)methoxy)acetamide

Prepared based on general procedure C. To a solution of N-(((1R,2S,3″S,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methoxy)acetamide (80 mg, 0.11 mmol) in THF (5 mL) was added TBAF (276 mg, 0.55 mmol). The reaction mixture was stirred at room temperature for 16 h. The reaction mixture was diluted with H₂O (5 mL) and extracted with EA (10 mL×3). The combined organic layers were washed with brine, dried over anhydrous Na₂SO₄, filtered and concentrated. The residue was purified by Pre-TLC (PE/EA=1/1) to afford the desired product (36 mg, 78% yield) as a white solid. ¹H NMR (400 MHz, CDCl₃) δ 3.89 (s, 1H), 3.73 (s, 2H), 2.40-2.37 (m, 4H), 2.17-2.11 (m, 1H), 1.98-1.86 (m, 4H), 1.84-1.72 (m, 5H), 1.66-1.61 (m, 2H), 1.55-1.51 (m, 3H), 1.44-1.39 (m, 2H), 1.17-1.15 (m, 1H).

### (1R,2S,3″S,4R,5′S,6R)-6-((acetamidooxy)methyl)dispiro[bicyclo[2.2.1]heptane-2,3′-[1,2,4]trioxolane-5′,1″-cyclohexan]-3″-yl (4-nitrophenyl) carbonate

Prepared based on general procedure D. To a solution of N-(((1R,2S,3″S,4R,5′S,6R)-3″-hydroxydispiro[bicyclo[2.2.1]heptane-2,3′-[1,2,4]trioxolane-5′,1″-cyclohexan]-6-yl)methoxy)acetamide (15 mg, 0.04 mmol) in THF (5 mL) was added DIEA (17 mg, 0.12 mmol,) and 4-nitrophenyl carbonochloridate (11 mg, 0.05). The reaction mixture was stirred at room temperature for 16 h. The reaction mixture was concentrated and the residue was directly used for the next step without further purification.

### (1R,2S,3″S,4R,5′S,6R)-6-((acetamidooxy)methyl)dispiro[bicyclo[2.2.1]heptane-2,3′-[1,2,4]trioxolane-5′,1″-cyclohexan]-3″-yl ((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamate

To a solution of (1R,2S,3″S,4R,5′S,6R)-6-((acetamidooxy)methyl)dispiro[bicyclo[2.2.1]heptane-2,3′-[1,2,4]trioxolane-5′,1″-cyclohexan]-3″-yl (4-nitrophenyl) carbonate (22 mg, 0.04 mmol) in THF (5 mL) was added DIEA (17 mg, 0.12 mmol,) and (S)—N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide (19 mg, 0.04). The reaction mixture was stirred at room temperature for 16 h. The reaction mixture was diluted with H₂O (5 mL) and extracted with EA (10 mL×3). The combined organic layers were washed with brine, dried over anhydrous Na₂SO₄, filtered and concentrated. The residue was purified by Prep-TLC (DCM/MeOH=30/1) to afford the desired product (8.5 mg, 11.2% yield over two steps) as a white solid. LCMS: Calculated Exact Mass=826.3, Found [M+H]⁺ (ESI+)=827.8. ¹H NMR (400 MHz, DMSO-d₆) δ 8.90 (d, J=8.0 Hz, 1H), 8.36 (s, 1H), 8.34-8.20 (m, 2H), 8.11 (d, J=1.2 Hz, 1H), 7.42-7.37 (m, 1H), 7.36-7.29 (m, 2H), 7.25 (d, J=20.0 Hz, 1H), 5.29-5.19 (m, 1H), 4.85-4.78 (m, 1H), 3.90-3.84 (m, 3H), 3.72-3.52 (m, 4H), 3.41-3.38 (m, 3H), 2.33-2.35 (m, 1H), 2.27 (s, 3H), 2.19 (s, 5H), 1.84-1.61 (m, 6H), 1.61-1.27 (m, 10H), 1.10-1.06 (m, 2H).

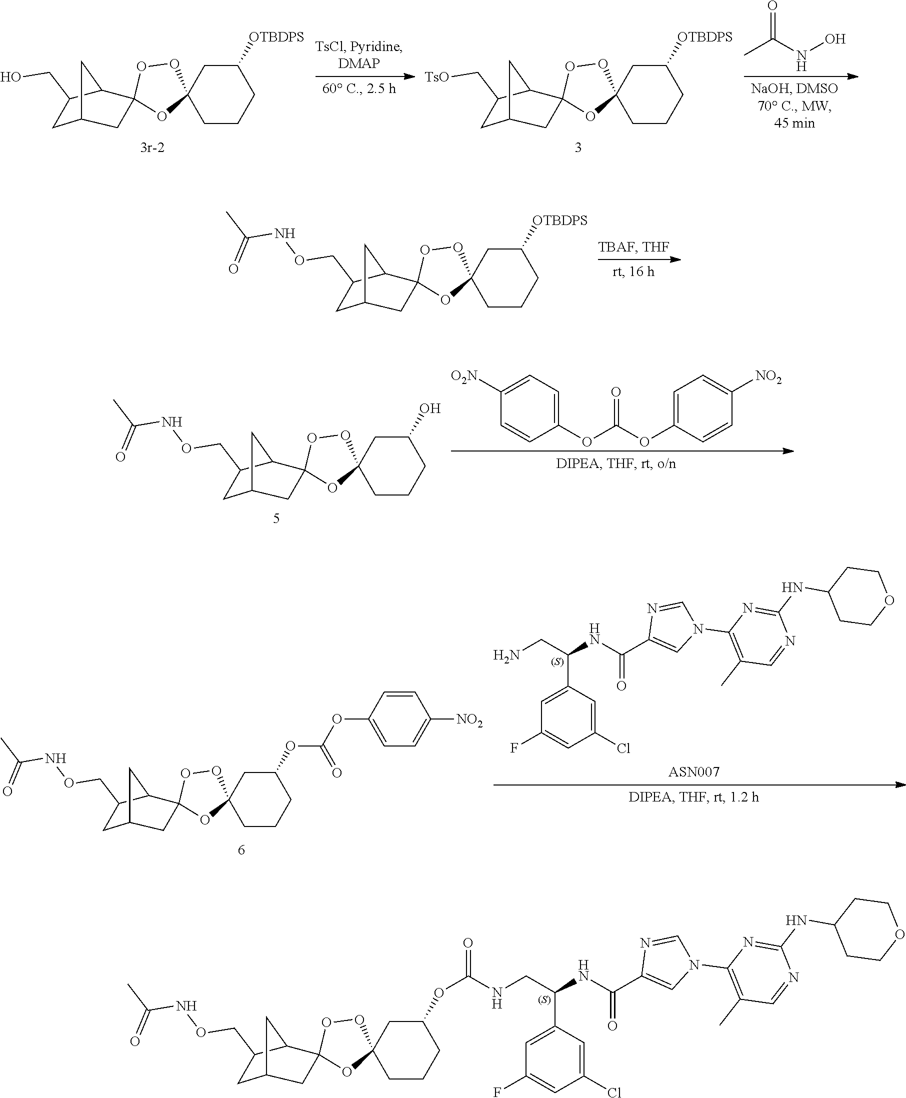

### ((1R,2S,3″R,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methyl 4-methylbenzenesulfonate

To a solution of ((1R,2S,3″R,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methanol (3r-2; 860 mg, 1.69 mmol) in pyridine (10 mL) was added tosyl chloride (970 mg, 5.08 mmol) and DMAP (21 mg, 0.17 mmol). The reaction mixture was stirred at 60° C. for 2.5 h. After completion, the reaction mixture was cooled down to room temperature and concentrated under reduced pressure. The residue was purified by column chromatography (PE/EA=10/1) to afford the desired product as a colorless oil (870 mg, 77.7% yield). LCMS: Calculated Exact Mass=662.9, Found [M+H]⁺ (ESI+)=663.7.

### N-(((1R,2S,3″R,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methoxy)acetamide

To a solution of ((1R,2S,3″R,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methyl 4-methylbenzenesulfonate (445 mg, 0.67 mmol) in DMSO (3.5 mL) was added NaOH (135 mg, 3.36 mmol,) and N-hydroxyacetamide (505 mg, 6.72 mmol). The reaction mixture was stirred at 70° C. for 45 min under microwave. The reaction mixture was diluted with H₂O (5 mL) and extracted with EA (10 mL×3). The combined organic layers were washed with brine, dried over anhydrous Na₂SO₄, filtered and concentrated. The residue was purified by column chromatography (DCM/MeOH=20/1) to afford the desired product (210 mg, 55.0% yield) as a light yellow solid. LCMS: Calculated Exact Mass=565.3, Found [M+Na]⁺ (ESI+)=588.5.

### N-(((1R,2S,3″R,4R,5′S,6R)-3″-hydroxydispiro[bicyclo[2.2.1]heptane-2,3′-[1,2,4]trioxolane-5′,1″-cyclohexan]-6-yl)methoxy)acetamide

Prepared according to general procedure C. To a solution of N-(((1R,2S,3″R,4R,5′S,6R)-3″-((tert-butyldiphenylsilyl)oxy)dispiro[bicyclo[2.2.1]heptane-2,3′-[1,2,4]trioxolane-5′,1″-cyclohexan]-6-yl)methoxy)acetamide (210 mg, 0.37 mmol) in THF (10 mL) was added TBAF (590 mg, 1.87 mmol). The reaction mixture was stirred at room temperature for 16 h. The reaction mixture was diluted with H₂O (5 mL) and extracted with EA (10 mL×3). The combined organic layers was washed with brine, dried over anhydrous Na₂SO₄, filtered and concentrated. The residue was purified by column chromatography (DCM/MeOH=20/1) to afford the desired product (115 mg, 94.3% yield) as a colorless oil. ¹H NMR (400 MHz, CDCl₃) δ 3.94 (dt, J=8.0, 4.0 Hz, 1H), 3.81-3.55 (m, 2H), 2.45-2.36 (m, 1H), 2.35-2.26 (m, 2H), 2.15-2.05 (m, 2H), 2.03-1.96 (m, 1H), 1.92 (s, 1H), 1.86-1.68 (m, 6H), 1.65-1.39 (m, 7H).

### (1R,2S,3″R,4R,5′S,6R)-6-((acetamidooxy)methyl)dispiro[bicyclo[2.2.1]heptane-2,3′-[1,2,4]trioxolane-5′,1″-cyclohexan]-3″-yl (4-nitrophenyl) carbonate

To a solution of N-(((1R,2S,3″R,4R,5′S,6R)-3″-hydroxydispiro[bicyclo[2.2.1]heptane-2,3′-[1,2,4]trioxolane-5′,1″-cyclohexan]-6-yl)methoxy)acetamide (30 mg, 0.09 mmol) in THF (5 mL) was added DIEA (36 mg, 0.28 mmol) and bis(4-nitrophenyl) carbonate (31 mg, 0.10 mmol). The reaction mixture was stirred at room temperature for 16 h. The reaction mixture was concentrated and directly used to the next step without further purification.

### (1R,2S,3″R,4R,5′S,6R)-6-((acetamidooxy)methyl)dispiro[bicyclo[2.2.1]heptane-2,3′-[1,2,4]trioxolane-5′,1″-cyclohexan]-3″-yl ((S)-2-(3-chloro-5-fluorophenyl)-2-(1-(5-methyl-2-((tetrahydro-2H-pyran-4-yl)amino)pyrimidin-4-yl)-1H-imidazole-4-carboxamido)ethyl)carbamate

To a solution of (1R,2S,3″R,4R,5′S,6R)-6-((acetamidooxy)methyl)dispiro[bicyclo[2.2.1]heptane-2,3′-[1,2,4]trioxolane-5′,1″-cyclohexan]-3″-yl (4-nitrophenyl) carbonate (30 mg, 0.09 mmol) in THF (5 mL) was added DIEA (36 mg, 0.28 mmol) and (S)—N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide (43 mg, 0.09 mmol). The reaction mixture was stirred at room temperature for 16 h. The reaction mixture was concentrated and purified by Prep-HPLC to afford the desired product (33.7 mg, 44.3% yield over two steps) as a white solid. LCMS: Calculated Exact Mass=826.3, Found [M+H]⁺ (ESI+)=827.8. ¹H NMR (400 MHz, DMSO-d₆) δ 8.89 (d, J=6.0 Hz, 1H), 8.36 (s, 1H), 8.32-8.20 (m, 2H), 8.11 (s, 1H), 7.39 (s, 1H), 7.33 (d, J=6.8 Hz, 2H), 7.25 (d, J=9.2 Hz, 1H), 5.28-5.18 (m, 1H), 4.73 (d, J=4.4 Hz, 1H), 3.86 (d, J=11.2 Hz, 3H), 3.71-3.52 (m, 4H), 3.49-3.39 (m, 3H), 2.35-2.28 (m, 1H), 2.26 (s, 3H), 2.19 (s, 5H), 2.07 (d, J=12.8 Hz, 1H), 1.92-1.74 (m, 4H), 1.71-1.57 (m, 3H), 1.56-1.27 (m, 7H), 1.19 (dd, J=23.8, 10.6 Hz, 1H), 1.13-1.01 (m, 2H).

### REFERENCES

(1) (a) Griesbaum, K.; Liu, X.; Kassiaris, A.; Scherer, M. Liebigs Ann. 1997, 1997, 1381; (b) Griesbaum, K.; Övez, B.; Huh, T. S.; Dong, Y. Liebigs Ann. 1995, 1995, 1571. (2) Vennerstrom, J. L.; Arbe-Barnes, S.; Brun, R.; Charman, S. A.; Chiu, F. C. K.; Chollet, J.; Dong, Y.; Dorn, A.; Hunziker, D.; Matile, H.; McIntosh, K.; Padmanilayam, M.; Santo Tomas, J.; Scheurer, C.; Scorneaux, B.; Tang, Y.; Urwyler, H.; Wittlin, S.; Charman, W. N. Nature 2004, 430, 900. (3) Charman, S. A.; Arbe-Barnes, S.; Bathurst, I. C.; Brun, R.; Campbell, M.; Charman, W. N.; Chiu, F. C. K.; Chollet, J.; Craft, J. C.; Creek, D. J.; Dong, Y.; Matile, H.; Maurer, M.; Morizzi, J.; Nguyen, T.; Papastogiannidis, P.; Scheurer, C.; Shackleford, D. M.; Sriraghavan, K.; Stingelin, L.; Tang, Y.; Urwyler, H.; Wang, X.; White, K. L.; Wittlin, S.; Zhou, L.; Vennerstrom, J. L. Proc. Natl. Acad. Sci. 2011, 108, 4400. (4) (a) Aron, A. T.; Reeves, A. G.; Chang, C. J. Curr. Opin. Chem. Biol. 2018, 43, 113; (b) Chang, C. J. Nat. Chem. Biol. 2015, 11, 744; (c) Bruemmer, K. J.; Crossley, S. W. M.; Chang, C. J. Angew. Chem. Int. Ed. 2020, 59, 13734; (d) Hirayama, T. Free Radical Biol. Med. 2019, 133, 38. (5) Hirayama, T.; Tsuboi, H.; Niwa, M.; Miki, A.; Kadota, S.; Ikeshita, Y.; Okuda, K.; Nagasawa, H. Chemical Science 2017, 8, 4858. (6) Hirayama, T.; Miki, A.; Nagasawa, H. Metallomics 2018, 11, 111. (7) Spangler, B.; Morgan, C. W.; Fontaine, S. D.; Vander Wal, M. N.; Chang, C. J.; Wells, J. A.; Renslo, A. R. Nat. Chem. Biol. 2016, 12, 680. (8) Aron, A. T.; Loehr, M. O.; Bogena, J.; Chang, C. J. J. Am. Chem. Soc. 2016, 138, 14338. (9) Aron, A. T.; Heffern, M. C.; Lonergan, Z. R.; Vander Wal, M. N.; Blank, B. R.; Spangler, B.; Zhang, Y.; Park, H. M.; Stahl, A.; Renslo, A. R.; Skaar, E. P.; Chang, C. J. Proc. Natl. Acad. Sci. 2017, 114, 12669. (10) Xu, S.; Liu, H.-W.; Chen, L.; Yuan, J.; Liu, Y.; Teng, L.; Huan, S.-Y.; Yuan, L.; Zhang, X.-B.; Tan, W. J. Am. Chem. Soc. 2020, 142, 2129. (11) Zhao, N.; Huang, Y.; Wang, Y. H.; Muir, R. K.; Chen, Y. C.; Hooshdaran, N.; Wei, J.; Viswanath, P.; Seo, Y.; Ruggero, D.; Renslo, A. R.; Evans, M. J. J Nucl Med 2020. (12) Muir, R. K.; Zhao, N.; Wei, J.; Wang, Y.-h.; Moroz, A.; Huang, Y.; Chen, Y.-C.; Sriram, R.; Kurhanewicz, J.; Ruggero, D.; Renslo, A. R.; Evans, M. J. ACS Cent. Sci. 2019, 5, 727. (13) (a) Ismail, H. M.; Barton, V. E.; Panchana, M.; Charoensutthivarakul, S.; Biagini, G. A.; Ward, S. A.; O'Neill, P. M. Angew. Chem. Int. Ed. 2016, 55, 6401; (b) Wei, C.; Zhao, C.-X.; Liu, S.; Zhao, J.-H.; Ye, Z.; Wang, H.; Yu, S.-S.; Zhang, C.-J. Chem. Commun. 2019, 55, 9535. (14) Chen, Y.-C.; Oses-Prieto, J. A.; Pope, L. E.; Burlingame, A. L.; Dixon, S. J.; Renslo, A. R. J. Am. Chem. Soc. 2020, 142, 19085. (15) Fontaine, S. D.; DiPasquale, A. G.; Renslo, A. R. Org. Lett. 2014, 16, 5776. (16) Wu, J.; Wang, X.; Chiu, F. C. K.; Häberli, C.; Shackleford, D. M.; Ryan, E.; Kamaraj, S.; Bulbule, V. J.; Wallick, A. I.; Dong, Y.; White, K. L.; Davis, P. H.; Charman, S. A.; Keiser, J.; Vennerstrom, J. L. J. Med. Chem. 2020, 63, 3723. (17) Yamansarov, E. Y.; Kazakova, O. B.; Medvedeva, N. I.; Kazakov, D. V.; Kukovinets, O. S.; Tolstikov, G. A. Russ. J. Org. Chem. 2014, 50, 1043. (18) Dong, Y.; Chollet, J.; Matile, H.; Charman, S. A.; Chiu, F. C. K.; Charman, W. N.; Scorneaux, B.; Urwyler, H.; Santo Tomas, J.; Scheurer, C.; Snyder, C.; Dorn, A.; Wang, X.; Karle, J. M.; Tang, Y.; Wittlin, S.; Brun, R.; Vennerstrom, J. L. J. Med. Chem. 2005, 48, 4953. (19) Kazakova, O. B.; Khusnutdinova, E. F.; Petrova, A. V.; Yamansarov, E. Y.; Lobov, A. N.; Fedorova, A. A.; Suponitsky, K. Y. J. Nat. Prod. 2019, 82, 2550. (20) Chhantyal-Pun, R.; Khan, M. A. H.; Taatjes, C. A.; Percival, C. J.; Orr-Ewing, A. J.; Shallcross, D. E. Int. Rev. Phys. Chem. 2020, 39, 385. (21) Blank, B. R.; Gonciarz, R. L.; Talukder, P.; Gut, J.; Legac, J.; Rosenthal, P. J.; Renslo, A. R. ACS Infect. Dis. 2020, 6, 1827. (22) Charman, S. A.; Perry, C. S.; Chiu, F. C.; McIntosh, K. A.; Prankerd, R. J.; Charman, W. N. J. Pharm. Sci. 2006, 95, 256. (2₃) Lauterwasser, E. M. W.; Fontaine, S. D.; Li, H.; Gut, J.; Katneni, K.; Charman, S. A.; Rosenthal, P. J.; Bogyo, M.; Renslo, A. R. ACS Med Chem Lett 2015, 6, 1145. (24) Sijwali, P. S.; Rosenthal, P. J. Proc Natl Acad Sci USA 2004, 101, 4384. (25) Fontaine, S. D.; Spangler, B.; Gut, J.; Lauterwasser, E. M.; Rosenthal, P. J.; Renslo, A. R. ChemMedChem 2015, 10, 47.

What is claimed is:

1. A compound, or a pharmaceutically acceptable salt thereof, having the formula: 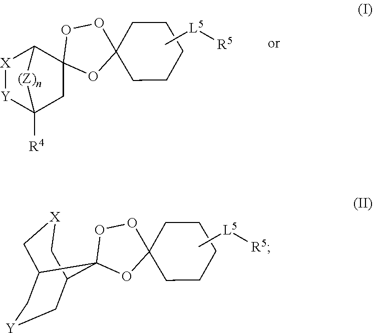
wherein
X is NR^{1.1} or C(R^{1.1}—R^{1.2});
Y is NR^{2.1} or C(R^{2.1}R^{2.2});
Z is C(R^{3.1}R^{3.2});
n is 1 or 2;
L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker;
L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1.1} and R^{1.2} are independently hydrogen, oxo, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3c}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, oxo, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a protein moiety, a detectable moiety, a siderophore moiety, or a drug moiety;
each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, X⁴, and X⁵ are independently —F, —Cl, —Br, or —I;
n1, n2, n3, n4, and n5 are independently an integer from 0 to 4; and
m1, m2, m3, m4, m5, v1, v2, v3, v4, and v5 are independently 1 or 2.

2. A compound, or a pharmaceutically acceptable salt thereof, having the formula: 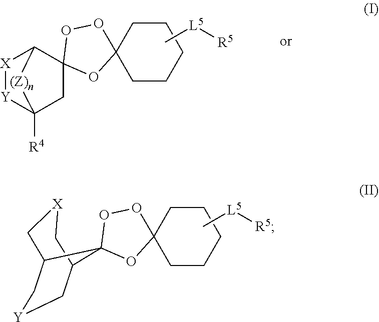
wherein
X is NR^{1.1} or C(R^{1.1}R^{1.2});
Y is NR^{2.1} or C(R^{2.1}R^{2.2});
Z is C(R^{3.1}R^{3.2});
n is 1 or 2;
L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, —N(R¹⁷)S(O)₂-L¹³-L¹⁴-, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker;
L¹³ and L¹⁴ are independently a bond, —N(R¹⁷)—, —N(R¹⁷)C(O)O—, —O—, —S—, —OC(O)—, —OC(O)N(R¹⁷)—, —OC(O)O—, —OSO₂—, —C(O)N(R¹⁷)—, —N(R¹⁷)C(O)—, —S(O)₂N(R¹⁷)—, —N(R¹⁷)S(O)₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{1.1} and R^{1.2} are independently hydrogen, oxo, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)^{m1}, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2.1} and R^{2.2} are independently hydrogen, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.1} and R^{3.2} are independently hydrogen, oxo, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SO^{v4}NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, oxo, halogen, —CX⁵₃, —CHX⁵₂, —CH₂X⁵, —OCX⁵₃, —OCH₂X⁵, —OCHX⁵₂, —CN, —SOₙ₅R^{5D}, —SO^{v5}NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a protein moiety, a detectable moiety, or a drug moiety;
each R¹⁷ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
X¹, X², X³, X⁴, and X⁵ are independently —F, —Cl, —Br, or —I;
n1, n2, n3, n4, and n5 are independently an integer from 0 to 4; and
m1, m2, m3, m4, m5, v1, v2, v3, v4, and v5 are independently 1 or 2;
wherein the compound is not

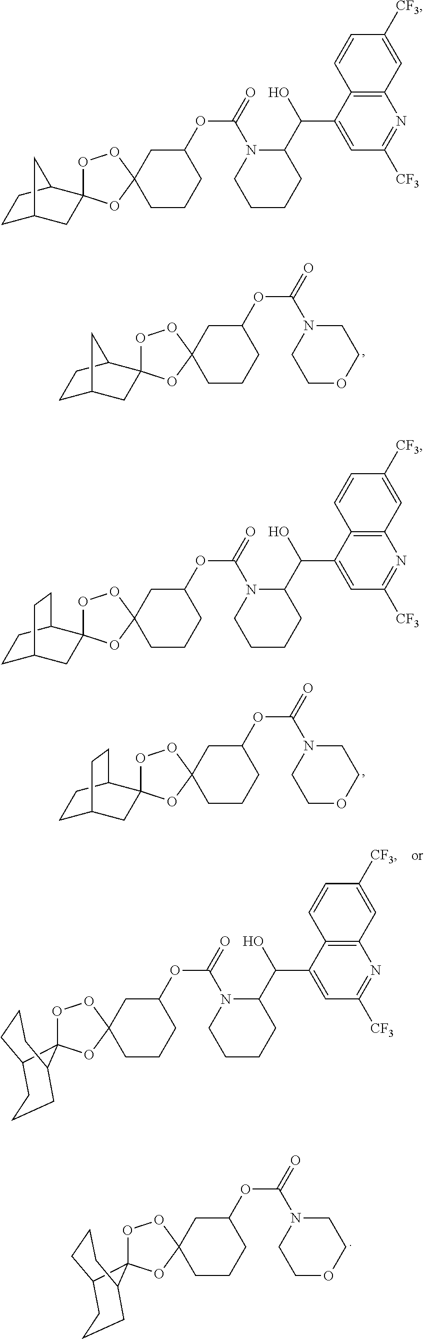 [SMILES: O=C(OC1CCCC2(C1)OOC1(CC3CCC1C3)O2)N1CCCCC1C(O)c1cc(C(F)(F)F)nc2cc(C(F)(F)F)ccc12 ; O=C(OC1CCCC2(C1)OOC1(CC3CCC1C3)O2)N1CCOCC1 ; O=C(OC1CCCC2(C1)OOC1(CC3CCC1CC3)O2)N1CCCCC1C(O)c1cc(C(F)(F)F)nc2cc(C(F)(F)F)ccc12 ; O=C(OC1CCCC2(C1)OOC1(CC3CCC1CC3)O2)N1CCOCC1 ; O=C(OC1CCCC2(C1)OOC1(O2)C2CCCC1CCC2)N1CCCCC1C(O)c1cc(C(F)(F)F)nc2cc(C(F)(F)F)ccc12 ; O=C(OC1CCCC2(C1)OOC1(O2)C2CCCC1CCC2)N1CCOCC1]

3. The compound of claim 2, wherein at least one of R^{1.1}, R^{1.2}, R^{2.1}, R^{2.2}, R^{3.1}, R^{3.2}, or R⁴ is not hydrogen.

4. The compound of claim 2, wherein X is NR^{1.1} and/or Y is NR^{2.1}.

5. The compound of claim 2, wherein X is not CH₂ and Y is not CH₂.

6. The compound of claim 2, wherein L⁵ is not —OC(O)—.

7. The compound of claim 2, wherein R⁵ is not

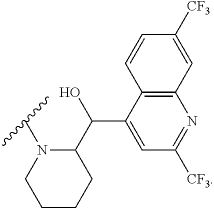 [SMILES: *N1CCCCC1C(O)c1cc(C(F)(F)F)nc2cc(C(F)(F)F)ccc12]

8. The compound of claim 2, wherein R⁵ is not morpholinyl.

9. The compound of claim 2, wherein R⁵ is not

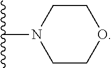 [SMILES: *N1CCOCC1]

10. The compound of claim 1, having the formula:

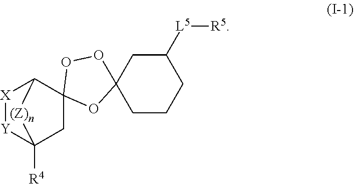

11. The compound of claim 1, having the formula:

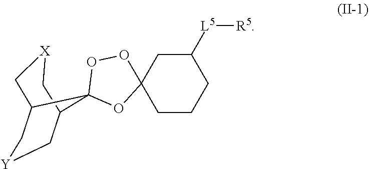

12. The compound of claim 1, wherein when X is NR^{1.1}, then Y is C(R^{2.1}R^{2.2}); and when Y is NR^{2.1}, then X is C(R^{1.1}R^{1.2}).

13. The compound of claim 1, wherein X is NH.

14. The compound of claim 1, wherein X is CHR^{1.2}.

15. The compound of claim 1, wherein Y is NH.

16. The compound of claim 1, wherein Y is CH₂.

17. The compound of claim 1, wherein Z is CH₂.

18. The compound of claim 1, wherein R^{1.1} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

19. The compound of claim 1, wherein R^{1.1} is hydrogen.

20. The compound of claim 1, wherein R^{1.2} is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

21. The compound of claim 1, wherein R^{1.2} is —C(O)OR^{1C}, —C(O)NR^{1A}R^{1B} or substituted 2 to 6 membered heteroalkyl.

22. The compound of claim 21, wherein R^{1C} is hydrogen or unsubstituted C₁-C₄ alkyl.

23. The compound of claim 1, wherein R^{1.2} is —C(O)OH, —C(O)NH(OH),

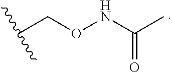 [SMILES: *CONC(C)=O]

24. The compound of claim 1, wherein R^{2.1} and R^{2.2} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

25. The compound of claim 1, wherein R^{2.1} and R^{2.2} are hydrogen.

26. The compound of claim 1, wherein R^{1.1} and R^{3.2} are independently hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

27. The compound of claim 1, wherein R^{3.1} and R^{3.2} are hydrogen.

28. The compound of claim 1, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

29. The compound of claim 1, wherein R⁴ is hydrogen.

30. The compound of claim 1, having the formula:

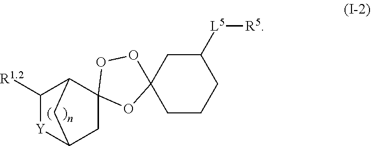

31. The compound of claim 1, having the formula:

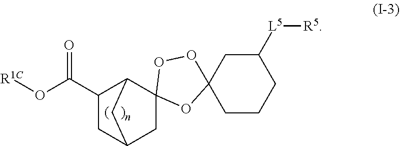

32. The compound of claim 1, having the formula:

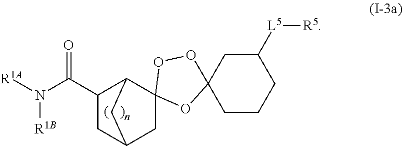

33. The compound of claim 1, having the formula:

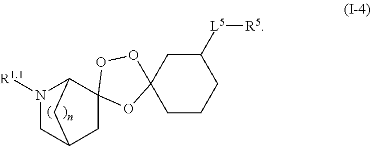

34. The compound of claim 1, having the formula:

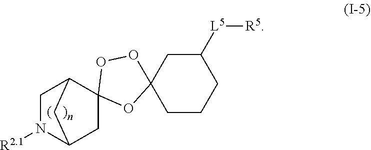

35. The compound of claim 1, having the formula:

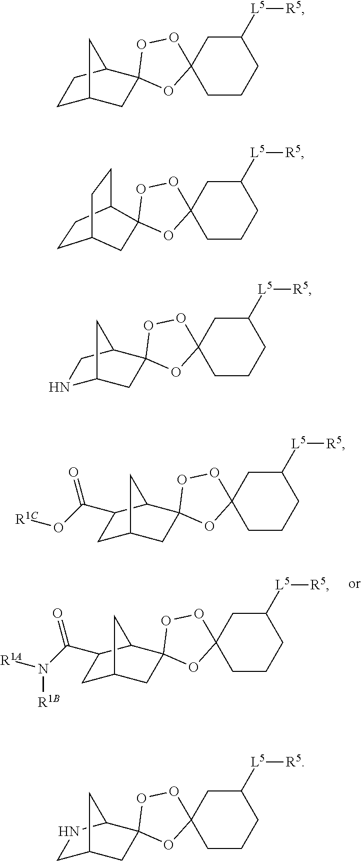

36. The compound of claim 1, having the formula:

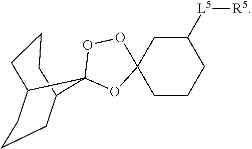

37. The compound of claim 1, having the formula:

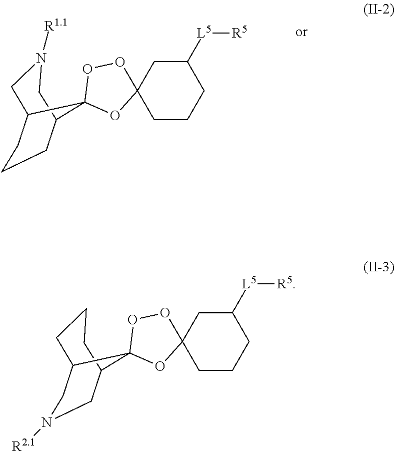

38. The compound of claim 1, having the formula:

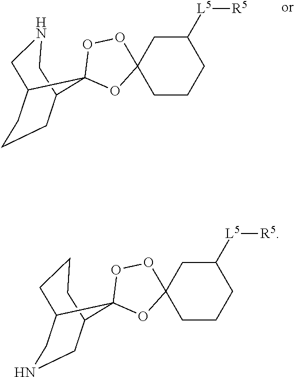

39. The compound of claim 1, wherein L⁵ is a bond, —N(R¹⁷)-L¹³-L¹⁴-, —N(R¹⁷)C(O)O-L¹³-L¹⁴-, —O-L¹³-L¹⁴-, —S-L¹³-L¹⁴-, —OC(O)-L¹³-L¹⁴-, —OC(O)N(R¹⁷)-L¹³-L¹⁴-, —OC(O)O-L¹³-L¹⁴-, —SO₂-L¹³-L¹⁴-, —OSO₂-L¹³-L¹⁴-, —C(O)N(R¹⁷)-L¹³-L¹⁴-, —N(R¹⁷)C(O)-L¹³-L¹⁴-, —S(O)₂N(R¹⁷)-L¹³-L¹⁴-, or —N(R¹⁷)S(O)₂-L¹³-L¹⁴-; and
R⁵ is a protein moiety, drug moiety, or a detectable moiety.

40. The compound of claim 1, wherein L⁵ is a bond, —N(R¹⁷)-L¹³-L¹⁴-, —O-L¹³-L¹⁴-, —OC(O)-L¹³-L¹⁴-, or —OC(O)N(R¹⁷)-L¹³-L¹⁴-.

41. The compound of claim 1, wherein L¹³ is a bond or substituted or unsubstituted arylene.

42. The compound of claim 1, wherein L¹³ is a bond or substituted or unsubstituted phenylene.

43. The compound of claim 1, wherein L¹⁴ is a bond, substituted or unsubstituted alkylene, or substituted or unsubstituted heteroalkylene.

44. The compound of claim 1, wherein L¹⁴ is a bond, —(CH₂)_{w}—, or —(CH₂)_{w}—OC(O)—; and w is an integer from 1 to 4.

45. The compound of claim 44, wherein w is 1.

46. The compound of claim 1, wherein -L¹³-L¹⁴- is a bond, -Ph-(CH₂)_{w}—, or -Ph-(CH₂)_{w}—OC(O)—; and w is an integer from 1 to 4.

47. The compound of claim 1, wherein -L¹³-L¹⁴- is a bond.

48. The compound of claim 1, wherein -L¹³-L¹⁴- is -Ph-(CH₂)_{w}—; and w is an integer from 1 to 4.

49. The compound of claim 1, wherein -L¹³-L¹⁴- is -Ph-(CH₂)_{w}—OC(O)—; and w is an integer from 1 to 4.

50. The compound of claim 1, wherein L⁵ is a bond, —N(R¹⁷)—, —O—, —OC(O)—, or —OC(O)N(R¹⁷)—.

51. The compound of claim 1, wherein R⁵ is a drug moiety.

52. The compound of claim 51, wherein the drug moiety is a monovalent form of an anti-cancer agent.

53. The compound of claim 51, wherein the drug moiety is a monovalent form of an anti-infective agent.

54. The compound of claim 53, wherein the anti-infective agent is an anti-parasitic agent.

55. The compound of claim 53, wherein the anti-infective agent is an anti-malarial drug.

56. The compound of claim 53, wherein the anti-infective agent is an anti-bacterial drug.

57. The compound of claim 1, wherein R⁵ is a detectable moiety.

58. The compound of claim 57, wherein the detectable moiety is a monovalent form of a fluorophore.

59. The compound of claim 1, wherein R⁵ is a protein moiety.

60. The compound of claim 59, wherein the protein moiety is a monovalent form of an antibody.

61. A pharmaceutical composition comprising a compound of one of claims 1 to 60, or a pharmaceutically acceptable salt thereof, and a pharmaceutically acceptable excipient.

62. A method of treating a disease in a subject in need thereof, said method comprising administering to the subject in need thereof a therapeutically effective amount of a compound of one of claims 1 to 60, or a pharmaceutically acceptable salt thereof.

63. The method of claim 62, wherein the disease is associated with a cell or organism having an increased Fe^{II} level compared to a standard control.

64. The method of claim 62, wherein the disease is cancer.

65. The method of claim 64, wherein the cancer is a hematological cancer.

66. The method of claim 64, wherein the cancer is a non-hematological cancer.

67. The method of claim 64, wherein the cancer is a pancreatic cancer, colon cancer, gastrointestinal cancer, lung cancer, or brain cancer.

68. The method of claim 62, wherein the disease is a parasitic disease.

69. The method of claim 68, wherein the parasitic disease is malaria.

70. The method of claim 68, wherein the parasitic disease is schistosomiasis.

71. The method of claim 68, wherein the parasitic disease is caused by blood-feasting parasites.

72. The method of claim 62, wherein the disease is a bacterial disease.

73. The method of claim 72, wherein the bacterial disease is an Enterococcus spp. bacterial disease, a Staphylococcus spp. bacterial disease, a Klebsiella spp. bacterial disease, an Acinetobacter spp. bacterial disease, a Pseudomonas spp. bacterial disease, or an Enterobacter spp. bacterial disease.

74. The method of claim 73, wherein the bacterial disease is an Enterococcus faecium bacterial disease.

75. The method of claim 73, wherein the bacterial disease is a Staphylococcus aureus bacterial disease.

76. The method of claim 73, wherein the bacterial disease is a Klebsiella pneumoniae bacterial disease.

77. The method of claim 73, wherein the bacterial disease is an Acinetobacter baumannii bacterial disease.

78. The method of claim 73, wherein the bacterial disease is a Pseudomonas aeruginosa bacterial disease.

79. A method of identifying a subject having a disease associated with a cell or organism having an increased Fe^{II} level compared to a standard control, said method comprising administering to the subject an effective amount of a compound of one of claims 1 to 60, or a pharmaceutically acceptable salt thereof.

80. A method of identifying a subject having a disease associated with an increased reductant level compared to a standard control, said method comprising:
(i) obtaining a biological sample from said subject;
(ii) contacting said biological sample with an effective amount of a compound of one of claims 1 to 60, or a pharmaceutically acceptable salt thereof, wherein said compound comprises a detectable moiety; and
(iii) detecting an increased level of said detectable moiety or a detectable agent resulting from cleavage of said detectable moiety relative to the level of said detectable moiety or detectable agent in the standard control.
